(19) United States
(12) Patent Application Publication
(10) Pub. No.: US 2025/0018037 A1
(43) Pub. Date: Jan. 16, 2025

(54) SAFETY CONTROL OF SWITCHABLE CHIMERIC ANTIGEN RECEPTOR T CELLS USING DOSE-ADJUSTABLE ADAPTORS

(71) Applicants: SEOUL NATIONAL UNIVERSITY R&DB FOUNDATION, Seoul (KR); TICAROS CO., LTD., Seoul (KR)

(72) Inventors: Kyungho CHOI, Seoul (KR); Junho CHUNG, Seoul (KR); Hyung Bae PARK, Seoul (KR); Ji Hwan KIM, Seoul (KR); Seoho LEE, Seoul (KR); Ki Hyun KIM, Seoul (KR); Sang Il KIM, Redwood City, CA (US); Sunyoung PARK, Seoul (KR); Ga Ram JEONG, Seoul (KR); Kangseung LEE, Gyeonggi-do (KR); Hyeonji LIM, Seoul (KR)

(21) Appl. No.: 18/669,176

(22) Filed: May 20, 2024

Related U.S. Application Data
(60) Provisional application No. 63/503,413, filed on May 19, 2023.

(51) Int. Cl. A61K 39/00; A61P 35/00; C07K 14/725; C07K 16/28; C07K 16/44

(52) U.S. Cl. CPC A61K 39/464417; A61K 39/4611; A61K 39/4613; A61K 39/4631; A61P 35/00; C07K 14/7051; C07K 16/2878; C07K 16/44; A61K 2039/62; C07K 2317/24; C07K 2317/92

(57) ABSTRACT

Chimeric antigen receptor-transduced T cells (CAR-T cells) show a remarkable efficacy for some hematological malignancies. However, CAR targets are restricted to a few antigens primarily due to on-target off-tumor toxicities of CAR-T cells. Although several strategies were proposed to avoid on-target off-tumor toxicities, most of them use complicated designs including dual gene expression for specificity. In this study, we show that switchable CAR immune cells (e.g., CAR-T cells) with a tumor-targeting adaptor can mitigate on-target off-tumor toxicity against the tumor antigen that cannot be targeted with conventional CAR immune cells due to this toxicity, such as CD40 and CS1. Therefore, a switchable CAR system is a valuable tool to control CAR-T cell toxicity while maintaining therapeutic efficacy, which enables CAR anti-tumor target expansion.

### CROSS-REFERENCE TO RELATED APPLICATIONS

This application claims priority to and the benefit of U.S. Provisional Patent Application Ser. No. 63/503,413, filed May 19, 2023, the contents of which is incorporated herein by this reference as if fully set forth herein.

### REFERENCE TO A “SEQUENCE LISTING” SUBMITTED AS XML FILE VIA PATENT CENTER

The instant application contains a Sequence Listing which has been submitted electronically via Patent Center and is hereby incorporated by reference in its entirety. Said .xml copy, created on Sep. 4, 2024, is named txt_110362-1446803-100USNP.txt and is 94,345 bytes in size.

### BACKGROUND

Chimeric antigen receptor-transduced T cells (CAR-T cells) are the antitumor therapeutic T cells that carry an artificial receptor, CAR, in which an extracellular tumor-targeting antibody moiety is linked to the intracellular signaling domains¹. Many targets, for example, CD40 or CS1, are attractive anti-cancer therapeutic targets that have been utilized for generation of therapeutic antibodies. Unfortunately, these antigens are also expressed in various hematopoietic and non-hematopoietic normal tissues, such as endothelial cells and parenchymal cells, and conventional CAR-T cells targeting these antigens (i.e., CD40 and CS1) may cause lethal on-target off-tumor toxicity. For example, CD40 is known to be expressed in various tumors, such as lymphoma, multiple myeloma, and acute myelocytic leukemia²⁰. CD40 is also expressed in various immune cells, such as monocytes, macrophages, and dendritic cells, acting as a stimulatory receptor for those cells²¹. Hence, antagonistic or agonistic anti-CD40 antibodies have been tried as anti-tumor immunotherapy modalities^{22, 23}. However, CD40 is also expressed in various non-hematopoietic normal tissues, such as endothelial cells and parenchymal cells, raising concerns on normal tissue toxicity of CD40-targeting strategies^{24, 25, 26}. CS-1 is known to be a tumor antigen of multiple myeloma⁵⁶. Therefore, anti-CS-1 antibody (elotuzumab) has been approved by FDA for treatment of multiple myeloma. However, CS-1 is also expressed in normal cells such as NK cells, B cells and activated T cells. Thus, anti-CS1 CAR T cells may have potential toxicities to normal cells⁵⁷.

A switchable CAR-T cell system is a strategy to reduce CAR-T cell toxicity against targets such as CD40¹³ and CS1. Here, CAR-T cells are against small epitope tags, such as chemicals and peptides, instead of the anti-tumor antigen. These anti-tag CAR-T cells are activated only when the separate anti-tumor antibody moiety coupled with this tag (also referred to here as an “adaptor”) is present. With these systems, the degree of CAR-T cell activation can be controlled by adjusting doses of the adaptors^{16, 17, 18}. However, the usage of such systems for avoiding on-target off-tumor toxicity is difficult to test because most in vivo CAR-T cell experiments are done with human CAR-T cells in xenogeneic mouse models, in which the CAR cannot recognize target antigens in normal mouse tissues. Additionally, switchable CAR-T systems against chemical tags (i.e., small molecules such as cotinine) present challenges in that a chemical conjugation is required, which can remain incomplete due to partial labeling and inconsistent due to varied labeling efficiency. Also, chemical tags may have unexpected biological activity that may interfere with normal physiology. There is a need to address the aforementioned deficiencies accordingly.

### BRIEF SUMMARY

At least described herein are switchable chimeric antigen receptor immune cell systems, methods of treating cancer, chimeric antigen receptor immune cells, and switchable chimeric antigen receptor immune cell pharmaceutical compositions.

In some embodiments of the present disclosure, a switchable chimeric antigen receptor immune cell system comprises a chimeric antigen receptor immune cell and an anti-tumor antibody conjugated to a peptide tag. In some embodiments, the chimeric antigen receptor immune cell can comprise a chimeric antigen receptor (CAR), wherein the CAR comprises an antigen recognition domain that recognizes the peptide tag. In some embodiments, the anti-tumor antibody can be an anti-CD40 or anti-CS1 antibody. In some embodiments, the anti-tumor antibody comprises a means for binding CD40 or a means for binding CS1.

In some embodiments of switchable chimeric antigen receptor immune cell systems described herein, the switchable CAR immune cell can be a T cell, a B cell, a natural killer (NK) cell, NKT cell, or a macrophage. In some embodiments, the CAR can further comprise a transmembrane domain and a signal transduction domain. In some embodiments, the antigen recognition domain can comprise an antibody that recognizes the peptide tag. In some embodiments, the peptide tag can be a His tag or a Myc tag. In some embodiments, the antigen recognition domain comprises a means for binding the peptide tag (for example, a His tag or a Myc tag). In some embodiments, the antibody that recognizes the peptide tag can be a scFv, Fab, Fab′, Fv, or single domain antibody variable region.

In some embodiments, the antigen recognition domain can recognize a His tag, and the antigen recognition domain can comprise a heavy chain variable region comprising HCDRs 1-3 and a light chain variable region comprises LCDRs 1-3. In some embodiments, HCDRs 1-3 can comprise (or comprise at least one of) sequences of SEQ ID NOs: 31-33, respectively, and the LCDRs 1-3 can comprise (or comprise at least one of) SEQ ID NOs 34-36, respectively; and/or or the HCDRs 1-3 can comprise can comprise (or comprise at least one of) SEQ ID NOs: 40-42, respectively, and the LCDRs 1-3 can comprise (or comprise at least one of) sequences of SEQ ID NOs 43-45, respectively.

In some embodiments of switchable chimeric antigen receptor immune cell systems described herein, the antigen recognition domain can recognize a Myc tag, and the antigen recognition domain can comprise a heavy chain variable region comprising HCDRs 1-3 and a light chain variable region comprising LCDRs 1-3; or the HCDRs 1-3 can comprise (or comprise at least one of) sequences of SEQ ID NOs: 49-51, respectively and the LCDRs 1-3 can comprise (or comprise at least one of) sequences of SEQ ID NOs: 52-54, respectively; and/or the HCDRs 1-3 can comprise (or comprise at least one of) sequences of SEQ ID NOs: 58-60, respectively and the LCDRs 1-3 can comprise (or comprise at least one of) sequences of SEQ ID NOs: 61-63, respectively; and/or the HCDRs 1-can comprise (or comprise at least one of) sequences of SEQ ID NOs: 67-69, respectively and the LCDRs 1-3 can comprise (or comprise at least one of) sequences of SEQ ID NOs: 70-72, respectively.

In some embodiments of switchable chimeric antigen receptor immune cell systems described herein, the peptide tag can comprise a Histidine multimer. In some embodiments, the peptide tag can comprise 2-25 histidines. In some embodiments, the peptide tag can comprise a Myc tag. In some embodiments, the Myc tag can comprise SEQ ID NO: 146.

In some embodiments of switchable chimeric antigen receptor immune cell systems described herein, the anti-CD40 antibody can comprise a heavy chain variable region comprising HCDRs 1-3 and a light chain variable region comprising LCDRs 1-3, wherein the HCDRs 1-3 can comprise (or comprise at least one of) sequences of SEQ ID NOs 4-6, respectively, and wherein the LCDRs 1-3 can comprise (or comprise at least one of) SEQ ID NOs: 7-9, respectively; or the HCDRs 1-3 can comprise (or comprise at least one of) sequences of SEQ ID NOs 13-15, respectively, and the LCDRs 1-3 can comprise (or comprise at least one of) one of SEQ ID NOs: 16-18, respectively; and/or the HCDRs 1-3 can comprise (or comprise at least one of) sequences of SEQ ID NOs 22-24, respectively, and the LCDRs 1-3 can comprise (or comprise at least one of) SEQ ID NOs: 25-27, respectively.

In some embodiments of switchable chimeric antigen receptor immune cell systems described herein, the anti-CS1 antibody can comprise a heavy chain variable region comprising HCDRs 1-3 and a light chain variable region comprising LCDRs 1-3, and the HCDRs 1-3 can comprise (or comprise at least one of) sequences of SEQ ID NOs 85-87, respectively, and the LCDRs 1-3 can comprise (or comprise at least one of) one of SEQ ID NOs:88-90, respectively; and/or the HCDRs 1-3 can comprise (or comprise at least one of) sequences of SEQ ID NOs 94-96, respectively, and the LCDRs 1-3 can comprise (or comprise at least one of) sequences SEQ ID NOs: 97-99, respectively; and/or the HCDRs 1-3 can comprise (or comprise at least one of) sequences of SEQ ID NOs 103-105, respectively, and the LCDRs 1-3 can comprise (or comprise at least one of) sequences SEQ ID NOs: 106-108, respectively.

In some embodiments, of switchable chimeric antigen receptor immune cell systems described herein, the peptide tag can consist essentially of a Histidine multimer or a Myc tag. In some embodiments, the peptide tag can be a His tag and the antibody that recognizes the peptide tag can be an anti-His antibody. In some embodiments, the peptide tag is a His tag and the antibody that recognizes the peptide tag is an anti-His antibody. In some embodiments, the peptide tag can be a Myc tag and the antibody that recognizes the peptide tag can be an anti-Myc antibody. In some embodiments, the peptide tag is a Myc tag and the antibody that recognizes the peptide tag is an anti-Myc antibody.

In some embodiments, described herein is a method of treating cancer, comprising administering, to a subject having or suspected of having a cancer, a plurality of chimeric antigen receptor immune (CAR) immune cells, wherein each of the plurality comprises a chimeric antigen receptor (CAR), and wherein the CAR comprises an antigen recognition domain that recognizes a peptide tag; and administering an anti-tumor antibody conjugated to the peptide tag to the subject.

In some embodiments of methods as described herein, the anti-tumor antibody can be an anti-CD40 antibody or an anti-CS1 antibody. In some embodiments, each of the plurality of switchable CAR immune cell can be a T cell, a B cell, a natural killer (NK) cell, NKT cell, or a macrophage. In some embodiments, the CAR further can further comprise a transmembrane domain and a signal transduction domain. In some embodiments, the peptide tag can be a His tag or a Myc tag. In some embodiments, the antigen recognition domain that recognizes the peptide tag can be a scFv, Fab, Fab′, Fv, or single domain antibody variable region. In some embodiments, the antigen recognition domain that recognizes a His tag can comprise: a heavy chain variable region comprising HCDRs 1-3 with sequences of (or at least one of) SEQ ID NOs: 31-33, respectively, and a light chain variable region comprising LCDRs 1-3 comprising sequences of (or at least one of) SEQ ID NOs 34-36, respectively; and/or a heavy chain variable region comprising HCDRs 1-3 with sequences of (or at least one of) SEQ ID NOs: 40-42, respectively, and a light chain variable region comprising LCDRs 1-3 with sequences of (or at least one of) SEQ ID NOs 43-45, respectively.

In some embodiments of methods described herein, the antigen recognition domain that recognizes the Myc tag can comprise a heavy chain variable region comprising HCDRs 1-3 and a light chain variable region comprising LCDRs 1-3, and the HCDRs 1-3 can comprise (or comprise at least one of) sequences of SEQ ID NOs: 49-51, respectively and the LCDRs 1-3 can comprise (or comprise at least one of) sequences of SEQ ID NOs: 52-54, respectively; and/or the HCDRs 1-3 can comprise (or comprise at least one of) sequences of SEQ ID NOs: 58-60, respectively and the LCDRs 1-3 can comprise (or comprise at least one of) sequences of SEQ ID NOs: 61-63, respectively; and/or wherein the HCDRs 1-3 can comprise (or comprise at least one of) sequences of SEQ ID NOs: 67-69, respectively and the LCDRs 1-3 can comprise (or comprise at least one of) sequences of SEQ ID NOs: 70-72, respectively.

In some embodiments of methods described herein, the peptide tag can comprise a Histidine multimer. In some embodiments, the peptide tag can be a Histidine multimer. In some embodiments, the peptide tag can comprise a Myc tag. In some embodiments, the peptide tag is a Myc tag. In some embodiments, the antibody can be an anti-CD40 antibody or anti-CS1 antibody.

In some embodiments, cancer is a hematological malignancy. In some embodiments, the CAR immune cells and anti-tumor antibody can be administered in an effective amount to reduce one or more symptoms of the cancer. In some embodiments of the methods disclosed herein, the anti-CD40 antibody comprises a heavy chain variable region comprising HCDRs 1-3 and a light chain variable region comprising LCDRs 1-3, wherein the HCDRs 1-3 comprise sequences of SEQ ID NOs 4-6, respectively, wherein the LCDRs 1-3 comprise one of SEQ ID NOs: 7-9, respectively; or wherein the HCDRs 1-3 comprise sequences of SEQ ID NOs 13-15, respectively, wherein the LCDRs 1-3 comprise one of SEQ ID NOs: 16-18, respectively; or wherein the HCDRs 1-3 comprise sequences of SEQ ID NOs 22-24, respectively, wherein the LCDRs 1-3 comprise one of SEQ ID NOs: 25-27, respectively.

In some embodiments of the methods disclosed herein, the anti-CS1 antibody comprises a heavy chain variable region comprising HCDRs 1-3 and a light chain variable region comprising LCDRs 1-3, wherein the HCDRs 1-3 comprise sequences of SEQ ID NOs 85-87, respectively, wherein the LCDRs 1-3 comprise one of SEQ ID NOs:88-90, respectively; wherein the HCDRs 1-3 comprise sequences of SEQ ID NOs 94-96, respectively, wherein the LCDRs 1-3 comprise one of SEQ ID NOs: 97-99, respectively; wherein the HCDRs 1-3 comprise sequences of SEQ ID NOs 103-105, respectively, wherein the LCDRs 1-3 comprise one of SEQ ID NOs: 106-108, respectively.

In some embodiments, described herein is a chimeric antigen receptor immune cell comprising an antibody or antigen binding portion thereof that binds to a His tag or a Myc tag.

In some embodiments of chimeric antigen receptor immune cells described herein, the antibody or antigen binding portion thereof that binds to a His tag comprising a heavy chain variable region comprising a heavy chain CDR1 (HCDR1) of SEQ ID NO: 31, a heavy chain CDR2 (HCDR2) of SEQ ID NO: 32, and a heavy chain CDR3 (HCDR3) of SEQ ID NO: 33; and wherein the antibody or antigen binding portion thereof comprises a light chain variable region comprising a light chain CDR1 (LCDR1) of SEQ ID NO: 34, a light chain CDR2 (LCDR2) of SEQ ID NO: 35, and a light chain CDR3 (LCDR3) of SEQ ID NO: 36.

In some embodiments of chimeric antigen receptor immune cells described herein, the antibody or antigen binding portion thereof that binds to a His tag can comprise a heavy chain variable region comprising a heavy chain variable region comprising HCDRs 1-3 and a light chain variable region comprising LCDRs 1-3, wherein the HCDRs 1-3 can comprise (or comprise at least one of) SEQ ID NOs 31-33 respectively and the LCDRs 1-3 can comprise (or comprise at least one of) SEQ ID NOs 34-36, respectively; and/or wherein the HCDRs 1-3 can comprise (or comprise at least one of) SEQ ID NOs 40-42 respectively and the LCDRs 1-3 can comprise (or comprise at least one of) SEQ ID NOs 43-45, respectively.

In some embodiments of chimeric antigen receptor immune cells described herein, the antibody or antigen binding portion thereof that binds to a Myc tag can comprise a heavy chain variable region comprising HCDRs 1-3 and a light chain variable region comprising LCDRs 1-3, wherein the HCDRs 1-3 can comprise (or comprise at least one of) sequences of SEQ ID NOs: 49-51, respectively and the LCDRs 1-3 can comprise (or comprise at least one of) sequences of SEQ ID NOs: 52-54, respectively; and/or wherein the HCDRs 1-3 can comprise (or comprise at least one of) sequences of SEQ ID NOs: 58-60, respectively and the LCDRs 1-3 can comprise (or comprise at least one of) sequences of SEQ ID NOs: 61-63, respectively; and/or wherein the HCDRs 1-3 can comprise (or comprise at least one of) sequences of SEQ ID NOs: 67-69, respectively and the LCDRs 1-3 can comprise (or comprise at least one of) sequences of SEQ ID NOs: 70-72, respectively.

In some embodiments, described herein is a switchable chimeric antigen receptor immune cell system comprising a chimeric antigen receptor immune cell described above and herein and an anti-tumor antibody conjugated to the His tag or the Myc tag.

In some embodiments, described herein is a switchable chimeric antigen receptor immune cell pharmaceutical composition, comprising a plurality of chimeric antigen receptor immune (CAR) immune cells, wherein each of the plurality can comprise a chimeric antigen receptor (CAR), and wherein the CAR can comprise an antigen recognition domain that recognizes a peptide tag; and a pharmaceutically acceptable carrier.

In some embodiments of pharmaceutical compositions as described herein, pharmaceutical compositions can further comprise an anti-tumor antibody conjugated to the peptide tag. In some embodiments, the anti-tumor antibody can be an anti-CD40 antibody or an anti-CS1 antibody. In some embodiments, the switchable CAR immune cell can be a T cell, a B cell, a natural killer (NK) cell, NKT cell, or a macrophage. In some embodiments, the CAR can further comprise a transmembrane domain and a signal transduction domain. In some embodiments, the antigen recognition domain can recognize the peptide tag. In some embodiments, the peptide tag can be a His tag or a Myc tag. In some embodiments, the antigen recognition domain that recognizes the peptide tag can be s a scFv, Fab, Fab′, Fv, or single domain antibody variable region.

In some embodiments of pharmaceutical compositions as described herein, the antigen recognition domain that recognizes the His tag can comprise a heavy chain variable region comprising HCDRs 1-3 and a light chain variable region comprising LCDRs 1-3, wherein the HCDRs 1-3 can comprise (or comprise at least one of) sequences of SEQ ID NOs: 31-33, respectively, and wherein the LCDRs 1-3 can comprise (or comprise at least one of) sequences of SEQ ID NOs 34-36, respectively; and/or wherein the HCDRs 1-3 can comprise (or comprise at least one of) sequences of SEQ ID NOs: 40-42, respectively, and wherein the LCDRs 1-3 can comprise (or comprise at least one of) sequences of SEQ ID NOs 43-45, respectively.

In some embodiments of pharmaceutical compositions as described herein, the antigen recognition domain that recognizes the Myc tag can comprise a heavy chain variable region comprising HCDRs 1-3 and a light chain variable region comprising LCDRs 1-3, wherein the HCDRs 1-3 can comprise (or comprise at least one of) sequences of SEQ ID NOs: 49-51, respectively and the LCDRs 1-3 can comprise (or comprise at least one of) sequences of SEQ ID NOs: 52-54, respectively; and/or wherein the HCDRs 1-3 can comprise (or comprise at least one of) sequences of SEQ ID NOs: 58-60, respectively and the LCDRs 1-3 can comprise (or comprise at least one of) sequences of SEQ ID NOs: 61-63, respectively; and/or wherein the HCDRs 1-3 can comprise (or comprise at least one of) sequences of SEQ ID NOs: 67-69, respectively and the LCDRs 1-3 can comprise sequences of SEQ ID NOs: 70-72, respectively.

In some embodiments of pharmaceutical compositions as described herein, the peptide tag can comprise a Histidine multimer. In some embodiments, the peptide tag can comprise a Histidine multimer. In some embodiments, the peptide tag can comprise a Myc tag. In some embodiments, the anti-tumor antibody can be an anti-CD40 antibody or an anti-CS1 antibody. In some embodiments, the anti-CD40 antibody can comprise a heavy chain variable region comprising HCDRs 1-3 and a light chain variable region comprising LCDRs 1-3, wherein the HCDRs 1-3 can comprise (or comprise at least one of) sequences of SEQ ID NOs 4-6, respectively, and wherein the LCDRs 1-3 can comprise (or comprise at least one of) sequences SEQ ID NOs: 7-9, respectively; and/or wherein the HCDRs 1-3 can comprise (or comprise at least one of) sequences of SEQ ID NOs 13-15, respectively, and the LCDRs 1-3 can comprise (or comprise at least one of) sequences SEQ ID NOs: 16-18, respectively; and/or wherein the HCDRs 1-3 can comprise (or comprise at least one of) sequences of SEQ ID NOs 22-24, respectively, and wherein the LCDRs 1-3 can comprise (or comprise at least one of) sequences SEQ ID NOs: 25-27, respectively.

In some embodiments of pharmaceutical compositions as described herein, the anti-CS1 antibody can comprise a heavy chain variable region comprising HCDRs 1-3 and a light chain variable region comprising LCDRs 1-3, wherein the HCDRs 1-3 can comprise (or comprise at least one of) sequences of SEQ ID NOs 85-87, respectively, and wherein the LCDRs 1-can comprise (or comprise at least one of) one of SEQ ID NOs:88-90, respectively; and/or wherein the HCDRs 1-3 can comprise (or comprise at least one of) sequences of SEQ ID NOs 94-96, respectively, and wherein the LCDRs 1-3 can comprise (or comprise at least one of) sequences SEQ ID NOs: 97-99, respectively; and/or wherein the HCDRs 1-3 can comprise (or comprise at least one of) sequences of SEQ ID NOs 103-105, respectively, and wherein the LCDRs 1-3 can comprise (or comprise at least one of) sequences SEQ ID NOs: 106-108, respectively. In some embodiments, the peptide tag can be a His tag or a Myc tag. In some embodiments, the peptide tag can be a His tag or a Myc tag.

### BRIEF DESCRIPTION OF THE DRAWINGS

FIGS. 1A-1M Murine CD40 CAR-T cells show on-target off-tumor lethal toxicity in a syngeneic lymphoma model. FIG. 1A: Schematic diagram of the murine CD40 CAR construct. EC, extracellular; TM, transmembrane; Cyt, cytoplasmic domain. FIG. 1B: A representative flow cytometry plot of CD40 CAR expression on mouse T cells four days after transduction. FIG. 1C: Cytotoxicity of CD40 CAR-T cells against A20 cells. Control T or CD40 CAR-T cells (effector) were mixed with PKH26-labeled A20 cells (target) at the indicated ratios. After 6 h incubation, viable A20 cells were counted with cell-counting beads via flow cytometry and the percent cytotoxicity was calculated as described in Methods. FIG. 1D: Control T or CD40 CAR-T cells were co-cultured with A20 cells for 24 h. The amount of IFN-γ in culture supernatants was measured by ELISA. Results are representative of 3 independent experiments. FIGS. 1E-1G: Balb/C mice were i.v. injected with A20-Luc cells (1×10⁶) on day 0, irradiated (2.5 Gy) for lymphodepletion on day 6, and i.v. injected with control T or CD40 CAR-T cells (5×10⁶) on day 7. Body weight change (n=5) (FIG. 1E) and survival (n=10) (FIG. 1F) were measured. 3 days after T cells injection, mice were sacrificed, and serum levels of IL-6 were measured by ELISA (n=9) (FIG. 1G). Each dot indicates the value of individual mouse. FIGS. 1H-1J: Same experiments as FIGS. 1E-1G: were performed, including the groups not injected with A20-Luc cells. Body weight change (n=5) (FIG. 1H), survival (n=10) (FIG. 1I), and serum levels of IL-6 (n=7) (FIG. 1J) were measured. FIGS. 1K-1M: Wild type and CD40 knockout B6 mice were irradiated (3 Gy) on day 6 and i.v. injected with CD40 CAR-T cells (5×10⁶) on day 7. Body weight change (n=5) (FIG. 1K), survival (n=10) (FIG. 1L), and serum levels of IL-6 (n=9) (FIG. 1M) were measured. Measuring body weights were halted when the mice began to die (red asterisks in FIGS. 1E, 1H, 1K). Data in FIGS. 1E, 1H, 1K are representative of at least two independent experiments. Data in FIGS. 1F, 1G, 1H, 1J, 1L, and 1M are pooled from 2 replicate experiments. Statistical significances were determined with either Log-rank (Mantel-Cox) test (FIGS. 1F, 1H, 1L; ***p<0.001, **p<0.01, n.s: not significant) or by unpaired two-tailed t-test (FIGS. 1G, 1J, 1M).

FIGS. 2A-2H Both hematopoietic and non-hematopoietic expression of CD40 contribute to the on-target off-tumor toxicity. FIG. 2A: CD40 CAR-T cells were co-cultured with A20 cells or peritoneal macrophages (Mφ) for 24 h. The amount of IL-6 or IL-1β in culture supernatants was measured by ELISA. FIGS. 2B-2F: Balb/C mice were irradiated (2.5 Gy) for lymphodepletion on day −1 and injected with 5×10⁶ (FIG. 2B, 2D, 2F) or 1×10⁶ (FIG. 2C, 2E) Control T or CD40 CAR-T cells on day 0. For IL-6 or IL-10 neutralization (FIG. 2B, FIG. 2C), anti-IL-6 or anakinra were injected daily intraperitoneally 5 times from the day of CAR T injection. For depleting phagocytes (FIGS. 2D-2F), clodronate liposome was injected intraperitoneally for three consecutive days before CAR-T cell infusion. Body weight change (FIGS. 2B-2E) and IL-6 serum levels (FIG. 2F) were measured. Each dot in FIG. 2F: indicates the value of individual mouse. FIGS. 2G, 2H: BM chimeras were setup using B6 WT and B6 CD40-knockout mice as BM donors or recipients (e.g., B6.CD40 KO>>B6 denotes that donor BMs from CD40-knockout mice were transferred to B6 wild type recipients.). After eight weeks, the chimeric mice were irradiated (2.5 Gy) on day −1 and injected with CAR-T cells (5×10⁶) on day 0. Body weight change (n=5) (FIG. 2G) and survival (n=10) (FIG. 2H) were measured. Measuring body weights was halted when the mice began to die (red asterisks in FIGS. 2B, 2D, 2G). FIG. 2I: Balb/C mice were irradiated (2.5 Gy) on day −1 and injected with GFP-effluc T (Control-Luc-T) or CD40 CAR-effluc T cells (CD40 CAR-Luc-T) (5×10⁶ or 1×10⁶) on day 0. In vivo distribution of injected T cells was visualized by bioluminescence imaging. Data in FIG. 2F and FIG. 2H are pooled from 2 replicate experiments. All other data are representative of at least two independent experiments. Statistical significances were determined either by an unpaired two-tailed t-test (FIG. 2F) or by Log-rank (Mantel-Cox) test (FIG. 2H; ***p<0.001, n.s: not significant).

FIGS. 3A-3I Chemical or peptide-labeled anti-CD40 scFv can be used as a probe for switchable CAR-T cells in vitro. FIG. 3A: Scheme of conventional CAR-T and switchable CAR-T cells. FIG. 3B: Schematic diagram of the murine Cot-CAR construct and a representative flow cytometry plot of Cot-CAR expression on mouse T cells at 4 days after transduction. FIG. 3C: Chemical structure of carboxy-cotinine (Top left). Schematic diagram of cotinine-labeled anti-mouse CD40 scFv-Cκ (C1C02-Cot) (Top right). A representative flow cytometry plot of binding of C1C02-Cot to A20 cells (Bottom). FIG. 3D: PKH26-labeled A20 cells (target) were pre-incubated with C1C02-Cot and then co-cultured with Cot CAR-T cells (effector) at the indicated ratios for 6 h. Percent cytotoxicity was calculated from flow cytometry-based viable cell counting. FIG. 3E: A20 cells were pre-incubated with C1C02-Cot and then co-cultured with Cot CAR-T cells for 24 h. The amount of IFN-γ in culture supernatants was measured by ELISA. FIG. 3F: Schematic diagram of the murine His 28z-CAR construct (Top) and a representative flow cytometry plot of His-28z CAR expression on mouse T cells at 4 days after transduction (Bottom). FIG. 3G: Schematic diagram of anti-mouse CD40 scFv-Cκ-6×His (C1C02-His) (Top). A representative flow cytometry plot of binding of C1C02-His to EL4 cell line overexpressing mouse CD40 (EL4-mCD40) (Bottom). FIG. 3H: PKH26-labeled EL4-mCD40 cells (target) were pre-incubated with C1C02-His and then co-cultured with His-28z CAR-T cells (effector) at the indicated ratios for six h. Percent cytotoxicity was calculated from flow cytometry-based viable cell counting. FIG. 3I: EL4-mCD40 cells were pre-incubated with C1C02-His and then co-cultured with His-28z CAR-T cells for 24 h. The amount of IFN-γ in culture supernatants was measured by ELISA. FIG. 3J: Schematic diagram of the murine His-BBz CAR construct and a representative flow cytometry plot of His-BBz CAR expression on mouse T cells at 4 days after transduction. FIG. 3K: PKH26-labeled EL4-mCD40 cells (target) were pre-incubated with C1C02-His and then co-cultured with His-BBz CAR-T cells (effector) at the indicated ratios for 6 h. Percent cytotoxicity was calculated from flow cytometry-based viable cell counting. FIG. 3L: EL4-mCD40 cells were pre-incubated with C1C02-His and then co-cultured with His-BBz CAR-T cells for 24 h. The amount of IFN-γ in culture supernatants was measured by ELISA.

FIGS. 4A-4D Anti-CD40 scFv can be used as a dose-adjustable probe for switchable CAR-T cells in vitro. FIG. 4A: CD40 expression levels in A20 cell and F4/80(+) peritoneal macrophages (Mφ) determined by a commercial anti-mouse CD40 antibody staining. FIGS. 4B, 4C: Comparison of dose-dependent cell-binding affinity of C1C02-Cot between A20 and macrophage. Mean fluorescence intensities (MFIs) of the binding are shown as values inside the plot (FIG. 4B) and also as a graph (FIG. 4C). FIG. 4D: For toxicity readouts, macrophages were pre-incubated with various concentrations of C1C02-Cot, and then co-cultured with Cot CAR-T or CD40 CAR-T cells for 24 h. The amounts of IL-6 in the culture supernatant were measured by ELISA (left axis and blue lines). For efficacy readouts, PKH26-labeled A20 cells (target) were pre-incubated with various concentrations of C1C02-Cot, and then co-cultured with Cot CAR-T cells (effector) at E:T ratio 5:1 for 6 h. CD40 CAR-T cells were co-cultured with target cells untreated with C1C02-Cot. Percent cytotoxicity was calculated from flow cytometry-based viable cell counting (right axis and red lines). Results are representative of at least two independent experiments.

FIGS. 5A-5F Anti-mouse CD40 switchable CAR-T cells eliminate lymphoma cells in vivo without overt toxicity. FIG. 5A: Experimental scheme for murine B cell lymphoma treatment using syngeneic Cot CAR-T cells. Balb/C mice were i.v. injected with A20-Luc cells (1×10⁶) on day 0, irradiated (2.5 Gy) for lymphodepletion on day six, and injected with CD40 CAR-T cell or Cot CAR-T cells (5×10⁶) on day 7. From the day of Cot CAR-T injection, C1C02-Cot (20 μg/head) was injected intravenously every other day for a total of 8 times. FIG. 5B, FIG. 5C: Body weight change (n=5) and survival (n=5) were measured. Measuring body weights were halted when the mice began to die (red asterisks). FIG. 5D: Serum levels of IL-6 were measured at three days after CAR-T injection. Each dot indicates the value of individual mouse. (p-value, unpaired two-tailed 1-test). FIG. 5E: Bioluminescence imaging of tumor burden after Cot CAR-T cell+C1C02-Cot treatment was conducted at indicated time points after A20-Luc cell injection (data not shown). Bioluminescence intensity was calculated by the mean flux (p/s, mean±SEM) of region of interest (ROI) of individual mouse (n=5) (FIG. 5E). The statistical significance at each time point between Cot CAR-T+C1C02-Cot group versus other control groups were determined by unpaired two-tailed t-test (*p<0.05). (FIG. 5F). In vivo CAR-T cell tracing was performed using luciferase-expressing CAR-T cells and bioluminescence imaging. Balb/C mice were s.c injected with A20 cells (2×10⁷) on the back (data not shown). After the tumor mass was detectable at the injection site, mice were irradiated (2.5 Gy) and injected with CD40 CAR-Luc-T cells or Cot CAR-Luc-T cells (5×10⁶) the next day with or without bidaily injection of C1C02-Cot. Bioluminescence imaging was performed at indicated time points after CAR-T cell injection. Results are representative of at least 3 (FIG. 5B, FIG. 5D, FIG. 5E) and 2 (FIG. 5D, FIG. 5F) independent experiments.

FIGS. 6A-6J Anti-CD40 human switchable CAR-T cells with anti-human CD40 probes show functional activities in vitro. FIG. 6A: Schematic diagram of the hCot CAR, hHis-CAR, and hMyc-CAR constructs. FIG. 6B: Representative flow cytometry plot of Cot-CAR expression on human T cells at five days after transduction (left); Cotinine-labeled 2B1-Cκ (2B1-Cot) binding to Daudi cells (right). FIG. 6C: PKH26-labeled Daudi cells (target) were pre-incubated with unlabeled 2B1-Cκ (free 2B1) or 2B1-Cot and then co-cultured with hCot CAR-T cells (effector) at the indicated ratios for six h. Viable Daudi cells were counted with cell-counting beads via flow cytometry and the percent cytotoxicity was calculated as described in Methods. FIG. 6D: Daudi cells were pre-incubated with free 2B1 or 2B1-Cot, and then co-cultured with hCot CAR-T cells for 24 h. The amount of IFN-γ in culture supernatants was measured by ELISA. FIG. 6E: Representative flow cytometry plot of His-CAR expression on human T cells at eight days after transduction (Top); 2B1-Cκ-His binding to Raji cells (Bottom). FIG. 6F: Raji-Luc cells (target) were pre-incubated with 2B1-Cκ-His, and then co-cultured with hHis CAR-T cells (effector) at the indicated ratios. Untransduced T cells co-cultured with target cells in the absence of 2B1-Cκ-His were used as a control. After 24 h incubation, Raji cell viability was measured based on remaining luciferase activity and calculated as described in Methods. FIG. 6G: Raji cells were pre-incubated with 2B1-Cκ-His and then co-cultured with hHis CAR-T cells for 24 h. Untransduced T cells and hHis CAR-T cells cultured without target cells were included as controls. The amount of IFN-γ in culture supernatants was measured by ELISA. FIG. 6H: Representative flow cytometry plots of Myc-CAR expression on human T cells at eight days after transduction (left); 2B1-Cκ-Myc binding to Raji cells (right). FIG. 6I: Raji-Luc cells (target) were pre-incubated with 2B1-Cκ-Myc and then co-cultured with three different hMyc CAR-T cells (effector) at the indicated ratios. Untransduced T cells co-cultured with target cells in the absence of 2B1-Cκ-Myc were included as a control. After 24 h incubation, Raji cell viability was measured based on remaining luciferase activity. FIG. 6J: Raji cells were pre-incubated with 2B1-Cκ-Myc, and then co-cultured with hMyc CAR-T cells for 24 h. The amount of IFN-γ in culture supernatants was measured by ELISA.

FIGS. 7A-7B Antitumor efficacy of anti-CD40 switchable CAR-T cells is recapitulated with an anti-human CD40 probe and human switchable CAR-T cells in vivo. FIG. 7A: Experimental scheme for treatment of human B cell lymphoma xenograft using hCot CAR-T cells. NSG mice were injected with Daudi-Luc cells (5×10⁵) on day 0 and hCot CAR-T cells (1×10⁷) on day 3. From the day of CAR-T cell injection, 2B1-Cot (25 μg/head) is injected intravenously every other day for a total of 8 times. FIG. 7B: Bioluminescence imaging of tumor burden after hCot CAR-T cell+2B1-Cot treatment was conducted at indicated time points after Daudi-Luc cell injection (data not shown). Bioluminescence intensity was calculated by the mean flux (p/s, mean±SEM) of the region of interest (ROI) of the individual mouse (n=5). The statistical significance at each time point between hCot CAR-T+2B1-Cot group versus other control groups were determined by unpaired two-tailed t-test (***p<0.001, *p<0.05). Results are representative of at least two independent experiments.

FIGS. 8A-8B anti-mouse CD40 antibody (clone C1C02) binds to both recombinant CD40 protein and cell-surface CD40 on A20 lymphoma cell line. FIG. 8A: Recombinant mouse CD40-Fc (2.5 μg/ml) was coated in ELISA plate and the serial diluents of C1C02-Cκ or the irrelevant scFv-Cκ were loaded. The bound scFv-Cκs were detected by a secondary anti-human Cκ-HRP followed by a chromogenic reaction with TMB substrate. The absorbance was measured at 450 nm. EC₅₀ of the antibody binding was calculated as the dose that showed 50% absorbance of that of the maximal binding. FIG. 8B: A20, a CD40-expressing mouse B lymphoma cell line, was stained with C1C02-Cκ (1 μg/1×10⁵ cells) or the irrelevant scFv-Cκ and then stained with a secondary anti-human Cκ-APC. The binding intensity of the scFv-Cκs was analyzed by flow cytometry. The numbers are the mean fluorescence intensities (MFIs) of the binding.

FIGS. 9A-9B Splenic dendritic cells produce IL-6 when co-cultured with CD40 CAR-T cells. CD40 CAR-T cells were co-cultured with A20 or splenic dendritic cells for 24 h. The amounts of IL-6 (FIG. 9A) or IL-10 (FIG. 9B) in culture supernatants were measured by ELISA. (n.d: not detected).

FIGS. 10A-10D Neutralization of IL-6 and IL-1β or depletion of macrophages cannot alleviate lethal toxicity of CD40 CAR-T cell. Balb/C mice were irradiated (2.5 Gy) for lymphodepletion on day −1 and injected 5×106 (FIGS. 10A, FIG. 10C) or 1×10⁶ (FIG. 10B, FIG. 10D) control T or CD40 CAR-T cells on day 0. For IL-6 or IL-1β neutralization (FIG. 10A, FIG. 10B), anti-IL-6 or anakinra were daily injected intraperitoneally a total of 5 times from the day of CAR-T injection. For depleting phagocytes (FIG. 10C, FIG. 10D), clodronate liposome was injected intraperitoneally for three consecutive days prior to CAR-T cell infusion. Survival was monitored daily and analyzed by Log-rank (Mantel-Cox) test (n.s: not significant). Results are representative of 2 independent experiments.

FIGS. 11A-11B CD40 is highly expressed in lung and spleen. FIG. 11A: Evaluation of CD40 mRNA expression level in mouse tissues. Total RNAs were extracted from various organs (lung, spleen, liver, intestine and kidney) of normal Balb/C mice and subjected to quantitative RT-PCR of CD40 or β-actin mRNA. CD40 mRNA expression level was determined from the triplicate samples from each tissue and normalized to that of β-actin. Relative levels were calculated by dividing each value by the average value of liver. The average relative CD40 mRNA levels from the triplicates were plotted. FIG. 11B: Evaluation of CD40 protein expression in mouse tissues by immunohistochemistry. Various organs of normal Balb/C mice were isolated and fixed. The paraffin-embedded tissues were deparaffinized, stained with anti-mouse CD40 antibody (C1C02-Cκ) as a primary antibody, anti-hCκ-biotin as a secondary antibody and avidin-HRP as a tertiary antibody, which subjected to color reaction with DAB substrate. The slides were counterstained with hematoxylin. Results are representative of two independent experiments.

FIG. 12 Schematic representation of toxicity regulation by switchable CAR-T cells. If one supposes that at least three CAR molecule engagement is the threshold for CAR-T cell activation, tumor cells expressing nine tumor antigens (TAs) and normal cells expressing three TAs would similarly activate conventional CAR-T cells, which would lead to normal cell toxicity (left). For switchable CAR-T cells, if the adaptors are present at a sufficiently high level, the adaptors would bind to all three TAs on normal cells, which will also lead to normal cell toxicity just as conventional CAR-T cells (upper right). However, at a moderate level of the adaptors which allows the adaptor to bind only ⅓ of TAs on the cell surface, tumor cells would have still three adaptors on the cell surface that can activate CAR-T cells, whereas normal cells would have only one adaptor on the cell surface that is not enough for CAR-T cell activation (middle right). Therefore, at this adaptor level, tumor cells will be killed by CAR-T cells, but normal cells will not, which opens a window for efficient tumor killing without normal cell toxicity. At a low level of adaptors which allows the adaptor to bind 1/9 of TAs on cell surface, the adaptors will bind only one TA on tumor cells that is not sufficient to activate T cells, whereas it will not bind normal cells at all. Hence, at this level, neither tumor cell killing nor normal cell killing will happen (lower right). Therefore, this adaptor level will not be appropriate for CAR-T cell efficacy. Thus, switchable CAR-T cells can have an optimal therapeutic window by adjusting the adaptor levels to moderate ones, while conventional CAR-T cells cannot.

FIGS. 13A-13B Anti-human CD40 antibodies bind to both recombinant human CD40 protein and cell surface CD40 on human Raji lymphoma cell. FIG. 13A: Recombinant human CD40-Fc (2.5 μg/ml) was coated in ELISA plate and the serial diluents of 2B1-Ck, 2E1-Ck and irrelevant scFv antibody-Ck were loaded. The bound scFv-Cκs were detected by a secondary anti-human Cκ-HRP followed by chromogenic reaction with TMB substrate. The absorbance was measured at 450 nm. EC₅₀ of the antibody binding was calculated as the dose that showed 50% absorbance of that of the maximal binding. FIG. 13B: Raji, a CD40-expressing human B lymphoma cell line, was stained with 2B1-Ck, 2E1-Ck or the irrelevant scFv-Cκ (1 μg/1×10⁵ cells), and then stained with secondary anti-human Ck-APC. The binding intensity of the scFv-Cκs were analyzed by flow cytometry. The numbers are mean fluorescence intensities (MFIs) of the binding.

FIGS. 14A-14C 2E1-Cot, another hCD40 adaptor, also activates hCot CAR-T cells to kill tumor in vitro. FIG. 14A: A representative flow cytometry plot of cotinine-labeled 2E1-Cκ (2E1-Cot) binding to Raji, a CD40-expressing human B lymphoma cell line. The numbers are mean fluorescence intensities (MFIs) of the binding. FIG. 14B: PKH26-labeled Raji cells (target) were pre-incubated with 2E1-Cot, and then co-cultured with hCot CAR-T cells (effector) at the indicated ratios for 6 hours. Viable Raji cells were counted with cell-counting beads via flow cytometry and the percent cytotoxicity was calculated as described in Methods. FIG. 14C: Raji cells were pre-incubated with 2E1-Cot, and then co-cultured with hCot CAR-T cells for 24 h. The amount of IFN-γ in culture supernatants were measured by ELISA.

FIGS. 15A-15I In vitro anti-tumor functional test of various 2B1-His or -Myc adaptors. FIG. 15A-C: In vitro anti-tumor functional test of hHis CAR-T cell plus 2B1-L1-His adaptor, an hCD40 adaptor. A representative flow cytometry plot of 2B1-L1-His binding to Raji, a CD40-expressing human B lymphoma cell line (FIG. 15A). The numbers are mean fluorescence intensities (MFIs) of the binding. Raji-Luc cells (target) were pre-incubated with 2B1-L1-His, and then co-cultured with hHis CAR-T cells (effector) at the indicated ratios. After 16-24 h, 30 μg D-Luciferin was added to each well and luminescent signals were analyzed by luminometer. Raji cell viability were calculated as described in Methods (FIG. 15B). Raji cells were pre-incubated with 2B1-L1-His, and then co-cultured with hHis CAR-T cells for 24 h. The amount of IFN-γ in culture supernatants were measured by ELISA (FIG. 15C). FIG. 15D-I: In vitro anti-tumor functional test of hMyc CAR-T cell plus various 2B1-Myc adaptors (2B1-Ck-L2-Myc, 2B1-Ck-Myc, 2B1-Ck-L1-Myc, 2B1-L1-Myc-Ck and 2B1(Fab)-Myc). A representative flow cytometry plot of 2B1-Myc adaptors binding to Raji cell (FIG. 15D, FIG. 15G). Raji-Luc cells (target) were pre-incubated with various 2B1-Myc adaptors, and then co-cultured with hMyc CAR-T cells (effector) at the indicated ratios. After 16-24 h, 30 μg D-Luciferin was added to each well and luminescent signals were analyzed by luminometer. Raji cell viability were calculated as described in Methods (FIG. 15E, FIG. 15H). Raji cells were pre-incubated with various 2B1-Myc adaptors, and then co-cultured with hMyc CAR-T cells for 24 h. The amount of IFN-γ in culture supernatants were measured by ELISA (FIG. 15F, FIG. 15I).

FIGS. 16A-16B Antitumor efficacy of hCot CAR-T cells with hCD40 adaptor, 2E1-Cot. FIG. 16A: Experimental scheme for treatment of human B cell lymphoma xenograft using hCot CAR-T cells. NSG mice were injected with Raji-Luc cells (1×105) on day 0 and hCot CAR-T cells (1×10⁷) on day 3. From the day of CAR-T cell injection, 2E1-Cot (25 μg/head) is injected intravenously every other day for a total of eight times. FIG. 16B: Bioluminescence imaging of tumor burden after hCot CAR-T cell+2E1-Cot treatment was conducted at indicated time points after Raji-Luc cell injection (data not shown). Bioluminescence intensity was calculated by the mean flux (p/s, mean±SEM) of region of interest (ROI) of individual mouse (n=5).

FIGS. 17A-17B Antitumor efficacy of hHis CAR-T cells with hCD40 adaptor, 2B1-Ck-His. FIG. 17A: Experimental scheme for treatment of human B cell lymphoma xenograft using hHis CAR-T cells. NSGA-SID mice were injected with Raji-Luc cells (1×105) on day 0 and hHis CAR-T cells (5×106) on day two. From the day of CAR-T cell injection, 2B1-Ck-His (25 μg/head) is injected intravenously every other day for a total of 8 times. FIG. 17B: Bioluminescence imaging of tumor burden after hHis CAR-T cell+2B1-Ck-His treatment was conducted at indicated time points after Raji-Luc cell injection (data not shown). Bioluminescence intensity was calculated by the mean flux (p/s, mean±SEM) of region of interest (ROI) of individual mouse (n=4-5). The statistical significance at each time point between hHis CAR-T+2B1-Ck-His group versus other control groups was determined by unpaired two-tailed t-test (**p<0.005, *p<0.05).

FIGS. 18A-18F Anti-His switchable CAR-NK cells with anti-human CD40 probes show functional activities in vitro. FIG. 18A: Representative flow cytometry plot of His-CAR expression on NK-92 cells after transduction. FIG. 18B: His-labeled 2B1-Cκ (2B1-Cκ-His) binding to Daudi and IM-9 cells. FIG. 18C: Daudi-Luc cells (target) were pre-incubated with 2B1-Cκ-His, and then co-cultured with hHis CAR-NK cells (effector) at the indicated ratios. Untransduced NK cells co-cultured with target cells in the absence of 2B1-Cκ-His were used as a control. After 2 h incubation, Daudi cell viability was measured based on remaining luciferase activity and calculated as described in Methods. FIG. 18D: Daudi-Luc cells were pre-incubated with 2B1-Cκ-His and then co-cultured with hHis CAR-NK cells for 24 hr. Untransduced NK cells and hHis CAR-T cells cultured without target cells were included as controls. The amount of IFN-7 in culture supernatants was measured by ELISA. FIG. 18E: IM-9-Luc cells (target) were pre-incubated with 2B1-C-His, and then co-cultured with hHis CAR-NK cells (effector) at the indicated ratios. Untransduced NK cells co cultured with target cells in the absence of 2B1-Cκ-His were used as a control. After 2 h incubation, IM-9 cell viability was measured based on remaining luciferase activity and calculated as described in Methods. FIG. 18F: IM-9-Luc cells were pre-incubated with 2B1-C-His and then co-cultured with hHis CAR-NK cells for 24 h. Untransduced NK cells and hHis CAR-T cells cultured without target cells were included as controls. The amount of IFN-γ in culture supernatants was measured by ELISA.

FIGS. 19A-19F Humanized anti-His CAR-T (huHis CAR-T) cells with Humanized anti-human CD40 (hu2B1-Cκ-His) probes show functional activities in vitro. FIG. 19A: Representative flow cytometry plot of Humanized anti-His CAR (huHis CAR) expression on human T cells at five days after transduction. FIG. 19B: Humanized 2B1-Cκ-His (hu2B1-Cκ-His) binding to Raji and IM9 cells. FIG. 19C, 19E: Raji-Luc or IM9-Luc cells (target) were pre-incubated with hu2B1-Cκ-His, and then co-cultured with huHis CAR-T cells (effector) at the indicated ratios. Untransduced T cells co-cultured with target cells in the absence of hu2B1-Cκ-His were used as a control. After 24 h incubation, target cell viability was measured based on remaining luciferase activity and calculated as described in Methods. FIG. 19D, 19F: Raji-Luc or IM9-Luc cells were pre-incubated with hu2B1-Cκ-His and then co-cultured with huHis CAR-T cells for 24 hr. Untransduced T cells and huHis CAR-T cells cultured without target cells were included as controls. The amount of IFN-γ in culture supernatants was measured by ELISA.

FIGS. 20A-C The individual specific binders are screened by ELISA and 23 different scFv clones were further characterized by sequencing. FIG. 20A: Reactivity of the selected scFv clones to human CS1 was assayed by measuring absorbance at 405 nm. The individual scFv clones were reactive to human CS1-His (▪) and human CS1-Fc ( [symbol] ), but not hFc ( [symbol] ) or BSA (□).Binding activity determination of selected clones specific to human CS1. The three clones selected from the phage ELISA were verified in a serial dilution ELISA after expression and purification as a form of scFv-Cκ-His. The y-axis of the graphs depicts the signal for serial dilutions of the antibodies, and the x-axis depicts the antibody concentration in the wells of the dilution series. The binding to human CS1-Fc is indicated as red, while the binding to BSA is indicated as blue. The error bars show one standard deviation based on three independent experiments. FIG. 20B: Binding activity of anti-human CS1 cFv-Cκ-His on J558/CS1 or J558 cells measured using flow cytometry. J558 cells were transfected with human CS1. Binding of scFv adapters were detected by adding APC conjugated anti-human Cκ antibodies or APC conjugated anti-His tag secondary antibodies. FIG. 20C: Binding activity of anti-human CS1 scFv-Cκ-His on MM1.s cells measured using flow cytometry. Binding of the antibodies were detected by adding either an APC conjugated anti-human Cκ antibodies, or an APC conjugated anti-His tag secondary antibodies, respectively.

FIGS. 21A-22B In vitro potency of anti-His CAR-T cells with CS1 target adaptors. FIG. 21A: MM.1s-luc cells (target) were pre-incubated with each anti-human CS1 scFc Cκ-His and co-cultured with His CAR-T (effector) cells at indicated ratios for 6h. Percent cytotoxicity was calculated from the average RLU of samples using the following formula: tumor cell viability (%)=average RLU in the well with CAR-T cells/average RLU in the well with without CAR-T cells×100. FIG. 21B: MM.1s-luc cells were pre-incubated with each anti-human CS1 scFv-Cκ-His and co-cultured with His CAR-T cells for 24 h. The amount of IFN-γ in culture supernatants was measured by ELISA.

FIGS. 22A-22B Analysis of humanized antibody binding affinities. SPR sensorgrams are shown for the humanized and affinity maturated anti-His scFv (FIG. 22A) and humanized anti-CD40 scFv (2B1 clone) (FIG. 22B).

### DETAILED DESCRIPTION

As described herein, switchable CAR-T systems can be utilized for a tumor antigen that cannot be targeted by conventional CAR-T cells due to on-target off-tumor toxicity. In some embodiments, the switchable CAR-T systems comprises a chimeric antigen receptor T cell that recognizes a peptide tag fused to an anti-tumor antibody, for example, an anti-CD40 antibody or an anti-CS1 antibody. This switchable system minimizes the on-target off-tumor toxicity due to the expression of antigens expressed on tumor cells (i.e., CD40 or CS1) that are also expressed on a large number of normal tissue cells. As compared to switchable system that uses chemical tag, the system disclosed herein avoids potential aberrant biological effect of the chemical and inefficiency and inconsistency associated with chemical tag, provides maximum control of killing activity of the CAR-T cells, and also allows ease of purification of the fusion protein adaptors. The switchable CAR-T systems also demonstrate remarkable therapeutic efficacy against tumors, for example, CD40-expressing tumors or CS1-expressing tumors.

Thus, the method can regulate CAR-T cell toxicity by adjusting doses of tumor-targeting adaptors (aka. anti-tumor adaptors) for switchable CAR-T cells, without the need for complicated design of CAR constructs. The anti-tumor adaptors are typically anti-tumor antibodies, for example, anti-CD40, anti CD3, anti-CS1, etc.

### I. Terminology

Unless otherwise defined, all terms of art, notations, and other scientific or medical terms or terminology used herein are intended to have the meanings commonly understood by those of ordinary skill in the art. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the present disclosure or features of the claims. See, for example, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

The terms “polynucleotide” and “nucleic acid” interchangeably refer to chains of nucleotides of any length and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10′, and most preferably less than about 10⁻²⁰.

Other terms used in the fields of recombinant nucleic acid technology, microbiology, immunology, antibody engineering, and molecular and cell biology as used herein will be generally understood by one of ordinary skill in the applicable arts.

### II. Introduction

Described herein are compositions, pharmaceutical compositions, methods, and kits relating to switchable chimeric antigen receptor (CAR) cells and systems that ultimately target antigens expressed on cancer cells that are also expressed on non-cancerous cells, for example, CD40 and/or CS1. In some embodiments of the present disclosure, CARs can comprise, for example, an antigen recognition domain (comprising an antibody or antigen binding fragment thereof that specifically binds to a peptide tag, such as a His or Myc tag), a hinge domain, a transmembrane domain, and a signal transduction domain. The antigen recognition domain of the CAR (expressed in an immune cell) can recognize a peptide tag, for example, a histidine (i.e., “His”) tag or a Myc tag, that is conjugated to an anti-tumor antibody or antigen binding fragment thereof (e.g., an anti-CD40 antibody or antigen binding fragment thereof or an anti-CS1 antibody or antigen binding fragment thereof). Switchable systems such as these enable the targeting of antigens that may be present in non-tumor tissues and non-cancerous cells. These CAR-expressing immune cells then only become active in the presence of an anti-tumor antibody that is fused to the peptide tag. In some embodiments, switchable CAR cells can comprise T-cells, natural killer (NK) cells, and macrophages.

Peptide tags for adaptors are beneficial over chemical tag as tags for adaptors in switchable CAR T cell system. Chemicals may have aberrant biological activities or toxicities whereas known peptides usually do not. Conjugation efficiency of chemical tags to antibody adaptors can never be 100% nor be consistent. Drug to antibody ratio (DAR) may also very variable between production batches. However, peptide tags can be linked to antibodies as a fusion tag using recombinant protein engineering. Therefore, labeling efficiency is 100% and consistent. DAR will be always 1 to 1 all the time. Especially, His tag and Myc tag have an advantage as a peptide tag for adaptors. Since both tags are used as affinity tags used for recombinant protein purification, His tag or Myc tag-labeled adaptor proteins can be purified using the affinity column against these tags without adding additional tag for being recognized by CAR-T cells. Particularly, His tag is used for clinical grade large scale antibody purification in the manufacturing process (as exemplified in its usage for blinatumomab), which makes this tag versatile for the commercial development. Also, His tag is known to be poorly immunogenic (Clin Vaccine Immunol. 2011 February; 18(2):289-97), which may prevent anti-adaptor immune response in the patient. Myc tag is also poorly immunogenic because it is a part of endogenous nuclear protein, c-Myc, to which generation of high affinity antibodies are prevented due to immunological tolerance.

### III. Antibodies

The present disclosure provides compositions, systems, kits, and methods for treating cancers that present an anti-tumor antigen (e.g., a CD40 antigen or CS1 antigen, and, for example in a subject having or suspected of having a cancer comprising cells that overexpress CD40 or CS1 or otherwise have elevated CD40 or CS1 activity or signaling, an animal model, an in vitro tissue culture model, and the like) that utilize antibodies or antigen binding portions thereof. In some embodiments, antibodies or antigen binding portions thereof that specifically or selectively bind anti-tumor antigens (e.g., CD40 and CS1) are provided herein. In some embodiments, antibodies, or antigen binding portions thereof, that specifically or selectively bind anti-tumor antigens (e.g., CD40 and CS1) are provided herein that are conjugated to a peptide tag that be recognized by another anti-peptide tag antibody (or antigen binding portion thereof). In some embodiments, antibodies, or antigen binding portions thereof, that specifically or selectively bind to peptide tags (e.g., a His tag or a Myc tag) are also provided herein that can be engineered into an antigen recognition domain of a chimeric antigen receptor (CAR). These CARs can engage and interact with anti-tumor antibodies (or antigen binding portions thereof) that are fused to a peptide tag as a switchable CAR immune cell system, further described in proceeding sections.

In certain aspects, antibodies as described herein are monoclonal antibodies. Antibodies of the present disclosure may also be engineered into other modalities, such as engineered chimeric antigen receptors (CAR), also known as chimeric immunoreceptors, chimeric T cell receptors, or artificial T cell receptors, such as, for example CAR-T, CAR-NK, or CAR macrophage. CARs for CAR T, CAR NK and CAR macrophage have similar structures: the extracellular domain including the antigen binding domain and, optionally, a spacer also referred to herein as a “hinge” or “hinge domain”) that is involved in engagement of target cells; a transmembrane domain that docks the CAR to immune cells and is also involved in other functions of CAR, such as stability and interaction with other membrane proteins; and an intracellular signaling domain that is involved in signaling transduction and activation of immune cells.

As used herein, the terms “specifically bind,” “specifically binds to,” “specific for,” “selectively binds,” and “selective for” an anti-tumor antigen (e.g. a CD40 antigen or an epitope on a protein related to CD40, a CS1 antigen or an epitope on a protein related to CS1, and/or an anti-peptide tag, e.g., a His or Myc tag, antigen or an epitope thereof) mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

An antibody, as used herein, can refer to an intact antibody (e.g., an intact immunoglobulin) and antibody fragment, for example, an antigen binding fragment, or a bispecific antibody. Antigen binding fragments can comprise at least one antigen binding domain. One example of an antigen binding domain is an antigen binding domain formed by a V_{H}-V_{L} dimer. Antibodies and antigen binding fragments can be described by the antigen to which they specifically bind. In some embodiments, the antigen binding fragments provided herein can comprise any of the antigen binding portions (also referred to as antigen binding domains) described below.

The V_{H} and V_{L} regions can be further subdivided into regions of hypervariability (hypervariable regions (HVRs), also called complementarity determining regions (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. (See Kabat et al. (1991) Sequences of Proteins of Immunological Interest 5th ed., Public Health Service, National Institutes of Health, Bethesda, MD.) CDR sequences on the heavy chain (V_{H}) may be designated as HCDR1, 2, 3 while CDR sequences on the light chain (V_{L}) may be designated as LCDR1, 2, 3.

Provided herein are antibodies or antigen binding portions thereof that specifically bind to antigens related to cancers (e.g., CD40 or CS1), and the antibodies or antibody binding portions are fused to peptide tags, e.g., a His or Myc tag, antigen or an epitope thereof that are not themselves related to cancers. Such antibodies may be, for example, monoclonal antibodies (mAbs) or recombinant/chimeric antibodies (i.e., synthetic antibodies derived from synthetic nucleic acid constructs, such as viral vectors, that may also contain monoclonal Ab sequences as disclosed herein). Anti-His and Anti-Myc antibodies according to the present disclosure can further be incorporated as or into the antigen recognition domain of a CAR as described in Section IV below.

### A. Anti-CD40 Antibodies

In each case, where a specific amino acid sequence is recited, embodiments of anti-CD40 antibodies comprising a sequence having at least 90% (e.g. 90%, 91%, 92%, 93%, 94%, 95%, 96%, 97%, 98%, or 99%) identity to the recited sequence (e.g., SEQ ID NOs: 1, 10, 19) are also provided.

In some embodiments, the anti-CD40 antibody used herein is an scFv and comprises a sequence that share at least 90%, e.g., at least 91%, at least 92%, at least 93%, at least 94%, at least 95%, at least 96%, at least 97%, at least 98%, or at least 99% identity to any one of SEQ ID NOs 1, 10, or 19.

In some embodiments, an anti-CD40 antibody or antigen binding portion thereof provided herein comprises an HCDR1 of any one of SEQ ID NOs: 4, 13, and 22, an HCDR2 of any one of SEQ ID NOS: 5, 14, and 23, an HCDR3 of any one of SEQ ID NOS: 6, 15, and 24, an LCDR1 of any one of SEQ ID NOS: 7, 16, and 25, an LCDR2 of any one of SEQ ID NOS: 8, 17, and 26, an LCDR3 of any one of SEQ ID NOS: 9, 18, and 27; and the FW regions in the VH region are at least 80% identical to the FW regions present in the VH region of SEQ ID NO 2, 11, and 20, and wherein the FW regions in the VL region are at least 80% identical to the FW regions present in the VL region of SEQ ID NO: 3, 12, and 21. In some embodiments, an anti-CD40 antibody or antigen binding fragment thereof can comprise an anti-CD40 scFV having a sequence at least 80% identical to one of 1, 10, or 19.

The disclosure also provides an antibody or antigen binding portion thereof that specifically binds to a CD40 antigen, wherein the antibody or antigen binding portion thereof comprises a heavy chain variable region comprising an amino acid sequence that is at least 90% identical (for example, at least 91%, at least 92%, at least 93%, at least 94%, at least 95%, at least 96%, at least 97%, at least 98% or at least 99%, or 100% identical) to any of SEQ ID NOs: 2, 11, or 20 and/or a light chain variable region comprising an amino acid sequence that is at least 90% identical, for example, at least 91%, at least 92%, at least 93%, at least 94%, at least 95%, at least 96%, at least 97%, at least 98% or at least 99%, or 100% identical to any of SEQ ID NOs: 3, 12, or 21. Sequence details can be found in Table 1.

The disclosure also provides an antibody or antigen binding portion thereof that specifically binds to a CD40 antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 2 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 3.

The disclosure also provides an antibody or antigen binding portion thereof that specifically binds to a CD40 antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 11 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 12.

The disclosure also provides an antibody or antigen binding portion thereof that specifically binds to a CD40 antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 20 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 21.

The disclosure also provides an antibody or antigen binding portion thereof that specifically binds to a CD40 antigen, wherein the antibody or antigen binding portion thereof comprises a heavy chain variable region comprising a heavy chain CDR1 (HCDR1) of SEQ ID NO: 4 or a HCDR1 having no more than 1, 2, 3, or 4 amino acid substitutions as compared to SEQ ID NO: 4, a heavy chain CDR2 (HCDR2) of SEQ ID NO: 5 or a HCDR2 having no more than 1, 2, 3, or 4 amino acid substitutions as compared to SEQ ID NO: 5, and a heavy chain CDR3 (HCDR3) of SEQ ID NO: 6 or a HCDR3 having no more than 1, 2, 3, or 4 amino acid substitutions as compared to SEQ ID NO: 6; and wherein the antibody or antigen binding portion thereof comprises a light chain variable region comprising a light chain CDR1 (LCDR1) of SEQ ID NO: 7 or a LCDR1 having no more than 1, 2, 3, or 4 amino acid substitutions as compared to SEQ ID NO: 7, a light chain CDR2 (LCDR2) of SEQ ID NO: 8 or a LCDR2 having no more than 1, 2, 3, or 4 amino acid substitutions as compared to SEQ ID NO: 8, and a light chain CDR3 (LCDR3) of SEQ ID NO: 9 or a LCDR3 having no more than 1, 2, 3, or 4 amino acid substitutions as compared to SEQ ID NO: 9.

The disclosure also provides an antibody or antigen binding portion thereof that specifically binds to a CD40 antigen, wherein the antibody or antigen binding portion thereof comprises a heavy chain variable region comprising a heavy chain CDR1 (HCDR1) of SEQ ID NO: 13 or a HCDR1 having no more than 1, 2, 3, or 4 amino acid substitutions as compared to SEQ ID NO: 13, a heavy chain CDR2 (HCDR2) of SEQ ID NO: 14 or a HCDR2 having no more than 1, 2, 3, or 4 amino acid substitutions as compared to SEQ ID NO:14, and a heavy chain CDR3 (HCDR3) of SEQ ID NO: 15 or a HCDR3 having no more than 1, 2, 3, or 4 amino acid substitutions as compared to SEQ ID NO:15; and wherein the antibody or antigen binding portion thereof comprises a light chain variable region comprising a light chain CDR1 (LCDR1) of SEQ ID NO: 16 or a LCDR1 having no more than 1, 2, 3, or 4 amino acid substitutions as compared to SEQ ID NO: 16, a light chain CDR2 (LCDR2) of SEQ ID NO: 17 or a LCDR2 having no more than 1, 2, 3, or 4 amino acid substitutions as compared to SEQ ID NO: 17, and a light chain CDR3 (LCDR3) of SEQ ID NO: 18 or a LCDR3 having no more than 1, 2, 3, or 4 amino acid substitutions as compared to SEQ ID NO: 18.

The disclosure also provides an antibody or antigen binding portion thereof that specifically binds to a CD40 antigen, wherein the antibody or antigen binding portion thereof comprises a heavy chain variable region comprising a heavy chain CDR1 (HCDR1) of SEQ ID NO: 22 or a HCDR1 having no more than 1, 2, 3, or 4 amino acid substitutions as compared to SEQ ID NO: 22, a heavy chain CDR2 (HCDR2) of SEQ ID NO: 23 or a HCDR2 having no more than 1, 2, 3, or 4 amino acid substitutions as compared to SEQ ID NO: 23, and a heavy chain CDR3 (HCDR3) of SEQ ID NO: 24 or a HCDR3 having no more than 1, 2, 3, or 4 amino acid substitutions as compared to SEQ ID NO: 24; and wherein the antibody or antigen binding portion thereof comprises a light chain variable region comprising a light chain CDR1 (LCDR1) of SEQ ID NO: 25 or a LCDR1 having no more than 1, 2, 3, or 4 amino acid substitutions as compared to SEQ ID NO: 25, a light chain CDR2 (LCDR2) of SEQ ID NO: 26 or a LCDR2 having no more than 1, 2, 3, or 4 amino acid substitutions as compared to SEQ ID NO: 26, and a light chain CDR3 (LCDR3) of SEQ ID NO: 27 or a LCDR3 having no more than 1, 2, 3, or 4 amino acid substitutions as compared to SEQ ID NO: 27.

In examples, the antigen is CD40, Uniprot P25942-1

MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPI
IFGILFAILLVLVFIKKVAKKPTNKAPHPKQEPQEINFPDDLPGSNTAAP
VQETLHGCQPVTQEDGKESRISVQERQ

### B. Anti-CS1 Antibodies

In each case, where a specific amino acid sequence is recited, embodiments of anti-CS1 antibodies comprising a sequence having at least 90% (e.g., at least 91%, at least 92%, at least 93%, at least 94%, at least 95%, at least 96%, at least 97%, at least 98%, at least 99%, or 100%) identity to the recited sequence (e.g., SEQ ID NOs: 82, 91, or 100) are also provided.

In some embodiments, the anti-CD40 antibody used herein is an scFv and comprises a sequence that share at least 90%, e.g., at least 91%, at least 92%, at least 93%, at least 94%, at least 95%, at least 96%, at least 97%, at least 98%, or at least 99% identity to any one of SEQ ID NOs: 82, 91, or 100.

In some embodiments, an anti-CS1 antibody provided herein comprises an HCDR1 of any one of SEQ ID NOs: 85, 94, and 103, an HCDR2 of any one of SEQ ID NOS: 86, 95, and 104, an HCDR3 of any one of SEQ ID NOS: 87, 96, and 105, an LCDR1 of any one of SEQ ID NOS: 88, 97, and 106, an LCDR2 of any one of SEQ ID NOS: 89, 98, and 107, an LCDR3 of any one of SEQ ID NOS: 90, 99, and 108; and the FW regions in the VH region are at least 80% identical to the FW regions present in the VH region of SEQ ID NO 83, 92, and 101, and wherein the FW regions in the VL region are at least 80% identical to the FW regions present in the VL region of SEQ ID NO: 84, 93, and 102. In some embodiments, an anti-CS1 antibody or antigen binding fragment thereof can comprise an anti-CS1 scFV having a sequence at least 80% identical to one of 82, 91, or 100.

The disclosure also provides an antibody or antigen binding portion thereof that specifically binds to a CS1 antigen, wherein the antibody or antigen binding portion thereof comprises a heavy chain variable region comprising an amino acid sequence that is at least 90% identical (for example, at least 91%, at least 92%, at least 93%, at least 94%, at least 95%, at least 96%, at least 97%, at least 98% or at least 99%, or 100% identical) to any of SEQ ID NOs: 83, 92, or 101 and/or a light chain variable region comprising an amino acid sequence that is at least 90% identical, for example, at least 91%, at least 92%, at least 93%, at least 94%, at least 95%, at least 96%, at least 97%, at least 98% or at least 99%, or 100% identical to any of SEQ ID NOs: 84, 93, or 102. Sequence details can be found in Table 1 below.

The disclosure also provides an antibody or antigen binding portion thereof that specifically binds to a CS1 antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 83 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 84.

The disclosure also provides an antibody or antigen binding portion thereof that specifically binds to a CS1 antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 92 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 93.

The disclosure also provides an antibody or antigen binding portion thereof that specifically binds to a CS1 antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 101 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 102.

The disclosure also provides an antibody or antigen binding portion thereof that specifically binds to a CS1 antigen, wherein the antibody or antigen binding portion thereof comprises a heavy chain variable region comprising a heavy chain CDR1 (HCDR1) of SEQ ID NO: 85 or a HCDR1 having no more than 1, 2, 3, or 4 amino acid substitutions as compared to SEQ ID NO: 85, a heavy chain CDR2 (HCDR2) of SEQ ID NO: 86 or a HCDR2 having no more than 1, 2, 3, or 4 amino acid substitutions as compared to SEQ ID NO: 86, and a heavy chain CDR3 (HCDR3) of SEQ ID NO: 87 or a HCDR3 having no more than 1, 2, 3, or 4 amino acid substitutions as compared to SEQ ID NO: 87; and wherein the antibody or antigen binding portion thereof comprises a light chain variable region comprising a light chain CDR1 (LCDR1) of SEQ ID NO: 88 or a LCDR1 having no more than 1, 2, 3, or 4 amino acid substitutions as compared to SEQ ID NO: 88, a light chain CDR2 (LCDR2) of SEQ ID NO: 89 or a LCDR2 having no more than 1, 2, 3, or 4 amino acid substitutions as compared to SEQ ID NO: 89, and a light chain CDR3 (LCDR3) of SEQ ID NO: 90 or a LCDR3 having no more than 1, 2, 3, or 4 amino acid substitutions as compared to SEQ ID NO: 90.

The disclosure also provides an antibody or antigen binding portion thereof that specifically binds to a CS1 antigen, wherein the antibody or antigen binding portion thereof comprises a heavy chain variable region comprising a heavy chain CDR1 (HCDR1) of SEQ ID NO: 94 or a HCDR1 having no more than 1, 2, 3, or 4 amino acid substitutions as compared to SEQ ID NO: 94, a heavy chain CDR2 (HCDR2) of SEQ ID NO: 95 or a HCDR2 having no more than 1, 2, 3, or 4 amino acid substitutions as compared to SEQ ID NO: 95, and a heavy chain CDR3 (HCDR3) of SEQ ID NO: 96 or a HCDR3 having no more than 1, 2, 3, or 4 amino acid substitutions as compared to SEQ ID NO: 96; and wherein the antibody or antigen binding portion thereof comprises a light chain variable region comprising a light chain CDR1 (LCDR1) of SEQ ID NO: 97 or a LCDR1 having no more than 1, 2, 3, or 4 amino acid substitutions as compared to SEQ ID NO: 97, a light chain CDR2 (LCDR2) of SEQ ID NO: 98 or a LCDR2 having no more than 1, 2, 3, or 4 amino acid substitutions as compared to SEQ ID NO: 98, and a light chain CDR3 (LCDR3) of SEQ ID NO: 99 or a LCDR3 having no more than 1, 2, 3, or 4 amino acid substitutions as compared to SEQ ID NO: 99.

The disclosure also provides an antibody or antigen binding portion thereof that specifically binds to a CS1 antigen, wherein the antibody or antigen binding portion thereof comprises a heavy chain variable region comprising a heavy chain CDR1 (HCDR1) of SEQ ID NO: 103 or a HCDR1 having no more than 1, 2, 3, or 4 amino acid substitutions as compared to SEQ ID NO: 103, a heavy chain CDR2 (HCDR2) of SEQ ID NO: 104 or a HCDR2 having no more than 1, 2, 3, or 4 amino acid substitutions as compared to SEQ ID NO: 104, and a heavy chain CDR3 (HCDR3) of SEQ ID NO: 105 or a HCDR3 having no more than 1, 2, 3, or 4 amino acid substitutions as compared to SEQ ID NO: 105; and wherein the antibody or antigen binding portion thereof comprises a light chain variable region comprising a light chain CDR1 (LCDR1) of SEQ ID NO: 106 or a LCDR1 having no more than 1, 2, 3, or 4 amino acid substitutions as compared to SEQ ID NO: 106, a light chain CDR2 (LCDR2) of SEQ ID NO: 107 or a LCDR2 having no more than 1, 2, 3, or 4 amino acid substitutions as compared to SEQ ID NO: 107, and a light chain CDR3 (LCDR3) of SEQ ID NO: 108 or a LCDR3 having no more than 1, 2, 3, or 4 amino acid substitutions as compared to SEQ ID NO: 108.

In examples, the antigen is CS1, (Uniprot Q9NQ25-1):

MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEHLSKPKVTMGLQSNKNGTCVTNLT
CCMEHGEEDVIYTWKALGQAANESHNGSILPISWRWGESDMTFICVARNP
VSRNFSSPILARKLCEGAADDPDSSMVLLCLLLVPLLLSLFVLGLFLWFL
KRERQEEYIEEKKRVDICRETPNICPHSGENTEYDTIPHTNRTILKEDPA
NTVYSTVEIPKKMENPHSLLTMPDTPRLFAYENVI

### C. Anti-His Antibodies

Anti-His antibodies as described herein are intended to be engineered into a CAR as described in Section IV below. In each case, where a specific amino acid sequence is recited, embodiments of anti-His antibodies comprising a sequence having at least 90% (e.g. at least 91%, at least 92%, at least 93%, at least 94%, at least 95%, at least 96%, at least 97%, at least 98%, at least 99%, or 100%) identity to the recited sequence (e.g., SEQ ID NOs: 28 or 37)

In some embodiments, the anti-His antibody used herein is an scFv and comprises a sequence that share at least 90%, e.g., at least 91%, at least 92%, at least 93%, at least 94%, at least 95%, at least 96%, at least 97%, at least 98%, or at least 99% identity to any one of SEQ ID NOs 28 or 37.

In some embodiments, an anti-His antibody provided herein comprises an HCDR1 of any one of SEQ ID NOS: 31 and 40, an HCDR2 of any one of SEQ ID NOS: 32 and 41, an HCDR3 of any one of SEQ ID NOS: 33 and 42, an LCDR1 of any one of SEQ ID NOS: 34 and 43, an LCDR2 of any one of SEQ ID NOS: 33 and 44, an LCDR3 of any one of SEQ ID NOS: 36 and 45; and the FW regions in the VH region are at least 80% identical to the FW regions present in the VH region of SEQ ID Nos. 29 or 38, and wherein the FW regions in the VL region are at least 80% identical to the FW regions present in the VL region of SEQ ID NO: 30 or 39. In some embodiments, an anti-His antibody or antigen binding fragment thereof can comprise an anti-His scFV having a sequence at least 80% identical to one of 28 or 37.

The disclosure also provides an antibody or antigen binding portion thereof that specifically binds to a His antigen, wherein the antibody or antigen binding portion thereof comprises a heavy chain variable region comprising an amino acid sequence that is at least 90% identical (for example, at least 91%, at least 92%, at least 93%, at least 94%, at least 95%, at least 96%, at least 97%, at least 98% or at least 99%, or 100% identical) to any of SEQ ID NOs: 29 or 38 and/or a light chain variable region comprising an amino acid sequence that is at least 90°/a identical, for example, at least 91%, at least 92%, at least 93%, at least 94%, at least 95%, at least 96%, at least 97%, at least 98% or at least 99%, or 100% identical to any of SEQ ID NOs: 30 or 39. Sequence details can be found in Table 1 below.

The disclosure also provides an antibody or antigen binding portion thereof that specifically binds to a His antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 29 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 30.

The disclosure also provides an antibody or antigen binding portion thereof that specifically binds to a His antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 38 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 39.

The disclosure also provides an anti-His antibody or antigen binding portion thereof that specifically binds to a His antigen, wherein the antibody or antigen binding portion thereof comprises a heavy chain variable region comprising a heavy chain CDR1 (HCDR1) of SEQ ID NO: 31 or a HCDR1 having no more than 1, 2, 3, or 4 amino acid substitutions as compared to SEQ ID NO: 31, a heavy chain CDR2 (HCDR2) of SEQ ID NO: 32 or a HCDR2 having no more than 1, 2, 3, or 4 amino acid substitutions as compared to SEQ ID NO: 32, and a heavy chain CDR3 (HCDR3) of SEQ ID NO: 33 or a HCDR3 having no more than 1, 2, 3, or 4 amino acid substitutions as compared to SEQ ID NO: 33; and wherein the antibody or antigen binding portion thereof comprises a light chain variable region comprising a light chain CDR1 (LCDR1) of SEQ ID NO: 34 or a LCDR1 having no more than 1, 2, 3, or 4 amino acid substitutions as compared to SEQ ID NO: 34, a light chain CDR2 (LCDR2) of SEQ ID NO: 35 or a LCDR2 having no more than 1, 2, 3, or 4 amino acid substitutions as compared to SEQ ID NO: 35, and a light chain CDR3 (LCDR3) of SEQ ID NO: 36 or a LCDR3 having no more than 1, 2, 3, or 4 amino acid substitutions as compared to SEQ ID NO: 36.

The disclosure also provides an antibody or antigen binding portion thereof that specifically binds to a His antigen, wherein the antibody or antigen binding portion thereof comprises a heavy chain variable region comprising a heavy chain CDR1 (HCDR1) of SEQ ID NO: 40 or a HCDR1 having no more than 1, 2, 3, or 4 amino acid substitutions as compared to SEQ ID NO: 40, a heavy chain CDR2 (HCDR2) of SEQ ID NO: 41 or a HCDR2 having no more than 1, 2, 3, or 4 amino acid substitutions as compared to SEQ ID NO: 41, and a heavy chain CDR3 (HCDR3) of SEQ ID NO: 42 or a HCDR3 having no more than 1, 2, 3, or 4 amino acid substitutions as compared to SEQ ID NO: 42; and wherein the antibody or antigen binding portion thereof comprises a light chain variable region comprising a light chain CDR1 (LCDR1) of SEQ ID NO: 43 or a LCDR1 having no more than 1, 2, 3, or 4 amino acid substitutions as compared to SEQ ID NO: 43, a light chain CDR2 (LCDR2) of SEQ ID NO: 44 or a LCDR2 having no more than 1, 2, 3, or 4 amino acid substitutions as compared to SEQ ID NO: 44, and a light chain CDR3 (LCDR3) of SEQ ID NO: 45 or a LCDR3 having no more than 1, 2, 3, or 4 amino acid substitutions as compared to SEQ ID NO: 45.

In examples, the antigen is a His tag, in particular HHHHHH (SEQ ID NO:118), for example).

### D. Anti-Myc Antibodies

Anti-Myc antibodies as described herein are intended to be engineered into a CAR as described in Section IV below. In each case, where a specific amino acid sequence is recited, embodiments of anti-Myc antibodies comprising a sequence having at least 90% (e.g. at least 91%, at least 92%, at least 93%, at least 94%, at least 95%, at least 96%, at least 97%, at least 98%, at least 99%, or 100%) identity to the recited sequence (e.g., SEQ ID NOs: 46, 55, or 64).

In some embodiments, the anti-Myc antibody used herein an scFv and comprises a sequence that share at least 90%, e.g., at least 91%, at least 92%, at least 93%, at least 94%, at least 95%, at least 96%, at least 97%, at least 98%, or at least 99% identity to any one of SEQ ID NOs 46, 55, or 64.

In some embodiments, an anti-Myc antibody provided herein comprises an HCDR1 of any one of SEQ ID NOS: 49, 58, and 67, an HCDR2 of any one of SEQ ID NOS: 50, 59, and 68, an HCDR3 of any one of SEQ ID NOS: 51, 60, and 69, an LCDR1 of any one of SEQ ID NOS: 52, 61, and 70, an LCDR2 of any one of SEQ ID NOS: 53, 62, and 71, an LCDR3 of any one of SEQ ID NOS: 54, 63, and 72; and the FW regions in the VH region are at least 80% identical to the FW regions present in the VH region of SEQ ID Nos. 47, 56, and 65, and wherein the FW regions in the VL region are at least 80% identical to the FW regions present in the VL region of SEQ ID NO: 48, 57, and 66. In some embodiments, an anti-Myc antibody or antigen binding fragment thereof can comprise an anti-Myc scFV having a sequence at least 80% identical to one of 46, 55, or 64.

The disclosure also provides an antibody or antigen binding portion thereof that specifically binds to a Myc antigen, wherein the antibody or antigen binding portion thereof comprises a heavy chain variable region comprising an amino acid sequence that is at least 90% identical (for example, at least 91%, at least 92%, at least 93%, at least 94%, at least 95%, at least 96%, at least 97%, at least 98% or at least 99%, or 100% identical) to any of SEQ ID NOs: 47, 56, or 65 and/or a light chain variable region comprising an amino acid sequence that is at least 90% identical, for example, at least 91%, at least 92%, at least 93%, at least 94%, at least 95%, at least 96%, at least 97%, at least 98% or at least 99%, or 100% identical to any of SEQ ID NOs: 48, 57, or 66. Sequence details can be found in Table 1 below.

The disclosure also provides an antibody or antigen binding portion thereof that specifically binds to a Myc antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 47 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 48.

The disclosure also provides an antibody or antigen binding portion thereof that specifically binds to a Myc antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 56 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 57.

The disclosure also provides an antibody or antigen binding portion thereof that specifically binds to a Myc antigen, wherein the antibody or antigen binding portion thereof comprises a heavy chain variable region comprising an amino acid sequence that is at least 90% identical, for example, at least 90%, at least 91%, at least 92%, at least 93%, at least 94%, at least 95%, at least 96%, at least 97%, at least 98% or at least 99%, or 100% identical to SEQ ID NO: 65 and a light chain variable region at least 90% identical, for example, at least 90%, at least 91%, at least 92%, at least 93%, at least 94%, at least 95%, at least 96%, at least 97%, at least 98% or at least 99%, or 100% identical to SEQ ID NO: 66.

The disclosure also provides an antibody or antigen binding portion thereof that specifically binds to a Myc antigen, wherein the antibody or antigen binding portion thereof comprises a heavy chain variable region comprising a heavy chain CDR1 (HCDR1) of SEQ ID NO: 49 or a HCDR1 having no more than 1, 2, 3, or 4 amino acid substitutions as compared to SEQ ID NO: 49, a heavy chain CDR2 (HCDR2) of SEQ ID NO: 50 or a HCDR2 having no more than 1, 2, 3, or 4 amino acid substitutions as compared to SEQ ID NO: 50, and a heavy chain CDR3 (HCDR3) of SEQ ID NO: 51 or a HCDR3 having no more than 1, 2, 3, or 4 amino acid substitutions as compared to SEQ ID NO: 51; and wherein the antibody or antigen binding portion thereof comprises a light chain variable region comprising a light chain CDR1 (LCDR1) of SEQ ID NO: 52 or a LCDR1 having no more than 1, 2, 3, or 4 amino acid substitutions as compared to SEQ ID NO: 52, a light chain CDR2 (LCDR2) of SEQ ID NO: 53 or a LCDR2 having no more than 1, 2, 3, or 4 amino acid substitutions as compared to SEQ ID NO: 53, and a light chain CDR3 (LCDR3) of SEQ ID NO: 54 or a LCDR3 having no more than 1, 2, 3, or 4 amino acid substitutions as compared to SEQ ID NO: 54.

The disclosure also provides an antibody or antigen binding portion thereof that specifically binds to a Myc antigen, wherein the antibody or antigen binding portion thereof comprises a heavy chain variable region comprising a heavy chain CDR1 (HCDR1) of SEQ ID NO: 58 or a HCDR1 having no more than 1, 2, 3, or 4 amino acid substitutions as compared to SEQ ID NO: 58, a heavy chain CDR2 (HCDR2) of SEQ ID NO: 59 or a HCDR1 having no more than 1, 2, 3, or 4 amino acid substitutions as compared to SEQ ID NO: 59, and a heavy chain CDR3 (HCDR3) of SEQ ID NO: 60 or a HCDR3 having no more than 1, 2, 3, or 4 amino acid substitutions as compared to SEQ ID NO: 60; and wherein the antibody or antigen binding portion thereof comprises a light chain variable region comprising a light chain CDR1 (LCDR1) of SEQ ID NO: 61 or a LCDR1 having no more than 1, 2, 3, or 4 amino acid substitutions as compared to SEQ ID NO: 61, a light chain CDR2 (LCDR2) of SEQ ID NO: 62 or a LCDR2 having no more than 1, 2, 3, or 4 amino acid substitutions as compared to SEQ ID NO: 62, and a light chain CDR3 (LCDR3) of SEQ ID NO: 63 or a LCDR3 having no more than 1, 2, 3, or 4 amino acid substitutions as compared to SEQ ID NO: 63.

The disclosure also provides an antibody or antigen binding portion thereof that specifically binds to a Myc antigen, wherein the antibody or antigen binding portion thereof comprises a heavy chain variable region comprising a heavy chain CDR1 (HCDR1) of SEQ ID NO: 67 or a HCDR1 having no more than 1, 2, 3, or 4 amino acid substitutions as compared to SEQ ID NO: 67, a heavy chain CDR2 (HCDR2) of SEQ ID NO: 68 or a HCDR2 having no more than 1, 2, 3, or 4 amino acid substitutions as compared to SEQ ID NO: 68, and a heavy chain CDR3 (HCDR3) of SEQ ID NO: 69 or a HCDR3 having no more than 1, 2, 3, or 4 amino acid substitutions as compared to SEQ ID NO: 69; and wherein the antibody or antigen binding portion thereof comprises a light chain variable region comprising a light chain CDR1 (LCDR1) of SEQ ID NO: 70 or a LCDR1 having no more than 1, 2, 3, or 4 amino acid substitutions as compared to SEQ ID NO: 70, a light chain CDR2 (LCDR2) of SEQ ID NO: 71 or a LCDR2 having no more than 1, 2, 3, or 4 amino acid substitutions as compared to SEQ ID NO: 71, and a light chain CDR3 (LCDR3) of SEQ ID NO: 72 or a LCDR3 having no more than 1, 2, 3, or 4 amino acid substitutions as compared to SEQ ID NO: 72.

In examples, the antigen is Myc, for example, EQKLISEEDL (SEQ ID NO:146).

The amino acid residue sequences provided herein are set forth in single-letter amino acid code which can be used interchangeably with three-letter amino acid code. An amino acid refers to any monomer unit that can be incorporated into a peptide, polypeptide, or protein. The twenty natural or genetically encoded alpha-amino acids are as follows: alanine (Ala or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., Biochemistry, 5^{th} ed., Freeman and Company (2002). The term amino acid also includes unnatural amino acids, modified amino acids (e.g., having modified side chains and/or backbones), and amino acid analogs.

The terms identical or percent identity, in the context of two or more nucleic acids or polypeptide sequences, refer to two or more sequences or subsequences that are the same or have a specified percentage of nucleotides or amino acid residues that are the same (e.g., 90%, or 95% or greater identity over a specified region), when compared and aligned for maximum correspondence over a comparison window, or designated region as measured using one of the following sequence comparison algorithms or by manual alignment and visual inspection.

Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

As with all peptides, polypeptides, and proteins, including fragments thereof, it is understood that additional modifications in the amino acid sequence of the anti-tumor antigen-specific antibodies or antigen binding fragments thereof described herein (e.g., CD40 or CS1), the His antigen-specific antibodies or antigen binding fragments thereof described herein, and the Myc antigen-specific antibodies or antigen binding fragments thereof described herein, for example, in the heavy chain variable region and/or light chain variable region, can occur that do not alter the nature or function of the antibodies or antigen binding fragments thereof. Such modifications include conservative amino acids substitutions, such that each recited sequence optionally contains one or more conservative amino acid substitutions. The list provided below identifies examples of groups that contain amino acids that are conservative substitutions for one another; these groups are exemplary as other conservative substitutions are known to those of skill in the art.
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

By way of example, when an aspartic acid at a specific residue is mentioned, also contemplated is a conservative substitution at the residue, for example, glutamic acid. Non-conservative substitutions, for example, substituting a proline with glycine, are also contemplated.

In some instances, the affinity of anti-tumor antigen (e.g., CD40 or CS1) specific antibodies or antigen binding fragments thereof (or the His antigen-specific antibodies or antigen binding fragments thereof described herein and the Myc antigen-specific antibodies or antigen binding fragments thereof described herein) may be optimized through mutations to increase or decrease affinity as desired based on one or more of the known characteristics of the binding interaction with the cognant tumor antigen (or His or Myc antigens), the structure of either or both of the antibodies or fragments thereof, or the tumor antigen (or His or Myc antigens). In some instances, the mutations permit facile elution of purified antibodies or fragments thereof under desirable elution conditions during isolation and purification.

Methods of generating and screening for antibodies and antigen binding fragments thereof as provided in this disclosure are described in the Examples and are well-known in the art. Methods of further modifying antibodies for enhanced properties (e.g., enhanced affinity, chimerization, humanization) as well as generating antigen binding fragments, as described herein, are also well-known in the art.

The present disclosure also encompasses antibodies or fragments thereof that bind to the same epitope of anti-tumor antigens (e.g., CD40 or CS1) as the antibodies disclosed herein (or His or Myc antigens). Such antibodies can be identified using routine techniques known in the art, including, for example, competitive binding assays.

The present disclosure also encompasses bi-specific antibodies or fragments thereof that bind to the same epitope of anti-tumor antigens (e.g., CD40 or CS1) as the antibodies disclosed herein (or His or Myc antigens), as well as other antigens. Such antibodies can be identified using routine techniques known in the art, including, for example, competitive binding assays.

The term epitope, as used herein, means a component of an antigen capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

The present disclosure also provides chimeric antibodies. The term chimeric antibody refers to an antibody in which a component of the heavy and/or light chain is derived from a particular source or species, while the remainder of the heavy and/or light chain is derived from a different source or species.

The term “humanized antibody” refers to antibodies in which CDR sequences derived from the germline of another mammalian species, such as a mouse, have been grafted onto human framework sequences. The term “framework” refers to variable domain residues other than hypervariable region residues. The framework of a variable domain generally consists of four FR domains: FR1, FR2, FR3, and FR4. framework region modifications may be made within the human framework sequences.

In some embodiments, the antibody or antigen binding fragment thereof provided herein can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to as a half antibody). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv, for example), single variable domain antibodies, diabodies (Dab) (bivalent and bispecific), and chimeric (e.g., humanized) antibodies, which may be produced by the modification of whole antibodies or synthesized de novo using recombinant DNA technologies. These functional antibody fragments retain the ability to selectively bind with their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains.

As used herein, the term monoclonal antibody refers to an antibody from a population of substantially homogeneous antibodies. A population of substantially homogeneous antibodies comprises antibodies that are the same or substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target, for example, by affinity maturation, to humanize the antibody, to improve its production in cell culture, and/or to reduce its immunogenicity in a subject.

Antigen binding fragments of an antibody molecule are well known in the art, and include, for example, (i) a Fab fragment, a monovalent fragment consisting of the VL, VH, CL and CHi domains; (ii) a F(ab′)2 fragment, a bivalent fragment comprising two Fab fragments linked by a disulfide bridge at the hinge region; (iii) a Fd fragment consisting of the VH and CHi domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv) (see e.g., Bird et al. (1988) Science 242:423-426; Huston et al. (1988) Proc. Natl. Acad. Sci. USA 85:5879-5883); (viii) a single domain antibody. These antibody fragments are obtained using conventional techniques known to those skilled in the art, and the fragments are screened for utility in the same manner as are intact antibodies.

In certain embodiments, antibodies and antibody compositions as provided herein are distinguishable from naturally occurring antibodies and compositions in one or more respects. Such distinguishable antibodies and compositions may be referred to as “synthetic,” or may be identified by the proviso that the antibody or composition “is not naturally occurring” or affirmatively as “non-naturally occurring.” As used herein the terms “corresponding antibody,” and “corresponding to” describes the relationship between (1) an antibody characterized by six specific CDR sequences of the antibodies described in the Examples below and (2) a synthetic antibody comprising the same six CDR sequences. Synthetic antibodies of this disclosure may differ in structure from naturally occurring antibodies with the same CDRs. That is, synthetic antibodies identified by specified CDRs may be structurally different from antibodies comprising the specified CDRs that are described in the Examples below. Possible differences for synthetic antibodies include variable region sequences that differ corresponding naturally occurring antibodies, different light chain sequences (i.e. lambda type instead of kappa type or vice versa), different isotypes, different allotypes, and different constant domain variants. These differences are discussed in more detail below. In some embodiments, the synthetic antibody is an engineered polypeptide, also referred to as a recombinant polypeptide, that is made using conventional protein and antibody engineering molecular biology, chemical, and biochemical methods as described below, including, but not limited to, those described in the Examples below.

In one approach, an antibody heavy chain of an antibody as provided in this disclosure comprises one or more CDRs of a clone described in Table 1.

In some embodiments, the antibody comprises a heavy chain variable region sequence and a light chain variable region sequence that are derived from an immunoglobulin producing human B cell, and further comprises a kappa or lambda light chain constant region. In some embodiments, the light chain constant region (kappa or lambda) is from the same type of light chain (i.e., kappa or lambda) as the light chain variable region that was derived from the immunoglobulin producing human B cell; as a non-limiting example, if an IgE-producing human B cell comprises a kappa light chain, then the antibody that is produced can comprise the light chain variable region from the IgE-producing B cell and further comprises a kappa light chain constant region.

In some embodiments, the antibody comprises a heavy chain variable region sequence and a light chain variable region sequence that are derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the antibody comprises a native (i.e., wild-type) human IgG, IgA, IgM, or IgD constant region. In some embodiments, the antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., J Biol Chem, 2015, 280:5462-5469.

In some embodiments, the antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. In some embodiments the antibody comprises a human IgM constant region, human IgD constant region, human IgG constant region that is derived from IgG1, IgG2, IgG3, or IgG4, or human IgA constant region that is derived from IgA1 or IgA2. In some embodiments, the antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten or more modifications (e.g., amino acid substitutions). In some embodiments the constant regions include variations (e.g., one, two, three, four, five, six, seven, eight, nine, ten or more amino acid substitutions) that reduce effector function.

Synthetic antibodies of this disclosure may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-life, modulate co-engagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fc engineering to modulate antibody effector functions” Protein Cell 2018, 9(1):63-73; Schlothauer, 2016, “Novel human IgG1 and IgG4 Fc-engineered antibodies with completely abolished immune effector functions,” Protein Engineering, Design and Selection 29(10):457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

In some embodiments, the heavy chain variable region and/or the light chain variable region of the antibody has an identical sequence to the heavy chain variable region and/or the light chain variable region encoded by the immunoglobulin producing single B cell from the human subject having a cancer comprising cells overexpressing anti-tumor antigens (or His or Myc antigens) or otherwise having elevated anti-tumor antigen activity (e.g., CD40 or CS1). In some embodiments, the heavy chain variable region and/or the light chain variable region of the antibody comprises one or more modifications, e.g., amino acid substitutions, deletions, or insertions.

The heavy chain variable region sequence and/or light chain variable region sequence of an antibody described herein can be engineered to comprise one or more variations in the heavy chain variable region sequence and/or light chain variable region sequence. In some embodiments, the engineered variation(s) improves the binding affinity of the antibody for the anti-tumor antigen (e.g., CD40 or CS1), His antigens, or Myc antigens.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., PLoS Negl Trop Dis, 2017, 11:e0005655, doi:10.1371/journal.pntd.0005655.

The amino acid sequences of the CDRs and framework regions can be determined using various well-known definitions in the art, e.g., Kabat, Chothia, international ImMunoGeneTics database (IMGT), AbM, and observed antigen contacts (“Contact”) (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al, Sequences of Proteins of Immunological Interest, 5^{th} Ed. Public Health Service, National Institute of Health, Bethesda, MD (1991)). Chothia CDRs are determined as defined by Chothia (see, e.g., Chothia and Lesk J. Mol. Biol. 196:901-917 (1987)). In some embodiments, CDRs are determined by a combination of Kabat, Chothia, and/or Contact CDR definitions. In some embodiments, CDRs are determined by a combination of Kabat, Chothia, AbM, and/or Contact CDR definitions. The CDR sequences shown in Table 1 are determined using Kabat.

In some embodiments, affinity maturation is used to engineer further mutations that enhance the binding affinity of the antibody for the anti-tumor antigen (e.g., CD40 or CS1, or His or Myc antigens, or enhance the cross-reactivity of the antibody for a second cancer-related antigen that is not the anti-tumor antigen (e.g., CD40 or CS1). Methods for performing affinity maturation are known in the art. See, e.g., Renaut et al., Methods Mol Biol, 2012, 907:451-461.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca and guanaco) or other species besides Camelidae.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is screened, and in accordance with the methods described herein, against a target antigen of interest to identify those variants having optimal selectivity and affinity for the target antigen. See, e.g., Hey et al. (2005) TRENDS Biotechnol 23(10):514-522.

Synthetic antibodies of this disclosure may differ from naturally occurring compositions in at least one or more of the following respects: (i) composition comprises antibodies that are purified, i.e., separated from tissue or cellular material with which they are associated in the human body, and optionally in an manufactured excipient or medium; and/or (ii) antibody compositions according to the present disclosure contain a single species of antibody (are monoclonal) such that all antibodies in the composition have the same structure and specificity.

### E. Expression and Purification of Antibodies

The anti-tumor antigen-specific antibodies or antigen binding fragments thereof (e.g., CD40 or CS1), or anti-His or anti-Myc antigen-specific antibodies or antigen binding fragments thereof, disclosed herein may be produced by recombinant expression in a human or non-human cell. For antibodies or antigen-binding fragments thereof comprising a peptide tag, a nucleotide sequence encoding the anti-tumor antibody (or antigen binding fragment thereof), the anti-Myc antibody (or antigen binding fragment thereof), or the anti-His antibody (or antigen binding fragment thereof) can be cloned into an expression vector with an in-frame peptide tag (e.g., a Myc tag or a His tag).

Synthetic antibody-producing cells include non-human cells expressing heavy chains, light chains, or both heavy and light chains; human cells that are not immune cells; heavy chains, light chains, or both heavy and light chains; and human B cells that produce heavy chains or light chains, but not both heavy and light chains. Synthetic antibodies of this disclosure may be heterologously expressed, in vitro or in vivo, in cells other than human B cells, such as non-human cells and human cells other than B cells, optionally other than immune cells, and optionally in cells other than cells in a B cell lineage.

The anti-tumor antigen-specific antibodies or antigen binding fragments thereof (or His or Myc antigen-specific antibodies or antigen binding fragments thereof) disclosed herein can be produced using a variety of techniques known in the art of molecular biology and protein chemistry. For example, a nucleic acid encoding the antibody or antigen binding fragment thereof can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

In vitro methods are also suitable for preparing monovalent antibodies. or fragments thereof. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or fragments is to link two or more peptides or polypeptides together by protein chemistry techniques (or recombinant DNA techniques). For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides, or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Recombinant techniques can also be used to modify antibodies or antigen binding fragments thereof. For example, amino acids found to not contribute to either the activity or the binding specificity or affinity of the antibody can be deleted without a loss in the respective activity. Insertions, deletions, substitutions, or other selected modifications of particular regions or specific amino acids residues can also be made (and are contemplated by the present disclosure), provided the activity of the fragment is not significantly altered or impaired compared to the non-modified antibody, or antigen binding fragment thereof can be made. Such methods are readily apparent to a skilled practitioner in the art and can include site specific mutagenesis of the nucleic acid encoding the antibody or fragment thereof. (Zoller et al., Nucl. Acids Res. 10:6487-500 (1982)).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### F. Modification of Antibodies

Any of the anti-tumor antibodies or antigen binding fragments thereof described herein can be modified, in particular, by fusion with a peptide tag (i.e., a His or a Myc tag). In embodiments as described herein, antibodies or antigen binding fragments thereof are not chemically conjugated to a small molecule (i.e., a non-peptide or non-polypeptide molecule with a molecular weight of less than about 1000 daltons or less than about 2500 daltons). In some embodiments, antibodies or antigen binding fragments thereof are not conjugated to cotinine. In some embodiments, antibodies or antigen binding fragments thereof are not conjugated to a chemical tag.

In embodiments, a Myc tag as described herein can have a polypeptide sequence of EQKLISEEDL (SEQ ID NO:146).

In embodiments, a His tag as described herein comprises a polypeptide having 2 or more, 3 or more, 4 or more, 5 or more, 6 or more, 7 or more, 8 or more, 9 or more, or 10 or more histidine residues linked by peptide bonds. In some embodiments, the number of histidines in the His tag is in the range of 2 to 15, for example, 3 to 10, 4 to 8. In embodiments, a His tag comprises a polypeptide with 6 histidine residues.

The modifications can be covalent or non-covalent modifications and can include one or more amino acid substitutions that change the properties of the antigen-specific antibodies or antigen binding fragments thereof. Such modifications can be introduced into the antibodies or antigen binding fragments by, e.g., reacting targeted amino acid residues of the polypeptide with an organic derivatizing agent that can react with selected side chains or terminal residues, or base-pair mutations in a nucleotide sequence encoding the antigen-specific antibodies or antigen binding fragments thereof. Suitable sites for modification can be chosen using any of a variety of criteria including, e.g., structural analysis or amino acid sequence analysis of the antibodies or fragments. In some instances, the anti-tumor antigen-specific antibodies or antigen binding fragments (or His or Myc antigen-specific antibodies or antigen binding fragments) may be labeled by a variety of means for use in diagnostic and/or pharmaceutical applications.

In some embodiments, the antibodies or antigen binding fragments thereof described herein may have a modification comprising one or more amino acid substitutions that provide reduced hydrophobicity and reduce the potential for aggregation, thereby improving the binding or therapeutic capacity of an antibody or an antigen-binding fragment thereof. Such amino acid substitutions can be introduced by changing one or more nucleotides in the polynucleotide encoding the antibody or antigen-binding fragment such that the triplet codon for the amino acid residue position where the modification is to be introduced is replaced with the triplet codon encoding the amino acid substitution. In some embodiments, the modification may comprise a single amino acid substitution. In some embodiments, the modification may comprise multiple amino acid substitutions. In some embodiments, the modification may be a substitution of 1 amino acid, 2 amino acids, 3 amino acids, 4 amino acids, or 5 amino acids. In some embodiments, the amino acid substitution may be located in a CDR. For example, the amino acid substitution may be located in heavy chain variable region CDR2 sequence of an antibody or antigen binding fragment provided in this disclosure. In some embodiments, the amino acid substitution can be at the 5^{th} or 6^{th} position (from N terminus to C terminus) of the heavy chain variable region CDR2 sequence of an antibody or antigen binding fragment provided in this disclosure. In some embodiments, the amino acid substitution can be at the 5^{th} or 6^{th} position (from N terminus to C terminus) of the heavy chain variable region CDR2 sequence of an antibody or antigen binding fragment provided in this disclosure. In some embodiments, the amino acid substitution can be at the 5^{th} or 6^{th} position (from N terminus to C terminus) of the heavy chain variable region CDR2 sequence of an antibody or antigen binding fragment provided in this disclosure. In some embodiments, the amino acid mutations may include aspartic acid to serine modification. In some embodiments, the modification may include an alanine to glycine. One skilled in the art may understand that any modification to the antibody sequence that reduced the hydrophobicity of an amino acid sequence may fall under the scope of such modifications. Described above modifications are provided herein not to limit the scope of the invention but merely provided as examples of modifying an amino acid sequence for altering the hydrophobicity.

In some embodiments, the antibodies or antigen binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK)), polyhistidine (6-His; HHHHHH (SEQ ID NO: 118)), hemagglutinin (HA; YPYDVPDYA, glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase. Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵I]mIPNHS), which binds to free amino groups to form meta-iodophenyl (mIP) derivatives of relevant proteins (see, e.g., Rogers et al. (1997) J Nucl Med 38:1221-1229) or chelate (e.g., to DOTA or DTPA), which is in turn bound to the protein backbone. Methods of conjugating the radioactive labels or larger molecules/chelates containing them to the antibodies or antigen binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisić et al. (2010) Int J Pharm 387(1-2):110-119). The stabilization moiety can improve the stability, or retention of, the antibody (or fragment) by at least 1.5 (e.g., at least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### IV. Chimeric Antigen Receptors

Also provided herein are chimeric antigen receptors comprising an antigen recognition domain (e.g., an antibody or an antigen-binding fragment thereof) that recognizes (or targets) and specifically binds to a peptide tag (e.g., in some embodiments, a His tag (e.g., HHHHHH (SEQ ID NO: 118)) or a myc tag (e.g., EQKLISEEDL (SEQ ID NO:146)). Chimeric antigen receptors (CARs, also known as chimeric T cell receptors) as described herein are designed to be expressed in host effector cells, e.g., T cells, NK cells, and macrophages, and to induce an immune response against anti-tumor antigens and cells expressing them (in particular, tumor cells) when engaged with an antitumor antibody as described herein (also referred to as an “adaptor”) that specifically binds to the anti-tumor antibody and is fused with a peptide tag. For example, adoptive T cell immunotherapy, in which a patient's own T lymphocytes are engineered to express CARs, has shown great promise in treating hematological malignancies. CARs can be engineered and used as described, for example, in Sadelain et al., 2013, Cancer Discov. 3:388-398. A CAR typically comprises an extracellular target-binding module (also referred to herein as “extracellular targeting domain,” “antigen recognition domain,” or “antibody domain”), a transmembrane (TM) domain, and an intracellular signaling domain (ICD, also referred to herein as a “signal transduction domain”). The CAR domains can be joined via flexible hinge and/or spacer regions, for example, between the extracellular targeting domain and the transmembrane domain and/or the transmembrane domain and the intracellular signaling domain. The extracellular target-binding module generally comprises an antibody or antigen binding fragment thereof. In some embodiments, multiple binding specificities can be included in the extracellular target-binding module. For example, multiple antibodies or antigen binding fragments thereof that target different antigens can be included to produce bi-specific, tri-specific, or quad-specific CARs. Transmembrane (TM) domains are primarily considered a structural requirement, anchoring the CAR in the cell membrane, and are most commonly derived from molecules regulating T cell function, such as CD8 and CD28. The intracellular module typically consists of the T cell receptor CD3ζ chain and one or more costimulatory domains from either the Ig (CD28-like) or TNF receptor (TNFR) superfamilies. CARs containing either CD28 or 4-1BB costimulatory domains have been the most widely used, to date, and both have yielded dramatic responses in clinical trials. CAR domains are discussed in more detail below.

Provided herein are chimeric antigen receptors (CARs) comprising: (a) an extracellular target-binding domain comprising an anti-peptide tag antibody or antigen binding portion thereof (e.g., an anti-Myc or anti-His antibody or antigen binding portion or fragment thereof); (b) a transmembrane domain; and (c) a signal transduction domain. In some embodiments, CARs can further comprise one or more hinge domains or linkers that can join domains of the CAR and provide steric flexibility (i.e., flexibility of movement in the three-dimensional space).

In an embodiment, a backbone of a CAR construct as described herein can comprise a mouse CD28 extracellular, transmembrane, and cytoplasmic domain linked to mouse CD3zeta cytoplasmic domain (GenBank HM754222.1) in addition to an antigen-recognition domain that recognizes a peptide tag (e.g. a Myc tag or a His tag as described herein). In some embodiments, a CAR construct can comprise a anti-His murine BBz CAR ORF consists of mouse CD8 leader, anti-6×His scFv (or anti-Myc scFv), and the 4-1BB-based CAR backbone (mouse CD8 extracellular and transmembrane domain, human 4-1BB cytoplasmic domain linked to mouse CD3zeta cytoplasmic domain). In an embodiment, a CAR construct as described herein a human GM-CSFR leader, human codon-optimized anti-cotinine scFv and the human CD28-based CAR backbone (human CD28 extracellular and transmembrane domain linked to human CD3zeta cytoplasmic domain (from GenBank HM852952.1), in addition to an antigen-recognition domain that recognizes a peptide tag (e.g. a Myc tag or a His tag as described herein). In some embodiments, a CAR construct as described herein can comprise an anti-His (or anti-Myc) human CAR ORF comprising a human CD8 leader, human codon-optimized anti-6×His scFv (or anti-Myc scFv) and the human 4-1BB-based CAR backbone (human CD8 extracellular, transmembrane domain and human 4-1BB cytosolic domain linked to human CD3zeta cytoplasmic domain).

In some embodiments, an anti-His CAR provided herein comprises an amino acid sequence selected from SEQ ID NO: 202 and 207. In some embodiments, an anti-His CAR provided herein comprises an amino acid sequence selected from SEQ ID NO: 219 and 224. In some embodiments, the anti-His CAR comprises a sequence having at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, or at least 99% to any of SEQ ID NOs 202, 207, 219, or 224.

In some embodiments, an anti-Myc CAR provided herein comprises an amino acid sequence selected from any one of SEQ ID NO: 230 and 235. Various structural components of the exemplary anti-Myc CARs are shown in Table 4. In some embodiments, the anti-Myc CAR comprises a sequence having at least 70%, at least 75%, at least 80%, at least 85%, at least 88%, at least 89%, at least 90%, at least 91%, at least 92%, at least 93%, at least 94%, at least 95%, at least 96%, at least 97%, at least 98%, or at least 99% to any of SEQ ID NOs 230 and 235.

Various structural components of exemplary CARs are further described below and also shown in Table 4 (murine CARs) and Table 5 (human CARs).

### A. Extracellular Targeting Domain

The extracellular target-binding domain (also referred to herein as an “antibody domain,” “antibody domain,” or “antigen recognition domain”) of a CAR may comprise an antibody or an antigen-binding fragment thereof that specifically binds to or otherwise recognizes a peptide tag. In some embodiments, the extracellular target-binding domain of the CARs provided herein specifically binds to a His tag (e.g., HHHHHH (SEQ ID NO:118)) or a myc tag (e.g., EQKLISEEDL (SEQ ID NO: 146)). Examples of antibodies and antigen binding-fragments thereof that can specifically bind to His or Myc and be incorporated into antigen recognition domains (for example, by translational fusion with other hinge, linker, transmembrane, and intracellular domain sequences).

In certain embodiments, the extracellular target-binding domain targeting a peptide tag can be a single-chain variable fragment derived from an antibody (scFv), a tandem scFv, a single-domain antibody fragment (V_{H}Hs or sdAbs), a single domain bispecific antibody (BsAbs), an intrabody, a nanobody, an immunokine in a single chain format, and Fab, Fab′, or (Fab′)₂ in a single chain format. In other embodiments, the extracellular target-binding domain can be an antibody moiety that comprises covalently bound multiple chains of variable fragments. In some embodiments, the extracellular target-binding domain comprises any of the antibodies or antigen-binding portions thereof described above.

In some embodiments, an anti-His CAR provided herein comprises an extracellular targeting domain comprising: a HCDR1 of any one of SEQ ID NOS: 31 and 40, a HCDR2 of any one of SEQ ID NOS: 32 and 41, a HCDR3 of any one of SEQ ID NOS: 33 and 42, an LCDR1 of any one of SEQ ID NOS: 34 and 43, an LCDR2 of any one of SEQ ID NOS: 33 and 44, an LCDR3 of any one of SEQ ID NOS: 36 and 45; and the FW regions in the VH region are at least 80% identical to the FW regions present in the VH region of SEQ ID Nos. 29 or 38, and wherein the FW regions in the VL region are at least 80% identical to the FW regions present in the VL region of SEQ ID NO: 30 or 39. In some embodiments, an anti-His CAR can comprise an extracellular targeting domain comprising an anti-His scFV having a sequence at least 80% identical to one of 28, 37, 203, 208, 220, or 225.

In some embodiments, an anti-Myc CAR provided herein comprises an extracellular targeting domain comprising: HCDR1 of any one of SEQ ID NOS: 49, 58, and 67, an HCDR2 of any one of SEQ ID NOS: 50, 59, and 68, an HCDR3 of any one of SEQ ID NOS: 51, 60, and 69, an LCDR1 of any one of SEQ ID NOS: 52, 61, and 70, an LCDR2 of any one of SEQ ID NOS: 53, 62, and 71, an LCDR3 of any one of SEQ ID NOS: 54, 63, and 72; and the FW regions in the VH region are at least 80% identical to the FW regions present in the VH region of SEQ ID Nos. 47, 56, and 65, and wherein the FW regions in the VL region are at least 80% identical to the FW regions present in the VL region of SEQ ID NO: 48, 57, and 66. In some embodiments, an anti-Myc CAR can comprise an extracellular targeting domain comprising an anti-Myc scFV having a sequence at least 80% identical to one of 46, 55, 64, 230, 236, or 242.

In some embodiments, the extracellular target-binding domain recognizes and specifically binds to a His tag (i.e., a peptide sequence comprising a His multimer, such as a 6×His multimer having the sequence HHHHHH (SEQ ID NO:118), or other His multimer comprising 2 or 3 or 4 or 5 or 7 or 8 or 9 or 10 or 11 or 12 His residues in sequence). In some embodiments, the extracellular target-binding domain targeting a His tag comprises a scFv comprising a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 29 or 30 and a light chain variable region comprising an amino acid sequence that is at least 90% identical (for example, at least 90%, 91%, 92%, 93%, 94%, 95%, 96%, 97%, 98% or 99% identical) to any of SEQ ID NOs: 30 or 39. In some embodiments, the scFv targeting a His tag comprises a linker polypeptide between the heavy chain and light chain sequences. In some embodiments, the CAR comprises a scFv targeting a His tag and where the scFv comprising an amino acid sequence that is at least 90% identical to SEQ ID NOs: 28 or 37. In certain embodiments, the extracellular target-binding domain comprises at least one or more VH CDRs of any one of SEQ ID NOs: 31-33 and 40-42 (or any other VH CDR sequence as described herein) (or any other VH CDR sequence as described herein, or any combination of any CDR1, CDR2, CDR3 thereof). In certain embodiments, the extracellular target-binding domain targeting a His tag comprises at least one or more VL CDRs of any one of SEQ ID NOs: 34-36 and 43-45 (or any other VL CDR sequence as described herein, or any combination of any CDR1, CDR2, CDR3 thereof). In some embodiments, these anti-His CARs can also comprise one or more hinge domains linking two domains of the CAR together.

In some embodiments, the extracellular target-binding domain recognizes and specifically binds to a myc tag (i.e., a peptide sequence comprising a myc multimer, such as myc multimer having the sequence EQKLISEEDL (SEQ ID NO: 146)). In some embodiments, the extracellular target-binding domain targeting a Myc tag comprises a scFv comprising a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 47, 56, or 65 and a light chain variable region comprising an amino acid sequence that is at least 90% identical (for example, at least 90%, 91%, 92%, 93%, 94%, 95%, 96%, 97%, 98% or 99% identical) to any of SEQ ID NOs: 48, 57, or 66. In some embodiments, the scFv targeting a Myc tag comprises a linker polypeptide between the heavy chain and light chain sequences. In some embodiments, the CAR comprises a scFv targeting a Myc tag, where the scFc comprises an amino acid sequence that is at least 90% identical to SEQ ID NOs: 46, 55, or 64. In certain embodiments, the extracellular target-binding domain comprises at least one or more VH CDRs of any one of SEQ ID NOs:49-51, 58-60, and 67-69 (or any other VH CDR sequence as described herein) (or any other VH CDR sequence as described herein, or any combination of any CDR1, CDR2, CDR3 thereof). In certain embodiments, the extracellular target-binding domain targeting a Myc tag comprises at least one or more VL CDRs of any one of SEQ ID NOs: 52-54, 61-63. Or 70-72 (or any other VL CDR sequence as described herein, or any combination of any CDR1, CDR2, CDR3 thereof). In some embodiments, these anti-Myc CARs can also comprise one or more hinge domains linking two domains of the CAR together. In certain embodiments, CARs according to the present disclosure can comprise one or more at least one hinge, linker domain, or both domain sequences as set forth in Table 4 or Table 5.

### B. Transmembrane Domain

CARs as described herein that target a peptide tag can also comprise a transmembrane domain. In certain aspects, the transmembrane domain is a short hydrophobic region that can anchor the CAR in the cell membrane and helps to stabilize its structure. The transmembrane domain of a CAR provided herein may be derived from either a natural or from a synthetic source.

Where the source is natural, the domain may be derived from any membrane-bound or transmembrane protein. In some embodiments, the transmembrane domain is derived from (i.e., comprises at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In embodiments, the transmembrane domain is a CD8a or CD28 transmembrane domain. In some embodiments, a transmembrane domain can be chosen based on, for example, the nature of the various other proteins or trans-elements that bind the transmembrane domain or the cytokines induced by the transmembrane domain. In some embodiments, the transmembrane domain comprises a CD8α transmembrane domain. When a transmembrane domain is synthetic, it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of a CAR described herein. In some embodiments, the linker is a glycine-serine doublet.

In certain embodiments, CARs according to the present disclosure can comprise one or more transmembrane domain sequences as set forth in Table 4 or Table 5.

### C. Intracellular Signaling Domain

The intracellular signaling domain of the CAR targeting a peptide tag is responsible for activation of at least one of the normal effector functions of the immune cell in which the CAR has been placed in or is designed to be placed in. An effector function of a T cell may be, for example, cytolytic activity or helper activity, including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CARs targeting a peptide tag provided herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability. In certain aspects, the intracellular signaling domain for use in CARs targeting a peptide tag as described herein is a region of the CAR that can be located inside a T cell and is responsible for transmitting signals to the cell. There can be one or more signaling domains, which can vary depending on the design of the CAR. In certain aspects, the signaling domain is the CD3ζ domain, which is found in the T cell receptor (TCR) complex and is responsible for activating T cells, and one or more co-stimulatory domains, such as CD28, 4-1BB, or OX40. These co-stimulatory domains can enhance the activation and proliferation of CAR-T cells, as well as improve their persistence and anti-tumor activity.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. In some embodiments, the CARs described herein comprise one or more ITAMs.

Examples of ITAM containing primary signaling sequences that are of particular use in the invention include those derived from TCRζ, FcRγ, FcRβ, CD3γ, CD3δ, CD3ε, CD3ζ, CD5, CD22, CD79a, CD79b, and CD66d. In some embodiments, an ITAM containing primary signaling sequence is derived from CD3ζ.

In some embodiments, the CAR targeting a peptide tag comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR of the invention. In some embodiments, the intracellular signaling domain of a CAR provided herein comprises a CD3ζ primary intracellular signaling sequence and a 4-1BB costimulatory signaling sequence.

The CARs targeting a peptide tag provided herein may include additional elements, such a signal peptide to ensure proper export of the fusion protein to the cells surface, a leader sequence, and a hinge domain that imparts flexibility to the recognition region and allows strong binding to the targeted moiety. In some embodiments, a spacer domain may be present between any of the domains of the CAR. The spacer domain can be any polypeptide that functions to link two parts of the CAR. A spacer domain may comprise up to about 300 amino acids, including for example about 10 to about 100, or about 25 to about 50 amino acids. Methods of identifying and selecting suitable spacer domains are known. In certain aspects, signal peptides can be used to direct the expression of the CAR to the cell surface. Embodiments of signal peptides that can be utilized in CAR-T cell design according to the present disclosure include (without intending to be limiting) the human CD8a signal peptide, the Igκ signal peptide, and the CMV immediate-early promoter/enhancer signal peptide. In certain aspects, hinge domains can be flexible regions that connect the antigen-recognition domain and the intracellular signaling domains of the CAR. They can have a significant impact on the function and efficacy of the CAR-T cells, by affecting the flexibility and stability of the receptor, as well as its ability to recognize and engage with antigens. Embodiments of hinge domains that can be utilized in CAR-T cell designs according to the present disclosure include the IgG4 hinge, the CD8α hinge, and the CD28 hinge.

In some embodiments, the CAR targeting a peptide tag comprises at least one anti-peptide tag scFv domain as described in this disclosure; at least one hinge and/or transmembrane domain comprising CD8a, CD28 and/or, IgG4 Fc-CD28 (as the hinge and transmembrane domain); at least one costimulatory domain comprising CD28, 4-1BB, and/or OX40 (e.g., as described in this disclosure); and a CD3ζ signaling domain (e.g., as described in this disclosure).

In some embodiments, a CAR targeting a His tag provided herein comprises a sequence that is at least 90% identical (for example, at least 90%, 91%, 92%, 93%, 94%, 95%, 96%, 97%, 98% 99%, or 100% identical identical) to SEQ ID NOs: 28 or 37.

In some embodiments, a CAR targeting a Myc tag provided herein comprises a sequence that is at least 90% identical (for example, at least 90%, 91%, 92%, 93%, 94%, 95%, 96%, 97%, 98% 99%, or 100% identical identical) to SEQ ID NOs: 46, 55, or 64.

In certain embodiments, CARs according to the present disclosure can comprise an intracellular signaling domain comprising one or more sequences as set forth in Table 4 or Table 5.

### V. Antibody Expression and Purification, Nucleic Acids, Vectors, and Cells

The anti-tumor antibodies, antigen binding fragments thereof (e.g., anti-CD40 or anti-CS1), anti-His antibodies, antigen binding fragments thereof, anti-Myc antibodies, antigen binding fragments thereof, and molecules comprising such antibodies and antigen binding fragments thereof discussed above, as well as anti-peptide tag CARs, may be produced by recombinant expression in a human or non-human cell. Synthetic antibody-producing cells include non-human cells expressing heavy chains, light chains, or both heavy and light chains; human cells that are not immune cells expressing heavy chains, light chains, or both heavy and light chains; and human B cells that produce heavy chains or light chains, but not both heavy and light chains. Synthetic antibodies of this disclosure may be heterologously expressed, in vitro or in vivo, in cells other than human B cells, such as non-human cells and human cells other than B cells, optionally other than immune cells, and optionally in cells other than cells in a B cell lineage.

The anti-tumor antibodies and antigen binding fragments thereof, as well as anti-peptide tag CARs (i.e., CARs with an antigen recognition domain targeting and specifically binding to a His or Myc peptide tag), and molecules comprising them described herein can be produced using a variety of techniques known in the art of molecular biology and protein chemistry. For example, a nucleic acid encoding the antibody or antigen binding fragment thereof or anti-peptide tag CAR can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

### A. Expression Vectors

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments or anti-peptide tag CARs thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof or anti-peptide tag CAR can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof and anti-peptide tag CARs can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

There area number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein.

As used herein, plasmid or viral vectors are agents that transport the disclosed nucleic acids into the cell without undesired degradation and include a promoter yielding expression of the nucleic acid molecule and/or adapter polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors. In some instances, the nucleic acid molecules encoding the anti-tumor antigen-specific antibody or antigen binding fragment thereof (or His antigen-specific antibody or antigen binding fragment thereof, or Myc antigen-specific antibody or antigen binding fragment thereof) can be delivered via virus-like particles.

Non-viral based delivery methods, can include expression vectors comprising nucleic acid molecules and nucleic acid sequences encoding the adapter polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, WI), Clonetech (Pal Alto, CA), Stratagene (La Jolla, CA), and Invitrogen/Life Technologies (Carlsbad, CA). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters (e.g., β-actin promoter or EF1α promoter), or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the beta-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, CT) sequences typically are expressed as a fusion with the encoded polypeptide. Such tags can be inserted anywhere within the polypeptide including at either the carboxyl or amino terminus.

### B. Nucleic Acids

Also provided herein are nucleic acid molecules encoding anti-tumor antibody or antigen binding portion thereof (e.g., anti-CD40 or anti-CS1), a His antibody or antigen binding portion thereof, a Myc antibody or antigen binding portion thereof. Also provided herein are nucleic acid molecules encoding anti-peptide tag CAR that binds specifically to a peptide antigen as described in this disclosure (e.g., a His or Myc peptide antigen). In some embodiments, the nucleic acid molecules encoding the anti-tumor antibody or antigen binding portion thereof are operably linked to a promoter capable of directing expression in a bacterial cell or a eukaryotic cell.

In some embodiments, the nucleic acid molecules encoding the Myc antibodies, His antibodies, or antigen binding fragments thereof (or CARs comprising such) are synthetic sequences designed for expression in a host cell (for example, a human cell).

In some embodiments, the nucleic acid molecules encoding the Myc antibodies, His antibodies, or antigen binding fragments thereof (or CARs comprising such) are operably linked to a promoter capable of directing expression in a bacterial cell or a eukaryotic cell.

Also provided herein are DNA constructs comprising a promoter that drives expression in a host cell operably linked to a recombinant nucleic acid molecule comprising a nucleotide sequence that encodes an anti-tumor antigen or peptide tag specific antibody or antigen binding fragment thereof or CAR.

Also provided herein are vectors, discussed further below, comprising a DNA construct comprising a promoter that drives expression in a host cell operably linked to a recombinant nucleic acid molecule comprising a nucleotide sequence that encodes an anti-tumor antigen or peptide tag specific antibody or antigen binding fragment thereof or CAR.

Also provided herein are host cells, including bacterial host cells and eukaryotic host cells, comprising a recombinant nucleic acid molecule encoding an anti-tumor antigen or peptide tag specific antibody or antigen binding fragment thereof or CAR.

### C. CAR-Expressing Immune Cell Hosts

Also provided herein are immune cells (e.g., T cells) expressing any of the CARs described herein. In some embodiments, the immune cell expresses the CAR on its surface, in particular, the antigen recognition domain. In some embodiments, the immune cell comprises a nucleic acid encoding the CAR, wherein the CAR is expressed from the nucleic acid and localized (at least partially) to the immune cell surface. In some embodiments, the immune cell is a B-lymphocyte, T-lymphocyte, thymocyte, dendritic cell, natural killer (NK) cell, natural killer T (NKT) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In some embodiments, the immune cell is a T cell. In some embodiments, the T cell is a cytotoxic T cell, a helper T cell, a natural killer T cell, a suppressor T cell, a CD8⁺ T cell, a CD4⁺ T cell, a CD8⁺/CD4⁺ T cell, a CD8−/CD4− T cell (CD4 CD8 double negative T cell), αβ T cell, γδ T cell, or a T-regulatory (T-reg) cell.

In some embodiments, immune cells expressing a CAR provided herein are obtained from a subject. Where the immune cells are used to treat (e.g., according to the treatment methods described herein below) the same subject from which they are obtained, they are referred to as autologous cells. Where they are obtained from a different subject, they are referred to as heterologous cells or allogeneic (if derived from the same species as the subject). Immune cells can be isolated from peripheral blood using techniques well known in the art, include Ficoll density gradient centrifugation followed by negative selection to remove undesired cells. In some embodiments, heterologous immune cells useful for the methods provided herein comprise allogeneic T cells, as described in, e.g., Bedoya et al., 2021, Front. Immunol. 12:640082.

### D. Preparation of Expressed Antibodies and Antigen-Binding Fragments Thereof

In vitro methods are also suitable for preparing monovalent antibodies or antigen binding fragments thereof or CARs. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or fragments or CARs is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

### E. Purification of Expressed Antibodies and Antigen-Binding Fragments Thereof

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### VI. Systems, Kits, and Packaging

Described Herein are Switchable Chimeric Antigen Receptor Immune Cell Systems comprising a chimeric antigen receptor immune cell and an anti-tumor antibody conjugated to a peptide tag. The chimeric antigen receptor immune cell comprises a chimeric antigen receptor (CAR), and wherein the CAR comprises an antigen recognition domain that recognizes the peptide tag. Nonlimiting examples of the peptide tag include His tag and Myc tag. Nonlimiting examples of the anti-tumor antibodies include anti-CD40, anti-CS1 antibody, and anti CD3 antibody.

Described herein are systems comprising an immune cell (e.g., a T-cell, B-cell, NK cell, NKT cell, or macrophage) expressing CARs as described herein (i.e., CARs that target and specifically bind to peptide tags, for example, His and Myc tags) and anti-tumor antibodies (e.g., anti-CD40 or anti-CS1) fused to a peptide tag (e.g., a His or Myc tag). Components of a system as described herein can include immune cells, CAR peptides, nucleic acids encoding CARs, antibodies and antigen binding fragments thereof, nucleic acids encoding antibodies and antigen binding fragments thereof, and anti-tumor antibodies (e.g., anti-CD40 or anti-CS1) conjugated to a peptide tag (e.g., a His tag or a Myc tag)

The anti-tumor antibodies (e.g., anti-CD40 or anti-CS1) and antigen binding fragments thereof or CARs disclosed herein are suited for the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In some instances, one of the containers may comprise an anti-tumor antibody (e.g., anti-CD40 or anti-CS1) or antigen binding fragment thereof or CAR as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises a container containing a labeled anti-tumor antibody or antigen binding fragment thereof. In some embodiments, the kit comprises separate containers containing an anti-tumor antibody or antigen binding fragment thereof and a detectable label.

An anti-tumor antibody or antigen binding fragment thereof or CAR as described in this disclosure for use in treating cancer patients may be delivered in a pharmaceutical package or kit to doctors and cancer patients. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of an anti-tumor antibody or antigen binding fragment or CAR. In some embodiments, the pharmaceutical package or kit further comprises unit dose forms of one or more of a chemotherapeutic agent, a cytotoxic agent, a radiotherapeutic agent, or an immunotherapeutic agent.

In one embodiment, the kit or pharmaceutical package comprises an anti-tumor antibody or antigen binding fragment or CAR in a defined, therapeutically effective dose in a single unit dosage form or as separate unit doses. The dose and form of the unit dose (e.g., tablet, capsule, immediate release, delayed release, etc.) can be any doses or forms as described herein. In some embodiments, the dose form of the CAR is suitable for introduction into one or more immune cells of a subject as described herein.

### VII. Pharmaceutical Compositions and Formulations

Compositions comprising a anti-tumor antigen-specific antibody (i.e. an anti-tumor antibody) or antigen binding fragment thereof conjugated to a peptide tag or CAR-expressing immune cell of the present disclosure and a pharmaceutically acceptable carrier are also provided. The compositions may further comprise a diluent, solubilizer, emulsifier, preservative, and/or adjuvant to be used with the methods disclosed herein. Such compositions can be used in a subject with a cancer comprising cells that overexpress an anti-tumor antigen (e.g., CD40 or CS1), or otherwise have elevated anti-tumor antigen activity or signaling, that would benefit from any of the anti-tumor antigen-specific antibodies or antigen binding fragments thereof described herein.

In some embodiments, described herein are pharmaceutical compositions where the CAR-expressing immune cell and the antitumor antibody conjugated to a peptide tag are present in individual, discrete, compositions (i.e. two separate compositions that are administered to a subject separately, either contemporaneously or at different times). In some embodiments, pharmaceutical compositions can comprise both a CAR-expressing immune cell and the antitumor antibody conjugated to a peptide tag.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition[s] can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Allen (2012) Remington—The Science and Practice of Pharmacy, 22d Edition, Lloyd V, Allen, ed., The Pharmaceutical Press). In certain embodiments, the optimal pharmaceutical composition is determined by one skilled in the art depending upon, for example, the intended route of administration, delivery format and desired dosage. See, for example, Allen (2012) Remington—The Science and Practice of Pharmacy, 22d Edition, Lloyd V, Allen, ed., The Pharmaceutical Press. In certain embodiments, such compositions may influence the physical state or stability, rate of in vivo release and/or rate of in vivo clearance of the anti-tumor antigen-specific antibody (e.g., CD40 or CS1) or antigen binding fragment thereof conjugated to a peptide tag and/or CAR-expressing immune cell.

In certain embodiments, the primary vehicle or carrier in a pharmaceutical composition[s] can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising an anti-tumor antigen-specific antibody (e.g., anti-CD40 or CS1) or antigen binding fragment thereof conjugated to a peptide tag disclosed herein can be prepared for storage by mixing the selected composition having the desired degree of purity with optional formulation agents (see Allen (2012) Remington—The Science and Practice of Pharmacy, 22d Edition, Lloyd V, Allen, ed., The Pharmaceutical Press) in the form of a lyophilized cake or an aqueous solution. Further, in certain embodiments, a composition comprising an anti-tumor antigen-specific antibody (e.g., anti-CD40 or anti-CS1) or antigen binding fragment thereof conjugated to a peptide tag disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80. In certain embodiments, CAR-expressing immune cells may be cryopreserved.

In certain embodiments, the pharmaceutical composition[s] can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition[s] at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition[s] may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition[s] may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition[s] may be in the range of 4.0-5.5 such as, for example, 4.0-4.3, 4.0-4.5, 4.2-4.8, 4.5-4.8, 4.5-5.0, 4.8-5.2, or 5.0-5.5. In an embodiment, the pH is 7.2.

In certain embodiments when parenteral administration is contemplated, a therapeutic composition[s] can be in the form of a pyrogen-free, parenterally acceptable aqueous solution comprising an anti-tumor antigen-specific antibody (e.g. anti-CD40 or anti-CS1) or antigen binding fragment thereof (conjugated to a peptide tag) and/or CAR-expressing immune cell in a pharmaceutically acceptable vehicle. In certain embodiments, a vehicle for parenteral injection is sterile distilled water in which anti-tumor antigen-specific antibody or antigen binding fragment thereof (conjugated to a peptide tag) and/or CAR-expressing immune cell is formulated as a sterile, isotonic solution and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition[s] can be formulated for inhalation. In certain embodiments, an anti-tumor antigen-specific antibody or antigen binding fragment thereof (conjugated to a peptide tag) can be formulated as a dry powder for inhalation. In certain embodiments, an inhalation solution comprising an anti-tumor antigen-specific antibody or antigen binding fragment thereof (conjugated to a peptide tag) can be formulated with a propellant for aerosol delivery. In certain embodiments, solutions can be nebulized. Pulmonary administration is further described in International Application Publication No. WO/1994/020069, which describes pulmonary delivery of chemically modified proteins.

In certain embodiments, it is contemplated that formulations can be administered orally. In certain embodiments, an anti-tumor antigen-specific antibody or antigen binding fragment thereof (conjugated to a peptide tag) that is administered in this fashion can be formulated with or without carriers customarily used in compounding solid dosage forms, such as tablets and capsules. In certain embodiments, a capsule can be designed to release the active portion of the formulation at the point in the gastrointestinal tract when bioavailability is maximized, and pre-systemic degradation is minimized. In certain embodiments, at least one additional agent can be included to facilitate absorption of an anti-tumor antigen-specific antibody or antigen binding fragment thereof conjugated to a peptide tag. In certain embodiments, diluents, flavorings, low melting point waxes, vegetable oils, lubricants, suspending agents, tablet disintegrating agents, and binders can also be employed.

In certain embodiments, a pharmaceutical composition[s] can involve an effective quantity of an anti-tumor antigen-specific antibody (i.e., anti-tumor antibody) or antigen binding fragment thereof conjugated to a peptide tag in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving an anti-tumor antigen-specific antibody or antigen binding fragment thereof conjugated to a peptide tag in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E. et al. (2015) Eur. Polymer J65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15: 167-277; and Langer (1982) Chem Tech 12:98-105), ethylene vinyl acetate (Hsu and Langer (1985) J Biomed Materials Res 19(4):445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition[s] to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition[s] has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes are included. In certain embodiments, the kit can contain both a first container having a dried protein and a second container having an aqueous formulation in addition to a CAR-expressing immune cells that have been cryopreserved and stored in a suitable container for cryopreservation.

In certain embodiments, the effective amount of a pharmaceutical composition comprising an anti-tumor antigen-specific antibody or antigen binding fragment thereof (conjugated to a peptide tag) and/or CAR-expressing immune cells to be employed therapeutically depends, for example, upon the therapeutic context and objectives. One skilled in the art will appreciate that the appropriate dosage levels for treatment, according to certain embodiments, vary depending, in part, upon the molecule delivered, the indication for which an anti-tumor antigen-specific antibody or antigen binding fragment thereof (conjugated to a peptide tag) and CAR-expressing immune cell is being used, the route of administration, and the size (body weight, body surface or organ size) and/or condition (the age and general health) of the patient. The clinician can titer the dosage and modify the route of administration to obtain the optimal therapeutic effect. In particular, the antitumor antibody or antigen binding fragment thereof can be tittered while the dose, amount, or concentration of CAR-expressing immune cell[s] remains constant.

The clinician also selects the frequency of dosing, taking into account the pharmacokinetic parameters of the anti-tumor antigen-specific antibody or antigen binding fragment thereof (conjugated to a peptide tag) in the formulation used, as well as aspects of the CAR-expressing immune cell. In certain embodiments, a clinician administers the composition[s] until a dosage is reached that achieves the desired effect. In certain embodiments, the composition[s] can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition[s] is in accord with known methods, e.g., orally, through injection by intravenous, intraperitoneal, intracerebral (intra-parenchymal), intracerebral, intraventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of a combination therapy may be administered by different routes.

In certain embodiments, the composition[s] can be administered locally, e.g., during surgery or topically. Optionally local administration is via implantation of a membrane, sponge, or another appropriate material onto which the desired molecule (in particular antibody or CAR-expressing immune cells) has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition[s] comprising an anti-tumor antigen-specific antibody or antigen binding fragment thereof in an ex vivo manner. In such instances, cells that have been removed from a subject may be modified to express a CAR as described herein or otherwise exposed to growth factors or other molecules that may enhance administration of the cells. In certain embodiments, a pharmaceutical composition comprising an anti-tumor antigen-specific antibody or antigen binding fragment thereof (conjugated to a peptide tag) can be co-administered with the cells after the cells are modified to express the CAR and then subsequently implanted back into the subject.

In certain embodiments, an anti-tumor antigen-specific antibody or antigen binding fragment thereof (conjugated to a peptide tag) can be delivered along with the implantation of certain cells that have been genetically engineered, using methods such as those described herein, to express CARs as described herein. In certain embodiments, such cells can be animal or human cells, and can be autologous, heterologous, or xenogeneic. In certain embodiments, the cells can be immortalized.

### VIII. Method of Use of Switchable Car-T Cells

### A. Therapeutic Methods

As described herein, the present disclosure provides a method of treating a subject with an anti-tumor antigen-expressing cancer as described herein (e.g., a CD40 or CS1 expressing tumor), comprising administering to the subject a therapeutically effective amount of compositions, switchable CAR-T cells (i.e., immune cells that have been engineered to express CARs as described herein), and switchable CAR-T cell components (i.e., antitumor antibodies conjugated to a peptide tag and immune cells expressing CARs as described herein) and/or systems. In some embodiments, the subject has or is determined to have anti-tumor antigen-expressing cancer.

Compositions, pharmaceutical compositions, and methods according to the present disclosure can be utilized, for example, to treat a cancer in a subject that presents an anti-tumor antigen (i.e., expresses). In some embodiments, such cancers include hematological malignancies, for example, B-cell malignancies, lymphoma, leukemia, and multiple myeloma. In some embodiments, such cancers include solid tumors, for example, bladder cancer, breast cancer, cervical cancer, colon cancer, gastric cancer, head and neck cancer, lung cancer, melanoma, osteosarcoma, ovarian cancer, pancreatic cancer, renal cell carcinoma, non-small cell lung carcinoma.

The compositions described herein are useful in, inter alia, methods for treating an anti-tumor antigen-expressing cancer in a subject. As used herein, the term subject means a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected to have an anti-tumor antigen-expressing cancer. In some embodiments, the subject is diagnosed with an anti-tumor antigen-expressing cancer. In some embodiments, the subject is a human that is suspected of having an anti-tumor antigen-expressing cancer, for example, a cancer as described herein.

As used herein, administer or administration refers to the act of injecting or otherwise physically delivering a substance as it exists outside the body (e.g., a switchable CAR-T cell or switchable CAR-T cell components as described herein) into a patient, such as by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing antibodies are well known to those of skill in the art. Generally, such systems should utilize components which will not significantly impair the biological properties of the antibodies, such as the paratope binding capacity (see, for example, Remington's Pharmaceutical Sciences, 18th edition, 1990, Mack Publishing). Those of skill in the art can readily determine the various parameters and conditions for producing antibody injectate or infusates without resort to undue experimentation.

Administration can be achieved by, e.g., local infusion or injection, or by means of an implant. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems. In some embodiments, a switchable CAR-T cell or switchable CAR-T components of the present disclosure can be therapeutically delivered to a subject by way of local administration.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and the like.

Treating or treatment of any disease or disorder refers to ameliorating a disease or disorder that exists in a subject or a symptom thereof. The term ameliorating refers to any therapeutically beneficial result in the treatment of a disease state, e.g., an anti-tumor antigen-expressing cancer (e.g., CD40 or CS1-expressing cancer), lessening in the severity or progression, promoting remission or durations of remission, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying or preventing metastasis. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating an anti-tumor antigen-expressing cancer (e.g., CD40 or CS1-expressing cancer) in a subject by administering switchable CAR-T cells or switchable CAR-T cell components as described in this disclosure is considered to be a treatment if there is a 10% reduction in one or more symptoms of the cancer in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

The principal symptoms of an anti-tumor antigen-expressing cancer (e.g., CD40 or CS1-expressing cancer) can include (without intending to be limiting) bone pain, nausea, constipation, loss of appetite, mental fogginess or confusion, fatigue, frequent infections, weight loss, weakness or numbness in the legs, excessive thirst, easily fractured or broken bones, anemia, leukopenia, thrombocytopenia, excessive urination, hypercalcemia, spinal cord compression, kidney dysfunction, hyperviscosity, and the like.

As used herein, the term “therapeutically effective amount” or effective amount refers to an amount of anti-tumor antigen-specific antibody (e.g., anti-CD40 or anti-CS1) or antigen binding fragment thereof conjugated to a peptide, that, in conjunction with an amount of CAR-expressing immune cells as described herein (i.e. a CAR-T system as described herein), is effective to treat a disease or disorder such that the symptoms of cancer (e.g., a CD40-expressing or CS1-expressing cancer) are ameliorated, or the likelihood of developing or progressing a cancer as described herein is decreased. A therapeutically effective amount is not, however, a dosage so large as to cause adverse side effects, such as hyperviscosity syndromes, pulmonary edema, congestive heart failure, and the like A suitable dose of an antibody or fragment thereof described herein and CAR-expressing immune cells, which dose is capable of treating a cancer as described herein in a subject, can depend on a variety of factors including the particular construct used and whether it is used concomitantly with other therapeutic agents. For example, a different dose of a whole anti-tumor antigen-specific antibody (or antigen fragment thereof) and/or CAR-expressing immune cell may be required to treat a subject with a cancer as described herein as compared to the dose of a fragment of an anti-tumor antigen-specific antibody (or antigen binding fragment thereof, e.g., Fab′ antibody fragment) and CAR-expressing immune cells required to treat the same subject. Other factors affecting the dose administered to the subject include, e.g., the type or extent of cancer as described herein. For example, a subject that has had a previous cancer as described herein may require administration of a different dosage of anti-tumor antigen-specific antibody or antigen binding fragment thereof in conjunction with an amount of CAR-expressing immune cells than a subject who has not previously had a cancer as described herein. Generally, a therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication. In some instances, a therapeutically effective amount may vary from about 0.01 mg/kg to about 50 mg/kg, preferably from about 0.1 mg/kg to about 20 mg/kg, most preferably from about 0.2 mg/kg to about 2 mg/kg, in one or more dose administrations daily, for one or several days. In some instances, the anti-tumor antigen-specific antibody or antigen binding fragment thereof and/or CAR-expressing immune cells is administered for 2 to 5 or more consecutive days.

A pharmaceutical composition can include a therapeutically effective amount of a anti-tumor antigen-specific antibody or antigen binding fragment thereof described herein. A pharmaceutical composition can include a therapeutically effective amount of CAR-expressing immune cells as described herein. A pharmaceutical composition can include a therapeutically effective amount of an anti-tumor antigen-specific antibody or antigen binding fragment thereof described herein, as well as a therapeutically effective amount of CAR-expressing immune cells as described herein.

Such effective amounts can be readily determined by one of ordinary skill in the art as described above. Considerations include the effect of the administered anti-tumor antigen-specific antibody antigen-specific antibody or antigen binding fragment thereof, or the combinatorial effect of the anti-tumor antigen-specific antibody antigen-specific antibody or antigen binding fragment thereof with one or more additional active agents, if more than one agent is used in or with the pharmaceutical composition. In certain aspects, the doses can be about 1, about 0.5, about 0.1, about 0.05, or about 0.01 mg/kg, or any intervening dose between about 0.01 mg/kg and 1 mg/kg.

Suitable human doses of any of the anti-tumor antigen-specific antibody antigen-specific antibody or antigen binding fragment thereof and/or CAR-expressing immune cells described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such anti-tumor antigen-specific antibodies or antigen binding fragments thereof and/or CAR-expressing immune cells can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A anti-tumor antigen-specific antibody or antigen binding fragment thereof in conjunction with CAR-expressing immune cells that exhibit a high therapeutic index is preferred. While constructs that exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of a anti-tumor antigen-specific antibody or antigen binding fragment thereof lies generally within a range of circulating concentrations of the anti-tumor antigen-specific antibody or antigen binding fragment that include the ED₅₀ with little or no toxicity. The dosage may vary within this range depending upon the dosage form employed and the route of administration utilized. For anti-tumor antigen-specific antibodies or antigen binding fragments thereof described herein, the therapeutically effective dose can be estimated initially from cell culture assays. A dose can be formulated in animal models to achieve a circulating plasma concentration range that includes the EC₅₀ (i.e., the concentration of the construct—e.g., antibody—which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site. Varying amounts of CAR-expressing immune cells can also be evaluated.

In some embodiments, an anti-tumor antigen-specific antibody or antigen binding fragment thereof described herein can be administered, in conjunction with CAR-expressing immune cells as described herein, to a subject as a monotherapy. Alternatively, the anti-tumor antigen-specific antibody or antigen binding fragment thereof and CAR-expressing immune cells can be administered in conjunction with other therapies for cancer (combination therapy). For example, the composition can be administered to a subject at the same time, prior to, or after, a second therapy. In some embodiments, the anti-tumor antigen-specific antibody or antigen binding fragment thereof and/or CAR-expressing immune cells and the one or more additional active agents are administered at the same time. Optionally, the anti-tumor antigen-specific antibody or antigen binding fragment thereof and/or CAR-expressing immune cells are administered first in time and the one or more additional active agents are administered second in time. In some embodiments, the one or more additional active agents are administered first in time and the anti-tumor antigen-specific antibody or antigen binding fragment thereof and/or CAR-expressing immune cells are administered second in time. Optionally, the anti-tumor antigen-specific antibody or antigen binding fragment thereof and/or CAR-expressing immune cells and the one or more additional agents are administered simultaneously in the same or different routes. For example, a composition comprising the anti-tumor antigen-specific antibody or antigen binding fragment thereof and/or composition comprising CAR-expressing immune cells optionally contains one or more additional agents.

Switchable CAR-T systems described herein can replace or augment a previously or currently administered therapy. For example, upon treating with switchable CAR-T system, administration of the one or more additional active agents can cease or diminish, e.g., be administered at lower levels or dosages. In some embodiments, administration of the previous therapy can be maintained. In some embodiments, a previous therapy is maintained until the level of the switchable CAR-T system reaches a level sufficient to provide a therapeutic effect.

Also provided are cancer treatment methods using switchable CAR-T systems as described in this disclosure. In some embodiments, these methods comprise using the CAR-T system to redirect the specificity of an immune effector cell (e.g., a T cell) to target a cancer cell (e.g., a CD40 antigen-expressing cancer cell or CS1 antigen-expressing cancer cell). Thus, provided herein are methods of stimulating an effector cell-mediated response (such as a T cell-mediated immune response) to a target cell population or tissue comprising cancer cells in a mammal, comprising the step of administering to the mammal an effector cell (such as a T cell) that expresses a CAR as described herein. In some embodiments, “stimulating” an immune cell refers to eliciting an effector cell-mediated response (such as a T cell-mediated immune response), which is different from activating an immune cell. CAR-expressing effector cells described herein can be infused to a subject in need of treatment (e.g., a cancer patient). In some embodiments, the infused cell is able to kill (or lead to the killing of) cancer cells in the subject. Formulations and methods for making CAR-expressing effector cells and using them in therapeutic methods are known in the art (see, e.g., Feins et al., 2019, Am. J. Hematol. 94(S1):S3-S9).

Monitoring a subject (e.g., a human patient) for an improvement of cancer as described herein, means evaluating the subject for a change in a disease parameter, e.g., a reduction in one or more symptoms of a cancer as described herein exhibited by the subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancer as described herein.

In another aspect, provided is a method of treating a subject with cancer, the method comprising administering to the patient a CAR-T system as described in this disclosure.

In certain embodiments, the effective amount of a pharmaceutical composition comprising anti-tumor antigen-specific antibody or antigen binding fragment thereof to be employed therapeutically depends, for example, upon the therapeutic context and objectives. One skilled in the art will appreciate that the appropriate dosage levels for treatment, according to certain embodiments, vary depending, in part, upon the molecule delivered, the indication for which an anti-tumor antigen-specific antibody or antigen binding fragment thereof is being used, the route of administration, and the size (body weight, body surface or organ size) and/or condition (the age and general health) of the patient. The clinician can titer the dosage and modify the route of administration to obtain the optimal therapeutic effect. In embodiments, the dosage of CAR-expressing immune cells can be constant while the dosage of the anti-tumor antibody can be tittered.

The clinician also selects the frequency of dosing, taking into account the pharmacokinetic parameters of the CAR-T system or components thereof. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In some cases, the dosage (of the active component) ranges from about 0.0001 to 100 mg/kg, and more usually 0.01 to 20 mg/kg, of the patient's body weight. For example, dosages can be 0.3 mg/kg body weight, 1 mg/kg body weight, 3 mg/kg body weight, 5 mg/kg body weight, 10 mg/kg body weight or within the range of 0.1-20 mg/kg. In certain examples, the anti-tumor antigen-specific antibody or antigen binding fragment thereof can be administered at a dose of 1 mg/kg, 2 mg/kg, 3 mg/kg, 4 mg/kg, or 5 mg/kg once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering an anti-tumor antigen-specific antibody or antigen binding fragment thereof according to one of the aforementioned dosing regimens for a first period and another of the aforementioned dosing regimens for a second period. In some cases, the treatment discontinues for a period of time before the same or a different dosing regimen resumes. For example, a patient may be on an anti-tumor antigen-specific antibody or antigen binding fragment thereof—specific antibody dosing regimen for two weeks, off for a week, on for another two weeks, and so on. Dosage regimens for anti-tumor antigen-specific antibodies or antigen binding fragments thereof of this disclosure include 0.1 mg/kg body weight, 0.3 mg/kg body weight, 2 mg/kg body weight, 3 mg/kg body weight, or 10 mg/kg via intravenous administration, with the anti-tumor antigen-specific antibodies or antigen binding fragments thereof being given using one of the following dosing schedules: (i) every four weeks for six dosages, then every three months; (ii) every three weeks; (iii) 3 mg/kg body weight once followed by 1 mg/kg body weight every three weeks.

In some cases, the dosage of the CAR-expressing immune cells can be about 2×10⁵ to about 2×10¹¹ cells, for example, about 2×10⁶ to about 2×10¹¹ cells, about 2×10⁷ to about 2×10¹¹ cells, about 2×10⁵ to about 2×10⁸ cells, about 2×10⁶ to about 2×10⁹ cells, or about 2×10⁶ to about 2×10⁸ cells. In certain examples, the CAR-expressing immune cells can be administered once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering CAR-expressing immune cells according to one of the aforementioned dosing regimens for a first period and another of the aforementioned dosing regimens for a second period. In some cases, the treatment discontinues for a period of time before the same or a different dosing regimen resumes. For example, a patient may be on an anti-tumor antigen-specific antibody or antigen binding fragment thereof—specific antibody dosing regimen for two weeks, off for a week, on for another two weeks, and so on.

In certain embodiments, the route of administration of the pharmaceutical composition is in accord with known methods, e.g., orally, through injection by intravenous, intraperitoneal, intracerebral (intra-parenchymal), intracerebral, intraventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of a combination therapy may be administered by different routes.

In some instances, the provided methods may include administering to the subject one or more CAR-T system components as described herein that can be conjugated to a therapeutic agent. The therapeutic agent may be at least one of a cytotoxic agent, a chemotherapeutic agent, or an immunosuppressive agent. Such therapeutic agents are described below.

In some instances, the provided methods may include administering a CAR-T system as described herein and a second form of cancer therapy to the subject. The second form of cancer therapy may include a cytotoxic agent, a chemotherapeutic agent, an immunosuppressive agent (including immune checkpoint inhibitors), or radiation therapy. In some embodiments, the second form of cancer therapy is an antibody (e.g., a monoclonal antibody). Monoclonal antibodies which may be administered as a second form of cancer therapy include, but are not limited to, rituximab (e.g., for treatment of B-cell lymphomas), trastuzumab (e.g., for treatment of breast cancer), and cetuximab (e.g., for treatment of lung cancer).

In some instances, one or more components of a CAR-T system described herein can be labeled, conjugated, or fused with a therapeutic agent or diagnostic agent (such as an imaging agent). The linkage can be covalent or noncovalent (e.g., ionic). Such antibodies and antibody fragments are referred to antibody-drug conjugates (ADC) or immunoconjugates. The antibody conjugates are useful for the local delivery of therapeutic agents, particularly cytotoxic or cytostatic agents, i.e. drugs to kill or inhibit tumor cells in the treatment of cancer allows targeted delivery of the drug moiety to tumors, and intracellular accumulation therein, where systemic administration of these unconjugated drug agents may result in unacceptable levels of toxicity to normal cells as well as the tumor cells sought to be eliminated. Therapeutic agents include but are not limited to toxins, including but not limited to plant and bacterial toxins, small molecules, peptides, polypeptides and proteins. Genetically engineered fusion proteins, in which genes encoding for an antibody, or fragments thereof including the Fv region, or peptides can be fused to the genes encoding a toxin to deliver a toxin to the target cell are also provided. As used herein, a target cell or target cells are anti-tumor antigen-positive cells, e.g., CD40 or CS1 positive cells.

In certain aspects, CAR-T systems according to the present disclosure can be administered as a co-therapy with other therapeutic agents. Other examples of therapeutic agents include chemotherapeutic agents, a radiotherapeutic agent, and immunotherapeutic agent, as well as combinations thereof. In this way, the antibody or peptide complex or CAR (or cell comprising a CAR) delivered to the subject can be multifunctional, in that it exerts one therapeutic effect by binding to the anti-tumor antigen protein and a second therapeutic effect by delivering a supplemental therapeutic agent.

The therapeutic agent can act extracellularly, for example by initiating or affecting an immune response, or it can act intracellularly, either directly by translocating through the cell membrane or indirectly by, for example, affecting transmembrane cell signaling. The therapeutic agent is optionally cleavable from the CD40 antigen-specific antibody or antigen binding fragment thereof. Cleavage can be autolytic, accomplished by proteolysis, or affected by contacting the cell with a cleavage agent.

In some instances, the therapeutic agent is a cytotoxic agent. A cytotoxin or cytotoxic agent includes any agent that is detrimental to cells. Examples of toxins or toxin moieties include diphtheria, ricin, streptavidin, and modifications thereof. Additional examples include paclitaxel, cisplatin, carboplatin, cytochalasin B, gramicidin D, ethidium bromide, emetine, etoposide, tenoposide, colchicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. Therapeutic agents include, but are not limited to, antimetabolites (e.g., methotrexate, 6-mercaptopurine, 6-thioguanine, cytarabine, 5-fluorouracil, decarbazine), alkylating agents (e.g., mechlorethamine, thiotepa, chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e. g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Cytotoxic peptides such as auristatin (antineoplastic) peptides auristatin E (AE) and monomethylauristatin (MMAE), which are synthetic analogs of dolastatin, may also be conjugated to the anti-tumor antigen-specific antibody or antigen binding fragment thereof. In some instances, the anti-tumor antigen-specific antibody or antigen binding fragment thereof may be conjugated to a radioactive metal ion.

As referred to herein, a chemotherapeutic agent is a chemical compound useful in the treatment of cancer. Examples of chemotherapeutic agents include erlotinib (such as TARCEVA®, Genentech/OSI Pharm.), bortezomib (such as VELCADE®, Millenium Pharm.), fulvestrant (such as FASLODEX®, AstraZeneca), sutent (such as SU11248, Pfizer), letrozole (such as FEMARA®, Novartis), imatinib mesylate (such as GLEEVEC®, Novartis), PTK787/ZK222584 (Novartis), oxaliplatin (such as ELOXATIN®, Sanofi), 5-fluorouracil (5-FU), leucovorin, rapamycin (also known as sirolimus)(such as RAPAMUNE®, Wyeth), lapatinib (such as TYKERB®, GSK572016, GlaxoSmithKline), lonafarnib (such as SCH 66336), sorafenib (such as BAY43-9006, Bayer Labs.), capecitabine (such as XELODA®, Roche), docetaxel (such as TAXOTERE®), and gefitinib (such as IRESSA®, Astrazeneca), AG1478, AG1571 (such as SU 5271; Sugen Inc.), alkylating agents such as thiotepa and cyclosphosphamide (such as CYTOXAN®); alkyl sulfonates such as busulfan, improsulfan and piposulfan; aziridines such as benzodopa, carboquone, meturedopa, and uredopa; ethylenimines and methylamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, particularly calicheamicin γ₁^{I} and calicheamicin θ₁^{I}); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (such as ADRIAMYCIN®, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; Trametes versicolor polysaccharide-K (Krestin, PSK) (JHS Natural Products, Eugene, OR); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; cytarabine (cytosine arabinoside, “Ara-C”); cyclophosphamide; thiotepa; taxoids, e.g., paclitaxel (such as TAXOL®, Bristol-Myers Squibb Oncology, Princeton, N.J.), ABRAXANE™ (a Cremophor-free, albumin-engineered nanoparticle formulation of paclitaxel (American Pharmaceutical Partners, Schaumberg, IL)), and doxetaxel (such as TAXOTERE®, Rhône-Poulenc Rorer, Antony, France); chloranbucil; gemcitabine (such as GEMZAR®); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (such as NAVELBINE®); novantrone; teniposide; edatrexate; daunomycin; aminopterin; xeloda; ibandronate; CPT-11; topoisomerase inhibitor RFS 2000; difluorometlhylomithine (DMFO); retinoids such as retinoic acid; capecitabine; and pharmaceutically acceptable salts, acids or derivatives of any of the above.

Chemotherapeutic agents, as used herein, also refers to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY 117018, onapristone, and toremifene (such as FARESTON®); (ii) aromatase inhibitors that inhibit the enzyme aromatase, which regulates estrogen production in the adrenal glands, such as, for example, 4(5)-imidazoles, aminoglutethimide, megestrol acetate (such as MEGASE®), exemestane (such as AROMASIN®), formestanie, fadrozole, vorozole (such as RIVISOR®), letrozole (such as FEMARA®), and anastrozole (such as ARIMIDEX®); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) VEGF receptor and angiogenesis inhibitors (including ribozymes such as ANGIOZYME®) and a HER2 expression inhibitor; (ix) vaccines such as gene therapy vaccines, for example, ALLOVECTIN-7® vaccine (plasmid/lipid complex containing the DNA sequences encoding HLA-B7 and ß2 microglobulin), LEUVECTIN® vaccine (plasmid DNA expression vector encoding interleukin-2 (IL-2) complexed with a lipid delivery vehicle (DMRIE/DOPE)), and VAXID® vaccine (patient-specific naked DNA vaccine); IL-2 or aldesleukin (such as PROLEUKIN®); topoisomerase 1 inhibitors (such as TOPOTECAN®); gonadotropin-releasing hormone antagonists (such as ABARELIX®); (x) anti-angiogenic agents such as bevacizumab (such as AVASTIN®, Genentech); and (xi) pharmaceutically acceptable salts, acids or derivatives of any of the above.

In some instances, the treatment methods provided herein may further comprise administering an immunosuppressive agent such as an immune checkpoint inhibitor as part of the method. These treatments work by “taking the brakes off” the immune system (are immunosuppressive), allowing it to mount a stronger and more effective attack against cancer. Several different types of checkpoint inhibitors, targeting different checkpoints or “brakes” on immune cells, are currently in use. Exemplary immunosuppressive agents are PD-1 inhibitors (such as nivolumab and pembrolizumab), PD-L1 inhibitors (such as atezolizumab, durvalumab, and avelumab), and CTLA-4 inhibitors (such as ipilimumab). In one example, the second form of cancer therapy comprises a PD-L1 inhibitor, a PD-1 inhibitor, or a CTLA4 inhibitor. In some instances, combinations of such inhibitors can be administered. In some instances, the PD-L1 inhibitor, the PD-1 inhibitor, and/or the CTLA4 inhibitor may be an inhibitory antibody that binds specifically to PD-L1, PD-1, or CTLA4, respectively.

In some instances, the treatment methods provided herein may further comprise administering radiation therapy to the subject. Radiation therapy uses high-energy radiation to shrink tumors and kill cancer cells. X-rays, gamma rays, and charged particles are types of radiation used for cancer treatment. The radiation may be delivered by a machine outside the body (external-beam radiation therapy), or it may come from radioactive material placed in the body near cancer cells (internal radiation therapy, also called brachytherapy). Systemic radiation therapy uses radioactive substances, such as radioactive iodine, that travel in the blood to kill cancer cells.

### Examples

The following examples are provided to further illustrate, but not to limit, aspects of the present disclosure.

### A. CD40 CAR-T Cells Show Lethal Toxicity in a Murine CAR-T Cell Therapy Model

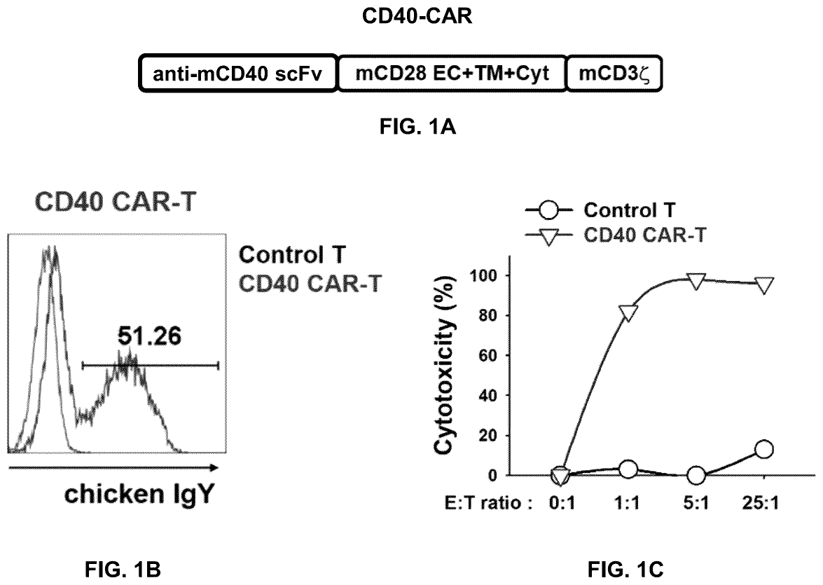

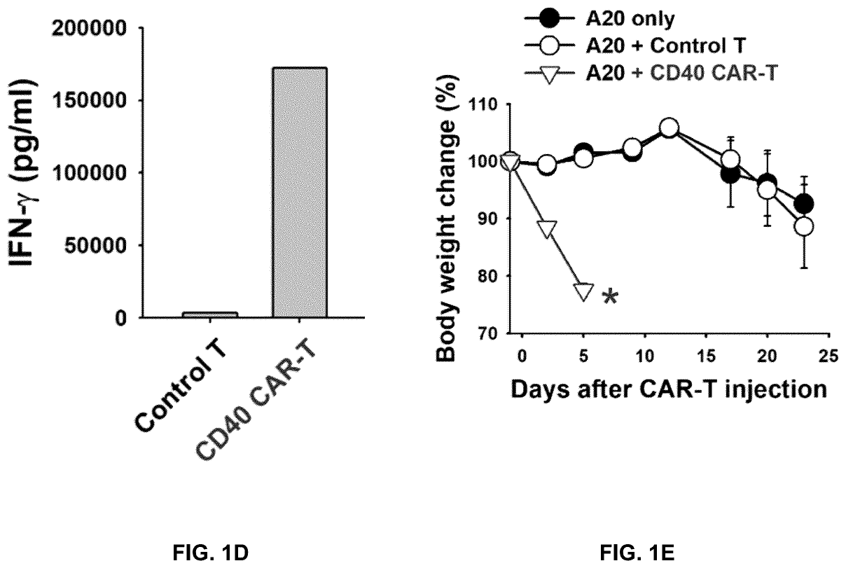

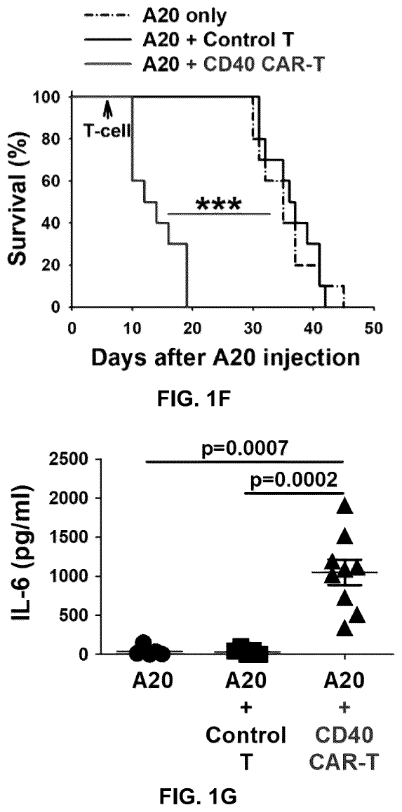

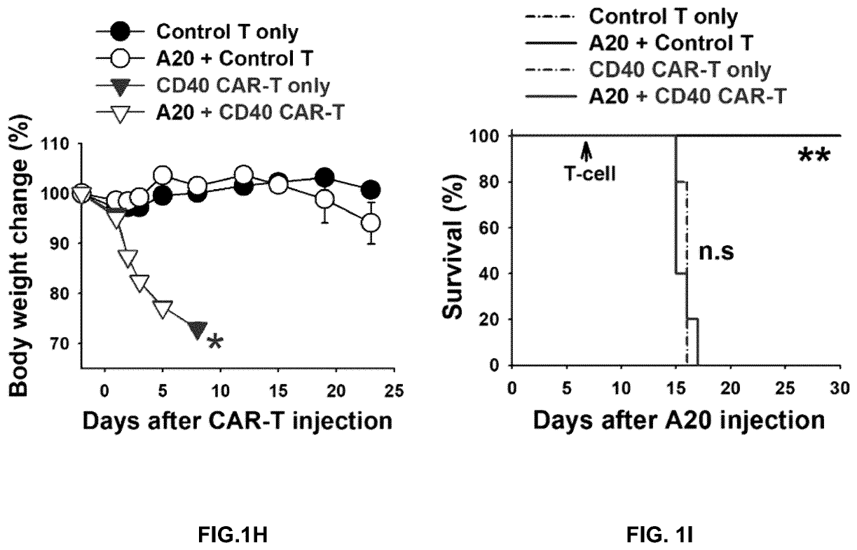

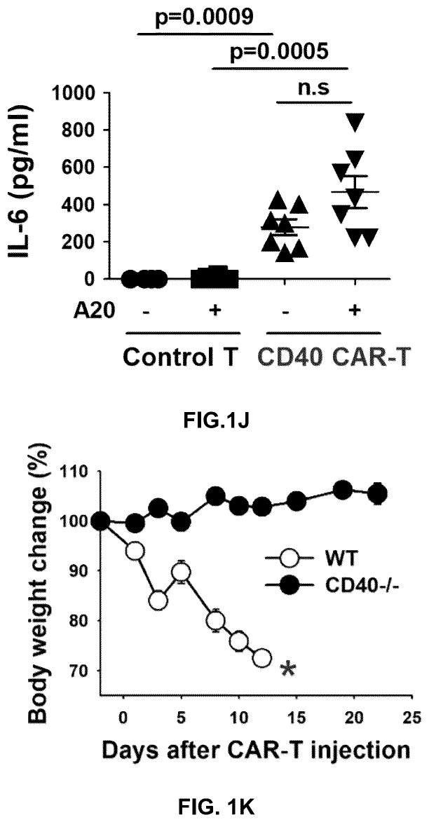

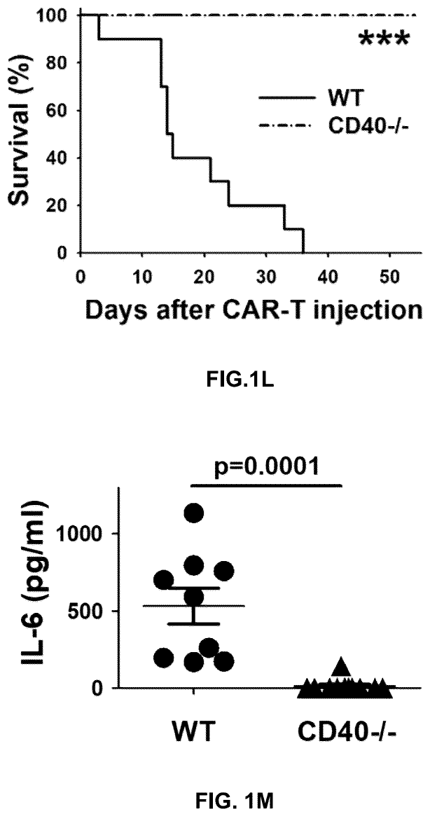

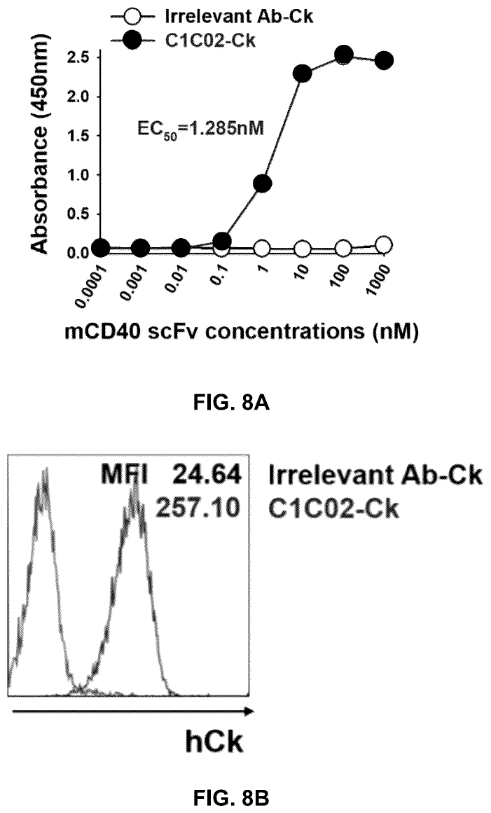

In order to evaluate both efficacy and toxicity of anti-CD40 CAR-T cells (herein, CD40 CAR-T cells) in a physiological setting, we designed a mouse syngeneic CAR-T cell therapy model against hematological malignancy. C1C02, an anti-mouse CD40 single chain variable fragment (scFv) antibody clone was selected out via screening of phage display scFv libraries from the immunized chickens. C1C02 scFv was confirmed to bind both recombinant CD40 proteins and cell surface-expressing CD40 (FIGS. 8A-8B). Then, the retroviral murine CAR constructs were generated by linking C1C02 with murine CD28/CD3zeta CAR backbone (FIG. 1A). The retrovirus-transduced Balb/C CAR-T cells well-expressed CAR molecules on the surface, and showed the strong in vitro antitumor toxicity and cytokine production against A20, a mouse B cell lymphoma cell on Balb/C background (FIGS. 1B-1D). Therefore, the murine CD40 CAR-T cells were efficiently generated.

Next, we examined whether these CAR-T cells would show in vivo antitumor efficacy by transferring the CAR-T cells into A20 tumor-bearing Balb/C mice. Surprisingly, the CAR-T cell-treated mice lost body weight rapidly and succumbed to death within several days (FIGS. 1E, 1F). As this lethality far preceded tumor-induced lethality, the CAR-T cell-induced acute toxicity was highly suspected. Consistently, serum IL-6, a well-known biomarker of acute CAR-T cell toxicity such as cytokine-release syndrome²⁷, was elevated significantly in the CAR-T cell-treated mice (FIG. 1G). Since CD40 was known to be expressed in various normal tissues, it was possible that this CAR toxicity might be against endogenous CD40 rather than tumor-expressed CD40. Therefore, we checked if the CAR-T cell toxicity occurs in the absence of tumor in normal mice. The CAR-T cell-treated tumor-non-bearing mice showed the similar degree of toxicity (the weight loss, lethality, and serum IL-6 elevation) to the tumor-bearing mice (FIGS. 1H-1J). In contrast, this toxicity was completely disappeared in CD40-deficient mice (FIGS. 1K-1M). Thus, the conventional CD40 CAR-T cells elicit on-target off-tumor toxicity against endogenous CD40 in normal tissues, which could be lethal.

### B. Both Hematopoietic and Non-Hematopoietic Expression of CD40 Cause CAR-T Cell Toxicity

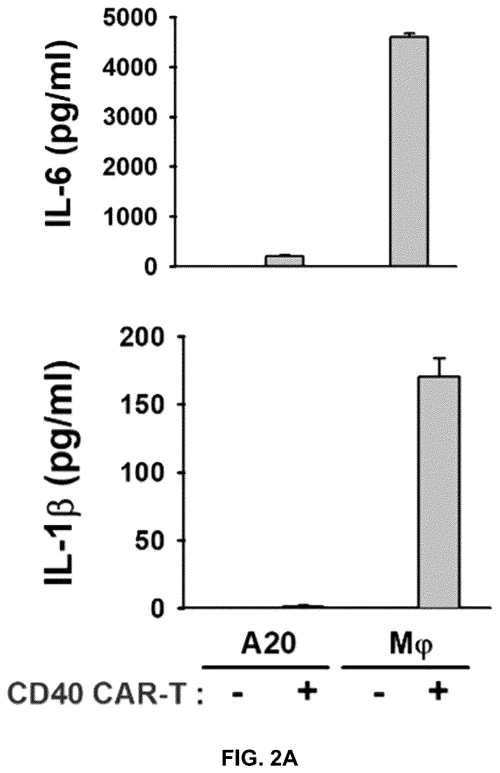

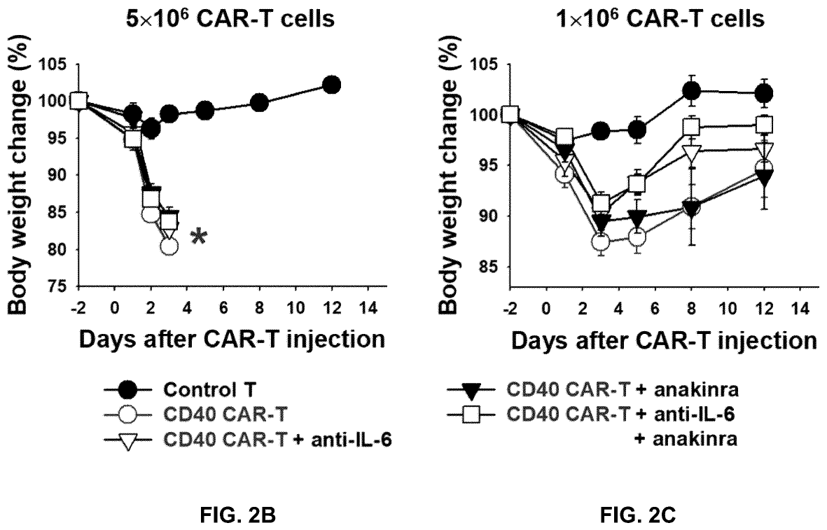

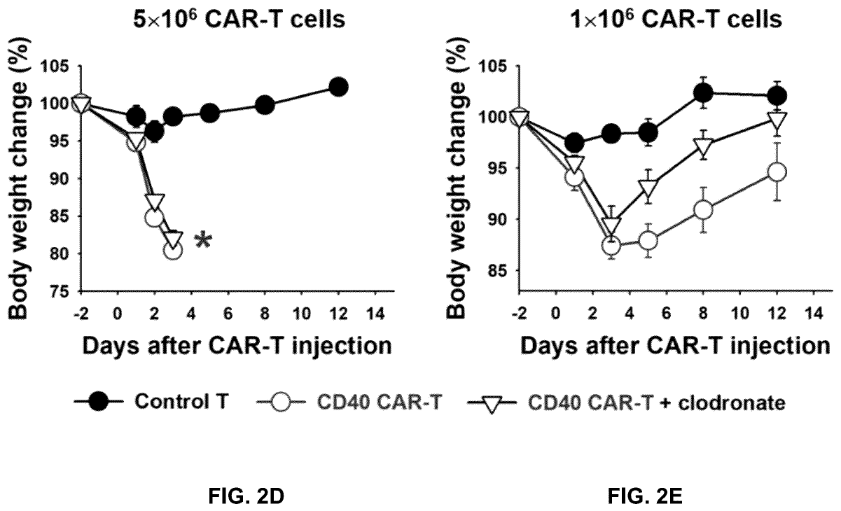

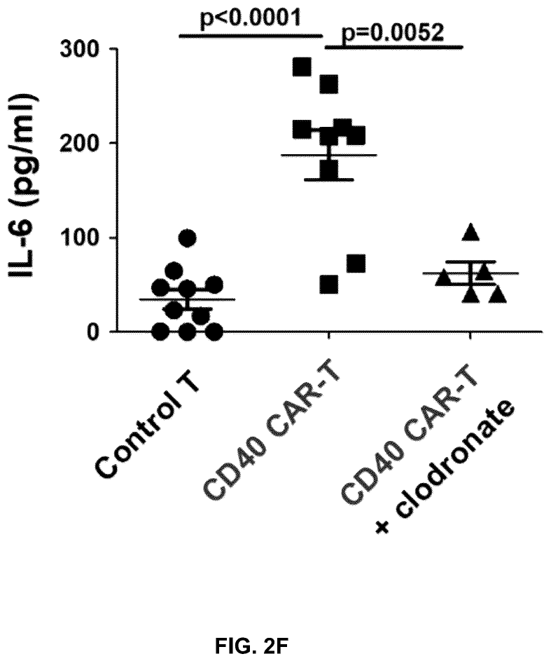

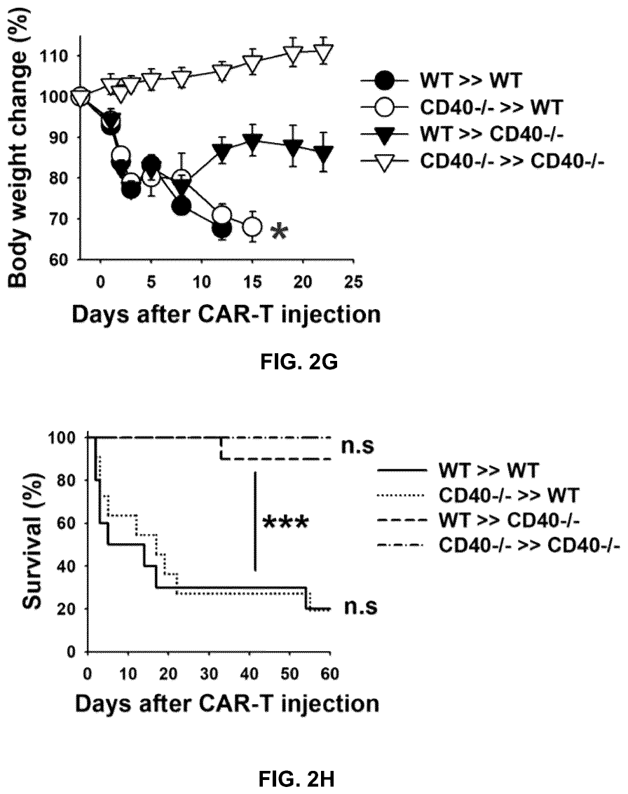

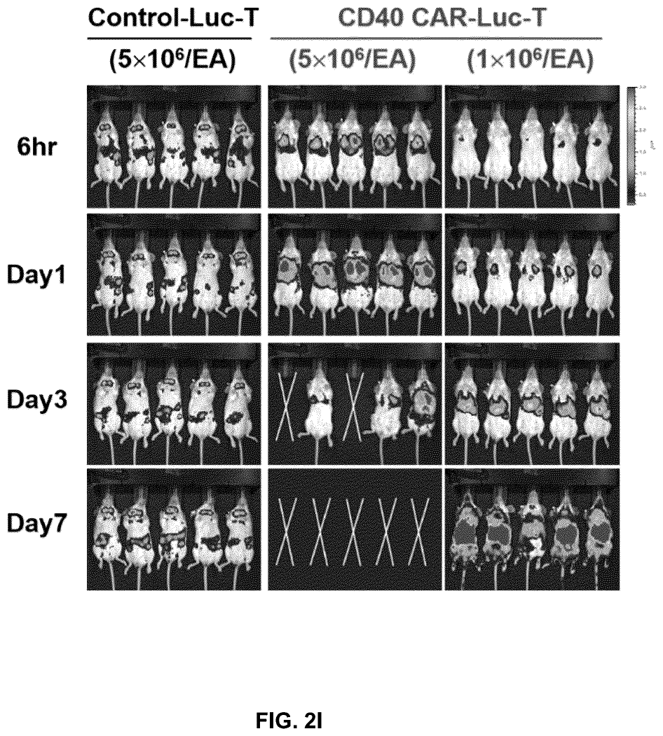

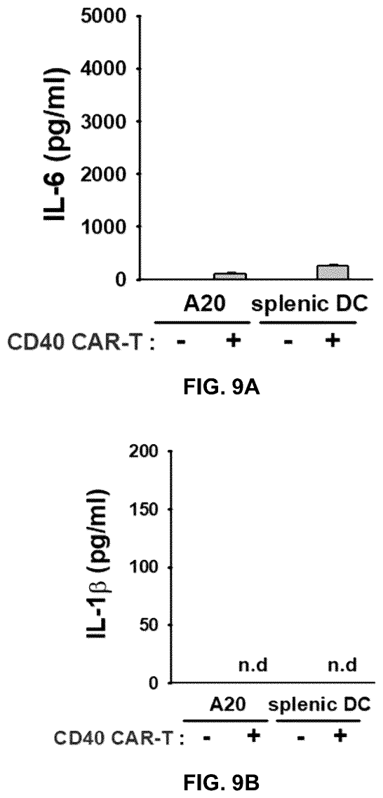

Next, we tried to identify CD40-expressing normal tissues that contribute to this toxicity. CD40 is well-known to be expressed in macrophages and dendritic cells. Because these cell types produce proinflammatory cytokines such as IL-6 and IL-1 upon CD40 engagement²¹, and the serum IL-6 levels were correlated with the CD40 CAR-T cell toxicity as above, we suspected that the CD40 CAR-T cells might engage CD40 on these cells and trigger their production of IL-6/IL-1, which mediates the toxicity. When the peritoneal macrophages or splenic dendritic cells were incubated with CD40 CAR-T cells in vitro, IL-6 and IL-1 secretion was profoundly increased compared to that of the unstimulated cells (FIG. 2A; FIGS. 9A-9B). Of note, the macrophages produced much more cytokines than the dendritic cells. Since A20 tumor cells produced a significantly lower amount of IL-6 and IL-1 in the same co-culture experiment (FIG. 2A), most of IL-6 and IL-1 in the serum of CAR-T cell-treated mice is very likely to be produced by these innate immune cells.

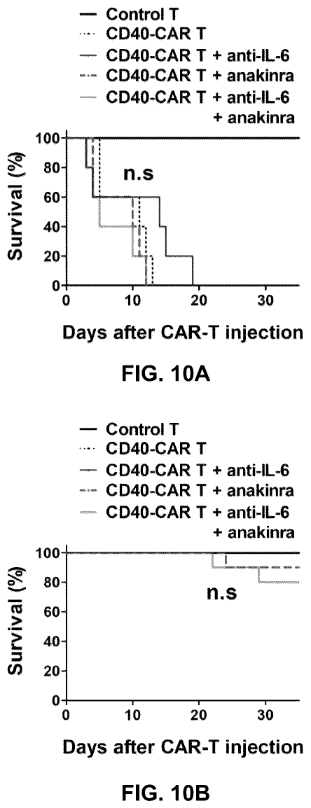

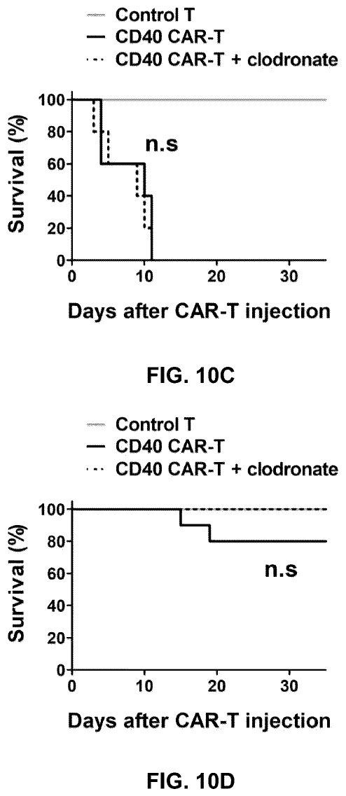

It has been reported that IL-6 and IL-1 produced by macrophages are responsible for CAR-T cell toxicities, and neutralizing these cytokines with the antagonists alleviates toxicities^{28,29}. Therefore, we examined whether neutralizing the cytokines could alleviate CD40 CAR-T cell toxicity. Treatment of anti-IL-6 or IL-1 antagonist, anakinra, did not prevent weight loss, nor lethality for the mice treated with high-dose CAR-T cells (5×10⁶ cells) (FIG. 2B; FIG. 10A). However, for the mice treated with sublethal, low-dose CAR-T cells (1×10⁶ cells), the anti-IL-6 treatment led faster, partial recovery of body weight loss triggered by CAR-T cells (FIG. 2C; FIG. 10B). Anakinra alone or co-treatment with anti-IL-6 did not facilitate this recovery. As macrophages are the main producers of IL-6, we also tried macrophage depletion by clodronate treatment. Although this treatment was not able to prevent weight loss and lethality for the high-dose CAR-T cell-treated mice, for the low-dose CAR-T cell-treated mice, it showed faster, partial recovery of weight loss (FIGS. 2D, 2E; FIGS. 10C, 10D). Consistently, serum IL-6 level was significantly lowered by macrophage depletion (FIG. 2F). Thus, IL-6, most likely produced by macrophages, partially contributed to CD40 CAR-T cell toxicity, albeit IL-6 neutralization alone cannot block lethal toxicity.

Because hematopoietic cells such as macrophages were not entirely responsible for the lethality, we checked whether non-hematopoietic expression of CD40 contributed to the toxicity. To differentiate between non-hematopoietic and hematopoietic expression of CD40 in mice, we performed bone marrow chimera experiments using CD40 knockout mice. When CD40 is expressed exclusively in hematopoietic tissues in mice (lethally irradiated CD40 knockout mice reconstituted with the wild-type bone marrow), CD40 CAR-T cells led to partial weight loss, but not lethality, in accordance with the partial toxicity mediated by macrophages as above (FIG. 2G, 2H). On the other hand, When CD40 is expressed exclusively in non-hematopoietic tissues in mice (lethally irradiated wild-type mice reconstituted with CD40 knockout bone marrow), CD40 CAR-T cells elicited profound weight loss and lethality comparable to those of the wild-type control mice. Thus, non-hematopoietic CD40 expression is highly responsible for the lethal toxicity of the CAR-T cells.

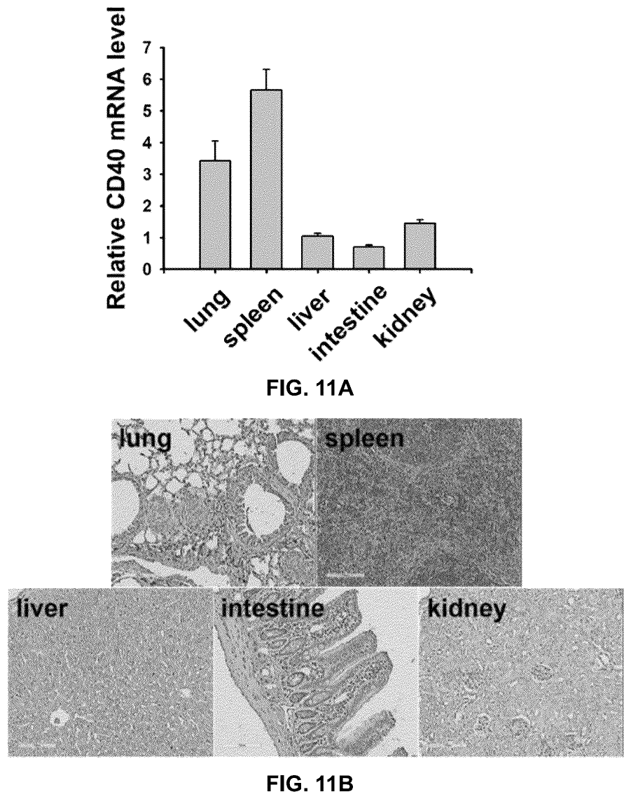

Then, we wanted to evaluate which tissues CD40 CAR-T cells attack for the lethality. To trace CAR-T cell migration and accumulation in vivo, we generated a new retroviral vector that simultaneously harbor CD40 CAR and the enhanced luciferase (effluc) gene, which enabled kinetic bioluminescence imaging of CD40 CAR-T cells in live mice. Whereas the control effluc-transduced T cells without CAR initially migrated to spleens and dispersed to various organs in low numbers (low bioluminescence signal), CD40 CAR-T cells rapidly and gradually accumulated in the lung with subsequent deaths at a high-dose of CAR-T cells (FIG. 2I). At a low-dose of CAR-T cells, CAR-T cells accumulated in the lung with the delayed kinetics and subsequently spread all over the body with increased cell expansion (high bioluminescence signal). Consistently, CD40 mRNA and protein were detected in various normal tissues with highest expression in lung and spleen (FIGS. 11A-11B). Thus, both hematopoietic and non-hematopoietic expression of CD40 contributed to CD40 CAR-T cell toxicity, which could not be avoided in the conventional CAR therapy model.

### C. Anti-CD40 Switchable CAR T Cells can be Generated Using Both Chemical and Peptide-Tagged Adaptors

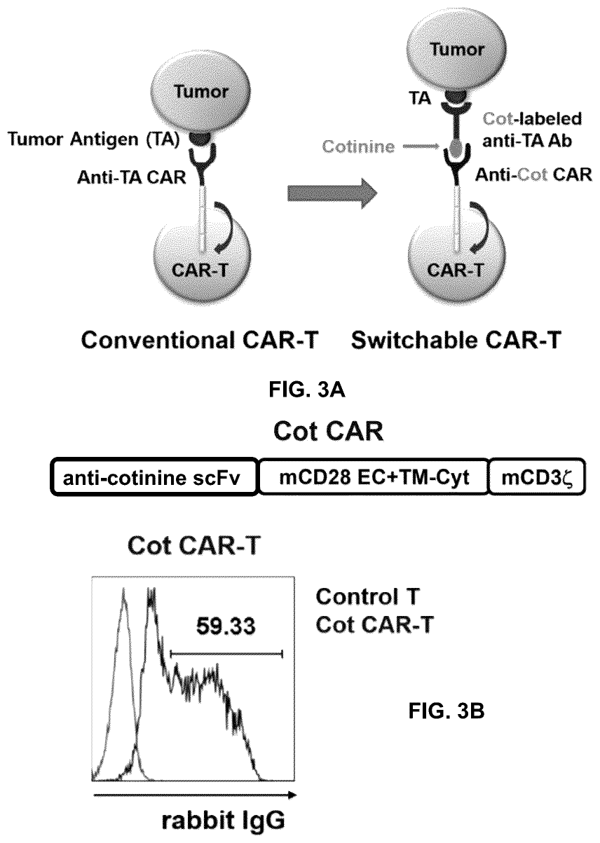

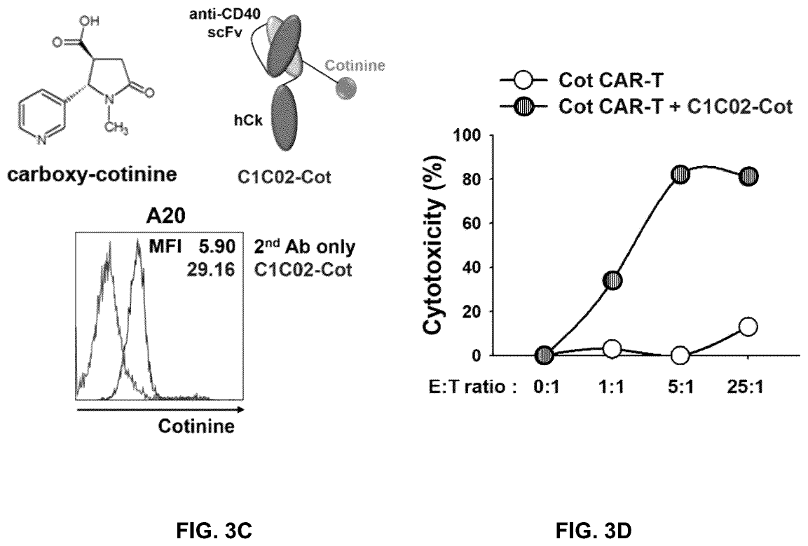

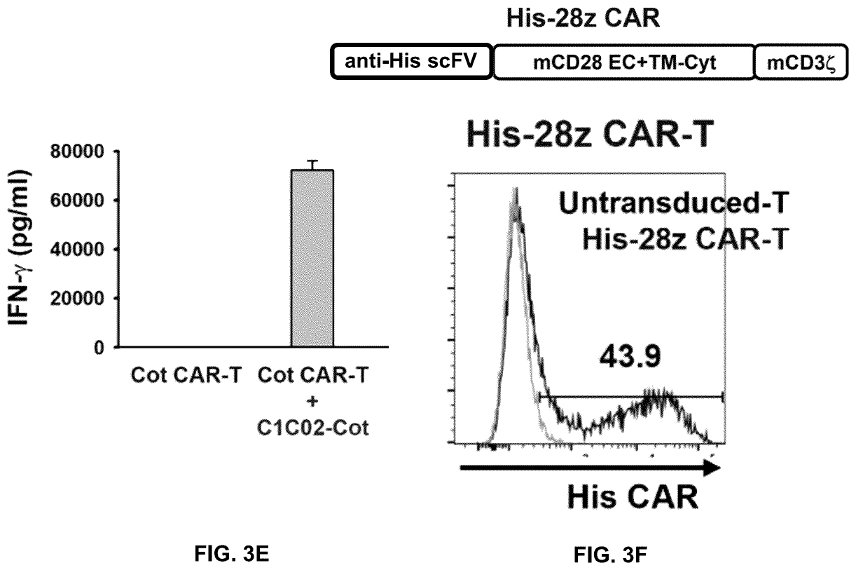

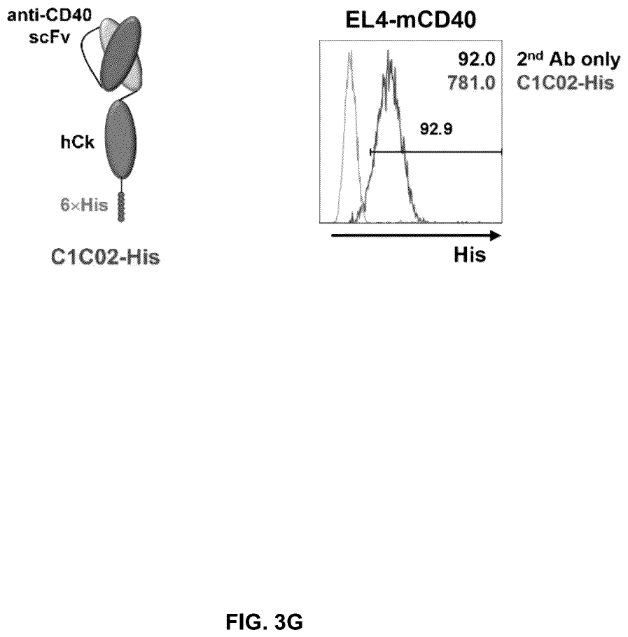

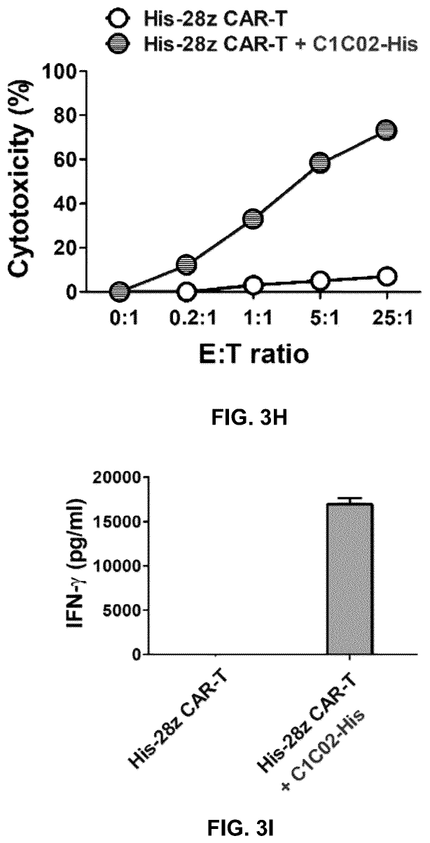

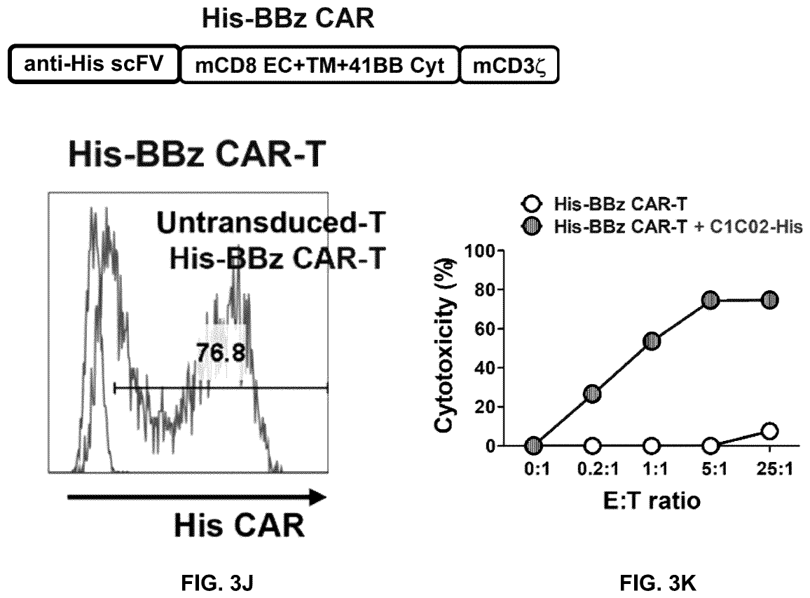

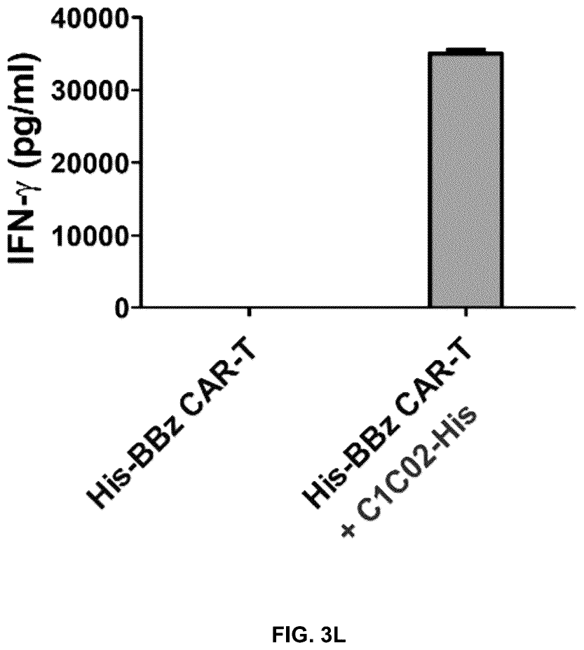

A switchable CAR system has been proposed as a tool for avoiding acute toxicities such as cytokine release syndrome. Therefore, we tried to test whether the on-target off-tumor toxicity of CD40 CAR-T cells could be overcome by adopting this strategy. To this end, we set up a CAR against a chemical tag, cotinine, along with a cotinine-tagged antibody as a tumor-targeting adaptor (FIG. 3A). Cotinine is an inert metabolite of nicotine that has been utilized as an epitope tag for anti-cotinine antibodies for various usages, and recently for anti-cotinine switchable CAR-NK cells^{30, 31, 32, 33}. Therefore, anti-cotinine murine CAR-T cells (Cot CAR-T cells) were generated using a retroviral vector harboring an anti-cotinine scFv linked to CD28/CD3zeta-based CAR backbone (FIG. 3B). Then, we generated a cotinine-labeled anti-mouse CD40 adaptor (CD40 adaptor), C1C02 scFv fused with human immunoglobulin kappa light chain constant region (Cκ). To ensure single cotinine-labeling per one adaptor molecule (DAR; drug antibody ratio, 1:1), a cysteine residue was introduced into the framework region of the scFv to allow maleimide linker-mediated monomeric cotinine conjugation. Cotinine-conjugated CD40 adaptor bound to A20 tumor cells mediated Cot CAR-T cell-dependent tumor cell cytotoxicity and activation in vitro (FIGS. 3C-3E). Therefore, CAR T cells against a chemical tag can act as switchable CAR-T cells in association with a CD40 adaptor conjugated with the chemical. Next, we examined whether CAR T cells against a peptide tag could be used for switchable CAR system along with a peptide-tagged CD40 adaptor generated as a fusion protein. The advantage of this peptide epitope-tagging as a fusion protein will be that each adaptor molecule is tagged with one peptide, which guarantees the equimolar labeling (DAR 1:1). Additionally, the labeling efficiency would be 100% because the peptide is genetically tagged to the adaptor. This degree of labeling efficiency is not readily achievable in a chemical conjugation system. Hence, we designed a novel CD28/CD3zeta backbone CAR (His-28z CAR) against histidine hexamer peptide (6×His) using a scFv derived from a murine anti-6×His antibody (clone 3D5)³⁴ (FIG. 3F). Histidine hexamer was epitope-tagged to the C-terminus of C1C02-Cκ fusion protein to generate 6×His-tagged CD40 adaptor (C1C02-His) (FIG. 3G). His-28z CAR-T cells were efficiently generated. They showed antitumor toxicity and cytokine production in conjunction with C1C02-His adaptor (FIGS. 3F, 3H, 3I). We also generated another 41BB/CD3zeta backbone CAR (His-BBz CAR) against 6×His. His-BBz CAR-T cells also showed similar in vitro activity to His-28z CAR-T cells, indicating that the switchable CAR system can be set up irrespective of the costimulatory domain in the CAR backbone (FIGS. 3J-3L). Thus, anti-CD40 switchable CAR T cells, using either a chemical tag or a peptide tag, could be generated efficiently in murine models.

### D. Separation of the Efficacy and Toxicity Using a Switchable CAR System with CD40 Adaptors

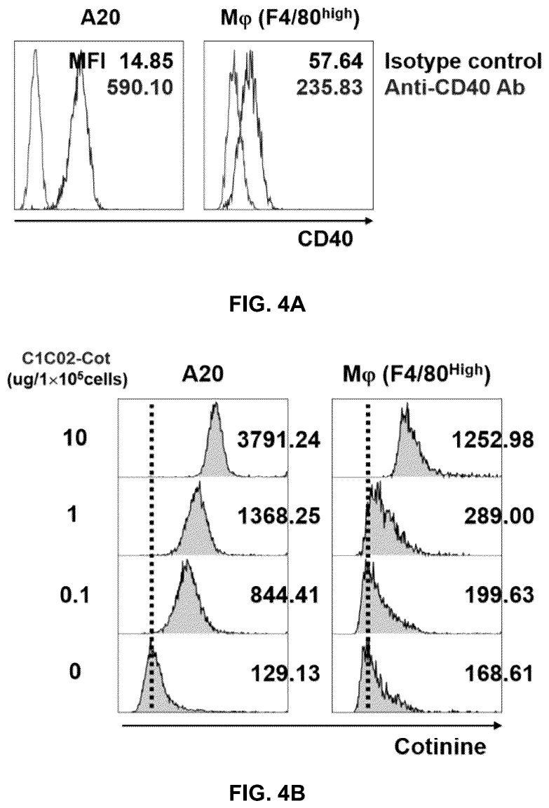

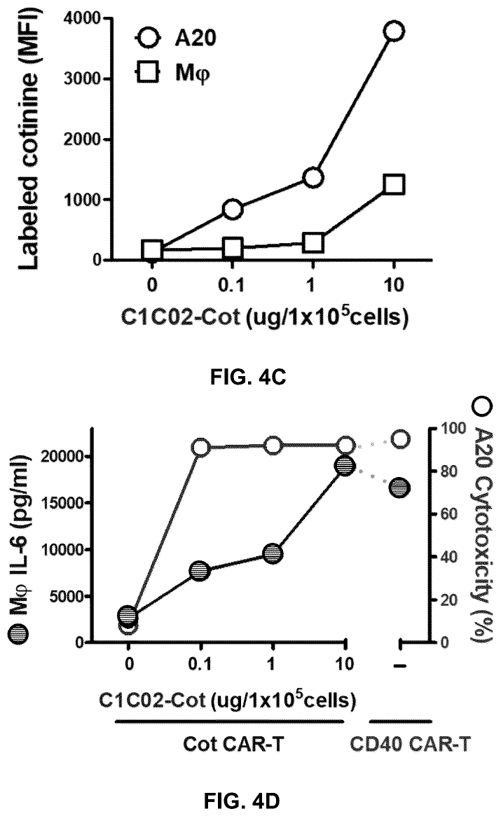

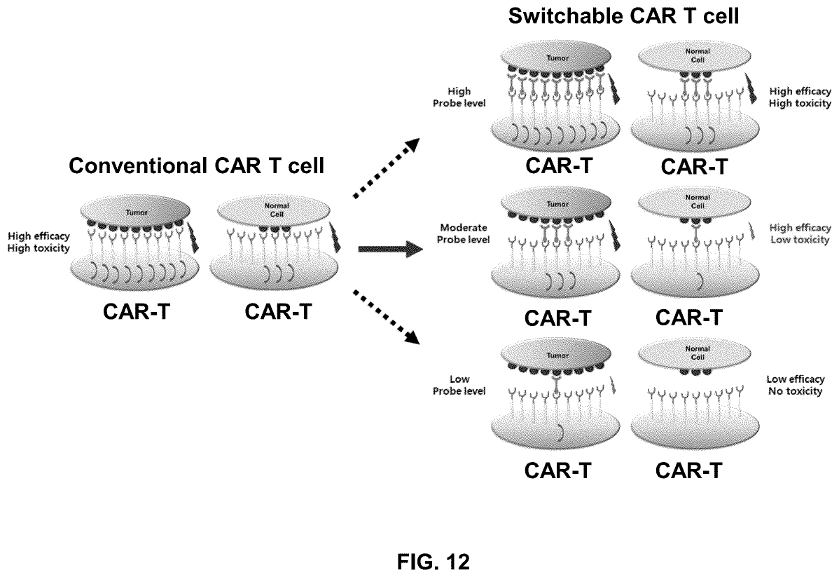

CAR-T cells require strong antigen engagement for proper activation, and the expression level of a tumor antigen is usually higher in tumor cells than in normal cells. Thus, it can be postulated that adjusting doses of the adaptor in a switchable CAR system would allow one to find an optimal adaptor dose range that does not elicit toxicity on normal cells and yet induces sufficient tumor cell-killing (FIG. 12). To test this concept in vitro, we co-cultured the Cot CAR-T cells either with normal macrophages or with A20 tumor cells treated with various doses of the cotinine-tagged adaptors. IL-6 production by macrophage was used as a readout for normal cell toxicity, and the cytotoxicity against A20 was used as a readout for antitumor efficacy. As expected, A20 tumor cells expressed CD40 on the cell surface at a much higher level than the macrophages (FIG. 4A). In accordance, when the cells were incubated with different doses of CD40 adaptors, the tumor cells showed much stronger binding with the adaptors than the macrophages at all dose range. Especially at the doses where the macrophages bound the adaptor very weakly (1 and 0.1 μg/10⁵ cells), the tumor cells still bound the adaptor significantly (FIGS. 4B, 4C). On the toxicity side, the macrophages treated with the highest dose (10 μg/10⁵ cells) secreted IL-6 at the similar level to the ones co-cultured with the conventional CD40 CAR-T cells, which implies the highest dose represents a toxic level of the adaptor (FIG. 4D). However, at 10 to 100-fold lower doses of the adaptor (1 and 0.1 μg/10⁵ cells), the amount of the secreted IL-6 decreased significantly, indicating the reduced toxicity. Interestingly, on the efficacy side, Cot CAR-T cells with these low doses of the adaptors (1 and 0.1 ug/10⁵ cells) still showed the equivalent level of tumor cell-killing to the Cot CAR-T cells with the highest dose of the adaptor (10 ug/10⁵ cells), and to the conventional CD40 CAR-T cells (FIG. 4D). Thus, Cot CAR-T cells equipped with the moderate to low doses of the adaptor are fully competent for tumor-killing but has much less potential to cause normal cell toxicity, implying that the optimal adaptor dose window exists for maintaining the efficacy without significant toxicity.

### E. Cot CAR-T Cells with the Adaptor Show Antitumor Efficacy without Overt Toxicity In Vivo

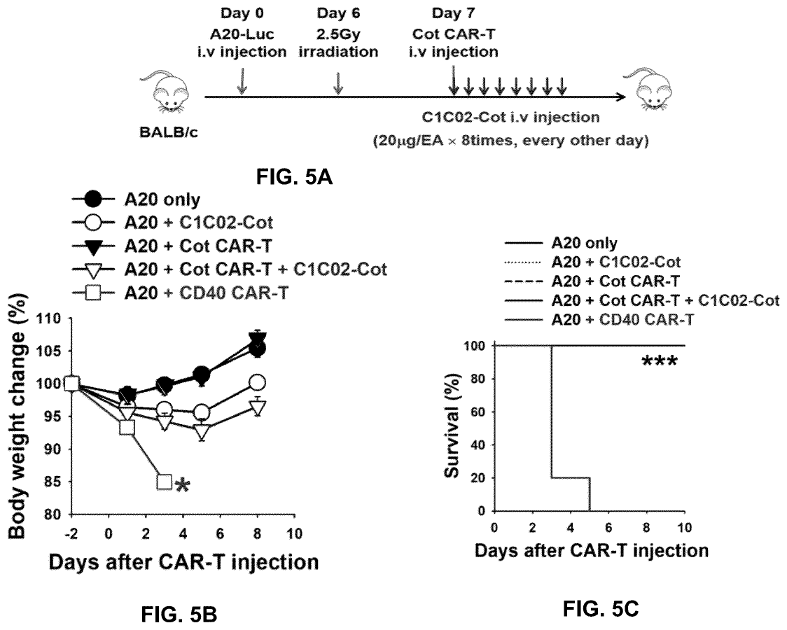

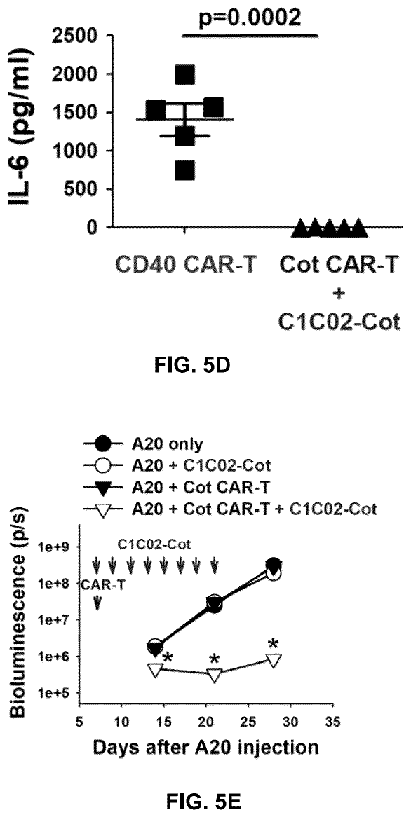

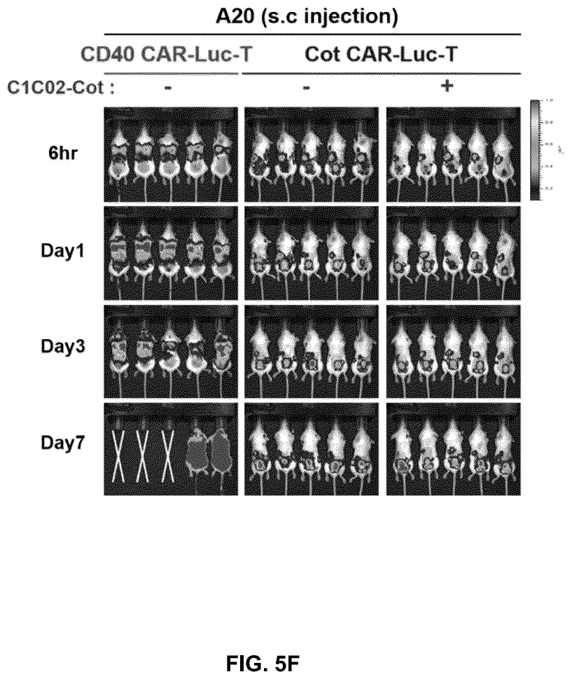

In order to evaluate whether the separation of the efficacy from the toxicity could be realized in vivo using Cot CAR system, Cot CAR-T cells were injected into A20-bearing mice together with cotinine-tagged CD40 adaptors. The adaptors used for switchable CAR systems are usually small antibody fragments such as scFv or Fab, having a short serum half-life, which enables rapid turn-on and -off of CAR-T cell activity. These adaptors are infused repeatedly during the effector phase of CAR-T cell therapy to maintain the CAR-T cell activity. In this study, we followed a regular adaptor treatment dose and schedule of the switchable CAR-T cells used for regulation of cytokine-release syndrome: 20 μg of the adaptor bidaily injected eight times (FIG. 5A)^{16, 35}. In contrast to the rapid weight losses and deaths of the conventional CD40 CAR-T cell-treated mice, the mice treated with Cot CAR-T cells and the CD40 adaptor underwent transient and small-degree weight losses, but recovered the normal weights within a few days, and all survived (FIGS. 5B, 5C). Consistently, the serum toxicity biomarker, IL-6, was not elevated in Cot CAR-T cell-treated mice, unlike CD40 CAR-T cell-treated mice (FIG. 5D). Next, we examined antitumor efficacy by tracing tumor burden via bioluminescence imaging of luciferase-expressing A20 cells in this treatment regimen. To our surprise, the tumor burden was drastically reduced by co-treatment of Cot CAR-T cells and the adaptors (FIG. 5E). To further confirm if Cot CAR-T cells bypassed CD40-expressing normal tissues and migrate to the tumor tissue, we traced the luciferase-expressing CAR-T cells in tumor-bearing mice (FIG. 5F). In this case, we injected tumors subcutaneously to locate the exact tumor sites. In contrast with the strong infiltration of conventional CD40 CAR-T cells to the lung, Cot CAR-T cells bypassed the lung, accumulated in the spleen initially, and migrated to tumor sites very specifically. Of note, Cot CAR-T cells migrated to tumor sites even without the adaptors, suggesting the inflammation in the tumor sites might have recruited the CAR-T cells^{36, 37}. Collectively, the switchable CAR-T cells were able to eliminate CD40-expressing tumors without on-target off-tumor toxicity that cannot be avoided in the conventional CD40 CAR-T cell therapy.

### F. A Switchable Anti-CD40 CAR System can be Applied to Human CAR-T Cell Therapy

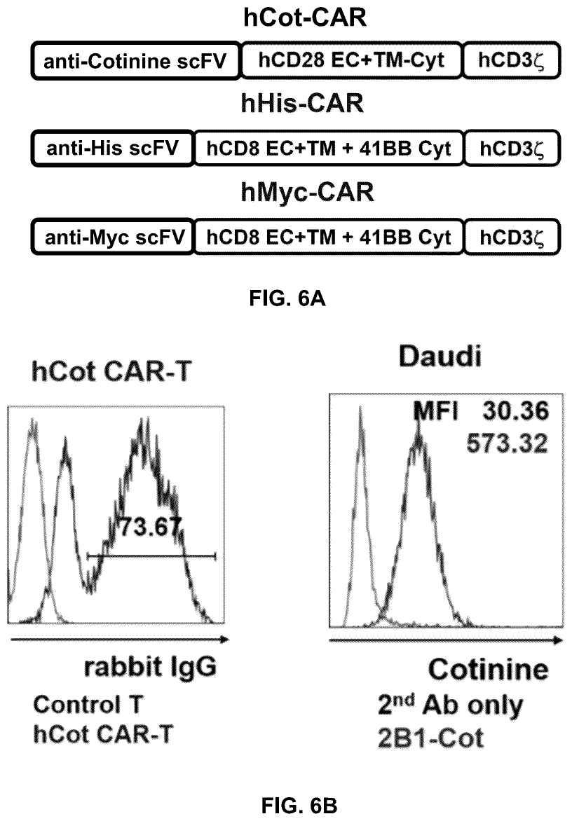

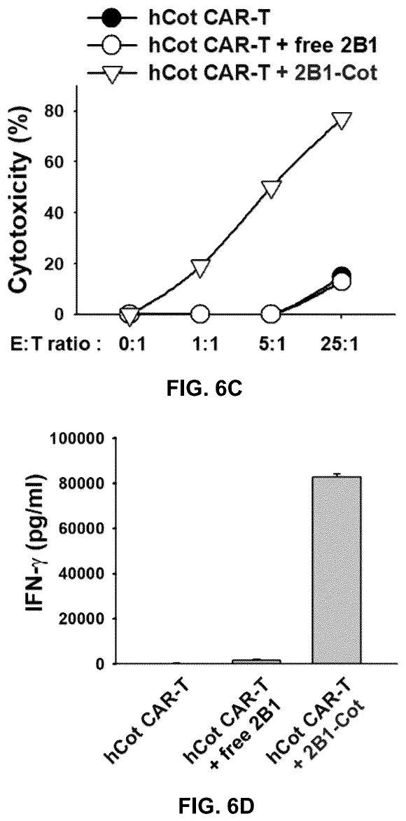

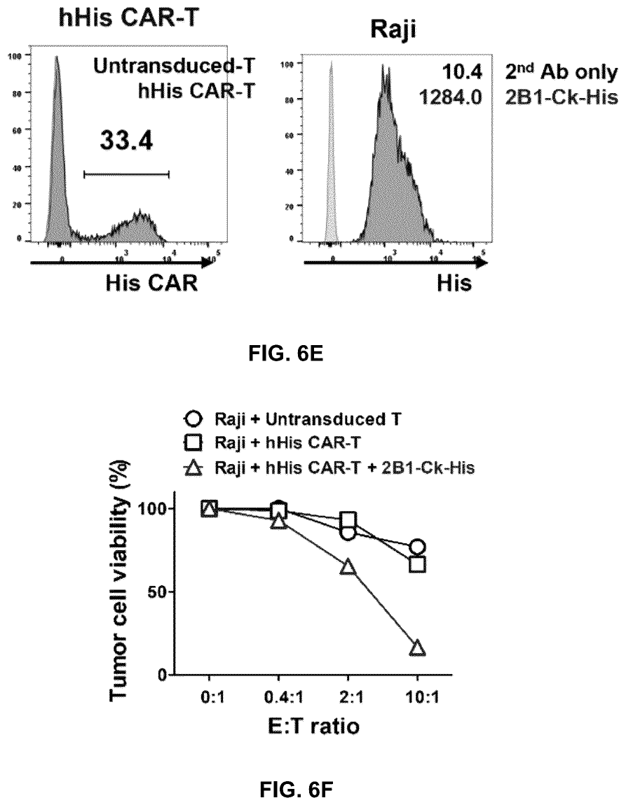

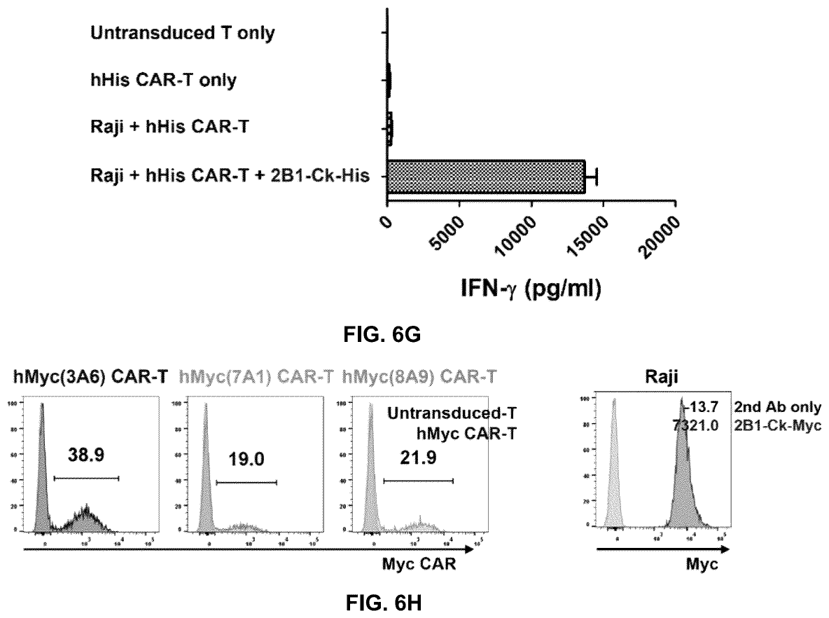

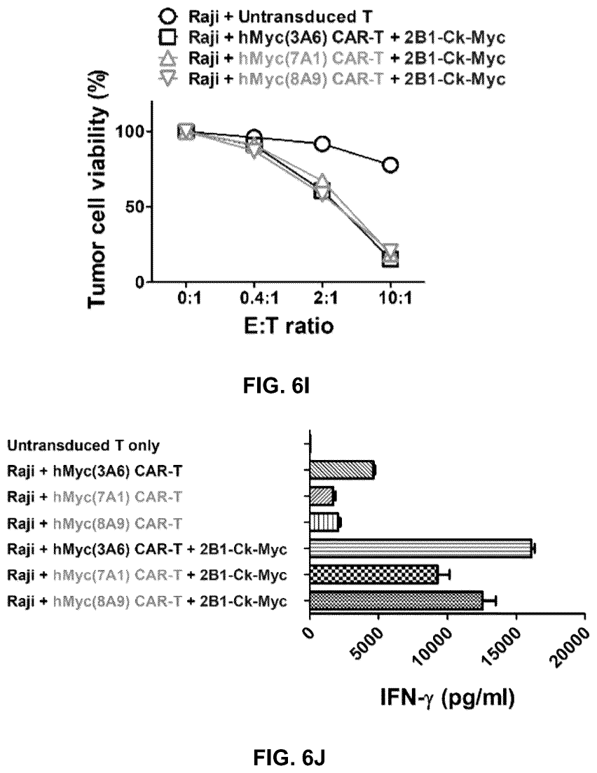

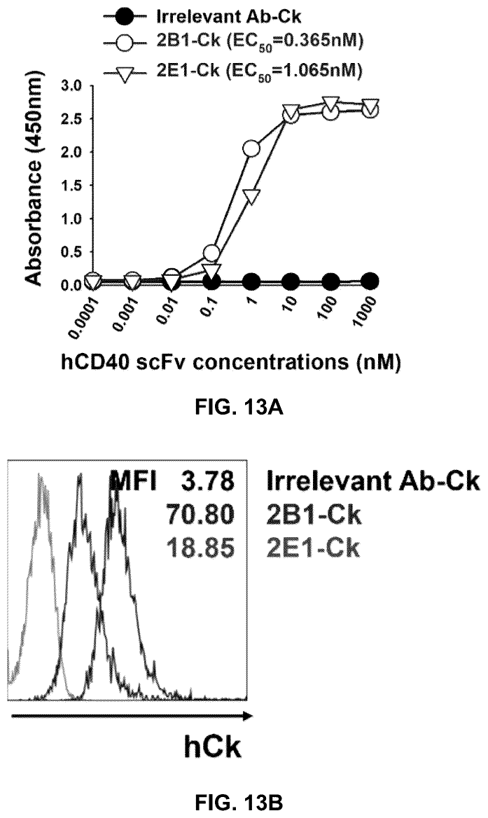

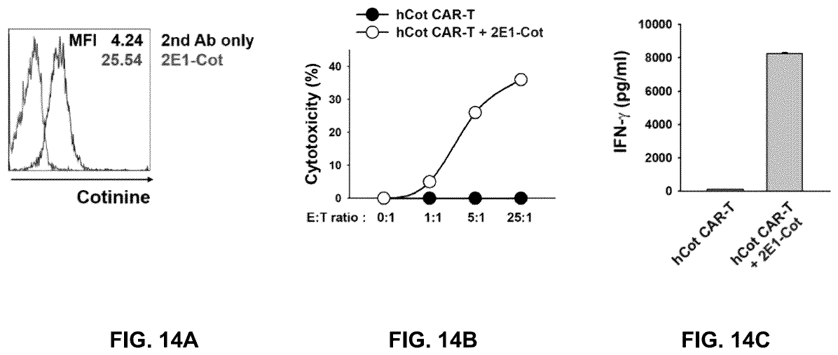

We next wanted to examine whether this therapeutic effect of anti-CD40 murine switchable CAR-T cells could be recapitulated in a human CAR-T cell therapy model. Anti-cotinine scFv was linked to human CD28/CD3zeta CAR backbone to generate an anti-cotinine human CAR retroviral construct (FIG. 6A). Anti-cotinine human CAR-T cells were produced by retroviral transduction of human peripheral blood T cells (hCot CAR-T cells) (FIG. 6B). For the adaptors, we screened a new phage display scFv library prepared from human CD40-immunized chickens, and selected two clones, 2B1 and 2E1. These scFvs were confirmed to bind both human CD40 recombinant proteins, and CD40 expressed on the cell surface (FIGS. 13A-13B). To use 2B1 or 2E1 as an anti-human CD40 adaptor (hCD40 adaptor), we conjugated cotinine to the framework region of the scFv-Cκs as done for anti-mouse CD40 adaptor. The binding of these adaptors to the cell surface was confirmed by flow cytometry (FIG. 6B and FIG. 14A). In vitro cytotoxicity and functional activity of hCot CAR-T cells in the presence of hCD40 adaptors were validated in in vitro co-culture experiments with Daudi or Raji cells, human CD40(+) lymphoma cell lines (FIGS. 6C, 6D and FIGS. 14B, 14C).

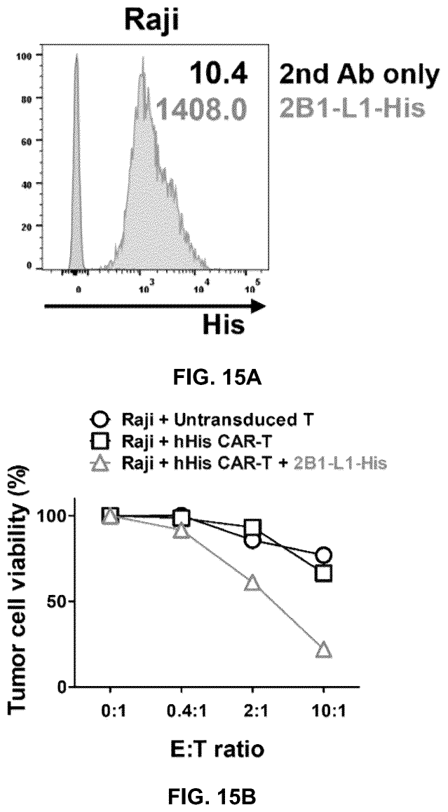

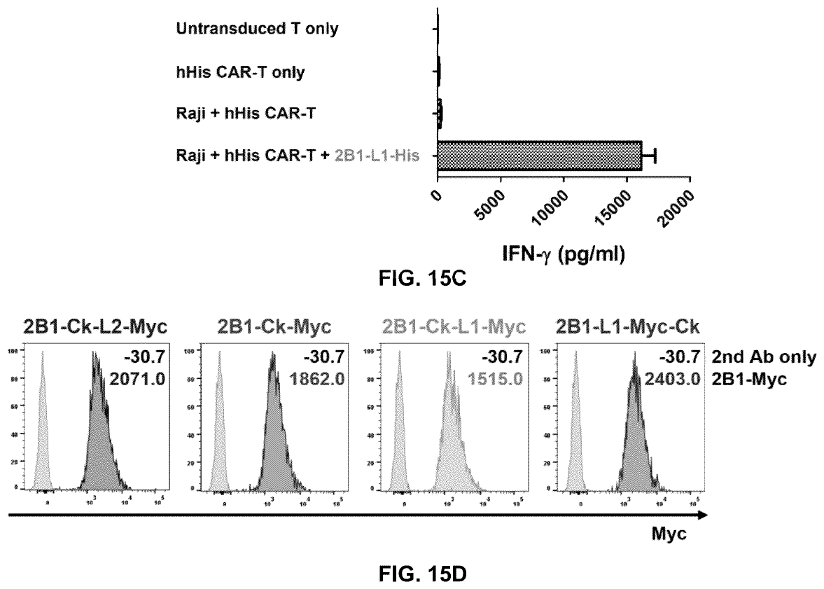

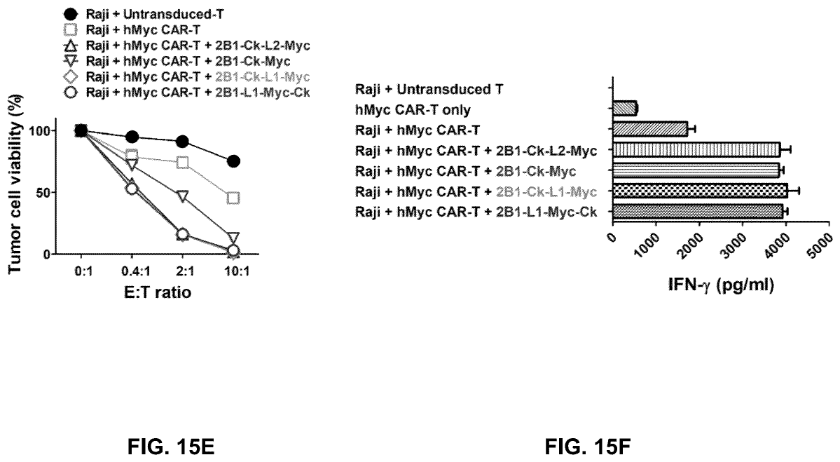

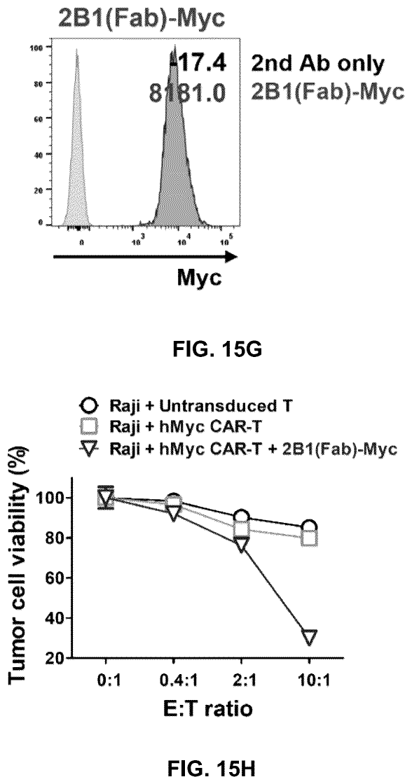

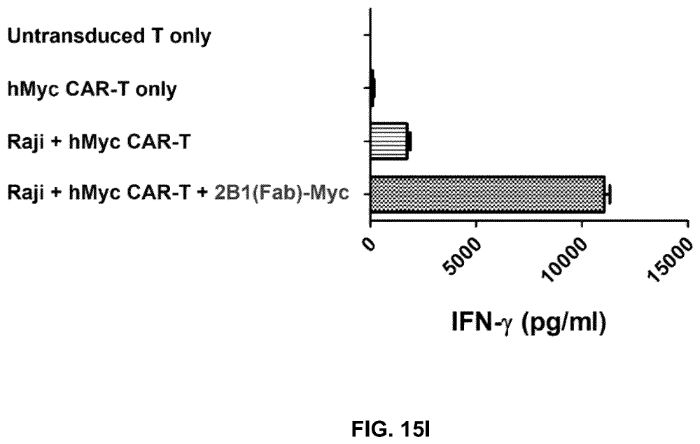

To confirm that peptide epitope-tagged adaptors and anti-peptide CAR-T cells are also functional in human switchable CAR system, anti-6×His CAR-T cells (hHis CAR-T cells) were generated by transduction of human T cells with lentivirus harboring the CAR cDNA in which anti-6×His scFv is linked with human 41BB/CD3zeta CAR backbone (FIGS. 6A,6E). Anti-human CD40 adaptor with 6×His tag (hCD40-Cκ-His) was constructed by linking 6×His peptide to the C-terminus of 2B1-Cκ and produced as a recombinant protein. The binding of hCD40-Cκ-His adaptor to tumor cells and in vitro functionality of hHis CAR-T cells in conjunction with hCD40-Cκ-His adaptor were clearly shown (FIGS. 6E-6G). As another peptide epitope tag system, anti-Myc peptide scFvs were newly generated from the Myc peptide-immunized chicken. Three scFv clones (3A6, 7A1, and 8A9) were selected and incorporated into the human 41BB/CD3 zeta CAR backbone to construct anti-Myc CAR cDNAs (FIG. 6A). Anti-Myc CAR-T cells (hMyc CAR-T cells) were efficiently generated from all three clones and also showed functional activity on co-culture with tumor cells and Myc peptide-tagged anti-hCD40 adaptor (2B1-Cκ linked with C-terminal Myc peptide) (FIGS. 6H-6J). Furthermore, when various forms and lengths of linkers that connect 2B1 and His/Myc peptides were tested for the adaptors, the most forms of the adaptors were functional in vitro (FIGS. 15A-15I). Therefore, it was reliably demonstrated that anti-CD40 switchable CAR-T cell system was able to be designed using both chemical and peptide tags.

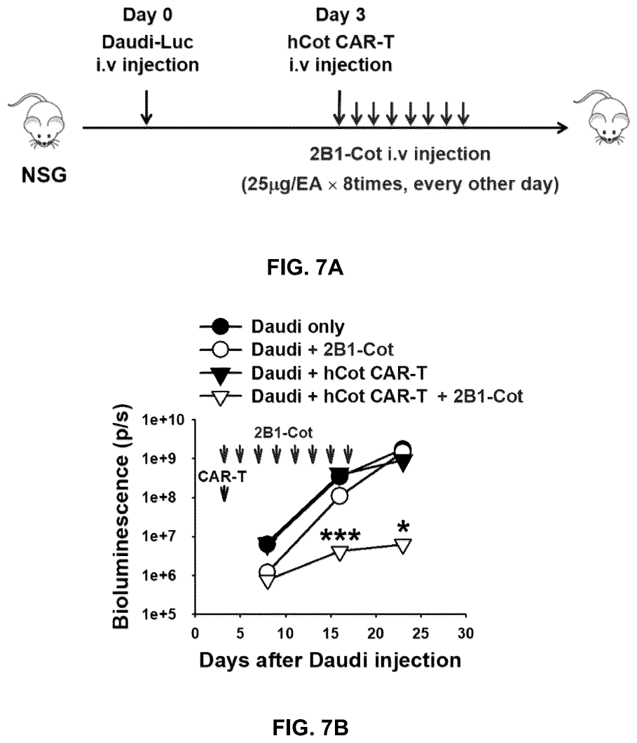

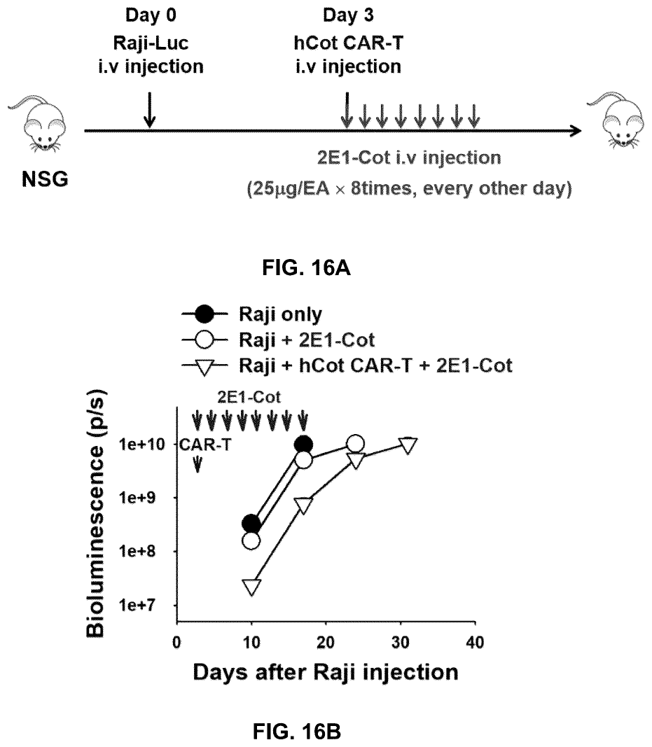

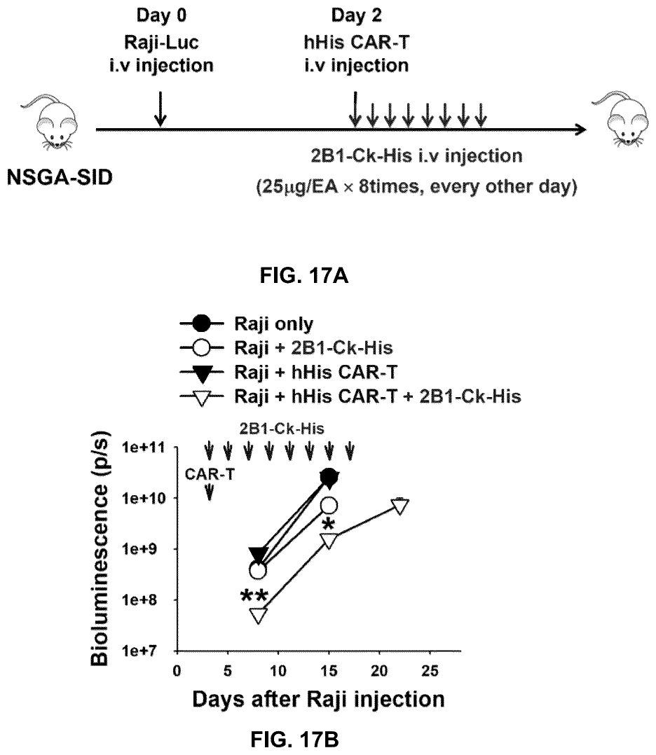

Based on in vitro effect of anti-CD40 switchable CAR-T cells, we evaluated the efficacy of these human CAR-T cells in an in vivo mouse model. First, hCot CAR-T cells were injected into the tumor (luciferase-transfected Daudi)-bearing immunodeficient NSG mice, along with bidaily infusion of hCD40 adaptor, 2B1-Cot (FIG. 7A). Similar to the murine CAR-T cell model, hCot CAR-T cells co-infused with hCD40 adaptor effectively eliminated tumor cells compared to hCot CAR-T cells in the absence of the adaptor (FIG. 7B). This in vivo efficacy was further demonstrated when another cotinine-labeled hCD40 adaptor, 2E1-Cot, was used (FIGS. 16A-16B). Also, when hHis CAR-T cells were infused with 2B1-Cκ-His adaptor to the tumor-bearing NSG mice, tumor growth was significantly repressed (FIGS. 17A-17B). Hence, the efficacy of CD40-targeting switchable CAR-T cells was also validated in human CAR system.

The result in this study is one of the examples that demonstrate that the optimal therapeutic window of a CAR-T cell can be set up for maintaining antitumor efficacy with minimal normal tissue toxicity by a single CAR system, without a need for dual CAR systems, such as SynNotch, Split or iCARs. A representative single-CAR strategy to achieve this goal is “affinity-tuned CAR-T cells,” in which the scFv in the CAR has a moderate affinity to the target antigen, thereby the CAR-T cells have enough reactivity to high-density antigens on tumor cells but do not respond to low-density antigens on normal tissues^{38, 39, 40}. Although the concept is interesting, finding a good antibody to fit into this “Goldilocks” affinity may be uneasy, and the affinity of the scFv needs to be individually tuned to different targets. This study demonstrates that the switchable CAR-T cells can achieve similar avidity tuning by adjusting the adaptor doses. Interestingly, the in vitro efficacy of the switchable CAR-T cells was maintained at a 100-fold lower dose than the toxic dose, indicating a wide therapeutic window (FIG. 4D). In this case, anti-CD40 scFv had a high affinity (at a nanomolar range), meaning that additional affinity-tuning was not necessary in the switchable CAR system (FIG. 8A). A similar avidity tuning by small molecule drugs also has been proposed. In this strategy, CAR molecule has degradable domains or protease-sensitive domains, which makes the expression level or activity of CARs regulatable by degradation inducers or protease inhibitors^{41, 42, 43, 44}. Recently, it has been directly shown that avidity-tuning of these drug-regulatable CARs by adjusting drug doses can mitigate on-target off-tumor toxicity while maintaining antitumor efficacy⁴⁴. Thus, the concept of avidity tuning of the CAR-T cells can be one of the general principles to solve on-target off-tumor toxicity problem.

Aside from the above avidity tuning effect, the switchable CAR system may have additional benefits in vivo kinetically and spatially. The scFv or Fab adaptors have very short serum half-life usually within several hours^{45, 46, 47}, thus have to be infused frequently. Although this short half-life has been highlighted by rapid turning-off of the acute CAR toxicity by stopping the infusion, the regular infusion of the short-acting adaptors may also lead to intermittent turning-off of excessive CAR-T cell activation in vivo. These effects may also contribute to low serum IL-6 and bypassing the normal tissues of the switchable CAR-T cells in the presence of the adaptor in this study. In contrast, the adaptors are known to stay much longer in the tumor sites than in the blood⁴⁵, which may lead to stronger activation of the CAR-T cells recruited to the inflamed tumor sites. Additionally, intermittent long-term rests (1 to 2 weeks) of the adaptor infusion may prevent CAR-T cell exhaustion and further enhance CAR-T cell efficacy in vivo as reported³⁵. The switchable CAR system also has shortcomings. The adaptors for the individual target need to be manufactured as a separate protein drug, which increases the cost of the overall treatment. Nonetheless, in the long run, once the number of adaptors manufactured have accumulated, multi-antigen targeting using one CAR-T cell product and personalized selection of the adaptors depending on the antigen expression in the individual patient would be advantageous.

Although the high expression of CD40 in tumors and CD40's stimulating role in dendritic cells and macrophages led to extensive development on both antagonistic and agonistic antibodies, the therapeutic efficacy of the anti-CD40 antibodies has not been very impressive in clinical trials⁴⁸. Therefore, CD40 can be a target of CAR-T cell development in terms of efficacy enhancement of antagonistic strategy. However, although anti-CD40 antibodies are relatively safe at current doses in clinical trials²³, increased doses of anti-CD40 antibodies may be potentially toxic due to hyperactivation of the immune cells or effects on CD40 expressing on parenchymal cells^{26, 48, 49}. These concerns may have prevented the emergence of CD40 as a CAR target in the research community. In this study, we confirmed that those concerns are valid in the mode of conventional CAR-T cell therapy. However, the switchable CAR system could dispel the toxicity concern, proving that CD40 is an attractive target for CAR-T cell therapy. In this setting, CD40 adaptor may kill tumor cells and stimulate innate immune cells to boost CAR-T cells and endogenous T cell reactivity as shown in CD40L-expressing CAR-T cells previously⁵⁰.

In conclusion, we have shown that the switchable CAR-T cells is a valid option for untargetable tumor antigens due to on-target off-tumor toxicity and anti-CD40 switchable CAR-T cells can be novel therapeutics for treating hematological malignancies.

### G. Materials and Methods for Examples A-F

### 1. Mice

Balb/C and C57BL/6 (B6) mice were purchased from Orient Bio, Inc., Korea. CD40 knockout (B6.129P2-Cd40^{tm1Kik}/J) on a B6 background and NSG (NOD.Cg-Prkdc^{scid} Il2rg^{tm1Wjl}/SzJ) mice were from Jackson Laboratory, USA. NSGA-SID (NOD/ShiLtJ-Prkdc^{em1Back} Il2rg^{em1Back}) mice were purchased from JA BIO, Inc., Korea. All mice were housed in a specific pathogen-free (SPF) animal facility at the Seoul National University College of Medicine and maintained in accordance with the guidelines of the Institutional Animal Care and Use Committee (IACUC). The experimental use of the animals was approved by the IACUC (SNU-160602-17, SNU-200713-4).

### 2. Cell Lines

A20 (B cell lymphoma on a Balb/C background), Raji (human B cell lymphoma), EL4 (T cell lymphoma on a C57BL6 background), and PG-13 retroviral packaging cell lines were purchased from the American Type Culture Collection (ATCC, USA). Daudi (human B cell lymphoma) was from the Korean Cell Line Bank. Phoenix GP and Phoenix Eco cell lines were provided by Garry Nolan (Stanford University, USA). Luciferase-GFP-expressing tumor cell lines (A20-Luc, EL4-mCD40-Luc, Raji-Luc, and Daudi-Luc) were generated by spin infection with either the retrovirus (pMP-LucGFP) or lentivirus (pLEF-LucGFP) harboring luciferase-P2A-EGFP expression cassette. The GFP-high populations were sorted with a FACS Aria II cell sorter (Becton Dickinson, USA).

3. Generation of Antibodies to Mouse or Human CD40 Proteins and Human c-Myc Peptide

White leghorn chickens were immunized four times with recombinant mouse CD40 (50324-M08H, Sino Biological, China) or human CD40 (10774-H08H, Sino Biological) protein. Total RNA from the bursa of Fabricius, spleen, and bone marrow was isolated using TRIzol reagent (Invitrogen, USA), and cDNA was synthesized using a SuperScript III first-strand cDNA synthesis kit with oligo (dT) primers (Invitrogen) according to the manufacturer's instructions. The scFv phage-display libraries were constructed from the cDNAs as described previously⁵¹. Four rounds of bio-panning were performed against mouse or human CD40 protein coated onto paramagnetic Dynabeads (Invitrogen). The scFv clones were selected by phage ELISA against mouse or human CD40 protein using horseradish peroxidase (HRP)-conjugated anti-M13 antibody (11973-MM05, Sino Biological). The recombinant scFv proteins fused with the constant region of human immunoglobulin kappa light chain (scFv-Cκ) (SEQ ID NO: 109, 113, 119, 123, 127, 132, 142, 147, 153, and 159) were produced from Expi293F cells (Invitrogen) and purified using anti-Cκ affinity chromatography (KappaSelect, 17545811, Cytiva, USA). The specific binding of the scFv-Cκ proteins to CD40 proteins was validated via ELISA. Briefly, the serial diluents of anti-CD40 scFv-Cκ or the irrelevant scFv-Cκ proteins are loaded on the wells of the 96-well plates coated with mouse CD40-Fc (1215-CD, R&D systems, USA) or human CD40-Fc (1493-CDB, R&D systems) proteins. The binding of the scFv-Cκs was measured with the secondary antibody (anti-human Cκ-HRP; AP502P, Sigma-Aldrich, USA).

To generate anti-c-Myc scFv antibodies for hMyc CAR-T, c-myc peptide (EQKLISEEDL; SEQ ID NO:146) were synthesized by Fmoc solid-phase synthesis method under flow conditions and then conjugated with keyhole limpet hemocyanin (KLH), ovalbumin (OVA), or bovine serum albumin (BSA) by thiol maleimide conjugation reaction. 3- to 4-week-old white leghorn chickens were injected with 100 μg of KLH- or OVA-conjugated c-myc peptides alternately three times every four weeks. The conjugated c-myc peptides were emulsified in complete or incomplete Freund's adjuvant for each injection. Total RNA was isolated, and the cDNA was synthesized as above. The scFv phage-display libraries were constructed from the cDNAs, and four rounds of bio-panning were performed against BSA-conjugated c-Myc peptide. High-affinity c-myc specific scFv clones (3A6, 7A1, and 8A9) (see Table 1 for the structural information of the clones) were selected by indirect ELISA against the antigen. The selected scFv cDNAs were subcloned into the mammalian expression vector pCEP4 (Invitrogen) with in-frame 3′ 8×His tag sequence. The 8×His-tagged proteins were expressed in mammalian cells using Expi293 expression system (Life Technologies) and purified using Ni-NTA column chromatography (Qiagen, Germany).

### 4. Preparation of Cotinine-Labeled Adaptors

For the site-specific conjugation of cotinine with the scFv-Cκ proteins, an artificial cysteine residue was introduced into the 13^{th} amino acid residue (kabat numbering) of the framework of heavy chain variable region (VH T13C). This variant scFv-Cκ proteins were incubated with 100 equivalents of TCEP (Tris (2-carboxyethyl) phosphine) in PBS with 25 mM EDTA at 37° C. for 18 h. To remove excess TCEP, the mixture was subjected to size-exclusion chromatography (Zeba™ spin desalting columns, 7 K cut-off, Thermo Scientific). Then, for the maleimide-thiol alkylation reaction, the reduced scFv-Cκ proteins were incubated with 100 equivalents of maleimide-PEG8-cotinine in PBS with 25 mM EDTA at 25° C. for 18 h. The cotinine-conjugated scFv-Cκs were purified by size-exclusion chromatography for further use. The binding of the cotinine-conjugated antibodies to the cell surface was detected with anti-cotinine antibody labeled in-house with APC (LNK032APC, Bio-Rad Laboratories) or anti-Kappa antibody labeled with APC (341108, BD Biosciences, USA) via flow cytometry.

5. Preparation of his- or Myc-Tagged Adaptors

For His-tagged adaptors, the cDNAs of scFv or scFv-Cκ fusion protein was subcloned into mammalian expression vector pCEP4 (Invitrogen) with in-frame 3′ 6×His tag sequence. The proteins (SEQ ID NO: 137) were expressed in mammalian cells using Expi293 expression system (Life Technologies) and purified using kappa-select affinity column chromatography (Cytiva) or Ni-NTA column chromatography (Qiagen). The binding of the His-tagged adaptors to the cell surface was detected with an anti-6×His antibody labeled with APC (Biolegend) via flow cytometry.

For Myc-tagged adaptors, 2B1 scFv cDNA encoding SEQ ID NO: 1 were subcloned into mammalian expression vector pCEP4 (Invitrogen) with in-frame 3′ Myc tag sequence with various lengths of linker and configurations. The proteins (SEQ ID NO: 142, 147, 153, 159 and 165/167) were expressed and purified as above. The binding of the Myc-tagged adaptors to the cell surface was detected with anti-Myc antibody (9E10) labeled with APC (R&D systems) or with anti-Myc scFv-8×His fusion protein (in-house 3A6 clone; Table 1) as a primary and anti-6×His antibody labeled with APC (Biolegend) as a secondary antibody via flow cytometry.

### 6. Preparation of CAR Construct

The anti-CD40 murine CAR ORF consists of mouse Ig kappa leader, anti-mouse CD40 scFv (clone C1C02) (Table 1), and the murine CD28-based CAR backbone previously reported (mouse CD28 extracellular, transmembrane, and cytoplasmic domain linked to mouse CD3zeta cytoplasmic domain, GenBank HM754222.1)⁵². The leader and scFv portions were PCR-amplified and linked to the synthesized CAR backbone DNA (Bioneer Inc., Korea) by blunt-end ligation. The CAR ORF was cloned into the downstream of the PGK promoter, replacing the PuroR gene of the pMSCV-puro retroviral vector (Clontech, USA) (pMP-CD40-Rm28z) for the murine CAR-T cell production. The anti-cotinine murine CAR retroviral vector (pMP-Cot-Rm28z) and anti-His murine 28z CAR retroviral vector (pMP-His-28z) were constructed in a similar way using an anti-cotinine scFv⁵³ and anti-6×His scFv³⁴. The anti-His murine BBz CAR ORF consists of mouse CD8 leader, anti-6×His scFv, and the 41BB-based CAR backbone (mouse CD8 extracellular and transmembrane domain, human 41BB cytoplasmic domain linked to mouse CD3zeta cytoplasmic domain). The leader and scFv portions were synthesized and linked to the synthesized CAR backbone DNA (GeneArt, Germany; Integrated DNA Technologies, USA) by blunt-end ligation. The CAR ORF was cloned into pMSCV-puro retroviral vector (Clontech) (pMP-His-BBz) as above. For in vivo tracing of CAR-T cells, the retroviral vectors encoding both CAR and enhanced firefly luciferase (effluc) ORFs simultaneously were generated. PCR-amplified Cot-Rm28z or CD40-Rm28z ORF was blunt-end ligated with T2A-effluc ORF amplified from pDONR222-eGFP-effLuc (a gift from Dr. Rabinovsky, M. D. Anderson Cancer Center, USA) and cloned into pMSCV-puro as described above (pMP-CD40-Rm28z-T2A-effluc, pMP-Cot-Rm28z-T2A-effluc). For a control effluc plasmid, GFP ORF from pEGFP-C1 (Clontech) and T2A-effluc ORF was ligated and cloned into pMSCV-puro (pMP-GFP-T2A-effluc).

The anti-cotinine human CAR ORF consists of a human GM-CSFR leader, human codon-optimized anti-cotinine scFv and the human CD28-based CAR backbone (human CD28 extracellular and transmembrane domain linked to human CD3zeta cytoplasmic domain from GenBank HM852952.1)⁵⁴. The CAR ORF DNA was synthesized (GeneArt; Integrated DNA Technologies) and cloned into pMSGV retroviral vector (Addgene plasmid #64269) (pMSGV-hCot-28z) for the human CAR-T cell production. The anti-His human CAR ORF consists of a human CD8 leader, human codon-optimized anti-6×His scFv and the human 41BB-based CAR backbone (human CD8 extracellular, transmembrane domain and human 41BB cytosolic domain linked to human CD3zeta cytoplasmic domain). The CAR ORF DNA was synthesized (GeneArt; Integrated DNA Technologies) and cloned into the lentiviral vector slightly modified from pCDH-EF1 (Addgene plasmid #72266). The anti-Myc human CAR lentiviral vectors were prepared similarly as anti-His human CAR lentiviral vector using anti-Myc scFvs (3A6, 7A1, and 8A9; see Table 1).

### 7. Preparation of CAR Viruses

To produce ecotropic retroviruses for mouse T cell transduction, CAR retroviral plasmid was transfected into Phoenix GP cell line, along with the expression plasmid of VSV-G envelop (pMD2.G, Addgene plasmid #12259) using Lipofectamine 3000 (Invitrogen). After 48 h, the VSV-G-pseudotyped retrovirus culture supernatant was harvested and incubated with a Phoenix Eco cell line to transduce the retroviral cDNA stably. Three to five days after infection, the transduced Phoenix Eco cells that expressed the CAR molecules on the cell surface were sorted to establish retrovirus-producing cell lines (FACS Aria II, Becton Dickinson). The retroviral culture supernatant produced from this cell line was concentrated 5 to 10-fold using a centrifugal filter device (Amicon Ultra-100 kDa cut-off, Millipore, USA) for further use. The CAR retroviral plasmid for human T cells was transfected into Phoenix Eco cell line using Lipofectamine 3000 to obtain an amphotropic retrovirus for human Cot CAR-T cell transduction. After 48 h, the culture supernatant was harvested and incubated with PG13 cell line (derived from a mouse fibroblast) for stable transduction of the retroviral cDNA. The retrovirus-producing cell line was established via cell sorting based on CAR expression, and the retrovirus was produced and concentrated similarly as the murine virus-producing cell lines.

To produce lentivirus for human anti-tag CAR-T cell transduction, each lentiviral plasmid for His- or Myc-CAR was transfected into 293T cell (ATCC) with packaging plasmids (pMD.2G, pMDLg/pRRE, pRSV-rev) using Lipofectamine 3000 (Invitrogen). Culture supernatants were collected twice at 24 h and 48 h later, filtered (0.45 pm filter, Sartorius, Germany) to remove cell residual particles, and concentrated 100-fold via ultra-high-speed centrifugation for further use.

### 8. Generation of CAR-T Cells

For CAR retroviral transduction of mouse T cells, spleen, and lymph node cells were stimulated with plate-bound anti-CD3 (10 μg/ml; 145-2C11, BioXCell, USA) and anti-CD28 (2 μg/ml; 37.51, BD Biosciences) antibodies. The next day, the retrovirus particles were attached to retronectin-coated plates by centrifugation at 2,000 g for two h, and then activated T cells were added and transduced by centrifugation at 1,000 g for 10 min. After two days, transduced T cells were transferred into a fresh medium containing 20 U/ml recombinant human IL-2 (Proleukin, Novartis, Switzerland) and expanded for 2 to 3 days without further stimulation. Transduction efficiency of the CAR-T cells was estimated by surface CAR staining with fluorescein isothiocyanate (FITC)-conjugated Fab against rabbit IgG (Jackson ImmunoResearch, USA) for Cot-CAR or FITC-conjugated Fab against chicken IgY (LSBio, Seattle, USA) for CD40-CAR or biotin-labeled 6×His peptide (Biotin-GGGGSHHHHHH; Peptron, Korea) plus PE-labeled streptavidin (Biolegend) for His-CAR, and analyzed by flow cytometry with FlowJo software (TreeStar, Inc., USA).

For human Cot CAR-T cells, human peripheral blood was obtained from healthy volunteers according to the approved protocol from the Seoul National University Hospital Institutional Review Board (IRB No. 1805-153-948). PBMCs prepared by Ficoll-gradient centrifugation (2,000 rpm, 20 min, RT) were stimulated with plate-bound anti-CD3 (10 μg/ml; OKT3, BioXCell) and anti-CD28 (2 μg/ml; CD28.2, BD Biosciences) antibodies. After two days, activated human T cells were transduced similarly as mouse T cells. After two days, transduced T cells were transferred into a fresh medium containing 20 U/ml recombinant human IL-2 and expanded for three days without further stimulation. For in vivo experiments, the human CAR-T cells were further expanded in fresh medium containing 20 U/ml human IL-2 for additional three days. The transduction efficiency of human Cot CAR-T cells was also measured by CAR staining as done with mouse Cot CAR-T cells. Control T cells were generated using the same protocol for CAR-T cell generation except for retroviral transduction.

For human anti-tag CAR-T cells, PBMCs obtained through leukapheresis from healthy volunteers were stimulated with TransAct reagent (10 μl/ml, Miltenyi Biotec) in TexMACS™ media (Miltenyi Biotec) containing human IL-7 (12.5 ng/ml, Miltenyi Biotec) and human IL-15 (12.5 ng/ml, Miltenyi Biotec). After 24 h, the activated T cells were harvested, resuspended (1×10⁶/ml) in a TM media containing human IL-7, human IL-15, and the lentiviral concentrate, and cultured for two days for CAR transduction. After washing the transduced T cells, the cells were expanded in TM media with human IL-7 and human IL-15 for eight days before use. CAR expression on the cell surface was determined by flow cytometry using biotin-labeled 6×His peptide (Biotin-GGGGSHHHHHH; Peptron) for hHis CAR-T or biotin-labeled c-Myc peptide (Biotin-GGGGSEQKLISEEDL; Peptron) for hMyc CAR-T and PE-labeled streptavidin (Biolegend).

### 9. In Vitro Functional Activity of CAR-T Cells

For the FACS-based cytotoxicity assay, target tumor cells were labeled with PKH26 (Sigma-Aldrich) according to the manufacturer's instructions. The tumor cells (target; 5×10⁴) and CAR-T cells (effector) were mixed at various effector: target (E: T) ratios (0:1 to 25:1) in 500 μl culture media per reaction and incubated for six h with subsequent 7-AAD staining (Biolegend). The number of viable tumor cells (7-AAD⁻, PKH⁺) was determined using the cell-counting beads (123count eBeads, ThermoFisher Scientific) via flow cytometry. The percent cytotoxicity was calculated using the formula: (the number of viable tumor cells in the tube without CAR-T cells−the number of viable tumor cells in the tube with CAR-T cells)/the number of viable tumor cells in the tube without CAR-T cells×100. For luminescence-based cytotoxicity assay, luciferase-transfected tumor cells (target) were placed in 96-well white plates at a concentration of 2×10⁴ cells/100 μl in triplicates, and various numbers of CAR-T cells (effector) were added at effector: target (E:T) ratios of 0:1 to 10:1. After co-culture for 16 to 24 h, 30 μg D-luciferin (Promega, USA) was added to each well, and the luminescent signals (relative light units, RLU) were measured for 10 sec using SpectraMax i3x plate reader (Molecular Devices, USA). The tumor cell viability was calculated from the average RLU of the triplicate samples using the following formula: tumor cell viability (%)=average RLU in the tube with CAR-T cells/average RLU in the tube without CAR-T cells×100.

CAR-T cells (2×10⁴) were co-cultured with target tumor cells (1×10⁵) for 24 h to evaluate CAR-T cell activation. The culture supernatants were harvested, and the amounts of IFN-γ produced were measured with mouse or human IFN-γ ELISA set (BD Bioscience) according to the manufacturer's instruction.

For the switchable CAR T cells, the target tumor cells were pre-incubated with the adaptors for 1 h and washed to remove the free adaptors prior to incubation with CAR-T cells for both cytotoxicity and IFN-γ production assays.

### 10. Cytokine Production by Macrophages and Dendritic Cells

For isolation of macrophages, Balb/C mice were injected intraperitoneally with 1 ml 3% Brewer's thioglycolate medium (Difco, USA). After 5-7 days, peritoneal lavage fluid was harvested using 10 ml of RPMI-1640 media with 2.5% FBS, and the macrophages were purified using a macrophage isolation kit (Miltenyi Biotec, Germany). Approximately 70% of the cells were F4/80-positive. For isolation of dendritic cells (DC), spleens of Balb/C mice were dissected and treated with collagenase D. After Nycodenz (Sigma-Aldrich) gradient centrifugation, DCs were purified using pan DC microbeads (Miltenyi Biotec). Approximately 75% of the cells were CD11c-positive. The macrophages or DCs (5×10⁴) were co-cultured with CAR-T cells (1×10⁴) for 24 h, and the supernatants were harvested to measure the amounts of IL-6 (Biolegend) and IL-1β (ThermoFisher Scientific) via ELISA according to the manufacturer's instructions. For Cot CAR-T cells, the macrophages or DCs were pre-incubated with the cotinine-labeled adaptors for 1h and washed to remove the free adaptors prior to incubation with the CAR-T cells.

### 11. In Vivo CAR-T Cell Therapy Models

For syngeneic murine CAR-T cell therapy models, Balb/C mice were inoculated intravenously with A20-Luc cells (1×10⁶). After 6 days, the mice were irradiated (2.5 Gy) for lymphodepletion. CD40 CAR-T or Cot CAR-T cells (5×10⁶) were administered intravenously the next day. For Cot CAR-T cells, the cotinine-labeled adaptor (C1C02-Cot, SEQ ID NO: 109, 20 μg) was injected intravenously once every other day, beginning on the day of CAR-T cell administration, for 8 times. For a xenogeneic human Cot CAR-T cell therapy model, NSG mice were inoculated with Daudi-Luc cells (5×10⁵) intravenously. Three days later, hCot CAR-T cells (1×10⁷) were administered intravenously. The cotinine-labeled anti-human CD40 adaptors (2B1-Cot, SEQ ID NO: 119, or 2E1-Cot, SEQ ID NO: 123, 25 μg,) were injected intravenously once every other day from the day of CAR-T cell administration for a total of eight times. For xenogeneic hHis CAR-T therapy model, NSGA-SID mice were inoculated with Raji-Luc cells (1×10⁵) intravenously. 2 days later, hHis CAR-T cells (5×10⁶) were administered intravenously. 6×His-tagged anti-human CD40 adaptor (2B1-Cκ-His, 25 μg, SEQ ID NO: 127) was injected intravenously once every other day from the day of CAR-T cell administration, 8 times. For evaluating therapeutic efficacy, tumor burden was measured by weekly peritoneal injection of D-luciferin (2 mg/head, Promega) and bioluminescence imaging via IVIS 100 (PerkinElmer, USA).

For cytokine neutralization experiments, anti-mIL-6 (MP5-20F3, BioXCell) were administered intraperitoneally once a day for six consecutive days beginning at 5 h before CAR-T cell transfer (500 μg for the first dose and 250 μg for the subsequent doses for 5 days). Anakinra (Kineret, Swedish Orphan Biovitrum AB, Sweden) was administered intraperitoneally at a dose of 600 μg once a day for 5 days, beginning at 5 h before CAR-T cell transfer²⁹. For macrophage depletion, the mice were treated intraperitoneally with clodronate liposome (1 mg; Liposoma, The Netherlands) for three consecutive days before CAR-T cell infusion²⁸.

For in vivo CAR-T cell tracing, Balb/C T cells expressing both CAR and effluc (CAR-Luc-T cells) or the control T cells expressing both GFP and effluc (Control-Luc-T cells) (1×10⁶ or 5×10⁶) were transferred intravenously to the irradiated (2.5 Gy) Balb/C mice. CAR-T cell trafficking was monitored by bioluminescence imaging at 6 h, day 1, day 3, and day 7 after CAR-T cell injection. In some experiments, A20 cells (2×10⁷) were injected subcutaneously 13 days before the CAR-T cell transfer.

### 12. Generation of Bone Marrow Chimera Mice

Recipient B6 or CD40 knockout mice on a B6 background were lethally irradiated (total 7.5 Gy split into 4 Gy plus 3.5 Gy, separated by 4 h). The next day, the mice were injected intravenously with 5-6×10⁶ T cell-depleted donor (B6 or CD40 knockout) bone marrow (BM) cells. In detail, BM cells were isolated from the tibia and femur of the mice, and the T cells were depleted using a cocktail of anti-Thy1.2 (30-H12), anti-CD4 (GK1.5), and anti-CD8 (53-6.7) antibodies (BD Biosciences) and guinea pig complement (Cedarlane, Canada) as previously described⁵⁵. Chimerism in peripheral blood was assessed 5-7 weeks after BM transplantation (BMT) by flow cytometry. FITC-conjugated anti-mCD40 (3/23, BD Biosciences) was used to check the establishment of donor chimerism in recipient peripheral bloods. After 8 weeks following BMT, CD40 CAR-T cells generated from B6 lymphocytes were transferred to the recipients. Survival of the mice and the body weight change were monitored regularly.

### 13. Histological Analysis of Mouse Tissues

Balb/C mice were anesthetized and transcardially perfused with 10 ml PBS. Various organs (lung, liver, spleen, intestine and kidney) were isolated and fixed in 4% paraformaldehyde in PBS with subsequent paraffin embedding. For immunohistochemical staining for CD40, 4 pm paraffin sections were deparaffinized and rehydrated before the staining. Endogenous peroxidase activity was blocked with 3% hydrogen peroxide in methanol for 10 min. The slides were then blocked with 1% bovine serum albumin in PBS for 1 h and stained with C1C02-Cκ as a primary antibody at 4° C. for 24 h. CD40 expression was detected by secondary antibody staining using biotinylated goat anti-human kappa light chain (Invitrogen) and a third antibody staining using avidin-HRP (Biolegend) and subsequent chromogenic reaction using a DAB substrate kit (DAB057, Zytomed, Germany). The slides were counterstained with hematoxylin (Sigma-Aldrich).

14. qRT-PCT

The perfused and dissected Balb/C mouse organs (lung, liver, spleen, intestine and kidney) were homogenized with TissueLyser II (Qiagen), and total RNA was extracted using TRIzol reagent (Invitrogen). cDNA was generated by using the PrimeScript™ RT Reagent (Takara Bio, Japan). qRT-PCR was performed with KAPA SYBR FAST qPCR Master Mix (KAPA Biosystems, USA) in triplicates. CD40 mRNA level was normalized with that of β-actin mRNA. The primer sequences are as follows: for mouse CD40, forward: 5′-CTGTGAACCCAATCAAGGGC-3′ and reverse: 5′-GACGGTATCAGTGGTCTCAG-3′; for mouse β-actin, forward: 5′-CGTGAAAAGATGACCCAGATCA-3′ and reverse: 5′-TGGTACGACCAGAGGCATACA-3′.

### 15. Statistical Analysis

The significance for survival data was evaluated using the log-rank (Mantel-Cox) test and the other statistical analyses were performed with unpaired two-tailed t-test using the GraphPad Prism software. Asterisks denoting statistical significance represent p-values less than 0.05 (*), 0.01 (**) and 0.001 (***), respectively.

### H. Switchable CAR-NK Cells

1. Anti-his Switchable CAR-NK Cells with Anti-Human CD40 Probes Show Functional Activities In Vitro.

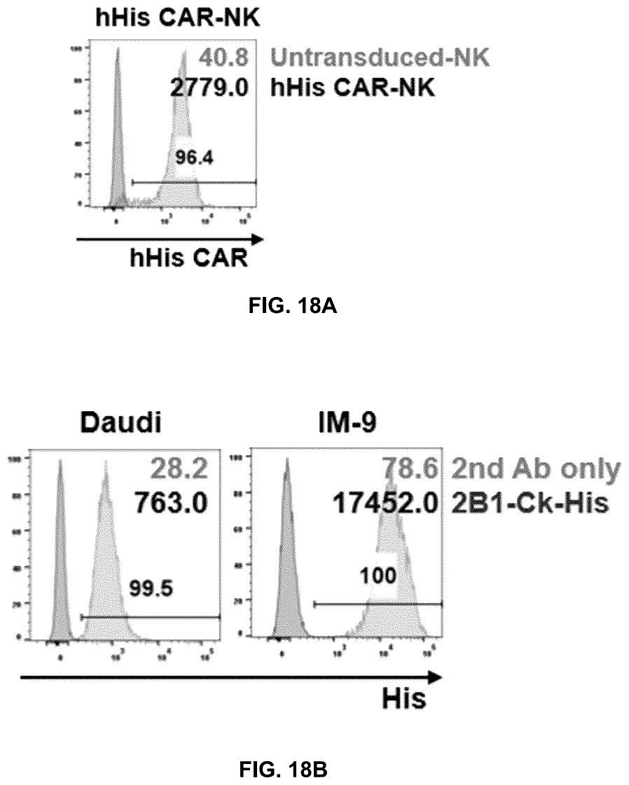

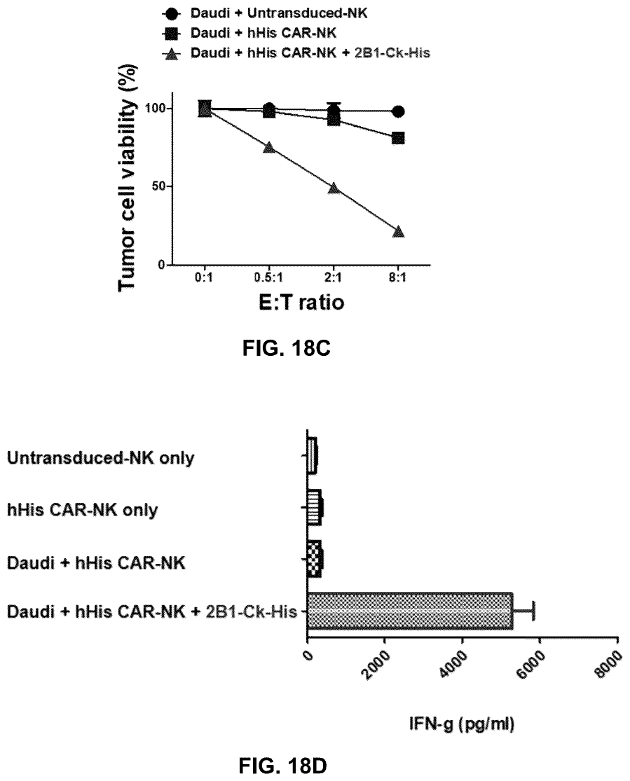

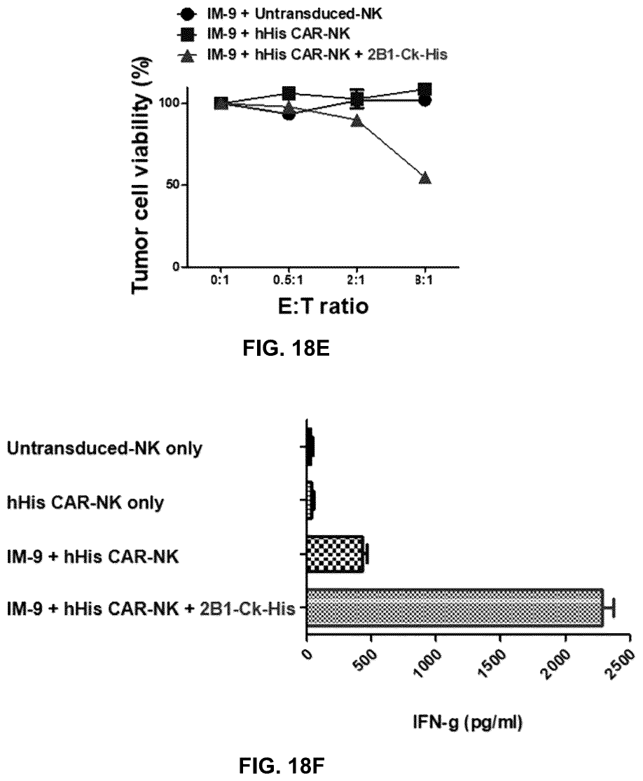

We next wanted to examine whether this therapeutic effect of anti-His CAR-T cells could be recapitulated in CAR-NK cell model. Anti-6×His CAR-NK cells (hHis CAR-NK cells) were generated by stable transduction of the lentiviral cDNA (FIG. 18A). The binding of hCD40-Cκ-His adaptor to the tumor cell surface was confirmed by flow cytometry (FIG. 18B). In vitro cytotoxicity and functional activity of hHis CAR-NK cells in the presence of hCD40 adaptors were validated in in vitro co-culture experiments with Daudi or IM-9 cells (FIGS. 18C-18F). Hence, we confirmed that the switchable anti-His CAR system is applicable not only to CAR-T cells but also to CAR-NK cells.

### 2. Materials and Methods for Example I

### a) Cell Lines

NK-92 (Interleukin-2 (IL-2) dependent natural killer cell), Raji (human B cell lymphoma) and IM-9 (human multiple myeloma) cell lines were purchased from the American Type Culture Collection (ATCC, USA).

b) Generation of CAR-NK cells

To produce lentivirus for human anti-tag CAR-NK cell transduction, each lentiviral plasmid for His-CAR was transfected into 293T cell (ATCC) with packaging plasmids (pSF-VSV-G, pSF-Rev, pSF-Gag/pol) using Lipofectamine 3000 (Invitrogen). Culture supernatants were collected twice at 24 h and 48 h later, filtered (0.45 μm filter, Satorius, Germany) to remove cell residual particles, and concentrated 10-fold using a centrifugal filter device (Amicon Ultra-100 kDa cut-off, Millipore, USA). Concentrated supernatants were transferred into NK-92 cells by spin infection with polybrene at twice. And then, The His CAR-high populations were sorted with a FACS Aria II cell sorter (Becton Dickinson, USA).

### c) In Vitro Functional Activity of CAR-NK Cells

For luminescence-based cytotoxicity assay, luciferase-transfected tumor cells (target) were placed in 96-well white plates at a concentration of 1×10⁴ cells/100 μl in triplicates, and various number of CAR-T cells (effector) were added at effector:target (E:T) ratios of 0:1 to 8:1. After co-culture for 2 h, 30 μg D-luciferin (Promega, USA) was added to each well, and the luminescent signals (relative light units, RLU) were measured for 10 sec using SpectraMax i3x plate reader (Molecular Devices, USA). The tumor cell viability was calculated from the average RLU of the triplicate samples using the following formula: tumor cell viability (%)=average RLU in the tube with CAR-NK cells/average RLU in the tube without CAR-NK cells×100.

CAR-NK cells (2×10³) were co-cultured with target tumor cells (1×10⁴) for 24 h to evaluate CAR-NK cell activation. The culture supernatants were harvested, and the amounts of IFN-γ produced were measured with human IFN-γ ELISA set (BD Bioscience) according to the manufacturer's instruction.

### I. Humanized his CAR-T Cells and Human Anti-CD40 Antibodies

1. Humanized Anti-his CAR-T (huHis CAR-T) Cells with Humanized Anti-Human CD40 (hu2B1-Cκ-his) Adaptors Show Functional Activities In Vitro.

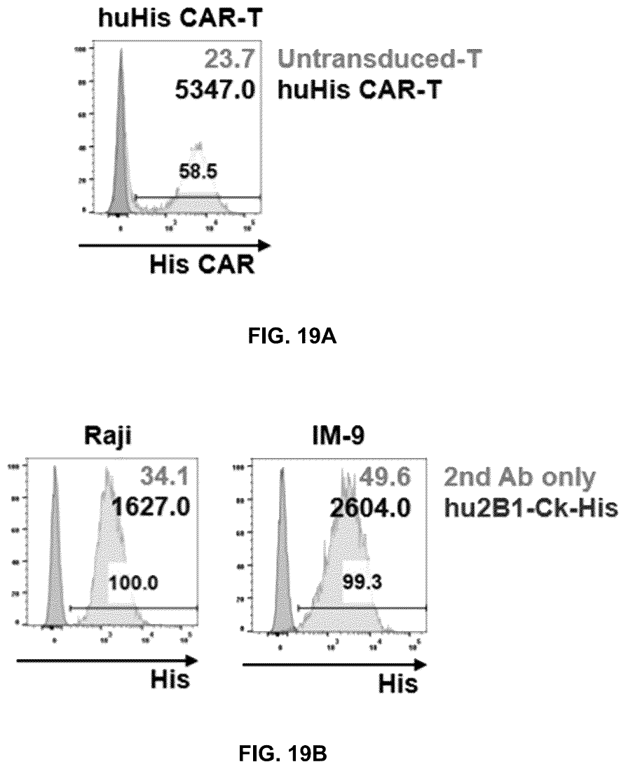

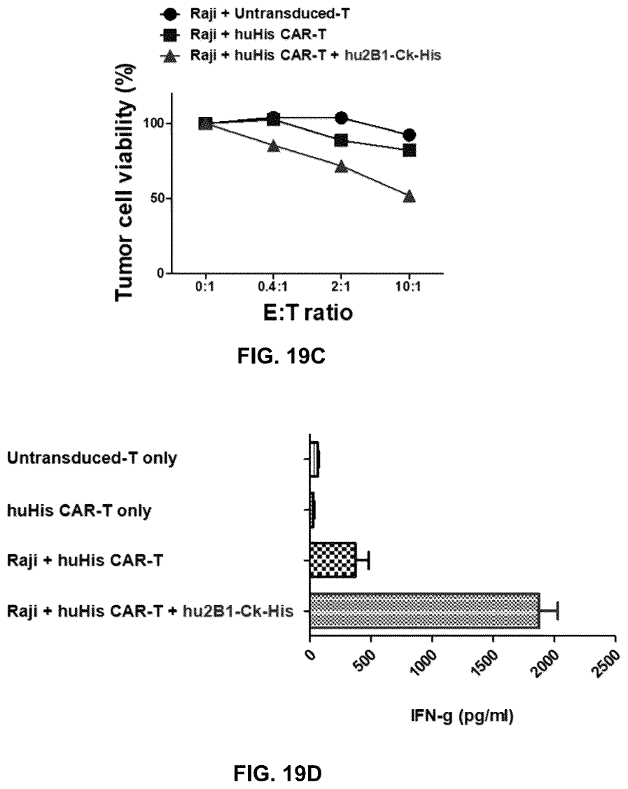

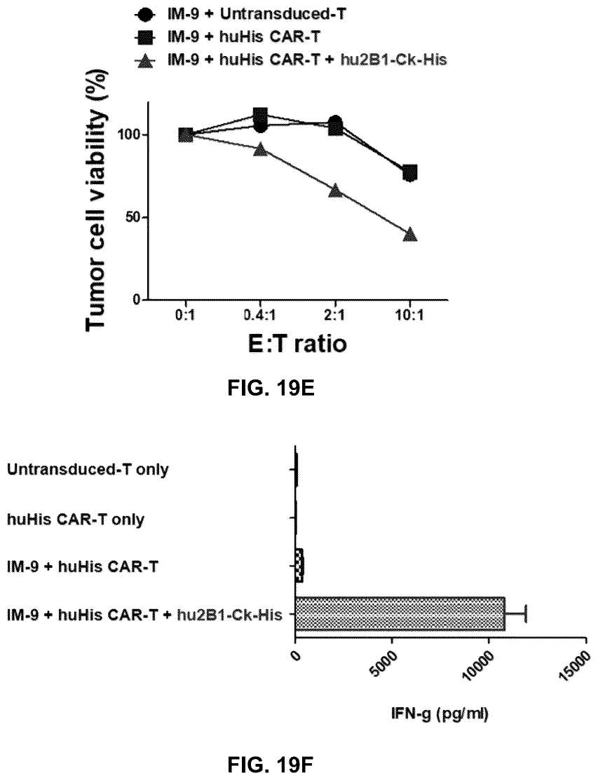

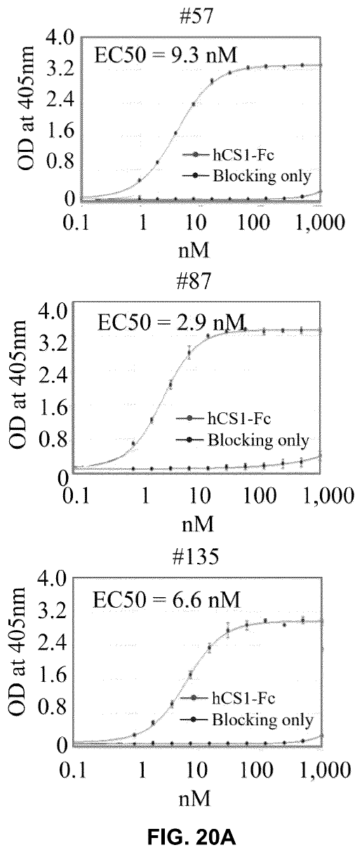

We generated a humanized anti-CD40-His adaptor (hu2B1-Cκ-His) and a humanized anti-His CAR-T cell with human 41BB/CD3zeta CAR backbone (huHis CAR-T cells) for potential clinical application. Then, we evaluated whether this humanized switchable CAR system shows anti-tumor functionality in vitro. HuHis CAR molecules were expressed efficiently on HuHis CAR-T cells (FIG. 19A), and hu2B1-Cκ-His adaptor binding to tumor cells was confirmed successfully (FIG. 19B). When HuHis CAR-T cells were co-cultured with Raji or IM9 tumor cells in the presence of hu2B1-Cκ-His adaptor, CAR-T cells showed anti-tumor cytotoxicity and produced IFN-γ similarly to the non-humanized switchable CAR-T cells (FIGS. 19C-19F). Thus, we confirmed that humanized switchable CAR-T cells maintain its anti-tumor efficacy.

### 2. Materials and Methods for Example J

All procedures were same as those for non-humanized CAR-T cells except for humanized scFvs were used for CAR construction and adaptor generation.

### J. Switchable CAR-T Cells Targeting CS1

1. Screening of Anti-CS1 Antibody from the Immune Library

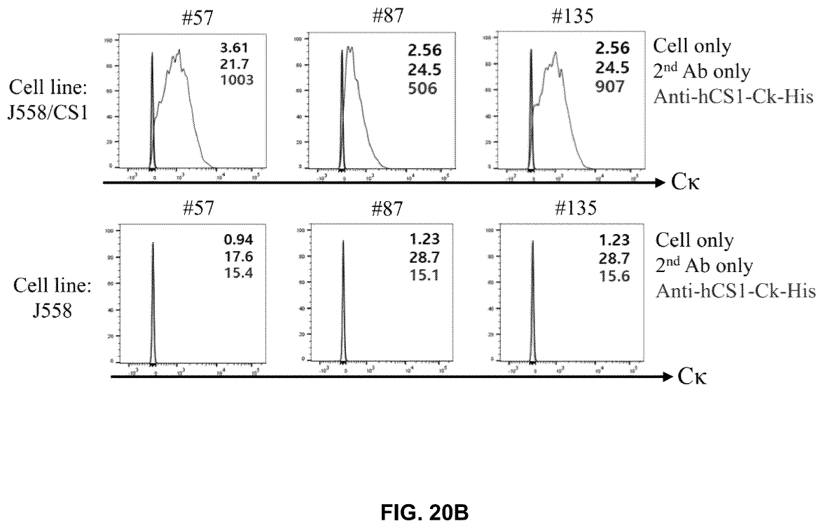

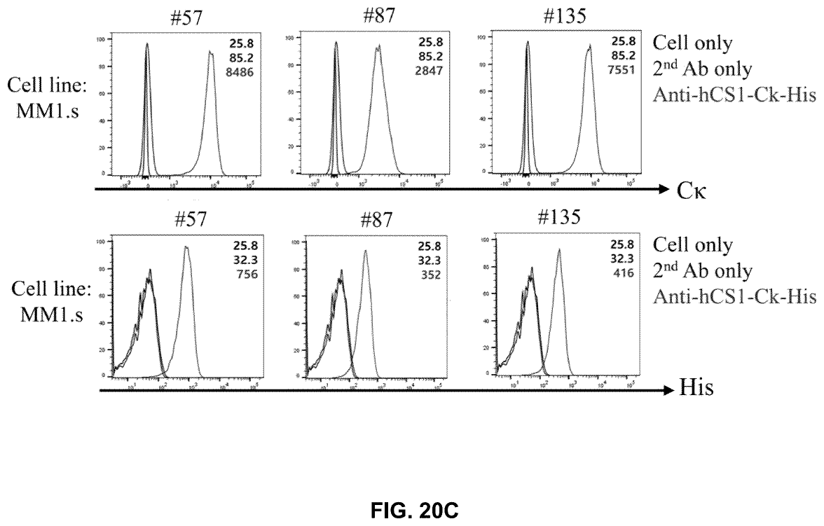

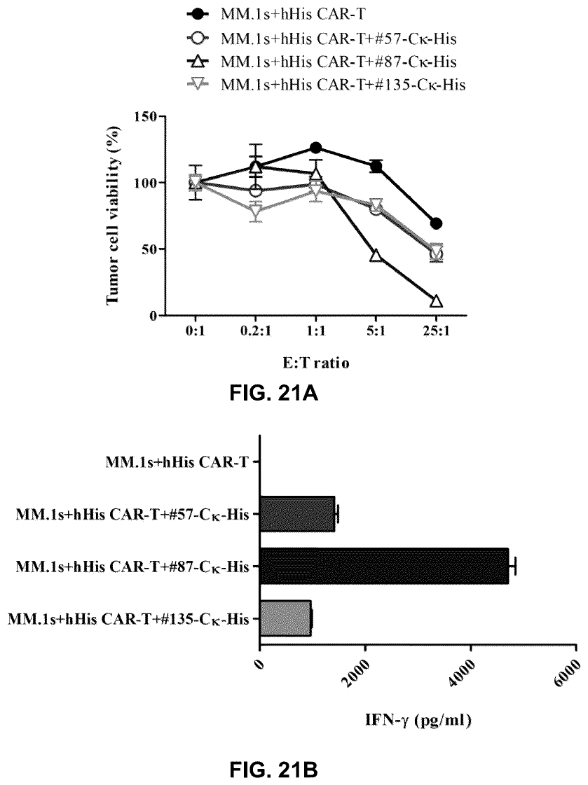

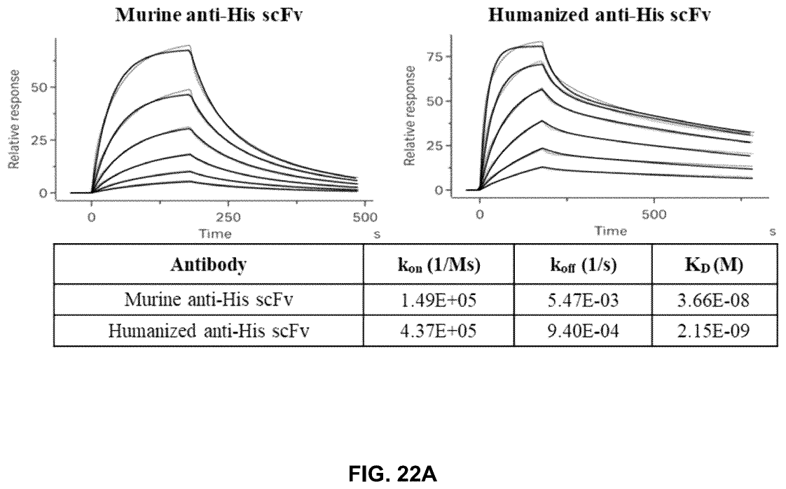

To generate anti-CS1 antibodies, chickens were immunized with the human CS1-Fc fusion protein. After 4^{th} round immunizations, we collected spleen, bone marrow, and bursa of fabricius, and extracted total RNA, and constructed a scFv phagemid library for phage display. ScFvs were selected by five rounds of panning on the human CS1-His fusion protein. In the panning, we used magnetic bead with the immobilized irrelevant Fc fusion protein to subtract Fc-specific binders. We evaluated the binding of selected scFv clones using phage ELISA (FIG. 20). Individual binders were identified by randomly picking more than 746 clones and checking their binding activity on CS1 protein by monoclonal phage ELISA. A total of 161 clones (22%) showed positive binding activity on CS1. The selection criterion for positive binders was an absorption value more than 1.0 without unwanted cross-reaction to Fc and background signal. The DNA representing ELISA positive clones were sequenced and aligned, representing 23 sequence-specific scFv clones. Considering sequence repeatability and ELISA signal strength, 3 scFv clones (#57, #87, #135) were selected and used for further analysis.

### 2. Evaluation of Selected Anti-CS1 Antibodies

In other to verify the binding activity of three phage-screened scFv clones, antibody format was changed to scFv-human Cκ-(G₄S)₂ linker-6×His tag (scFv-Cκ-His) and transferred to pCEP4 mammalian expression vector. Since scFv-Cκ-His will works as an adapter on its own, a (G₄S)₂ flexible linker was used between Cκ and 6×His tag to provide distance for anti-His CAR-T cells to access the 6×His tag. After purification of these 5 scFv-Cκ-His clones, we tested their CS1 binding activity by serial dilution ELISA against recombinant CS1-Fc fusion protein (FIG. 20A). They showed an increase in concentration-dependent responses in the concentration range of 0.078 to 40 nM. Although small differences in binding activity, all scFv antibodies bound to fusion proteins with similar activity, EC50 ranging from 2.9 nM to 9.3 nM. Among them, the #87 clone reached saturation state the fastest and the #57 clone reached saturation state the latest. To test each clone's binding ability to the native CS1 structure, we performed a FACS analysis using J558 cells transfected with CS1 gene (FIG. 20B). Three clones showed specific reactivity only with CS1 overexpressed J558 cells (not reacted with J558 cells), and #57 clone was shifted the most and #87 clone was shifted the least which is contrary to the ELISA data. This data was also reproduced in the FACS analysis using MM1.s cells naturally expressing CS1 (FIG. 20C). So, we confirmed that selected three clones were indeed bind to native CS1 protein and #57 clone was the highest binding activity with native CS1 protein.

3. In Vitro Potency of his Switchable CAR-T Cells with CS1 Target Adaptors

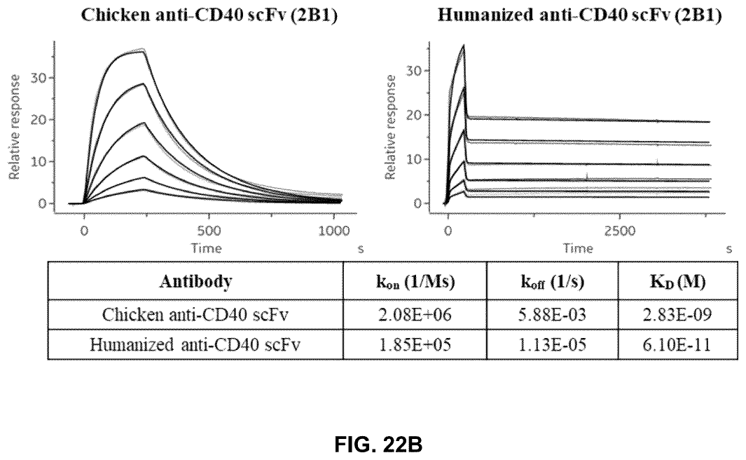

We wanted to examine whether anti-6×His CAR-T cells (hHis CAR-T cells) are also functional using with anti-human CS1 scFv-Ck-His adaptors (#57, 87, 135). The His tagged CS1 adaptor bound to MM.1s tumor cells, and mediated hHis CAR-T cells-dependent tumor cell cytotoxicity and CAR-T cell activation in vitro (FIGS. 22A, 22B). hHis CAR-T cells showed antitumor efficacy and cytokine production in presence of His tagged CD40 adaptor and CS1 adaptor. Therefore, hHis CAR-T cells could be used with various adaptors by tagging 6× His to the adaptors.

### 4. Materials and Methods for Example K

### a) Immunization and Phage Library Construction

The White Leghorns (Charles River), 3˜4 weeks old, were injected subcutaneously with purified CS1-Fc protein in Freund's adjuvant (Sigma, #F5881 and #F5506) four times with two-week interval. The initial immunization utilized 50 μg immunogen with complete Freund's adjuvant, whereas subsequent immunization utilized 25 μg immunogen with incomplete Freund's adjuvant. Total RNA was isolated from spleen, bone marrow, and bursa of fabricius using TRI reagent (Invitrogen, #AM9738) and cDNA was synthesized using a SuperScript™ III First-Strand Synthesis System (Invitrogen, #18080051) with Oligo(dT) primers. The Vλ and VH gene repertoires were amplified from cDNA and scFv gene repertoires were amplified by spliced together of Vλ and VL gene. The PCR products and pComb3×SS phagemid vector were digested with SfiI restriction enzyme and ligated, transformed into E. coli strain ER2738 by electroporation. The cells were plated overnight on LB (Lennow Broth) agar plates with 2% glucose and 100 μg/mL carbenicillin to harbor an enriched phagemid library, which is subsequently harvested to infect helper phage VCSM13 with >10¹¹ cfu/mL (Agilent, #200251) for 1 hour. ScFv-display phages were generated by overnight culture at 25° C. with SB (Super Broth) containing 100 μg/mL carbenicillin and 50 μg/mL Kanamycin. ScFv-display phage particles were purified from culture supernatants by precipitation with 4% polyethylene glycol-8000 (PEG-8000) and 3% NaCl and suspended in PBS containing 1% BSA.

### b) Phage Library Panning

The scFv-display phage library was panned against recombinant human CS1-6×His tag (CS1-His) protein (ACROBiosystems, #SL7-H5225) coated on M270-Epoxy Dynabead (Invitrogen, #14301). After subtraction of Fc binder by incubation of scFv-display phage library with human Fc coated bead for 2 hours at 37° C., the phages were incubated with human CS1-6×His tag coated Dynabead for 2 hours at 37° C. The beads were washed with 0.5% PBST (PBS containing 0.5% Tween-20) to remove phages which were not bound. The bound phages were eluted with 0.1 M glycine-HCl and neutralized with 2 M Tris-HCl (pH 9.0). Recovered phages were infected into ER2738 and plated overnight on LB agar plates with 2% glucose and 100 μg/mL carbenicillin. Subsequent phage rescue, amplification, and purification procedures were identical to those described in phage library construction. During 5 rounds panning, the concentration of antigen coated on the beads was adjusted from 1.5 μg to 0.15 μg per 5×10⁶ beads to ensure adequate stringency during the panning process.

### c) Enzyme-Linked Immunosorbent Assay (ELISA)

The entire ELISA procedure except antigen coating were carried out at room temperature. 96-well plates (Corning, #3690) were coated with 100 μL of 100 μg/mL antigens (CS1-Fc or CS1-His or Fc) at 4° C. overnight and blocked with PBS buffer containing 3% BSA for 1 hour. For phage ELISA, individual scFv-display phages were diluted 1:1 in PBS buffer containing 6% BSA, then incubated with immobilized antigens for 2 hours, and bound phages were detected with horseradish peroxidase (HRP)-conjugated monoclonal anti-M13 antibody (Sino Biological, #AB_2857928) for 1 hour. For the purified antibody binding assay, antibodies were two-fold serial diluted in PBS buffer containing 3% BSA, then incubated with immobilized antigens, and bound antibodies were detected with HRP-labeled goat anti-human Ck antibodies (Merck, #AP502P). The enzyme activity was measured with the subsequent addition of substrate 2, 2′-azino-bis (3-ethylbenzothiazoline-6-sulphonic acid) (ABTS) (Thermo) and signal reading was carried out at 450 nm using a Microplate Spectrophotometer.

### d) Protein Expression and Purification

For immunogen preparation, human CS1 ectodomain (S23˜M226) protein was expressed with the Expi293™ Expression System (Invitrogen). The corresponding gene of the protein were synthesized (GeneArt) and subcloned into a modified pCEP4 vector (Invitrogen), which contains a N-terminal secreting signal peptide sequence and a C-terminal human IgG1 Fc. The expression construct (CS1-Fc) was transfected into Expi293™ cells and cultured according to the manufacturer's protocol. After 96 hours of transfection, the cell culture containing the secreted proteins was collected and diluted 1:1 in PBS, then purified with protein A affinity column (Repligen, CativaA® Protein A affinity resin). The bound proteins were eluted with Pierce™ IgG Elution buffer (pH 2.8) (Thermo, #21004) and neutralized with 1 M Tris-HCl buffer (pH 9.2). After dialyzed in 1×PBS, the purity was assessed by protein gel analysis using 4-15% gradient SDS-PAGE and the concentration was determined by measuring the absorbance at 280 nm. For preparation of anti-CS1 antibodies, the gene sequences of the scFv were amplified with PCR from phagemid DNA and subcloned into a modified pCEP4 vector, which contains N-terminal secreting signal peptide sequence and C-terminal human kappa constant domain gene (Cκ), (G₄S)₂ linker and 6×His tag. The expression construct (scFv-Cκ-His) was expressed and purified with KappaSelect affinity column (Cytiva, #17545802).

### e) Cell Lines

293T cells, J558 (mouse B-lymphoblast Myeloma) and MM.1s (human B-lymphoblast Myeloma) were purchased from American Type Culture Collection (ATCC, USA). J558/CS1 overexpressed cells were generated by transfection of human CS1 gene. MM.1s-Luc cells were generated by spin infection with lentivirus (pCEF-LucGFP) harboring luciferase-P2A-EGFP expression cassette.

### f) Flow Cytometry Analysis

To detect cellular binding of purified antibodies, cells were first incubated with anti-CS1 antibodies for 30 minutes at 37° C. After the reaction, cells were washed and stained with mouse APC conjugated anti-human Cκ antibodies (Biolegend, #316509) or mouse APC conjugated anti-His tag secondary antibodies (Biolegend, #362605) for 30 minutes at 37° C. Finally, the fluorescence intensity of the samples was detected by FACS.

### g) Preparation of CAR Construct

The anti-His human CAR ORF consists of a human CD8 leader, human codon-optimized anti-6×His scFv and the human 41BB-based CAR backbone (human CD8 extracellular and transmembrane domain, human 41BB cytoplasmic domain linked to human CD3zeta cytoplasmic domain). The CAR ORF DNA was synthesized (GeneArt; Integrated DNA Technologies) and cloned into the lentiviral vector slightly modified from pCDH-EF1 (Addgene plasmid #72266).

### h) Preparation of CAR Viruses

To produce lentivirus for human anti-6×His CAR-T cell transduction, CAR lentiviral plasmid was transfected into 293T cell (ATCC) with packaging plasmids (pSF-VSV.G, pSF-REV, pSF-Gag/pol) using Lipofectamine 3000 (Invitrogen). After 6 h, the culture supernatants was replaced with 10 ml fresh medium. Culture supernatants were collected twice at 24 h and 48h later, filtered (0.45 μm filter, Sartorius, Germany) to remove cell residual particles, and concentrated 100-fold via ultra-high-speed centrifugation for further use.

### i) Generation of CAR-T Cells

PBMCs obtained through leukapheresis from healthy volunteers were stimulated with TransAct reagent (10 μl/ml, Miltenyi Biotec) in TexMACS™ media (Miltenyi Biotec) containing 200 U/ml recombinant human IL-2 (Proleukin, Novartis, Switzerland). After 24 h, the activated T cells were harvested, resuspended (1×10⁶/ml) in a TM media containing recombinant human IL-2 (200U/ml), and the lentiviral concentrate, and cultured for two days for CAR transduction. After washing the transduced T cells, the cells were expanded in TM media with recombinant human IL-2 (200U/ml) for five days before use. CAR expression on the cell surface was determined by flow cytometry using biotin-labeled 6×His peptide (Biotin-GGGGSHEHHHH; Peptron, Korea) plus PE-labeled streptavidin (Biolegend).

### j) In Vitro Functional Activity of CAR-T Cells

The target tumor cells were pre-incubated with the adaptors for 1 h and washed to remove the free adaptors prior to incubation with CAR-T cells for cytotoxicity and cytokine production assays. For luminescence-based cytotoxicity assay, the luciferase-transfected tumor cells (target) were placed in 96-well white plates at a concentration of 2×10⁴ cells/100 μl in triplicates, and various numbers of CAR-T cells (effector) were added at effector:target (E:T) ratios of 0:1 to 25:1. After co-culture for 6 h, 30 μg D-luciferin (Promega, USA) was added to each well, and the luminescent signals (relative light units, RLU) were measured for 10 sec using SpectraMax i3x plate reader (Molecular Devices, USA). The tumor cell viability was calculated from the average RLU of the triplicate samples using the following formula: tumor cell viability (%)=average RLU in the well with CAR-T cells/average RLU in the well without CAR-T cells×100. To evaluate CAR-T cell activation, CAR-T cells (2×10⁴) were co-cultured with target tumor cells (1×10⁵) for 24 h. The culture supernatants were harvested and the amounts of IFN-γ produced were measured using a human IFN-γ ELISA set (BD Bioscience) according to the manufacturer's instructions.

### K. Humanized Anti-His and Anti-CD40 Abs

Since the murine anti-His antibody had decreased binding affinity against 6×His peptide after humanization, affinity maturation was further performed to increase it and chicken anti-CD40 antibody (2B1) was only humanized. Serial dilution antibodies at 6 concentrations were used to characterize the kinetics of antibody-antigen binding by SPR. Humanized and affinity maturated anti-His scFv possessed an affinity of 2.15 nM, 5.8-fold higher than parental antibody (FIG. 22A). And humanized anti-CD40 scFv possessed an affinity of 6.10 nM, 21.6-fold higher than parental antibody. So, there was not affinity loss against their antigen after humanization (FIG. 22B).

### 1. Materials and Methods for Example L

### a) Antibody Humanization and Affinity Maturation

For humanization and affinity maturation of murine anti-His scFv antibodies, firstly, 6 CDRs (Complementarity Determining Regions) were grafted into IGHV1-18 and IGKV2-28 human germline genes, which exhibited the most appropriate sequence similarity. At the same time, several amino acid sites in the Framework Regions (FRs) were changed to remove post-translational modification and to perform back mutations. Subsequently, a sub-library of humanized scFv containing site-directed mutations in 6 CDRs was constructed for affinity maturation. The antibodies were produced using Expi293 expression system and affinity purified, then ELISA was performed to confirm binding affinity to antigen (WuXi Biologics). In case of anti-CD40 scFv antibodies, only humanization was performed, and 6 CDRs were grafted into IGHV3-23 and IGKV1-1 human germline genes.

### b) SPR Binding Affinity Measurement of Humanized Antibody

A Biacore 8K SPR system (Cytiva) equipped with CM5 sensor chip (Cytiva) was used to generate binding kinetic rate and affinity constants. To prepare the capture surfaces for anti-His antibodies, streptavidin was immobilized, followed by biotin conjugated 6×His peptide was captured. The antibodies were 2-fold serial diluted with 1×PBS, then injected at a flow rate of 30 μL/min for 3 minutes, allowing 10 minutes dissociation phase. The surface was regenerated using 10 mM glycine, pH 1.5. The kinetics were analyzed using Biacore 8K evaluation software, version 1.0 (Cytiva). In case of anti-CD40 antibodies, anti-human Fc IgG (Jackson ImmunoResearch) was immobilized, and then CD40-hFc protein (Acro Biosystems, #CD0-H5253) was captured. The antibodies were 2-fold serial diluted with HBS-EP+ buffer (10 mm HEPES, 150 mm sodium chloride, 3 mm EDTA, 0.05% Polysorbate 20), then injected at a flow rate of 30 μL/min for 4 minutes, allowing >15 minutes dissociation phase.

### L. References of the Present Disclosure

1. Kershaw M H, Teng M W, Smyth M J, Darcy P K. Supernatural T cells: genetic modification of T cells for cancer therapy. Nat Rev Immunol 5, 928-940 (2005).
2. Maude S L, et al. Chimeric antigen receptor T cells for sustained remissions in leukemia. N Engl J Med 371, 1507-1517 (2014).
3. Gill S, Brudno J N. CAR T-Cell Therapy in Hematologic Malignancies: Clinical Role, Toxicity, and Unanswered Questions. Am Soc Clin Oncol Educ Book 41, 1-20 (2021).
4. Rafiq S, Hackett C S, Brentjens R J. Engineering strategies to overcome the current roadblocks in CAR T cell therapy. Nat Rev Clin Oncol 17, 147-167 (2020).
5. Lamers C H, et al. Treatment of metastatic renal cell carcinoma with CAIX CAR-engineered T cells: clinical evaluation and management of on-target toxicity. Mol Ther 21, 904-912 (2013).
6. Morgan R A, Yang J C, Kitano M, Dudley M E, Laurencot C M, Rosenberg S A. Case report of a serious adverse event following the administration of T cells transduced with a chimeric antigen receptor recognizing ERBB2. Mol Ther 18, 843-851 (2010).
7. Thistlethwaite F C, et al. The clinical efficacy of first-generation carcinoembryonic antigen (CEACAM5)-specific CAR T cells is limited by poor persistence and transient pre-conditioning-dependent respiratory toxicity. Cancer Immunol Immunother 66, 1425-1436 (2017).
8. Roybal K T, et al. Precision Tumor Recognition by T Cells With Combinatorial Antigen-Sensing Circuits. Cell 164, 770-779 (2016).
9. Srivastava S, et al. Logic-Gated ROR1 Chimeric Antigen Receptor Expression Rescues T Cell-Mediated Toxicity to Normal Tissues and Enables Selective Tumor Targeting. Cancer Cell 35, 489-503 e488 (2019).
10. Kloss C C, Condomines M, Cartellieri M, Bachmann M, Sadelain M. Combinatorial antigen recognition with balanced signaling promotes selective tumor eradication by engineered T cells. Nat Biotechnol 31, 71-75 (2013).
11. Lanitis E, et al. Chimeric antigen receptor T Cells with dissociated signaling domains exhibit focused anti-tumor activity with reduced potential for toxicity in vivo. Cancer Immunol Res 1, 43-53 (2013).
12. Fedorov V D, Themeli M, Sadelain M. PD-1- and CTLA-4-based inhibitory chimeric antigen receptors (iCARs) divert off-target immunotherapy responses. Sci Transl Med 5, 215ra172 (2013).
13. Hong M, Clubb J D, Chen Y Y. Engineering CAR-T Cells for Next-Generation Cancer Therapy. Cancer Cell 38, 473-488 (2020).
14. Urbanska K, et al. A universal strategy for adoptive immunotherapy of cancer through use of a novel T-cell antigen receptor. Cancer Res 72, 1844-1852 (2012).
15. Tamada K, et al. Redirecting gene-modified T cells toward various cancer types using tagged antibodies. Clin Cancer Res 18, 6436-6445 (2012).
16. Ma J S, et al. Versatile strategy for controlling the specificity and activity of engineered T cells. Proc Natl Acad Sci USA 113, E450-458 (2016).
17. Rodgers D T, et al. Switch-mediated activation and retargeting of CAR-T cells for B-cell malignancies. Proc Natl Acad Sci USA 113, E459-468 (2016).
18. Cho J H, Collins J J, Wong W W. Universal Chimeric Antigen Receptors for Multiplexed and Logical Control of T Cell Responses. Cell 173, 1426-1438 e1411 (2018).
19. Lee Y G, et al. Regulation of CAR T cell-mediated cytokine release syndrome-like toxicity using low molecular weight adapters. Nat Commun 10, 2681 (2019).
20. Costello R T, Gastaut J A, Olive D. What is the real role of CD40 in cancer immunotherapy?Immunol Today 20, 488-493 (1999).
21. Grewal I S, Flavell R A. CD40 and CD154 in cell-mediated immunity. Annu Rev Immunol 16, 111-135 (1998).
22. Hassan S B, Sorensen J F, Olsen B N, Pedersen A E. Anti-CD40-mediated cancer immunotherapy: an update of recent and ongoing clinical trials. Immunopharmacol Immunotoxicol 36, 96-104 (2014).
23. Vonderheide R H. CD40 Agonist Antibodies in Cancer Immunotherapy. Annu Rev Med 71, 47-58 (2020).
24. Hollenbaugh D, et al. Expression of functional CD40 by vascular endothelial cells. J Exp Med 182, 33-40 (1995).
25. Young L S, Eliopoulos A G, Gallagher N J, Dawson C W. CD40 and epithelial cells: across the great divide. Immunol Today 19, 502-506 (1998).
26. Zhang Y, Cao H J, Graf B, Meekins H, Smith T J, Phipps R P. CD40 engagement up-regulates cyclooxygenase-2 expression and prostaglandin E2 production in human lung fibroblasts. J Immunol 160, 1053-1057 (1998).
27. Neelapu S S, et al. Chimeric antigen receptor T-cell therapy—assessment and management of toxicities. Nat Rev Clin Oncol 15, 47-62 (2018).
28. Norelli M, et al. Monocyte-derived IL-1 and IL-6 are differentially required for cytokine-release syndrome and neurotoxicity due to CAR T cells. Nat Med 24, 739-748 (2018).
29. Giavridis T, van der Stegen S J C, Eyquem J, Hamieh M, Piersigilli A, Sadelain M. CAR T cell-induced cytokine release syndrome is mediated by macrophages and abated by IL-1 blockade. Nat Med 24, 731-738 (2018).
30. Kim H, Yoon S, Chung J. In vitro and in vivo application of anti-cotinine antibody and cotinine-conjugated compounds. BMB Rep 47, 130-134 (2014).
31. Heo K, et al. An aptamer-antibody complex (oligobody) as a novel delivery platform for targeted cancer therapies. J Control Release 229, 1-9 (2016).
32. Yu B, et al. A Hybrid Platform Based on a Bispecific Peptide-Antibody Complex for Targeted Cancer Therapy. Angew Chem Int Ed Engl 58, 2005-2010 (2019).
33. Kang H Y, et al. A modifiable universal cotinine-chimeric antigen system of NK cells with multiple targets. Font Immunol 13, 1089369 (2022).
34. Kaufmann M, et al. Crystal structure of the anti-His tag antibody 3D5 single-chain fragment complexed to its antigen. J Mol Biol 318, 135-147 (2002).
35. Viaud S, et al. Switchable control over in vivo CAR T expansion, B cell depletion, and induction of memory. Proc Natl Acad Sci USA 115, E10898-E10906 (2018).
36. Greten F R, Grivennikov S I. Inflammation and Cancer: Triggers, Mechanisms, and Consequences. Immunity 51, 27-41 (2019).
37. Weninger W, Crowley M A, Manjunath N, von Andrian U H. Migratory properties of naive, effector, and memory CD8(+) T cells. J Exp Med 194, 953-966 (2001).
38. Liu X, et al. Affinity-Tuned ErbB2 or EGFR Chimeric Antigen Receptor T Cells Exhibit an Increased Therapeutic Index against Tumors in Mice. Cancer Res 75, 3596-3607(2015).
39. Park S, et al. Micromolar affinity CAR T cells to ICAM-1 achieves rapid tumor elimination while avoiding systemic toxicity. Sci Rep 7, 14366 (2017).
40. Castellarin M, et al. A rational mouse model to detect on-target, off-tumor CAR T cell toxicity. JCI Insight 5, (2020).
41. Juillerat A, et al. Modulation of chimeric antigen receptor surface expression by a small molecule switch. BMC Biotechnol 19, 44 (2019).
42. Richman S A, Wang L C, Moon E K, Khire U R, Albelda S M, Milone M C. Ligand-Induced Degradation of a CAR Permits Reversible Remote Control of CAR T Cell Activity In vitro and In vivo. Mol Ther 28, 1600-1613 (2020).
43. Jan M, et al. Reversible ON- and OFF-switch chimeric antigen receptors controlled by lenalidomide. Sci Transl Med 13, (2021).
44. Labanieh L, et al. Enhanced safety and efficacy of protease-regulated CAR-T cell receptors. Cell 185, 1745-1763 e1722 (2022).
45. Pishali Bejestani E, et al. Characterization of a switchable chimeric antigen receptor platform in a pre-clinical solid tumor model. Oncoimmunology 6, e1342909 (2017).
46. Adams R, et al. Extending the half-life of a fab fragment through generation of a humanized anti-human serum albumin Fv domain: An investigation into the correlation between affinity and serum half-life. MAbs 8, 1336-1346 (2016).
47. Wermke M, et al. Proof of concept for a rapidly switchable universal CAR-T platform with UniCAR-T-CD123 in relapsed/refractory AML. Blood 137, 3145-3148 (2021).
48. Li D K, Wang W. Characteristics and clinical trial results of agonistic anti-CD40 antibodies in the treatment of malignancies. Oncol Lett 20, 176 (2020).
49. Bonnans C, et al. CD40 agonist-induced IL-12p40 potentiates hepatotoxicity. J Immunother Cancer 8, (2020).
50. Kuhn N F, et al. CD40 Ligand-Modified Chimeric Antigen Receptor T Cells Enhance Anti-tumor Function by Eliciting an Endogenous Anti-tumor Response. Cancer Cell 35, 473-488 e476 (2019).
51. Andris-Widhopf J, Steinberger P, Fuller R, Rader C, Barbas CF, 3rd. Generation of human scFv antibody libraries: PCR amplification and assembly of light- and heavy-chain coding sequences. Cold Spring Harb Protoc 2011, (2011).
52. Kochenderfer J N, Yu Z, Frasheri D, Restifo N P, Rosenberg S A. Adoptive transfer of syngeneic T cells transduced with a chimeric antigen receptor that recognizes murine CD19 can eradicate lymphoma and normal B cells. Blood 116, 3875-3886 (2010).
53. Park S, Lee D H, Park J G, Lee Y T, Chung J. A sensitive enzyme immunoassay for measuring cotinine in passive smokers. Clin Chim Acta 411, 1238-1242 (2010).
54. Kochenderfer J N, et al. Eradication of B-lineage cells and regression of lymphoma in a patient treated with autologous T cells genetically engineered to recognize CD19. Blood 116, 4099-4102 (2010).
55. Chakraverty R, et al. Host MHC class II+ antigen-presenting cells and CD4 cells are required for CD8-mediated graft-versus-leukemia responses following delayed donor leukocyte infusions. Blood 108, 2106-2113 (2006).
56. Veillette A. and Guo H, CS1, a SLAM family receptor involved in immune regulation, is a therapeutic target in multiple myeloma. Critical Reviews in Oncology/Hematology 88 (2013) 168-177.
57. Gogishvili et al., SLAMF7-CAR T cells eliminate myeloma and confer selective fratricide of SLAMF71 normal lymphocytes. Blood. 2017; 130(26):2838-2847.

TABLE 1
Antibody sequences

| | | | SEQ |
|---|---|---|---|
| Description | | | ID |
| Anti- | scFv | QAALTQPSSVSANPGETVKITCSGGDSYAGSYYYGWFQQKAPGSAPVTVIYY | 1 |
| hCD40 | | NDKRPSDIPSRFSGSTSGSTSTLTITGVQAEDEAVYYCGSIDTSSGTGIFGAGTT | |
| (2B1) | | LTVLGQSSRSSGGGGSSGGGGSAVTLDESGGGLQTPGGALSLVCKGSGFTFSS | |
| | | YQMFWVRQAPGKGLEYVAEISGSGLSTWYAPAVKGRATISRDNGQSTVRLQ | |
| | | LNNLRAEDTGTYYCAKAAGSCGVGTCAGGIVDGIDAWGHGTEVIVSS | |
| | VH | AVTLDESGGGLQTPGGALSLVCKGSGFTFSSYQMFWVRQAPGKGLEYVAEIS | 2 |
| | | GSGLSTWYAPAVKGRATISRDNGQSTVRLQLNNLRAEDTGTYYCAKAAGSC | |
| | | GVGTCAGGIVDGIDAWGHGTEVIVSS | |
| | VL | QAALTQPSSVSANPGETVKITCSGGDSYAGSYYYGWFQQKAPGSAPVTVIYY | 3 |
| | | NDKRPSDIPSRFSGSTSGSTSTLTITGVQAEDEAVYYCGSIDTSSGTGIFGAGTT | |
| | | LTVL | |
| | HCDR | GFTFSSYQMF | 4 |
| | 1 | | |
| | HCDR | EISGSGLSTWYAPAVKG | 5 |
| | 2 | | |
| | HCDR | AAGSCGVGTCAGGIVDGIDA | 6 |
| | 3 | | |
| | LCDR | SGGDSYAGSYYYG | 7 |
| | 1 | | |
| | LCDR | YNDKRPS | 8 |
| | 2 | | |
| | LCDR | GSIDTSSGTGI | 9 |
| | 3 | | |
| Anti- | scFv | QAALTQPSSVSANPGETVKITCSGSSSSYYGWYQQKAPGSAPVTLIYANDKRP | 10 |
| hCD40 | | SDIPSRFSGSKSGSTHTLTITGVQADDEAVYFCGSADSTDSGFGAGTTLTVLGQ | |
| (2E1) | | SSRSSGGGGSSGGGGSAVTLDESGGGLQTPGGALSLVCKASGFSFSSYSMHW | |
| | | VRQAPGKGLEFVAAISSDGSRYTHYGPAVKGRATISRDNGQSTVRLQLNNLR | |
| | | PEDTGTYYCAKTSNTWTWNTGWIDAWGHGTEVIVSS | |
| | VH | AVTLDESGGGLQCPGGALSLVCKASGFSFSSYSMHWVRQAPGKGLEFVAAIS | 11 |
| | | SDGSRYTHYGPAVKGRATISRDNGQSTVRLQLNNLRPEDTGTYYCAKTSNT | |
| | | WTWNTGWIDAWGHGTEVIVSS | |
| | VL | QAALTQPSSVSANPGETVKITCSGSSSSYYGWYQQKAPGSAPVTLIYANDKRP | 12 |
| | | SDIPSRFSGSKSGSTHTLTITGVQADDEAVYFCGSADSTDSGFGAGTTLTVL | |
| | HCDR | GFSFSSYSMH | 13 |
| | 1 | | |
| | HCDR | AISSDGSRYTHYGPAVKG | 14 |
| | 2 | | |
| | HCDR | TSNTWTWNTGWIDA | 15 |
| | 3 | | |
| | LCDR | SGSSSSYYG | 16 |
| | 1 | | |
| | LCDR | ANDKRPS | 17 |
| | 2 | | |
| | LCDR | GSADSTDSG | 18 |
| | 3 | | |
| Human- | scFv | AIQLTQSPSSLSASVGDRVTITCSGGDSYAGSYYYGWYQQKPGKAPKTLIYYN | 19 |
| ized | | DKRPSGVPSRFSGSTSGTDSTLTISSLQPEDFATYYCGSIDTSSGTGIFGQGTKL | |
| anti- | | EIKGQSSRSSGGGGSSGGGGSEVQLLESGGGLVQPGGSLRLSCAASGFTFSSY | |
| hCD40 | | QMFWVRQAPGKGLEYVSEISGSGLSTWYAPAVKGRFTISRDNSKNTLYLQM | |
| (hu2B1) | | NSLRAEDTAVYYCAKAAGSCGVGTCAGGIVDAIDAWGQGTTVTVSS | |
| | VH | EVQLLESGGGLVQPGGSLRLSCAASGFTFSSYQMFWVRQAPGKGLEYVSEIS | 20 |
| | | GSGLSTWYAPAVKGRFTISRDNSKNTLYLQMNSLRAEDTAVYYCAKAAGSC | |
| | | GVGTCAGGIVDAIDAWGQGTTVTVSS | |
| | VL | AIQLTQSPSSLSASVGDRVTITCSGGDSYAGSYYYGWYQQKPGKAPKTLIYYN | 21 |
| | | DKRPSGVPSRFSGSTSGTDSTLTISSLQPEDFATYYCGSIDTSSGTGIFGQGTKL | |
| | | EIK | |
| | HCDR | GFTFSSYQMF | 22 |
| | 1 | | |
| | HCDR | EISGSGLSTWYAPAVKG | 23 |
| | 2 | | |
| | HCDR | AAGSCGVGTCAGGIVDAIDA | 24 |
| | 3 | | |
| | LCDR | SGGDSYAGSYYYG | 25 |
| | 1 | | |
| | LCDR | YNDKRPS | 26 |
| | 2 | | |
| | LCDR | GSIDTSSGTGI | 27 |
| | 3 | | |
| Anti- | scFv | DILMTQTPSSLPVSLGDQASISCRSSQSIVHSNGNTYLEWYLQKPGQSPKLLIY | 28 |
| His | | KVSNRFSGVPDRFSGSGSGTDFTLKISRVEAEDLGVYYCFQGSHVPFTFGSGT | |
| (3D5M) | | KLEIKRGGGGSGGGGSGGGGSGGGGSQVQLQQSGPEDVKPGASVKISCKASG | |
| | | YTFTDYYMNWVKQSPGKGLEWIGDINPNNGGTSYNQKFKGRATLTVDKSSS | |
| | | TAYMELRSLTSEDSSVYYCESQSGAYWGQGTTVTVSA | |
| | VH | QVQLQQSGPEDVKPGASVKISCKASGYTFTDYYMNWVKQSPGKGLEWIGDI | 29 |
| | | NPNNGGTSYNQKFKGRATLTVDKSSSTAYMELRSLTSEDSSVYYCESQSGAY | |
| | | WGQGTTVTVSA | |
| | VL | DILMTQTPSSLPVSLGDQASISCRSSQSIVHSNGNTYLEWYLQKPGQSPKLLIY | 30 |
| | | KVSNRFSGVPDRFSGSGSGTDFTLKISRVEAEDLGVYYCFQGSHVPFTFGSGT | |
| | | KLEIK | |
| | HCDR | GYTFTDYYMN | 31 |
| | 1 | | |
| | HCDR | DINPNNGGTSYNQKFKG | 32 |
| | 2 | | |
| | HCDR | QSGAY | 33 |
| | 3 | | |
| | LCDR | RSSQSIVHSNGNTYLE | 34 |
| | 1 | | |
| | LCDR | KVSNRFS | 35 |
| | 2 | | |
| | LCDR | FQGSHVPFT | 36 |
| | 3 | | |
| Human- | scFv | DIVMTQSPLSLPVTPGEPASISCRSSQSIVHSNRNTYLEWYLQKPGQSPQLLIY | 37 |
| ized | | KVSNRFSGVPDRFSGSGSGTDFTLKISRVEAEDVGVYYCFQGSHVPFTFGQGT | |
| anti-His | | KLEIKRGGGGSGGGGSGGGGSGGGGSQVQLVQSGAEVKKPGASVKVSCKAS | |
| (hu3D5M) | | GYTFTDYYMNWVRQAPGQGLEWMGDINPNHGGTYYNHKFKGRVTMTVDT | |
| | | STSTAYMELRSLRSDDTAVYYCESQSGAYWGQGTTVTVSS | |
| | VH | QVQLVQSGAEVKKPGASVKVSCKASGYTFTDYYMNWVRQAPGQGLEWMG | 38 |
| | | DINPNHGGTYYNHKFKGRVTMTVDTSTSTAYMELRSLRSDDTAVYYCESQS | |
| | | GAYWGQGTTVTVSS | |
| | VL | DIVMTQSPLSLPVTPGEPASISCRSSQSIVHSNRNTYLEWYLQKPGQSPQLLIY | 39 |
| | | KVSNRFSGVPDRFSGSGSGTDFTLKISRVEAEDVGVYYCFQGSHVPFTFGQGT | |
| | | KLEIK | |
| | HCDR | GYTFTDYYMN | 40 |
| | 1 | | |
| | HCDR | DINPNHGGTYYNHKFKG | 41 |
| | 2 | | |
| | HCDR | QSGAY | 42 |
| | 3 | | |
| | LCDR | RSSQSIVHSNRNTYLE | 43 |
| | 1 | | |
| | LCDR | KVSNRFS | 44 |
| | 2 | | |
| | LCDR | FQGSHVPFT | 45 |
| | 3 | | |
| Anti- | scFv | QAALTQPSSVSANLGGTVKITCSGGGGSDDGSYYYSWHQQKSPGSAPVTVIY | 46 |
| Myc | | ENNNRPSDIPSRFSGSKSGSTATLTITGVQAEDEAVYYCGSRDSSYVDYVGIFG | |
| (3A6) | | AGTTLTVLGQSSRSSGGGGSSGGGGSAVTLDESGGGLQTPGRALSLVCKASG | |
| | | FTFSSYDMAWVRQEPGKGLEYVASISRSGRYTRYGPAVKGRATISRDNGQST | |
| | | VRLQLNNLRAEDTATYFCTRGGAVSIDTWGHGTEVIVSS | |
| | VH | AVTLDESGGGLQTPGRALSLVCKASGFTFSSYDMAWVRQEPGKGLEYVASIS | 47 |
| | | RSGRYTRYGPAVKGRATISRDNGQSTVRLQLNNLRAEDTATYFCTRGGAVSI | |
| | | DTWGHGTEVIVSS | |
| | VL | QAALTQPSSVSANLGGTVKITCSGGGGSDDGSYYYSWHQQKSPGSAPVTVIY | 48 |
| | | ENNNRPSDIPSRFSGSKSGSTATLTITGVQAEDEAVYYCGSRDSSYVDYVGIFG | |
| | | AGTTLTVL | |
| | HCDR | GFTFSSYDMA | 49 |
| | 1 | | |
| | HCDR | SISRSGRYTRYGPAVKG | 50 |
| | 2 | | |
| | HCDR | GGAVSIDT | 51 |
| | 3 | | |
| | LCDR | SGGGGSDDGSYYYS | 52 |
| | 1 | | |
| | LCDR | ENNNRPS | 53 |
| | 2 | | |
| | LCDR | GSRDSSYVDYVGI | 54 |
| | 3 | | |
| Anti- | scFv | QAALTQPSSVSVNPGETVKITCSGGGSSSYYGWYQQKSPGSAPVTLIYQNTNR | 55 |
| Myc | | PSNIPSRFSGSTSGSTGTLTITGVQAEDEAVYFCGSRDSSGDGIFGAGTTLTVLG | |
| (7A1) | | QSSRSSGGGGSSGGGGSAVTLDESGGGLQTPGRALSLVCKASGFTFSSYDMA | |
| | | WVRQEPGKGLEYVASISRSGRYTRYGPAVKGRATISRDNGQSTVRLQLNNLR | |
| | | AEDTATYFCTRGGAVSIDTWGHGTEVIVSS | |
| | VH | AVTLDESGGGLQTPGRALSLVCKASGFTFSSYDMAWVRQEPGKGLEYVASIS | 56 |
| | | RSGRYTRYGPAVKGRATISRDNGQSTVRLQLNNLRAEDTATYFCTRGGAVSI | |
| | | DTWGHGTEVIVSS | |
| | VL | QAALTQPSSVSVNPGETVKITCSGGGSSSYYGWYQQKSPGSAPVTLIYQNTNR | 57 |
| | | PSNIPSRFSGSTSGSTGTLTITGVQAEDEAVYFCGSRDSSGDGIFGAGTTLTVL | |
| | HCDR | GFTFSSYDMA | 58 |
| | 1 | | |
| | HCDR | SISRSGRYTRYGPAVKG | 59 |
| | 2 | | |
| | HCDR | GGAVSIDT | 60 |
| | 3 | | |
| | LCDR | SGGGSSSYYG | 61 |
| | 1 | | |
| | LCDR | QNTNRPS | 62 |
| | 2 | | |
| | LCDR | GSRDSSGDGI | 63 |
| | 3 | | |
| Anti- | scFv | QAALTQPSSVSANPGETVEITCSGGSSGYYGWHQQKSPGSAPVTVIYENTNRP | 64 |
| Myc | | SDIPSRFSGSKSGSTATLTITGVQADDEAVYFCGSYDSSSASGIFGAGTTLTVL | |
| (8A9) | | GQSSRSSGGGGSSGGGGSAVTLDESGGGLQTPGGGLSLVCKASGFTFSSYDM | |
| | | AWVRQEPGKGLEYVASISRSGRYTRYGPAVKGRATISRDNGQSTVRLQLNNL | |
| | | RAEDTATYFCTRGGAVSIDTWGHGTEVIVSS | |
| | VH | AVTLDESGGGLQTPGGGLSLVCKASGFTFSSYDMAWVRQEPGKGLEYVASIS | 65 |
| | | RSGRYTRYGPAVKGRATISRDNGQSTVRLQLNNLRAEDTATYFCTRGGAVSI | |
| | | DTWGHGTEVIVSS | |
| | VL | QAALTQPSSVSANPGETVEITCSGGSSGYYGWHQQKSPGSAPVTVIYENTNRP | 66 |
| | | SDIPSRFSGSKSGSTATLTITGVQADDEAVYFCGSYDSSSASGIFGAGTTLTVL | |
| | HCDR | GFTFSSYDMA | 67 |
| | 1 | | |
| | HCDR | SISRSGRYTRYGPAVKG | 68 |
| | 2 | | |
| | HCDR | GGAVSIDT | 69 |
| | 3 | | |
| | LCDR | SGGSSGYYG | 70 |
| | 1 | | |
| | LCDR | ENTNRPS | 71 |
| | 2 | | |
| | LCDR | GSYDSSSASGI | 72 |
| | 3 | | |
| Anti- | scFv | QAALTQPSSVSANPGETVKITCSGGSSNYGWYQQKSPGSAPATVIYDNTNRPS | 73 |
| mCD40 | | DIPSRFSGALSGSTATLTITGVQVEDEAVYYCGGYDSGTYAGIFGAGTTLTVL | |
| (C1C02A) | | GQSSRSSGGGGSSGGGGSAVTLDESGGGLQTPGGTLSLACKGSGFTFSSVNM | |
| | | QWVRQAPGKGLEWVAGISSDGSYTAYGAAVKGRATISRDNGQSTVRLQLNN | |
| | | LRAEDTAIYYCAKIAGGYYWDAAYSIDMWGHGTEVIVSSTS | |
| | VH | AVTLDESGGGLQCPGGTLSLACKGSGFTFSSVNMQWVRQAPGKGLEWVAGI | 74 |
| | | SSDGSYTAYGAAVKGRATISRDNGQSTVRLQLNNLRAEDTAIYYCAKIAGGY | |
| | | YWDAAYSIDMWGHGTEVIVSSTS | |
| | VL | QAALTQPSSVSANPGETVKITCSGGSSNYGWYQQKSPGSAPATVIYDNTNRPS | 75 |
| | | DIPSRFSGALSGSTATLTITGVQVEDEAVYYCGGYDSGTYAGIFGAGTTLTVL | |
| | HCDR | GFTFSSVNMQ | 76 |
| | 1 | | |
| | HCDR | GISSDGSYTAYGAAVKG | 77 |
| | 2 | | |
| | HCDR | JAGGYYWDAAYSIDM | 78 |
| | 3 | | |
| | LCDR | SGGSSNYG | 79 |
| | 1 | | |
| | LCDR | DNTNRPS | 80 |
| | 2 | | |
| | LCDR | YDSGTYAGI | 81 |
| | 3 | | |
| Anti- | scFv | QAALTQPSSVSANLGETVKITCSGSSGNNYGWYQQKSPGSAPVTVIYYNDNR | 82 |
| hCS1 | | PSDIPSRFSGSKSGSTHTLTITGVQADDEAVYFCGSGDSSSNPGIFGAGTTLTVL | |
| (#57) | | GQSSRSSGGGGSSGGGGSAVTLDESGGGLQTPGGALSLVCKASGFTFSSHGM | |
| | | AWVRQAPGKGLEWVAGIDDSGSSTGYGSAVKGRATISRDNGQSTVRLQLNN | |
| | | LRAEDTGIYYCAKAYDTGCGWAGYNVYDCSADQIDAWGHGTEVIVSS | |
| | VH | AVTLDESGGGLQTPGGALSLVCKASGFTFSSHGMAWVRQAPGKGLEWVAGI | 83 |
| | | DDSGSSTGYGSAVKGRATISRDNGQSTVRLQLNNLRAEDTGIYYCAKAYDTG | |
| | | CGWAGYNVYDCSADQIDAWGHGTEVIVSS | |
| | VL | QAALTQPSSVSANLGETVKITCSGSSGNNYGWYQQKSPGSAPVTVIYYNDNR | 84 |
| | | PSDIPSRFSGSKSGSTHTLTITGVQADDEAVYFCGSGDSSSNPGIFGAGTTLTVL | |
| | HCDR | SHGMA | 85 |
| | 1 | | |
| | HCDR | GIDDSGSSTGYGSAVKG | 86 |
| | 2 | | |
| | HCDR | AYDTGCGWAGYNVYDCSADQIDA | 87 |
| | 3 | | |
| | LCDR | SGSSGNNYG | 88 |
| | 1 | | |
| | LCDR | YNDNRPS | 89 |
| | 2 | | |
| | LCDR | GSGDSSSNPGI | 90 |
| | 3 | | |
| Anti- | scFv | QAALTQPSSVSANPGETVKITCSGGGSYYGWYQQKSPGSAPVTVIYGNDKRP | 91 |
| hCS1 | | SNIPSRFSGSKSGSTATLTITGVQVEDEAVYFCGGWDSTDAGTFGAGTTLTVL | |
| (#87) | | GQSSRSSGGGGSSGGGGSAVTLDESGGGLQTPGGGLSLVCKASGFTFSSHGM | |
| | | AWVRQAPGKGLEWVAGIDDSGSSTGYGSAVKGRATISRDNGQSTVRLQLNN | |
| | | LRAEDTGIYYCAKAYDTGCGWAGYNVYDCSADQIDAWGHGTEVIVSS | |
| | VH | AVTLDESGGGLQTPGGGLSLVCKASGFTFSSHGMAWVRQAPGKGLEWVAGI | 92 |
| | | DDSGSSTGYGSAVKGRATISRDNGQSTVRLQLNNLRAEDTGIYYCAKAYDTG | |
| | | CGWAGYNVYDCSADQIDAWGHGTEVIVSS | |
| | VL | QAALTQPSSVSANPGETVKITCSGGGSYYGWYQQKSPGSAPVTVIYGNDKRP | 93 |
| | | SNIPSRFSGSKSGSTATLTITGVQVEDEAVYFCGGWDSTDAGTFGAGTTLTVL | |
| | HCDR | SHGMA | 94 |
| | 1 | | |
| | HCDR | GIDDSGSSTGYGSAVKG | 95 |
| | 2 | | |
| | HCDR | AYDTGCGWAGYNVYDCSADQIDA | 96 |
| | 3 | | |
| | LCDR | SGGGSYYG | 97 |
| | 1 | | |
| | LCDR | GNDKRPS | 98 |
| | 2 | | |
| | LCDR | GGWDSTDAGT | 99 |
| | 3 | | |
| Anti- | scFv | QAALTQPSSVSANSGETVKITCSGGGSNSYYGWYQQKSPGSAPVTVIYWDDE | 100 |
| hCS1 | | RPSGIPSRFSGALSGSTATLTITGVQVEDEAVYYCGIGDSSGTSLFGAGTTLTV | |
| (#135) | | LGQSSRSSGGGGSSGGGGSAVTLDESGGGLQTPGGALSLVCKASGFTFRSYE | |
| | | MQWVRQAPGKGLEWVAGIENDGSYIGYAPAVDGRATISRDNGQSTVRLQLN | |
| | | KLRAEDTGTYYCARDFGYGVGGIDAWGHGTEVIVSS | |
| | VH | AVTLDESGGGLQTPGGALSLVCKASGFTFRSYEMQWVRQAPGKGLEWVAGI | 101 |
| | | ENDGSYIGYAPAVDGRATISRDNGQSTVRLQLNKLRAEDTGTYYCARDFGYG | |
| | | VGGIDAWGHGTEVIVSS | |
| | VL | QAALTQPSSVSANSGETVKITCSGGGSNSYYGWYQQKSPGSAPVTVIYWDDE | 102 |
| | | RPSGIPSRFSGALSGSTATLTITGVQVEDEAVYYCGIGDSSGTSLFGAGTTLTV | |
| | | L | |
| | HCDR | SYEMQ | 103 |
| | 1 | | |
| | HCDR | GIENDGSYIGYAPAVDG | 104 |
| | 2 | | |
| | HCDR | DFGYGVGGIDA | 105 |
| | 3 | | |
| | LCDR | SGGGSNSYYG | 106 |
| | 1 | | |
| | LCDR | WDDERPS | 107 |
| | 2 | | |
| | LCDR | GIGDSSGTSL | 108 |
| | 3 | | |

Table 1_ (above): Antibody Sequences. Single underline +EE HCDRs, double underline +EE LCDRs.

TABLE 2
Adaptor against murine proteins

| | | | SEQ |
|---|---|---|---|
| Description | | Sequence | ID |
| C1C02-Cot | Adapter | QAALTQPSSVSANPGETVKITCSGGSSNYGWYQQKSPGSAPATVIYDNTNRPSDI | 109 |
| (C1C02- | | SRFSGALSGSTATLTITGVQVEDEAVYYCGGYDSGTYAGIFGAGTTLTVLGQSSRS | |
| Ck-Cot) | | SGGGGSSGGGGSAVTLDESGGGLQCPGGTLSLACKGSGFTFSSVNMQWVRQAPG | |
| | | KLEWVAGISSDGSYTAYGAAVKGRATISRDNGQSTVRLQLNNLRAEDTAIYYCA | |
| | | KIAGGYYWDAAYSIDMWGHGTEVIVSSTSGQAGQRTVAAPSVFIFPPSDEQLKSG | |
| | | TASVVCLLNNFYPREAKVQWKVDNALQSGNSQESVTEQDSKDSTYSLSSTLTLSK | |
| | | ADYEKHKVYACEVTHQGLSLPVTKSFNRGES | |
| | Spel | TS | n/a |
| | site | | |
| | Sfil site | GQAGQ | 110 |
| | scFv | QAALTQPSSVSANPGETVKITCSGGSSNYGWYQQKSPGSAPATVIYDNTNRPSDIP | 111 |
| | | SRFSGALSGSTATLTITGVQVEDEAVYYCGGYDSGTYAGIFGAGTTLTVLGQSSRS | |
| | | SGGGGSSGGGGSAVTLDESGGGLQCPGGTLSLACKGSGFTFSSVNMQWVRQAPG | |
| | | KGLEWVAGISSDGSYTAYGAAVKGRATISRDNGQSTVRLQLNNLRAEDTAIYYCA | |
| | | KIAGGYYWDAAYSIDMWGHGTEVIVSS | |
| | hCk | RTVAAPSVFIFPPSDEQLKSGTASVVCLLNNFYPREAKVQWKVDNALQSGNSQES | 112 |
| | | VTEQDSKDSTYSLSSTLTLSKADYEKHKVYACEVTHQGLSLPVTKSFNRGES | |
| C1C02- | Adapter | QAALTQPSSVSANPGETVKITCSGGSSNYGWYQQKSPGSAPATVIYDNTNRPSDIP | 113 |
| His | | SRFSGALSGSTATLTITGVQVEDEAVYYCGGYDSGTYAGIFGAGTTLTVLGQSSRS | |
| | | SGGGGSSGGGGSAVTLDESGGGLQTPGGTLSLACKGSGFTFSSVNMQWVRQAPG | |
| | | KGLEWVAGISSDGSYTAYGAAVKGRATISRDNGQSTVRLQLNNLRAEDTAIYYCA | |
| | | KIAGGYYWDAAYSIDMWGHGTEVIVSSTSGQAGQRTVAAPSVFIFPPSDEQLKSG | |
| | | TASVVCLLNNFYPREAKVQWKVDNALQSGNSQESVTEQDSKDSTYSLSSTLTLSK | |
| | | ADYEKHKVYACEVTHQGLSSPVTKSFNRGESHHHHHH | |
| (C1C02 | Spel | TS | 114 |
| | site | | |
| -Ck- | Sfil site | GQAGQ | 115 |
| His)) | scFv | QAALTQPSSVSANPGETVKITCSGGSSNYGWYQQKSPGSAPATVIYDNTNRPSDIP | 116 |
| | | SRFSGALSGSTATLTITGVQVEDEAVYYCGGYDSGTYAGIFGAGTTLTVLGQSSRS | |
| | | SGGGGSSGGGGSAVTLDESGGGLQTPGGTLSLACKGSGFTFSSVNMQWVRQAPG | |
| | | KGLEWVAGISSDGSYTAYGAAVKGRATISRDNGQSTVRLQLNNLRAEDTAIYYCA | |
| | | KIAGGYYWDAAYSIDMWGHGTEVIVSS | |
| | hCk | RTVAAPSVFIFPPSDEQLKSGTASVVCLLNNFYPREAKVQWKVDNALQSGNSQES | 117 |
| | | VTEQDSKDSTYSLSSTLTLSKADYEKHKVYACEVTHQGLSSPVTKSFNRGES | |
| | His tag | HHHHHH | 118 |

Table 2(above): Murine Adaptor Sequences. Single underline +EE scFv, double underline +EE hCk.

TABLE 3
Adaptors against human proteins

| | | | SEQ |
|---|---|---|---|
| Description | | Sequence | ID |
| 2B1-cot | Adapter | QAALTQPSSVSANPGETVKITCSGGDSYAGSYYYGWFQQKAPGSAPVTVI | 119 |
| (2B1- | | YYNDKRPSDIPSRFSGSTSGSTSTLTITGVQAEDEAVYYCGSIDTSSGTGIFG | |
| Ck-cot) | | AGTTLTVLGQSSRSSGGGGSSGGGGSAVTLDESGGGLQCPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGES | |
| | scFv | QAALTQPSSVSANPGETVKITCSGGDSYAGSYYYGWFQQKAPGSAPVTVI | 120 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQCPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 121 |
| | site | | |
| | hCk | RTVAAPSVFIFPPSDEQLKSGTASVVCLLNNFYPREAKVQWKVDNALQSG | 122 |
| | | NSQESVTEQDSKDSTYSLSSTLTLSKADYEKHKVYACEVTHQGLSSPVTKS | |
| | | FNRGES | |
| 2E1-cot | Adapter | QAALTQPSSVSANPGETVKITCSGSSSSYYGWYQQKAPGSAPVTLIYANDK | 123 |
| (2E1- | | RPSDIPSRFSGSKSGSTHTLTITGVQADDEAVYFCGSADSTDSGFGAGTTLT | |
| Ck-cot) | | VLGQSSRSSGGGGSSGGGGSAVTLDESGGGLQCPGGALSLVCKASGFSFSS | |
| | | MHWVRQAPGKGLEFVAAISSDGSRYTHYGPAVKGRATISRDNGQSTV | |
| | | RLQLNNLRPEDTGTYYCAKTSNTWTWNTGWIDAWGHGTEVIVSSTSGQA | |
| | | GQRTVAAPSVEIFPPSDEQLKSGTASVVCLLNNFYPREAKVQWKVDNALQ | |
| | | SGNSQESVTEQDSKDSTYSLSSTLTLSKADYEKHKVYACEVTHQGLSSPVT | |
| | | KSFNRGES | |
| | scFv | QAALTQPSSVSANPGETVKITCSGSSSSYYGWYQQKAPGSAPVTLIYANDK | 124 |
| | (2E1) | RPSDIPSRFSGSKSGSTHTLTITGVQADDEAVYFCGSADSTDSGFGAGTTLT | |
| | | VLGQSSRSSGGGGSSGGGGSAVTLDESGGGLQCPGGALSLVCKASGFSFSS | |
| | Spel | TS | n/a |
| | site | | |
| | Sfil | GQAGQ | 125 |
| | site | | |
| | hCk | RTVAAPSVFIFPPSDEQLKSGTASVVCLLNNFYPREAKVQWKVDNALQSG | 126 |
| | | NSQESVTEQDSKDSTYSLSSTLTLSKADYEKHKVYACEVTHQGLSSPVTKS | |
| | | FNRGES | |
| 2B1- | Adapter | QAALTQPSSVSANPGETVKITCSGGDSYAGSYYYGWFQQKAPGSAPVTVI | 127 |
| Ck-His | | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGESHHHHHH | |
| | scFv | QAALTQPSSVSANPGETVKITCSGGDSYAGSYYYGWFQQKAPGSAPVTVI | 128 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 129 |
| | site | | |
| | hCk | RTVAAPSVFIFPPSDEQLKSGTASVVCLLNNFYPREAKVQWKVDNALQSG | 130 |
| | | NSQESVTEQDSKDSTYSLSSTLTLSKADYEKHKVYACEVTHQGLSSPVTKS | |
| | | FNRGES | |
| | His | HHHHHH | 131 |
| | tag | | |
| Hu2B1-Ck- | Adapter | AIQLTQSPSSLSASVGDRVTITCSGGDSYAGSYYYGWYQQKPGKAPKTLIY | 132 |
| His | | YNDKRPSGVPSRFSGSTSGTDSTLTISSLQPEDFATYYCGSIDTSSGTGIFGQ | |
| (humanized | | GTKLEIKGQSSRSSGGGGSSGGGGSEVQLLESGGGLVQPGGSLRLSCAASG | |
| 2B1- | | FTFSSYQMFWVRQAPGKGLEYVSEISGSGLSTWYAPAVKGRFTISRDNSK | |
| Ck-His) | | NTLYLQMNSLRAEDTAVYYCAKAAGSCGVGTCAGGIVDAIDAWGQGTT | |
| | | VTVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGESHHHHHH | |
| | scFv | AIQLTQSPSSLSASVGDRVTITCSGGDSYAGSYYYGWYQQKPGKAPKTLIY | 133 |
| | (hu2B1) | YNDKRPSGVPSRFSGSTSGTDSTLTISSLQPEDFATYYCGSIDTSSGTGIFGQ | |
| | | GTKLEIKGQSSRSSGGGGSSGGGGSEVQLLESGGGLVQPGGSLRLSCAASG | |
| | | FTFSSYQMFWVRQAPGKGLEYVSEISGSGLSTWYAPAVKGRFTISRDNSK | |
| | | NTLYLQMNSLRAEDTAVYYCAKAAGSCGVGTCAGGIVDAIDAWGQGTT | |
| | | VTVSS | |
| | Spel | TS | n/a |
| | site | | |
| | Sfil | GQAGQ | 134 |
| | site | | |
| | hCk | RTVAAPSVFIFPPSDEQLKSGTASVVCLLNNFYPREAKVQWKVDNALQSG | 135 |
| | | NSQESVTEQDSKDSTYSLSSTLTLSKADYEKHKVYACEVTHQGLSSPVTKS | |
| | | FNRGES | |
| | His | HHHHHH | 136 |
| | tag | | |
| 2B1- | Adapter | QAALTQPSSVSANPGETVKITCSGGDSYAGSYYYGWFQQKAPGSAPVTVI | 137 |
| L1-His | | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQGGGGSHHHHHH | |
| | scFv | QAALTQPSSVSANPGETVKITCSGGDSYAGSYYYGWFQQKAPGSAPVTVI | 138 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 139 |
| | site | | |
| | Linker | GGGGS | 140 |
| | His | HHHHHH | 141 |
| | tag | | |
| 2B1-Ck- | Adapter | QAALTQPSSVSANPGETVKITCSGGDSYAGSYYYGWFQQKAPGSAPVTVI | 142 |
| Myc | | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGESEQKLISEEDL | |
| | scFv | QAALTQPSSVSANPGETVKITCSGGDSYAGSYYYGWFQQKAPGSAPVTVI | 143 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 144 |
| | site | | |
| | hCk | RTVAAPSVFIFPPSDEQLKSGTASVVCLLNNFYPREAKVQWKVDNALQSG | 145 |
| | | NSQESVTEQDSKDSTYSLSSTLTLSKADYEKHKVYACEVTHQGLSSPVTKS | |
| | | FNRGES | |
| | Myc | EQKLISEEDL | 146 |
| | tag | | |
| 2B1- | Adapter | QAALTQPSSVSANPGETVKITCSGGDSYAGSYYYGWFQQKAPGSAPVTVI | 147 |
| Ck-L1- | | YYNDKRPSDIPSRFSGSTSGSTSTLTITGVQAEDEAVYYCGSIDTSSGTGIFG | |
| Myc | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGESGGGGSEQKLISEEDL | |
| | scFv | QAALTQPSSVSANPGETVKITCSGGDSYAGSYYYGWFQQKAPGSAPVTVI | 148 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 149 |
| | site | | |
| | hCk | RTVAAPSVFIFPPSDEQLKSGTASVVCLLNNFYPREAKVQWKVDNALQSG | 150 |
| | | NSQESVTEQDSKDSTYSLSSTLTLSKADYEKHKVYACEVTHQGLSSPVTKS | |
| | | FNRGES | |
| | Linker | GGGGS | 151 |
| | Myc | EQKLISEEDL | 152 |
| | tag | | |
| 2B1- | Adapter | QAALTQPSSVSANPGETVKITCSGGDSYAGSYYYGWFQQKAPGSAPVTVI | 153 |
| Ck-L2- | | YYNDKRPSDIPSRFSGSTSGSTSTLTITGVQAEDEAVYYCGSIDTSSGTGIFG | |
| Myc | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGESGGGGSGGGSEQKLISEEDL | |
| | scFv | QAALTQPSSVSANPGETVKITCSGGDSYAGSYYYGWFQQKAPGSAPVTVI | 154 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 155 |
| | site | | |
| | hCk | RTVAAPSVFIFPPSDEQLKSGTASVVCLLNNFYPREAKVQWKVDNALQSG | 156 |
| | | NSQESVTEQDSKDSTYSLSSTLTLSKADYEKHKVYACEVTHQGLSSPVTKS | |
| | | FNRGES | |
| | Linker | GGGGSGGGGS | 157 |
| | Myc | EQKLISEEDL | 158 |
| | tag | | |
| 2B1-L1- | Adapter | QAALTQPSSVSANPGETVKITCSGGDSYAGSYYYGWFQQKAPGSAPVTVI | 159 |
| Myc-Ck | | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSSTSGQAGQGGGGSEQKLISEEDLRTVAAPSVFIFPPSDEQLKSGTAS | |
| | | VVCLLNNFYPREAKVQWKVDNALQSGNSQESVTEQDSKDSTYSLSSTLTL | |
| | | SKADYEKHKVYACEVTHQGLSLPVTKSFNRGES | |
| | scFv | QAALTQPSSVSANPGETVKITCSGGDSYAGSYYYGWFQQKAPGSAPVTVI | 160 |
| | (2B1) | YYNDKRPSDIPSRFSGSTSGSTSTLTITGVQAEDEAVYYCGSIDTSSGTGIFG | |
| | | AGTTLTVLGQSSRSSGGGGSSGGGGSAVTLDESGGGLQTPGGALSLVCKG | |
| | | SGFTFSSYQMFWVRQAPGKGLEYVAEISGSGLSTWYAPAVKGRATISRDN | |
| | | GQSTVRLQLNNLRAEDTGTYYCAKAAGSCGVGTCAGGIVDGIDAWGHGT | |
| | | EVIVSS | |
| | Spel | TS | n/a |
| | site | | |
| | Sfil | GQAGQ | 161 |
| | site | | |
| | Linker | GGGGS | 162 |
| | Myc | EQKLISEEDL | 163 |
| | tag | | |
| | hCk | RTVAAPSVFIFPPSDEQLKSGTASVVCLLNNFYPREAKVQWKVDNALQSG | 164 |
| | | NSQESVTEQDSKDSTYSLSSTLTLSKADYEKHKVYACEVTHQGLSLPVTKS | |
| | | FNRGES | |
| 2B1(Fab)- | Adapter | AVTLDESGGGLQTPGGALSLVCKGSGFTFSSYQMFWVRQAPGKGLEYVA | 165 |
| Myc-HC | | EISGSGLSTWYAPAVKGRATISRDNGQSTVRLQLNNLRAEDTGTYYCAKA | |
| | | AGSCGVGTCAGGIVDGIDAWGHGTEVIVSSTSASTKGPSVFPLAPSSKSTS | |
| | | GGTAALGCLVKDYFPEPVTVSWNSGALTSGVHTFPAVLQSSGLYSLSSVV | |
| | | TVPSSSLGTQTYICNVNHKPSNTKVDKKVEPKSCGGGGSEQKLISEEDL | |
| | VH | AVTLDESGGGLQTPGGALSLVCKGSGFTFSSYQMFWVRQAPGKGLEYVA | 166 |
| | (2B1) | EISGSGLSTWYAPAVKGRATISRDNGQSTVRLQLNNLRAEDTGTYYCAKA | |
| | | AGSCGVGTCAGGIVDGIDAWGHGTEVIVSS | |
| | Spel | TS | n/a |
| | site | | |
| | hCH1 | ASTKGPSVFPLAPSSKSTSGGTAALGCLVKDYFPEPVTVSWNSGALTSGVH | 167 |
| | | TFPAVLQSSGLYSLSSVVTVPSSSLGTQTYICNVNHKPSNTKVDKK VEPKS | |
| | | C | |
| | Linker | GGGGS | 168 |
| | Myc | EQKLISEEDL | 169 |
| | tag | | |
| 2B1(Fab)- | Adapter | QAALTQPSSVSANPGETVKITCSGGDSYAGSYYYGWFQQKAPGSAPVTVI | 170 |
| Myc-LC | | YYNDKRPSDIPSRFSGSTSGSTSTLTITGVQAEDEAVYYCGSIDTSSGTGIFG | |
| | | AGTTLTVLRTVAAPSVFIFPPSDEQLKSGTASVVCLLNNFYPREAKVQWK | |
| | | VDNALQSGNSQESVTEQDSKDSTYSLSSTLTLSKADYEKHKVYACEVTHQ | |
| | | GLSSPVTKSFNRGECGGGGSEQKLISEEDL | |
| | VL | QAALTQPSSVSANPGETVKITCSGGDSYAGSYYYGWFQQKAPGSAPVTVI | 171 |
| | (2B1) | YYNDKRPSDIPSRFSGSTSGSTSTLTITGVQAEDEAVYYCGSIDTSSGTGIFG | |
| | | AGTTLTVL | |
| | hCk | RTVAAPSVFIFPPSDEQLKSGTASVVCLLNNFYPREAKVQWKVDNALQSG | 172 |
| | | NSQESVTEQDSKDSTYSLSSTLTLSKADYEKHKVYACEVTHQGLSSPVTKS | |
| | | FNRGEC | |
| | Linker | GGGGS | 173 |
| | Myc | EQKLISEEDL | 174 |
| | tag | | |
| hCS1(#57)- | Adapter | QAALTQPSSVSANLGETVKITCSGSSGNNYGWYQQKSPGSAPVTVIYYND | 175 |
| Ck-His | | NRPSDIPSRFSGSKSGSTHTLTITGVQADDEAVYFCGSGDSSSNPGIFGAGT | |
| | | TLTVLGQSSRSSGGGGSSGGGGSAVTLDESGGGLQTPGGALSLVCKASGF | |
| | | TFSSHGMAWVRQAPGKGLEWVAGIDDSGSSTGYGSAVKGRATISRDNGQ | |
| | | STVRLQLNNLRAEDTGIYYCAKAYDTGCGWAGYNVYDCSADQIDAWGH | |
| | | GTEVIVSSTSGQAGQRTVAAPSVFIFPPSDEQLKSGTASVVCLLNNFYPREA | |
| | | KVQWKVDNALQSGNSQESVTEQDSKDSTYSLSSTLTLSKADYEKHKVYA | |
| | | CEVTHQGLSSPVTKSFNRGESGGGGSGGGGSHHHHHH | |
| | scFv | QAALTQPSSVSANLGETVKITCSGSSGNNYGWYQQKSPGSAPVTVIYYND | 176 |
| | (#57) | NRPSDIPSRFSGSKSGSTHTLTITGVQADDEAVYFCGSGDSSSNPGIFGAGT | |
| | | TLTVLGQSSRSSGGGGSSGGGGSAVTLDESGGGLQTPGGALSLVCKASGF | |
| | | TFSSHGMAWVRQAPGKGLEWVAGIDDSGSSTGYGSAVKGRATISRDNGQ | |
| | | STVRLQLNNLRAEDTGIYYCAKAYDTGCGWAGYNVYDCSADQIDAWGH | |
| | | GTEVIVSS | |
| | Spel | TS | n/a |
| | site | | |
| | Sfil | GQAGQ | 177 |
| | site | | |
| | hCk | RTVAAPSVFIFPPSDEQLKSGTASVVCLLNNFYPREAKVQWKVDNALQSG | 178 |
| | | NSQESVTEQDSKDSTYSLSSTLTLSKADYEKHKVYACEVTHQGLSSPVTKS | |
| | | FNRGES | |
| | Linker | GGGGSGGGGS | 179 |
| hCS1(#87)- | Adapter | QAALTQPSSVSANPGETVKITCSGGGSYYGWYQQKSPGSAPVTVIYGNDK | 180 |
| Ck-His | | RPSNIPSRFSGSKSGSTATLTITGVQVEDEAVYFCGGWDSTDAGTFGAGTT | |
| | | LTVLGQSSRSSGGGGSSGGGGSAVTLDESGGGLQTPGGGLSLVCKASGFT | |
| | | FSSHGMAWVRQAPGKGLEWVAGIDDSGSSTGYGSAVKGRATISRDNGQS | |
| | | TVRLQLNNLRAEDTGIYYCAKAYDTGCGWAGYNVYDCSADQIDAWGHG | |
| | | TEVIVSSTSGQAGQRTVAAPSVFIFPPSDEQLKSGTASVVCLLNNFYPREAK | |
| | | VQWKVDNALQSGNSQESVTEQDSKDSTYSLSSTLTLSKADYEKHKVYAC | |
| | | EVTHQGLSSPVTKSFNRGESGGGGSGGGGSHHHHHH | |
| | scFv | QAALTQPSSVSANPGETVKITCSGGGSYYGWYQQKSPGSAPVTVIYGNDK | 181 |
| | (#87) | RPSNIPSRFSGSKSGSTATLTITGVQVEDEAVYFCGGWDSTDAGTFGAGTT | |
| | | LTVLGQSSRSSGGGGSSGGGGSAVTLDESGGGLQTPGGGLSLVCKASGFT | |
| | | FSSHGMAWVRQAPGKGLEWVAGIDDSGSSTGYGSAVKGRATISRDNGQS | |
| | | TVRLQLNNLRAEDTGIYYCAKAYDTGCGWAGYNVYDCSADQIDAWGHG | |
| | | TEVIVSS | |
| | Spel | TS | n/a |
| | site | | |
| | Sfil | GQAGQ | 182 |
| | site | | |
| | hCk | RTVAAPSVFIFPPSDEQLKSGTASVVCLLNNFYPREAKVQWKVDNALQSG | 183 |
| | | NSQESVTEQDSKDSTYSLSSTLTLSKADYEKHKVYACEVTHQGLSSPVTKS | |
| | | FNRGES | |
| | Linker | GGGGSGGGGS | 184 |
| | His | HHHHHH | 185 |
| | tag | | |
| hCS1(#135)- | Adapt | QAALTQPSSVSANPGETVKITCSGGGNYGWYQQKSPGSALVTVIYSNDNR | 186 |
| Ck-His | er | PSNIPSRFSGSKSGSTGTLTITGVQAEDEAVYFCGSYEDNSNPGIFGAGTTL | |
| | | TVLGQSSRSSGGGGSSGGGGSAVTLDESGGGLQTPGGALSLVCKASGFTFS | |
| | | SHGMAWVRQAPGKGLEWVAGIDDSGSSTGYGSAVKGRATISRDNGQSTV | |
| | | RLQLNNLRAEDTGIYYCAKAYDTGCGWAGYNVYDCSADQIDAWGHGTE | |
| | | VIVSSTSGQAGQRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGESGGGGSGGGGSHHHHHH | |
| | scFv | QAALTQPSSVSANPGETVKITCSGGGNYGWYQQKSPGSALVTVIYSNDNR | 187 |
| | (#135) | PSNIPSRFSGSKSGSTGTLTITGVQAEDEAVYFCGSYEDNSNPGIFGAGTTL | |
| | | TVLGQSSRSSGGGGSSGGGGSAVTLDESGGGLQTPGGALSLVCKASGFTFS | |
| | | SHGMAWVRQAPGKGLEWVAGIDDSGSSTGYGSAVKGRATISRDNGQSTV | |
| | | RLQLNNLRAEDTGIYYCAKAYDTGCGWAGYNVYDCSADQIDAWGHGTE | |
| | | VIVSS | |
| | Spel | TS | n/a |
| | site | | |
| | Sfil | GQAGQ | 188 |
| | site | | |
| | hCk | RTVAAPSVFIFPPSDEQLKSGTASVVCLLNNFYPREAKVQWKVDNALQSG | 189 |
| | | NSQESVTEQDSKDSTYSLSSTLTLSKADYEKHKVYACEVTHQGLSSPVTKS | |
| | | FNRGES | |
| | Linker | GGGGSGGGGS | 190 |
| | His | HHHHHH | 191 |
| | tag | | |

Table 3(above): Adaptor against human proteins Sequences. Single underline +EE scFv, double
underline +EE hCk or hCH1.

TABLE 4
Murine CAR

| | | | SEQ |
|---|---|---|---|
| Description | | Sequence | ID |
| CD40 | mouse | MDFQVQIFSFLLISASVIMSR | 192 |
| CAR | Ig | | |
| (C1C02- | kappa | | |
| 28z) | leader | | |
| | CAR | LTQPSSVSANPGETVKITCSGGSSNYGWYQQKSPGSAPATVIYDNTNRPSDIP | 193 |
| | | SRFSGALSGSTATLTITGVQVEDEAVYYCGGYDSGTYAGIFGAGTTLTVLGQ | |
| | | SSRSSGGGGSSGGGGSAVTLDESGGGLQTPGGTLSLACKGSGFTFSSVNMQ | |
| | | WVRQAPGKGLEWVAGISSDGSYTAYGAAVKGRATISRDNGQSTVRLQLNN | |
| | | LRAEDTAIYYCAKIAGGYYWDAAYSIDMWGHGTEVIVSSTSIEFMYPPPYLD | |
| | | NERSNGTIIHIKEKHLCHTQSSPKLFWALVVVAGVLFCYGLLVTVALCVIWT | |
| | | NSRRNRGGQSDYMNMTPRRPGLTRKPYQPYAPARDFAAYRPRAKFSRSAETA | |
| | | ANLQDPNQLFNELNLGRREEFDVLEKKRARDPEMGGKQQRRRNPQEGVYNALQ | |
| | | KDKMAEAYSEIGTKGERRRGKGHDGLFQGLSTATKDTFDALHMQTLAPR | |
| | scFv | LTQPSSVSANPGETVKITCSGGSSNYGWYQQKSPGSAPATVIYDNTNRPSDIP | 194 |
| | (C1C02) | SRFSGALSGSTATLTITGVQVEDEAVYYCGGYDSGTYAGIFGAGTTLTVLGQ | |
| | | SSRSSGGGGSSGGGGSAVTLDESGGGLQTPGGTLSLACKGSGFTFSSVNMQ | |
| | | WVRQAPGKGLEWVAGISSDGSYTAYGAAVKGRATISRDNGQSTVRLQLNN | |
| | | LRAEDTAIYYCAKIAGGYYWDAAYSIDMWGHGTEVIVSSTS | |
| | mCD28 | IEFMYPPPYLDNERSNGTIIHIKEKHLCHTQSSPKLFWALVVVAGVLFCYGLL | 195 |
| | TM/cyt | VTVALCVIWTNSRRNRGGQSDYMNMTPRRPGLTRKPYQPYAPARDFAAYR | |
| | | P | |
| | mCD3 | RAKFSRSAETAANLQDPNQLFNELNLGRREEFDVLEKKRARDPEMGGKQQR | 195 |
| | zeta | RRNPQEGVYNALQKDKMAEAYSEIGTKGERRRGKGHDGLFQGLSTATKDT | |
| | cyt | FDALHMQTLAPR | |
| Cot | mouse | MDFQVQIFSFLLISASVIMSR | 196 |
| CAR | Ig | | |
| (Cot- | kappa | | |
| 28z) | leader | | |
| | mouse | ELDLTQTPASVSAAVGGTVTINCQSSQSPYSNEWLSWYQQKPGQAPKVLISR | 197 |
| | Ig | ISTLASGVSSRFKGSGSGTQFTLTISDLECGDAATYFCAGGYNFGLFPFGGGT | |
| | kappa | ELEILSSGGGGSGGGGGGSSRSSQSVKESEGRLVTPGGSLTLTCTVSGIDLSR | |
| | leader | DWMNWVRQAPGEGLEWIGAIGRSGDTYYATWAKGRFTISKTSSRTVTLTVT | |
| | | DLQRSDTATYFCARIPYFGWNNGDIWGPGTLVTISSIEFMYPPPYLDNERSNG | |
| | | TIIHIKEKHLCHTQSSPKLFWALVVVAGVLFCYGLLVTVALCVIWTNSRRNR | |
| | | GGQSDYMNMTPRRPGLTRKPYQPYAPARDFAAYRPRAKFSRSAETAANLQDP | |
| | | NQLFNELNLGRREEFDVLEKKRARDPEMGGKQQRRRNPQEGVYNALQKDKMA | |
| | | EAYSEIGTKGERRRGKGHDGLFQGLSTATKDTFDALHMQTLAPR | |
| | scFv | ELDLTQTPASVSAAVGGTVTINCQSSQSPYSNEWLSWYQQKPGQAPKVLISR | 198 |
| | (Clone: | ISTLASGVSSRFKGSGSGTQFTLTISDLECGDAATYFCAGGYNFGLFPFGGGT | |
| | Anti- | ELEILSSGGGGSGGGGGGSSRSSQSVKESEGRLVTPGGSLTLTCTVSGIDLSR | |
| | cotinine) | DWMNWVRQAPGEGLEWIGAIGRSGDTYYATWAKGRFTISKTSSRTVTLTVT | |
| | | DLQRSDTATYFCARIPYFGWNNGDIWGPGTLVTISS | |
| | | IEFMYPPPYLDNERSNGTIIHIKEKHLCHTQSSPKLFWALVVVAGVLFCYGLL | 199 |
| | | VTVALCVIWTNSRRNRGGQSDYMNMTPRRPGLTRKPYQPYAPARDFAAYR | |
| | | P | |
| | mCD3 | RAKFSRSAETAANLQDPNQLFNELNLGRREEFDVLEKKRARDPEMGGKQQR | 200 |
| | zeta | RRNPQEGVYNALQKDKMAEAYSEIGTKGERRRGKGHDGLFQGLSTATKDT | |
| | cyt | FDALHMQTLAPR | |
| His-28z | mouse | MDFQVQIFSFLLISASVIMSR | 201 |
| CAR | Ig | | |
| | kappa | | |
| | leader | | |
| | CAR | DILMTQTPSSLPVSLGDQASISCRSSQSIVHSNGNTYLEWYLQKPGQSPKLLIY | 202 |
| | | MKVSNRFSGVPDRFSGSGSGTDFTLKISRVEAEDLGVYYCFQGSHVPFTFGSGT | |
| | | MKLEIKRGGGGSGGGGSGGGGSGGGGSQVQLQQSGPEDVKPGASVKISCKAS | |
| | | MGYTFTDYYMNWVKQSPGKGLEWIGDINPNNGGTSYNQKFKGRATLTVDKS | |
| | | SSTAYMELRSLTSEDSSVYYCESQSGAYWGQGTTVTVSAIEFMYPPPYLDNE | |
| | | RSNGTIIHIKEKHLCHTQSSPKLFWALVVVAGVLFCYGLLVTVALCVIWTNS | |
| | | RRNRGGQSDYMNMTPRRPGLTRKPYQPYAPARDFAAYRPRAKFSRSAETAAN | |
| | | LQDPNQLFNELNLGRREEFDVLEKKRARDPEMGGKQQRRRNPQEGVYNALQK | |
| | | DKMAEAYSEIGTKGERRRGKGHDGLFQGLSTATKDTFDALHMQTLAPR | |
| | His | DILMTQTPSSLPVSLGDQASISCRSSQSIVHSNGNTYLEWYLQKPGQSPKLLIY | 203 |
| | scFv | KVSNRFSGVPDRFSGSGSGTDFTLKISRVEAEDLGVYYCFQGSHVPFTFGSGT | |
| | (3D5M) | KLEIKRGGGGSGGGGSGGGGSGGGGSQVQLQQSGPEDVKPGASVKISCKAS | |
| | | GYTFTDYYMNWVKQSPGKGLEWIGDINPNNGGTSYNQKFKGRATLTVDKS | |
| | | SSTAYMELRSLTSEDSSVYYCESQSGAYWGQGTTVTVSA | |
| | mCD28 | IEFMYPPPYLDNERSNGTIIHIKEKHLCHTQSSPKLFWALVVVAGVLFCYGLL | 204 |
| | TM/cyt | VTVALCVIWTNSRRNRGGQSDYMNMTPRRPGLTRKPYQPYAPARDFAAYR | |
| | | P | |
| | mCD3 | RAKFSRSAETAANLQDPNQLFNELNLGRREEFDVLEKKRARDPEMGGKQQR | 205 |
| | zeta | RRNPQEGVYNALQKDKMAEAYSEIGTKGERRRGKGHDGLFQGLSTATKDT | |
| | cyt | FDALHMQTLAPR | |
| His- | mouse | MASPLTRFLSLNLLLLGESIILGSGEA | 206 |
| BBz | CD8 | | |
| CAR | leader | | |
| | CAR | DILMTQTPSSLPVSLGDQASISCRSSQSIVHSNGNTYLEWYLQKPGQSPKLLIY | 207 |
| | | KVSNRFSGVPDRFSGSGSGTDFTLKISRVEAEDLGVYYCFQGSHVPFTFGSGT | |
| | | KLEIKRGGGGSGGGGSGGGGSGGGGSQVQLQQSGPEDVKPGASVKISCKAS | |
| | | GYTFTDYYMNWVKQSPGKGLEWIGDINPNNGGTSYNQKFKGRATLTVDKS | |
| | | SSTAYMELRSLTSEDSSVYYCESQSGAYWGQGTTVTVSATTTKPVLRTPSPVH | |
| | | PTGTSQPQRPEDCRPRGSVKGTGLDFACDIYIWAPLAGICVALLLSLIITLICKRG | |
| | | RKKLLYIFKQPFMRPVQTTQEEDGCSCRFPEEEEGGCELRAKFSRSAETAA | |
| | | NLQDPNQLYNELNLGRREEYDVLEKKRARDPEMGGKQQRRRNPQEGVYNALQK | |
| | | DKMAEAYSEIGTKGERRRGKGHDGLYQGLSTATKDTYDALHMQTLAPR | |
| | His | DILMTQTPSSLPVSLGDQASISCRSSQSIVHSNGNTYLEWYLQKPGQSPKLLIY | 208 |
| | scFv | KVSNRFSGVPDRFSGSGSGTDFTLKISRVEAEDLGVYYCFQGSHVPFTFGSGT | |
| | (3D5M) | KLEIKRGGGGSGGGGSGGGGSGGGGSQVQLQQSGPEDVKPGASVKISCKAS | |
| | | GYTFTDYYMNWVKQSPGKGLEWIGDINPNNGGTSYNQKFKGRATLTVDKS | |
| | | SSTAYMELRSLTSEDSSVYYCESQSGAYWGQGTTVTVSA | |
| | mCD8a | TTTKPVLRTPSPVHPTGTSQPQRPEDCRPRGSVKGTGLDFACDIYIWAPLAGI | 209 |
| | EC/TM | CVALLLSLIITLIC | |
| | h41BB | KRGRKKLLYIFKQPFMRPVQTTQEEDGCSCRFPEEEEGGCEL | 210 |
| | cyt | | |
| | mCD3 | RAKFSRSAETAANLQDPNQLYNELNLGRREEYDVLEKKRARDPEMGGKQQ | 211 |
| | zeta | RRRNPQEGVYNALQKDKMAEAYSEIGTKGERRRGKGHDGLYQGLSTATKD | |
| | | TYDALHMQTLAPR | |

Table 4(above): Murine CAR Sequences. Single underline = scFv; double underline mCD28
Tm/cyt; italics = mCD3 zeta cyt; italics and underlined = mCD8a EC/TM, bold = h41BB cyt.

TABLE 5
Human CAR

| | | | SEQ |
|---|---|---|---|
| Description | | Sequence | ID |
| hCot | hGM- | MLLLVTSLLLCELPHPAFLLIP | 212 |
| CAR | CSFR | | |
| (hCot- | alpha | | |
| 28z) | leader | | |
| | CAR | ELDLTQTPASVSAAVGGTVTINCQSSQSPYSNEWLSWYQQKPGQAPKVLISRI | 213 |
| | | STLASGVSSRFKGSGSGTQFTLTISDLECGDAATYFCAGGYNFGLFPFGGGTE | |
| | | LEILSSGGGGSGGGGGGSSRSSQSVKESEGRLVTPGGSLTLTCTVSGIDLSRD | |
| | | WMNWVRQAPGEGLEWIGAIGRSGDTYYATWAKGRFTISKTSSRTVTLTVTD | |
| | | LQRSDTATYFCARIPYFGWNNGDIWGPGTLVTISSAAAIEVMYPPPYLDNEKS | |
| | | NGTIIHVKGKHLCPSPLFPGPSKPFWVLVVVGGVLACYSLLVTVAFIIFWVRSK | |
| | | RSRLLHSDYMNMTPRRPGPTRKHYQPYAPPRDFAAYRSRVKFSRSADAPAYQQ | |
| | | GQNQLYNELNLGRREEYDVLDKRRGRDPEMGGKPRRKNPQEGLYNEL | |
| | | QKDKMAEAYSEIGMKGERRRGKGHDGLYQGLSTATKDTYDALHMQAL | |
| | | PPR | |
| | scFv | ELDLTQTPASVSAAVGGTVTINCQSSQSPYSNEWLSWYQQKPGQAPKVLISRI | 214 |
| | (Anti- | STLASGVSSRFKGSGSGTQFTLTISDLECGDAATYFCAGGYNFGLFPFGGGTE | |
| | cotinine) | LEILSSGGGGSGGGGGGSSRSSQSVKESEGRLVTPGGSLTLTCTVSGIDLSRD | |
| | | WMNWVRQAPGEGLEWIGAIGRSGDTYYATWAKGRFTISKTSSRTVTLTVTD | |
| | | LQRSDTATYFCARIPYFGWNNGDIWGPGTLVTISS | |
| | NotI | AAA | n/1 |
| | site | | |
| | hCD28 | IEVMYPPPYLDNEKSNGTIIHVKGKHLCPSPLFPGPSKPFWVLVVVGGVLACY | 215 |
| | EC/TM | SLLVTVAFIIFWV | |
| | hCD28 | RSKRSRLLHSDYMNMTPRRPGPTRKHYQPYAPPRDFAAYRS | 216 |
| | cyt | | |
| | hCD3 | RVKFSRSADAPAYQQGQNQLYNELNLGRREEYDVLDKRRGRDPEMGGKPR | 217 |
| | zeta cyt | RKNPQEGLYNELQKDKMAEAYSEIGMKGERRRGKGHDGLYQGLSTATKDT | |
| | | YDALHMQALPPR | |
| hHis | hCD8 | MALPVTALLLPLALLLHAARP | 218 |
| CAR | leader | | |
| (3D5M- | CAR | DILMTQTPSSLPVSLGDQASISCRSSQSIVHSNGNTYLEWYLQKPGQSPKLLIY | 219 |
| BBz) | | KVSNRFSGVPDRFSGSGSGTDFTLKISRVEAEDLGVYYCFQGSHVPFTFGSGT | |
| | | KLEIKRGGGGSGGGGSGGGGSGGGGSQVQLQQSGPEDVKPGASVKISCKAS | |
| | | GYTFTDYYMNWVKQSPGKGLEWIGDINPNNGGTSYNQKFKGRATLTVDKSS | |
| | | STAYMELRSLTSEDSSVYYCESQSGAYWGQGTTVTVSATTTPAPRPPTPAPTI | |
| | | ASQPLSLRPEACRPAAGGAVHTRGLDFACDIYIWAPLAGTCGVLLLSLVITLY | |
| | | CKRGRKKLLYIFKQPFMRPVQTTQEEDGCSCRFPEEEEGGCELRVKFSRSADAP | |
| | | AYKQGQNQLYNELNLGRREEYDVLDKRRGRDPEMGGKPRRKNPQEGL | |
| | | YNELQKDKMAEAYSEIGMKGERRRGKGHDGLYQGLSTATKDTYDALH | |
| | | MQALPPR | |
| | scFv | DILMTQTPSSLPVSLGDQASISCRSSQSIVHSNGNTYLEWYLQKPGQSPKLLIY | 220 |
| | (3D5M) | KVSNRFSGVPDRFSGSGSGTDFTLKISRVEAEDLGVYYCFQGSHVPFTFGSGT | |
| | | KLEIKRGGGGSGGGGSGGGGSGGGGSQVQLQQSGPEDVKPGASVKISCKAS | |
| | | GYTFTDYYMNWVKQSPGKGLEWIGDINPNNGGTSYNQKFKGRATLTVDKSS | |
| | | STAYMELRSLTSEDSSVYYCESQSGAYWGQGTTVTVSA | |
| | hCD8 | TTTPAPRPPTPAPTIASQPLSLRPEACRPAAGGAVHTRGLDFACDIYIWAPLAG | |
| | EC/TM | TCGVLLLSLVITLYC | |
| | h41BB | KRGRKKLLYIFKQPFMRPVQTTQEEDGCSCRFPEEEEGGCEL | 221 |
| | cyt | | |
| | hCD3 | RVKFSRSADAPAYKQGQNQLYNELNLGRREEYDVLDKRRGRDPEMGGKPR | 222 |
| | zeta cyt | RKNPQEGLYNELQKDKMAEAYSEIGMKGERRRGKGHDGLYQGLSTATKDT | |
| | | YDALHMQALPPR | |
| huHis | hCD8 | MALPVTALLLPLALLLHAARP | 223 |
| CAR | leader | | |
| (humanized | CAR | DIVMTQSPLSLPVTPGEPASISCRSSQSIVHSNRNTYLEWYLQKPGQSPQLLIY | 224 |
| 3D5 | | KVSNRFSGVPDRFSGSGSGTDFTLKISRVEAEDVGVYYCFQGSHVPFTFGQGT | |
| M-BBz) | | KLEIKRGGGGSGGGGSGGGGSGGGGSQVQLVQSGAEVKKPGASVKVSCKAS | |
| | | GYTFTDYYMNWVRQAPGQGLEWMGDINPNHGGTYYNHKFKGRVTMTVDT | |
| | | STSTAYMELRSLRSDDTAVYYCESQSGAYWGQGTTVTVSSTTTPAPRPPTPA | |
| | | PTIASQPLSLRPEACRPAAGGAVHTRGLDFACDIYIWAPLAGTCGVLLLSLVIT | |
| | | LYCKRGRKKLLYIFKQPFMRPVQTTQEEDGCSCRFPEEEEGGCELRVKFSRSAD | |
| | | APAYKQGQNQLYNELNLGRREEYDVLDKRRGRDPEMGGKPRRKNPQE | |
| | | GLYNELQKDKMAEAYSEIGMKGERRRGKGHDGLYQGLSTATKDTYDA | |
| | | LHMQALPPR | |
| | scFv | DIVMTQSPLSLPVTPGEPASISCRSSQSIVHSNRNTYLEWYLQKPGQSPQLLIY | 225 |
| | (hu3D5M) | KVSNRFSGVPDRFSGSGSGTDFTLKISRVEAEDVGVYYCFQGSHVPFTFGQGT | |
| | | KLEIKRGGGGSGGGGSGGGGSGGGGSQVQLVQSGAEVKKPGASVKVSCKAS | |
| | | GYTFTDYYMNWVRQAPGQGLEWMGDINPNHGGTYYNHKFKGRVTMTVDT | |
| | | STSTAYMELRSLRSDDTAVYYCESQSGAYWGQGTTVTVSS | |
| | hCD8 | TTTPAPRPPTPAPTIASQPLSLRPEACRPAAGGAVHTRGLDFACDIYIWAPLAG | 226 |
| | EC/TM | TCGVLLLSLVITLYC | |
| | h41BB | KRGRKKLLYIFKQPFMRPVQTTQEEDGCSCRFPEEEEGGCEL | 227 |
| | cyt | | |
| | hCD3 | RVKFSRSADAPAYKQGQNQLYNELNLGRREEYDVLDKRRGRDPEMGGKPR | 228 |
| | zeta cyt | RKNPQEGLYNELQKDKMAEAYSEIGMKGERRRGKGHDGLYQGLSTATKDT | |
| | | YDALHMQALPPR | |
| hMyc | hCD8 | MALPVTALLLPLALLLHAARP | 229 |
| (3A6) | leader | | |
| CAR | CAR | LTQPSSVSANLGGTVKITCSGGGGSDDGSYYYSWHQQKSPGSAPVTVIYENN | 230 |
| (3A6- | | NRPSDIPSRFSGSKSGSTATLTITGVQAEDEAVYYCGSRDSSYVDYVGIFGAG | |
| BBz) | | TTLTVLGQSSRSSGGGGSSGGGGSAVTLDESGGGLQTPGRALSLVCKASGFTF | |
| | | SSYDMAWVRQEPGKGLEYVASISRSGRYTRYGPAVKGRATISRDNGQSTVRL | |
| | | QLNNLRAEDTATYFCTRGGAVSIDTWGHGTEVIVSSTTTPAPRPPTPAPTIASQ | |
| | | PLSLRPEACRPAAGGAVHTRGLDFACDIYIWAPLAGTCGVLLLSLVITLYCNKR | |
| | | GRKKLLYIFKQPFMRPVQTTQEEDGCSCRFPEEEEGGCELRVKFSRSADAPAY | |
| | | KQGQNQLYNELNLGRREEYDVLDKRRGRDPEMGGKPRRKNPQEGLYN | |
| | | ELQKDKMAEAYSEIGMKGERRRGKGHDGLYQGLSTATKDTYDALHMQ | |
| | | ALPPR | |
| | scFv | LTQPSSVSANLGGTVKITCSGGGGSDDGSYYYSWHQQKSPGSAPVTVIYENN | 230 |
| | (3A6) | NRPSDIPSRFSGSKSGSTATLTITGVQAEDEAVYYCGSRDSSYVDYVGIFGAG | |
| | | TTLTVLGQSSRSSGGGGSSGGGGSAVTLDESGGGLQTPGRALSLVCKASGFTF | |
| | | SSYDMAWVRQEPGKGLEYVASISRSGRYTRYGPAVKGRATISRDNGQSTVRL | |
| | | QLNNLRAEDTATYFCTRGGAVSIDTWGHGTEVIVSS | |
| | hCD8 | TTTPAPRPPTPAPTIASQPLSLRPEACRPAAGGAVHTRGLDFACDIYIWAPLAG | 231 |
| | EC/TM | TCGVLLLSL VITLYC | |
| | h41BB | KRGRKKLLYIFKQPFMRPVQTTQEEDGCSCRFPEEEEGGCEL | 232 |
| | cyt | | |
| | hCD3 | RVKFSRSADAPAYKQGQNQLYNELNLGRREEYDVLDKRRGRDPEMGGKPR | 233 |
| | zeta cyt | RKNPQEGLYNELQKDKMAEAYSEIGMKGERRRGKGHDGLYQGLSTATKDT | |
| | | YDALHMQALPPR | |
| hMyc | hCD8 | MALPVTALLLPLALLLHAARP | 234 |
| (7A1) | leader | | |
| CAR | CAR | LTQPSSVSVNPGETVKITCSGGGSSSYYGWYQQKSPGSAPVTLIYQNTNRPSN | 235 |
| (7A1- | | | |
| BBz) | | IPSRFSGSTSGSTGTLTITGVQAEDEAVYFCGSRDSSGDGIFGAGTTLTVLGQS | |
| | | SRSSGGGGSSGGGGSAVTLDESGGGLQTPGRALSLVCKASGFTFSSYDMAW | |
| | | VRQEPGKGLEYVASISRSGRYTRYGPAVKGRATISRDNGQSTVRLQLNNLRA | |
| | | EDTATYFCTRGGAVSIDTWGHGTEVIVSSTTTPAPRPPTPAPTIASQPLSLRPE | |
| | | ACRPAAGGAVHTRGLDFACDIYIWAPLAGTCGVLLLSLVITLYCKRGRKKLLY | |
| | | IFKQPFMRPVQTTQEEDGCSCRFPEEEEGGCELRVKFSRSADAPAYKQGQNQ | |
| | | LYNELNLGRREEYDVLDKRRGRDPEMGGKPRRKNPQEGLYNELQKDK | |
| | | MAEAYSEIGMKGERRRGKGHDGLYQGLSTATKDTYDALHMQALPPR | |
| | scFv | LTQPSSVSVNPGETVKITCSGGGSSSYYGWYQQKSPGSAPVTLIYQNTNRPSN | 236 |
| | (7A1) | IPSRFSGSTSGSTGTLTITGVQAEDEAVYFCGSRDSSGDGIFGAGTTLTVLGQS | |
| | | SRSSGGGGSSGGGGSAVTLDESGGGLQTPGRALSLVCKASGFTFSSYDMAW | |
| | | VRQEPGKGLEYVASISRSGRYTRYGPAVKGRATISRDNGQSTVRLQLNNLRA | |
| | | EDTATYFCTRGGAVSIDTWGHGTEVIVSS | |
| | hCD8 | TTTPAPRPPTPAPTIASQPLSLRPEACRPAAGGAVHTRGLDFACDIYIWAPLAG | 237 |
| | EC/TM | TCGVLLLSLVITLYC | |
| | h41BB | KRGRKKLLYIFKQPFMRPVQTTQEEDGCSCRFPEEEEGGCEL | 238 |
| | cyt | | |
| | hCD3 | RVKFSRSADAPAYKQGQNQLYNELNLGRREEYDVLDKRRGRDPEMGGKPR | 239 |
| | zeta cyt | RKNPQEGLYNELQKDKMAEAYSEIGMKGERRRGKGHDGLYQGLSTATKDT | |
| | | YDALHMQALPPR | |
| hMyc | hCD8 | MALPVTALLLPLALLLHAARP | 240 |
| (8A9) | leader | | |
| CAR | CAR | LTQPSSVSANPGETVEITCSGGSSGYYGWHQQKSPGSAPVTVIYENTNRPSDIP | 241 |
| (8A9- | | SRFSGSKSGSTATLTITGVQADDEAVYFCGSYDSSSASGIFGAGTTLTVLGQSS | |
| BBz) | | RSSGGGGSSGGGGSAVTLDESGGGLQTPGGGLSLVCKASGFTFSSYDMAWV | |
| | | RQEPGKGLEYVASISRSGRYTRYGPAVKGRATISRDNGQSTVRLQLNNLRAE | |
| | | DTATYFCTRGGAVSIDTWGHGTEVIVSSTTTPAPRPPTPAPTIASQPLSLRPEA | |
| | | CRPAAGGAVHTRGLDFACDIYIWAPLAGTCGVLLLSLVITLYCKRGRKKLLYIF | |
| | | KQPFMRPVQTTQEEDGCSCRFPEEEEGGCELRVKFSRSADAPAYKQGQNQL | |
| | | YNELNLGRREEYDVLDKRRGRDPEMGGKPRRKNPQEGLYNELQKDKM | |
| | | AEAYSEIGMKGERRRGKGHDGLYQGLSTATKDTYDALHMQALPPR | |
| | scFv | LTQPSSVSANPGETVEITCSGGSSGYYGWHQQKSPGSAPVTVIYENTNRPSDIP | 242 |
| | (8A9) | SRFSGSKSGSTATLTITGVQADDEAVYFCGSYDSSSASGIFGAGTTLTVLGQSS | |
| | | RSSGGGGSSGGGGSAVTLDESGGGLQTPGGGLSLVCKASGFTFSSYDMAWV | |
| | | RQEPGKGLEYVASISRSGRYTRYGPAVKGRATISRDNGQSTVRLQLNNLRAE | |
| | | DTATYFCTRGGAVSIDTWGHGTEVIVSS | |
| | hCD8 | TTTPAPRPPTPAPTIASQPLSLRPEACRPAAGGAVHTRGLDFACDIYIWAPLAG | 243 |
| | EC/TM | TCGVLLLSLVITLYC | |
| | h41BB | KRGRKKLLYIFKQPFMRPVQTTQEEDGCSCRFPEEEEGGCEL | 244 |
| | cyt | | |
| | hCD3 | RVKFSRSADAPAYKQGQNQLYNELNLGRREEYDVLDKRRGRDPEMGGKPR | 245 |
| | zeta cyt | RKNPQEGLYNELQKDKMAEAYSEIGMKGERRRGKGHDGLYQGLSTATKDT | |
| | | YDALHMQALPPR | |

Table 5(above): Human CAR Sequences. Single underline = scFv; double underline = hCD28 EC/TM;
italics = hCD28 cyt or h41BB cyt; bold = hCD3 zeta cyt.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of aspects of the present disclosure. However, it will be apparent to one of skill in the art that aspects of the present disclosure may be practiced without one or more of these specific details. In other instances, well-known features and procedures well known to those skilled in the art have not been described in order to avoid obscuring aspects of the present disclosure. Embodiments of the disclosure have been described for illustrative and not restrictive purposes. Although the aspects of the present disclosure are described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. A switchable chimeric antigen receptor immune cell system comprising:
a chimeric antigen receptor immune cell and an anti-tumor antibody conjugated to a peptide tag,
wherein the chimeric antigen receptor immune cell comprises a chimeric antigen receptor (CAR),
wherein the CAR comprises an antigen recognition domain that recognizes the peptide tag, and
wherein the anti-tumor antibody is an anti-CD40 or anti-CS1 antibody,
wherein the CAR further comprises a transmembrane domain and a signal transduction domain.

2. The switchable chimeric antigen receptor immune cell system of claim 1, wherein the switchable CAR immune cell is a T cell, a B cell, a natural killer (NK) cell, NKT cell, or a macrophage.

3. (canceled)

4. The switchable chimeric antigen receptor immune cell system of claim 1, wherein the antigen recognition domain comprises an antibody that recognizes the peptide tag.

5. The switchable chimeric antigen receptor immune cell system of claim 1, wherein the peptide tag is His tag or a Myc tag.

6. The switchable chimeric antigen receptor immune cell system of claim 1, wherein the antibody that recognizes the peptide tag is a scFv, Fab, Fab′, Fv, or single domain antibody variable region.

7. The switchable chimeric antigen receptor immune cell system of claim 1, wherein the antigen recognition domain recognizes a His tag, wherein the antigen recognition domain comprises a heavy chain variable region comprising HCDRs 1-3 and a light chain variable region comprising LCDRs 1-3,
wherein the HCDRs 1-3 comprise sequences of SEQ ID NOs: 31-33, respectively, and the LCDRs 1-3 comprises sequences of SEQ ID NOs 34-36, respectively; or
wherein the HCDRs 1-3 comprise sequences of SEQ ID NOs: 40-42, respectively, and the LCDRs 1-3 comprise sequences of SEQ ID NOs 43-45, respectively.

8. The switchable chimeric antigen receptor immune cell system of claim 1, wherein the antigen recognition domain recognizes a Myc tag, wherein antigen recognition domain comprises a heavy chain variable region comprising HCDRs 1-3 and a light chain variable region comprising LCDRs 1-3,
wherein the HCDRs 1-3 comprise sequences of SEQ ID NOs: 49-51, respectively and the LCDRs 1-3 comprise sequences of SEQ ID NOs: 52-54, respectively;
wherein the HCDRs 1-3 comprise sequences of SEQ ID NOs: 58-60, respectively and the LCDRs 1-3 comprise sequences of SEQ ID NOs: 61-63, respectively; or
wherein the HCDRs 1-3 comprise sequences of SEQ ID NOs: 67-69, respectively and the LCDRs 1-3 comprise sequences of SEQ ID NOs: 70-72, respectively.

9. The switchable chimeric antigen receptor immune cell system of claim 1, wherein the peptide tag comprises a Histidine multimer.

10. The switchable chimeric antigen receptor immune cell system of claim 1, wherein the peptide tag comprises 2-25 histidines.

11. The switchable chimeric antigen receptor immune cell system of claim 1, wherein the peptide tag comprises a Myc tag.

12. The switchable chimeric antigen receptor immune cell system of claim 11, wherein the Myc tag comprise SEQ ID NO: 146.

13. The switchable chimeric antigen receptor immune cell system of claim 1, wherein the anti-CD40 antibody comprises a heavy chain variable region comprising HCDRs 1-3 and a light chain variable region comprising LCDRs 1-3,
wherein the HCDRs 1-3 comprise sequences of SEQ ID NOs 4-6, respectively, wherein the LCDRs 1-3 comprise one of SEQ ID NOs: 7-9, respectively; or
wherein the HCDRs 1-3 comprise sequences of SEQ ID NOs 13-15, respectively, wherein the LCDRs 1-3 comprise one of SEQ ID NOs: 16-18, respectively; or
wherein the HCDRs 1-3 comprise sequences of SEQ ID NOs 22-24, respectively, wherein the LCDRs 1-3 comprise one of SEQ ID NOs: 25-27, respectively.

14. The switchable chimeric antigen receptor immune cell system of claim 1, wherein the anti-CS1 antibody comprises a heavy chain variable region comprising HCDRs 1-3 and a light chain variable region comprising LCDRs 1-3,
wherein the HCDRs 1-3 comprise sequences of SEQ ID NOs 85-87, respectively, wherein the LCDRs 1-3 comprise one of SEQ ID NOs:88-90 respectively;
wherein the HCDRs 1-3 comprise sequences of SEQ ID NOs 94-96, respectively, wherein the LCDRs 1-3 comprise one of SEQ ID NOs: 97-99, respectively;
wherein the HCDRs 1-3 comprise sequences of SEQ ID NOs 103-105, respectively, wherein the LCDRs 1-3 comprise SEQ ID NOs: 106-108, respectively.

15. The switchable chimeric antigen receptor immune cell system of claim 1, wherein the peptide tag consists essentially of a Histidine multimer or a Myc tag.

16. The switchable chimeric antigen receptor immune cell system of claim 1, wherein the peptide tag is a His tag and the antibody that recognizes the peptide tag is an anti-His antibody.

17. The switchable chimeric antigen receptor immune cell system of claim 1, wherein the peptide tag is a Myc tag and the antibody that recognizes the peptide tag is an anti-Myc antibody.

18. A method of treating cancer, the method comprising:
administering, to a subject having or suspected of having a cancer, a plurality of chimeric antigen receptor immune (CAR) immune cells, wherein each of the plurality comprises a chimeric antigen receptor (CAR), and wherein the CAR comprises an antigen recognition domain that recognizes a peptide tag, and wherein the anti-tumor antibody conjugated to the peptide tag, and wherein the anti-tumor antibody is an anti-CD40 antibody or an anti-CS1 antibody; and
administering an anti-tumor antibody conjugated to the peptide tag to the subject.

19.-21. (canceled)

22. The method of claim 18, wherein the peptide tag is a His tag or a Myc tag.

23.-32. (canceled)

33. A chimeric antigen receptor immune cell comprising an antibody or antigen binding portion thereof that binds to a His tag or a Myc tag.

34. (canceled)

35. The chimeric antigen receptor immune cell of claim 33, wherein the antibody or antigen binding portion thereof that binds to a His tag comprises a heavy chain variable region comprising a heavy chain variable region comprising HCDRs 1-3 and a light chain variable region comprising LCDRs 1-3,
wherein the HCDRs 1-3 comprise SEQ ID NOs 31-33 respectively and the LCDRs 1-3 comprise SEQ ID NOs 34-36, respectively, or
wherein the HCDRs 1-3 comprise SEQ ID NOs 40-42 respectively and the LCDRs 1-3 comprises SEQ ID NOs 43-45, respectively.

36. The chimeric antigen receptor immune cell of claim 33, wherein the antibody or antigen binding portion thereof that binds to a Myc tag comprises:
a heavy chain variable region comprising HCDRs 1-3 and a light chain variable region comprising LCDRs 1-3,
wherein the HCDRs 1-3 comprise sequences of SEQ ID NOs: 49-51, respectively and the LCDRs 1-3 comprise sequences of SEQ ID NOs: 52-54, respectively;
wherein the HCDRs 1-3 comprise sequences of SEQ ID NOs: 58-60, respectively and the LCDRs 1-3 comprise sequences of SEQ ID NOs: 61-63, respectively; and
wherein the HCDRs 1-3 comprise sequences of SEQ ID NOs: 67-69, respectively and the LCDRs 1-3 comprise sequences of SEQ ID NOs: 70-72, respectively.

37. A switchable chimeric antigen receptor immune cell system comprising the chimeric antigen receptor immune cell of claim 33 and an anti-tumor antibody conjugated to the His tag or the Myc tag.

38. A switchable chimeric antigen receptor immune cell pharmaceutical composition, comprising:
a plurality of chimeric antigen receptor immune (CAR) cells, wherein each of the plurality of chimeric antigen receptor immune (CAR) cells comprises the chimeric antigen receptor (CAR), and the anti-tumor antibody of claim 1; and a pharmaceutically acceptable carrier.

39.-43. (canceled)

44. The pharmaceutical composition of claim 38, wherein the peptide tag is a His tag or a Myc tag.

45.-54. (canceled)


SEQUENCE LISTING (246 sequences)

SEQ ID NO: 1 - protein, 258 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSS

SEQ ID NO: 2 - protein, 129 aa, synthetic construct
AVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSS

SEQ ID NO: 3 - protein, 111 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVL

SEQ ID NO: 4 - protein, 10 aa, synthetic construct
GFTFSSYQMF

SEQ ID NO: 5 - protein, 17 aa, synthetic construct
EISGSGLSTWYAPAVKG

SEQ ID NO: 6 - protein, 20 aa, synthetic construct
AAGSCGVGTCAGGIVDGIDA

SEQ ID NO: 7 - protein, 13 aa, synthetic construct
SGGDSYAGSYYYG

SEQ ID NO: 8 - protein, 7 aa, synthetic construct
YNDKRPS

SEQ ID NO: 9 - protein, 11 aa, synthetic construct
GSIDTSSGTGI

SEQ ID NO: 10 - protein, 247 aa, synthetic construct
QAALTQPSSVSANPGETVKITCSGSSSSYYGWYQQKAPGSAPVTLIYANDKRPSDIPSRFSGSKSGSTHTLTITGVQADDEAVYFCGSADSTDSGFGAGTTLTVLGQSSRSSGGGGSSGGGGSAVTLDESGGGLQTPGGALSLVCKASGFSFSSYSMHWVRQAPGKGLEFVAAISSDGSRYTHYGPAVKGRATISRDNGQSTVRLQLNNLRPEDTGTYYCAKTSNTWTWNTGWIDAWGHGTEVIVSS

SEQ ID NO: 11 - protein, 124 aa, synthetic construct
AVTLDESGGGLQCPGGALSLVCKASGFSFSSYSMHWVRQAPGKGLEFVAAISSDGSRYTHYGPAVKGRATISRDNGQSTVRLQLNNLRPEDTGTYYCAKTSNTWTWNTGWIDAWGHGTEVIVSS

SEQ ID NO: 12 - protein, 105 aa, synthetic construct
QAALTQPSSVSANPGETVKITCSGSSSSYYGWYQQKAPGSAPVTLIYANDKRPSDIPSRFSGSKSGSTHTLTITGVQADDEAVYFCGSADSTDSGFGAGTTLTVL

SEQ ID NO: 13 - protein, 10 aa, synthetic construct
GFSFSSYSMH

SEQ ID NO: 14 - protein, 18 aa, synthetic construct
AISSDGSRYTHYGPAVKG

SEQ ID NO: 15 - protein, 14 aa, synthetic construct
TSNTWTWNTGWIDA

SEQ ID NO: 16 - protein, 9 aa, synthetic construct
SGSSSSYYG

SEQ ID NO: 17 - protein, 7 aa, synthetic construct
ANDKRPS

SEQ ID NO: 18 - protein, 9 aa, synthetic construct
GSADSTDSG

SEQ ID NO: 19 - protein, 258 aa, synthetic construct
AIQLTQSPSSLSASVGDRVTITCSGGDSYAGSYYYGWYQQKPGKAPKTLIYYNDKRPSGVPSRFSGSTSGTDSTLTISSLQPEDFATYYCGSIDTSSGTGIFGQGTKLEIKGQSSRSSGGGGSSGGGGSEVQLLESGGGLVQPGGSLRLSCAASGFTFSSYQMFWVRQAPGKGLEYVSEISGSGLSTWYAPAVKGRFTISRDNSKNTLYLQMNSLRAEDTAVYYCAKAAGSCGVGTCAGGIVDAIDAWGQGTTVTVSS

SEQ ID NO: 20 - protein, 129 aa, synthetic construct
EVQLLESGGGLVQPGGSLRLSCAASGFTFSSYQMFWVRQAPGKGLEYVSEISGSGLSTWYAPAVKGRFTISRDNSKNTLYLQMNSLRAEDTAVYYCAKAAGSCGVGTCAGGIVDAIDAWGQGTTVTVSS

SEQ ID NO: 21 - protein, 111 aa, synthetic construct
AIQLTQSPSSLSASVGDRVTITCSGGDSYAGSYYYGWYQQKPGKAPKTLIYYNDKRPSGVPSRFSGSTSGTDSTLTISSLQPEDFATYYCGSIDTSSGTGIFGQGTKLEIK

SEQ ID NO: 22 - protein, 10 aa, synthetic construct
GFTFSSYQMF

SEQ ID NO: 23 - protein, 17 aa, synthetic construct
EISGSGLSTWYAPAVKG

SEQ ID NO: 24 - protein, 20 aa, synthetic construct
AAGSCGVGTCAGGIVDAIDA

SEQ ID NO: 25 - protein, 13 aa, synthetic construct
SGGDSYAGSYYYG

SEQ ID NO: 26 - protein, 7 aa, synthetic construct
YNDKRPS

SEQ ID NO: 27 - protein, 11 aa, synthetic construct
GSIDTSSGTGI

SEQ ID NO: 28 - protein, 247 aa, synthetic construct
DILMTQTPSSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSGGGGSQVQLQQSGPEDVKPGASVKISCKASGYTFTDYYMNWVKQSPGKGLEWIGDINPNNGGTSYNQKFKGRATLTVDKSSSTAYMELRSLTSEDSSVYYCESQSGAYWGQGTTVTVSA

SEQ ID NO: 29 - protein, 114 aa, synthetic construct
QVQLQQSGPEDVKPGASVKISCKASGYTFTDYYMNWVKQSPGKGLEWIGDINPNNGGTSYNQKFKGRATLTVDKSSSTAYMELRSLTSEDSSVYYCESQSGAYWGQGTTVTVSA

SEQ ID NO: 30 - protein, 112 aa, synthetic construct
DILMTQTPSSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 31 - protein, 10 aa, synthetic construct
GYTFTDYYMN

SEQ ID NO: 32 - protein, 17 aa, synthetic construct
DINPNNGGTSYNQKFKG

SEQ ID NO: 33 - protein, 5 aa, synthetic construct
QSGAY

SEQ ID NO: 34 - protein, 16 aa, synthetic construct
RSSQSIVHSNGNTYLE

SEQ ID NO: 35 - protein, 7 aa, synthetic construct
KVSNRFS

SEQ ID NO: 36 - protein, 9 aa, synthetic construct
FQGSHVPFT

SEQ ID NO: 37 - protein, 247 aa, synthetic construct
DIVMTQSPLSLPVTPGEPASISCRSSQSIVHSNRNTYLEWYLQKPGQSPQLLIYKVSNRFSGVPDRFSGSGSGTDFTLKISRVEAEDVGVYYCFQGSHVPFTFGQGTKLEIKRGGGGSGGGGSGGGGSGGGGSQVQLVQSGAEVKKPGASVKVSCKASGYTFTDYYMNWVRQAPGQGLEWMGDINPNHGGTYYNHKFKGRVTMTVDTSTSTAYMELRSLRSDDTAVYYCESQSGAYWGQGTTVTVSS

SEQ ID NO: 38 - protein, 114 aa, synthetic construct
QVQLVQSGAEVKKPGASVKVSCKASGYTFTDYYMNWVRQAPGQGLEWMGDINPNHGGTYYNHKFKGRVTMTVDTSTSTAYMELRSLRSDDTAVYYCESQSGAYWGQGTTVTVSS

SEQ ID NO: 39 - protein, 112 aa, synthetic construct
DIVMTQSPLSLPVTPGEPASISCRSSQSIVHSNRNTYLEWYLQKPGQSPQLLIYKVSNRFSGVPDRFSGSGSGTDFTLKISRVEAEDVGVYYCFQGSHVPFTFGQGTKLEIK

SEQ ID NO: 40 - protein, 10 aa, synthetic construct
GYTFTDYYMN

SEQ ID NO: 41 - protein, 17 aa, synthetic construct
DINPNHGGTYYNHKFKG

SEQ ID NO: 42 - protein, 5 aa, synthetic construct
QSGAY

SEQ ID NO: 43 - protein, 16 aa, synthetic construct
RSSQSIVHSNRNTYLE

SEQ ID NO: 44 - protein, 7 aa, synthetic construct
KVSNRFS

SEQ ID NO: 45 - protein, 9 aa, synthetic construct
FQGSHVPFT

SEQ ID NO: 46 - protein, 249 aa, synthetic construct
QAALTQPSSVSANLGGTVKITCSGGGGSDDGSYYYSWHQQKSPGSAPVTVIYENNNRPSDIPSRFSGSKSGSTATLTITGVQAEDEAVYYCGSRDSSYVDYVGIFGAGTTLTVLGQSSRSSGGGGSSGGGGSAVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 47 - protein, 117 aa, synthetic construct
AVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 48 - protein, 114 aa, synthetic construct
QAALTQPSSVSANLGGTVKITCSGGGGSDDGSYYYSWHQQKSPGSAPVTVIYENNNRPSDIPSRFSGSKSGSTATLTITGVQAEDEAVYYCGSRDSSYVDYVGIFGAGTTLTVL

SEQ ID NO: 49 - protein, 10 aa, synthetic construct
GFTFSSYDMA

SEQ ID NO: 50 - protein, 17 aa, synthetic construct
SISRSGRYTRYGPAVKG

SEQ ID NO: 51 - protein, 8 aa, synthetic construct
GGAVSIDT

SEQ ID NO: 52 - protein, 14 aa, synthetic construct
SGGGGSDDGSYYYS

SEQ ID NO: 53 - protein, 7 aa, synthetic construct
ENNNRPS

SEQ ID NO: 54 - protein, 13 aa, synthetic construct
GSRDSSYVDYVGI

SEQ ID NO: 55 - protein, 242 aa, synthetic construct
QAALTQPSSVSVNPGETVKITCSGGGSSSYYGWYQQKSPGSAPVTLIYQNTNRPSNIPSRFSGSTSGSTGTLTITGVQAEDEAVYFCGSRDSSGDGIFGAGTTLTVLGQSSRSSGGGGSSGGGGSAVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 56 - protein, 117 aa, synthetic construct; identical to SEQ ID NO: 47

SEQ ID NO: 57 - protein, 107 aa, synthetic construct
QAALTQPSSVSVNPGETVKITCSGGGSSSYYGWYQQKSPGSAPVTLIYQNTNRPSNIPSRFSGSTSGSTGTLTITGVQAEDEAVYFCGSRDSSGDGIFGAGTTLTVL

SEQ ID NO: 58 - protein, 10 aa, synthetic construct
GFTFSSYDMA

SEQ ID NO: 59 - protein, 17 aa, synthetic construct
SISRSGRYTRYGPAVKG

SEQ ID NO: 60 - protein, 8 aa, synthetic construct
GGAVSIDT

SEQ ID NO: 61 - protein, 10 aa, synthetic construct
SGGGSSSYYG

SEQ ID NO: 62 - protein, 7 aa, synthetic construct
QNTNRPS

SEQ ID NO: 63 - protein, 10 aa, synthetic construct
GSRDSSGDGI

SEQ ID NO: 64 - protein, 242 aa, synthetic construct
QAALTQPSSVSANPGETVEITCSGGSSGYYGWHQQKSPGSAPVTVIYENTNRPSDIPSRFSGSKSGSTATLTITGVQADDEAVYFCGSYDSSSASGIFGAGTTLTVLGQSSRSSGGGGSSGGGGSAVTLDESGGGLQTPGGGLSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 65 - protein, 117 aa, synthetic construct
AVTLDESGGGLQTPGGGLSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 66 - protein, 107 aa, synthetic construct
QAALTQPSSVSANPGETVEITCSGGSSGYYGWHQQKSPGSAPVTVIYENTNRPSDIPSRFSGSKSGSTATLTITGVQADDEAVYFCGSYDSSSASGIFGAGTTLTVL

SEQ ID NO: 67 - protein, 10 aa, synthetic construct
GFTFSSYDMA

SEQ ID NO: 68 - protein, 17 aa, synthetic construct
SISRSGRYTRYGPAVKG

SEQ ID NO: 69 - protein, 8 aa, synthetic construct
GGAVSIDT

SEQ ID NO: 70 - protein, 9 aa, synthetic construct
SGGSSGYYG

SEQ ID NO: 71 - protein, 7 aa, synthetic construct
ENTNRPS

SEQ ID NO: 72 - protein, 11 aa, synthetic construct
GSYDSSSASGI

SEQ ID NO: 73 - protein, 250 aa, synthetic construct
QAALTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQTPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSSTS

SEQ ID NO: 74 - protein, 126 aa, synthetic construct
AVTLDESGGGLQCPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSSTS

SEQ ID NO: 75 - protein, 106 aa, synthetic construct
QAALTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVL

SEQ ID NO: 76 - protein, 10 aa, synthetic construct
GFTFSSVNMQ

SEQ ID NO: 77 - protein, 17 aa, synthetic construct
GISSDGSYTAYGAAVKG

SEQ ID NO: 78 - protein, 15 aa, synthetic construct
IAGGYYWDAAYSIDM

SEQ ID NO: 79 - protein, 8 aa, synthetic construct
SGGSSNYG

SEQ ID NO: 80 - protein, 7 aa, synthetic construct
DNTNRPS

SEQ ID NO: 81 - protein, 9 aa, synthetic construct
YDSGTYAGI

SEQ ID NO: 82 - protein, 257 aa, synthetic construct
QAALTQPSSVSANLGETVKITCSGSSGNNYGWYQQKSPGSAPVTVIYYNDNRPSDIPSRFSGSKSGSTHTLTITGVQADDEAVYFCGSGDSSSNPGIFGAGTTLTVLGQSSRSSGGGGSSGGGGSAVTLDESGGGLQTPGGALSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSS

SEQ ID NO: 83 - protein, 132 aa, synthetic construct
AVTLDESGGGLQTPGGALSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSS

SEQ ID NO: 84 - protein, 107 aa, synthetic construct
QAALTQPSSVSANLGETVKITCSGSSGNNYGWYQQKSPGSAPVTVIYYNDNRPSDIPSRFSGSKSGSTHTLTITGVQADDEAVYFCGSGDSSSNPGIFGAGTTLTVL

SEQ ID NO: 85 - protein, 5 aa, synthetic construct
SHGMA

SEQ ID NO: 86 - protein, 17 aa, synthetic construct
GIDDSGSSTGYGSAVKG

SEQ ID NO: 87 - protein, 23 aa, synthetic construct
AYDTGCGWAGYNVYDCSADQIDA

SEQ ID NO: 88 - protein, 9 aa, synthetic construct
SGSSGNNYG

SEQ ID NO: 89 - protein, 7 aa, synthetic construct
YNDNRPS

SEQ ID NO: 90 - protein, 11 aa, synthetic construct
GSGDSSSNPGI

SEQ ID NO: 91 - protein, 255 aa, synthetic construct
QAALTQPSSVSANPGETVKITCSGGGSYYGWYQQKSPGSAPVTVIYGNDKRPSNIPSRFSGSKSGSTATLTITGVQVEDEAVYFCGGWDSTDAGTFGAGTTLTVLGQSSRSSGGGGSSGGGGSAVTLDESGGGLQTPGGGLSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSS

SEQ ID NO: 92 - protein, 132 aa, synthetic construct
AVTLDESGGGLQTPGGGLSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSS

SEQ ID NO: 93 - protein, 105 aa, synthetic construct
QAALTQPSSVSANPGETVKITCSGGGSYYGWYQQKSPGSAPVTVIYGNDKRPSNIPSRFSGSKSGSTATLTITGVQVEDEAVYFCGGWDSTDAGTFGAGTTLTVL

SEQ ID NO: 94 - protein, 5 aa, synthetic construct
SHGMA

SEQ ID NO: 95 - protein, 17 aa, synthetic construct
GIDDSGSSTGYGSAVKG

SEQ ID NO: 96 - protein, 23 aa, synthetic construct
AYDTGCGWAGYNVYDCSADQIDA

SEQ ID NO: 97 - protein, 8 aa, synthetic construct
SGGGSYYG

SEQ ID NO: 98 - protein, 7 aa, synthetic construct
GNDKRPS

SEQ ID NO: 99 - protein, 10 aa, synthetic construct
GGWDSTDAGT

SEQ ID NO: 100 - protein, 245 aa, synthetic construct
QAALTQPSSVSANSGETVKITCSGGGSNSYYGWYQQKSPGSAPVTVIYWDDERPSGIPSRFSGALSGSTATLTITGVQVEDEAVYYCGIGDSSGTSLFGAGTTLTVLGQSSRSSGGGGSSGGGGSAVTLDESGGGLQTPGGALSLVCKASGFTFRSYEMQWVRQAPGKGLEWVAGIENDGSYIGYAPAVDGRATISRDNGQSTVRLQLNKLRAEDTGTYYCARDFGYGVGGIDAWGHGTEVIVSS

SEQ ID NO: 101 - protein, 120 aa, synthetic construct
AVTLDESGGGLQTPGGALSLVCKASGFTFRSYEMQWVRQAPGKGLEWVAGIENDGSYIGYAPAVDGRATISRDNGQSTVRLQLNKLRAEDTGTYYCARDFGYGVGGIDAWGHGTEVIVSS

SEQ ID NO: 102 - protein, 107 aa, synthetic construct
QAALTQPSSVSANSGETVKITCSGGGSNSYYGWYQQKSPGSAPVTVIYWDDERPSGIPSRFSGALSGSTATLTITGVQVEDEAVYYCGIGDSSGTSLFGAGTTLTVL

SEQ ID NO: 103 - protein, 5 aa, synthetic construct
SYEMQ

SEQ ID NO: 104 - protein, 17 aa, synthetic construct
GIENDGSYIGYAPAVDG

SEQ ID NO: 105 - protein, 11 aa, synthetic construct
DFGYGVGGIDA

SEQ ID NO: 106 - protein, 10 aa, synthetic construct
SGGGSNSYYG

SEQ ID NO: 107 - protein, 7 aa, synthetic construct
WDDERPS

SEQ ID NO: 108 - protein, 10 aa, synthetic construct
GIGDSSGTSL

SEQ ID NO: 109 - protein, 362 aa, synthetic construct
QAALTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQCPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGES

SEQ ID NO: 110 - protein, 5 aa, synthetic construct
GQAGQ

SEQ ID NO: 111 - protein, 248 aa, synthetic construct
QAALTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQCPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSS

SEQ ID NO: 112 - protein, 107 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGES

SEQ ID NO: 113 - protein, 368 aa, synthetic construct
QAALTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQTPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESHHHHHH

SEQ ID NO: 114 - protein, 4 aa, synthetic construct
TSTS

SEQ ID NO: 115 - protein, 5 aa, synthetic construct
GQAGQ

SEQ ID NO: 116 - protein, 248 aa, synthetic construct
QAALTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQTPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSS

SEQ ID NO: 117 - protein, 107 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 118 - protein, 6 aa, synthetic construct
HHHHHH

SEQ ID NO: 119 - protein, 372 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQCPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 120 - protein, 258 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQCPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSS

SEQ ID NO: 121 - protein, 5 aa, synthetic construct
GQAGQ

SEQ ID NO: 122 - protein, 107 aa, synthetic construct; identical to SEQ ID NO: 117

SEQ ID NO: 123 - protein, 361 aa, synthetic construct
QAALTQPSSVSANPGETVKITCSGSSSSYYGWYQQKAPGSAPVTLIYANDKRPSDIPSRFSGSKSGSTHTLTITGVQADDEAVYFCGSADSTDSGFGAGTTLTVLGQSSRSSGGGGSSGGGGSAVTLDESGGGLQCPGGALSLVCKASGFSFSSYSMHWVRQAPGKGLEFVAAISSDGSRYTHYGPAVKGRATISRDNGQSTVRLQLNNLRPEDTGTYYCAKTSNTWTWNTGWIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 124 - protein, 247 aa, synthetic construct
QAALTQPSSVSANPGETVKITCSGSSSSYYGWYQQKAPGSAPVTLIYANDKRPSDIPSRFSGSKSGSTHTLTITGVQADDEAVYFCGSADSTDSGFGAGTTLTVLGQSSRSSGGGGSSGGGGSAVTLDESGGGLQCPGGALSLVCKASGFSFSSYSMHWVRQAPGKGLEFVAAISSDGSRYTHYGPAVKGRATISRDNGQSTVRLQLNNLRPEDTGTYYCAKTSNTWTWNTGWIDAWGHGTEVIVSS

SEQ ID NO: 125 - protein, 5 aa, synthetic construct
GQAGQ

SEQ ID NO: 126 - protein, 107 aa, synthetic construct; identical to SEQ ID NO: 117

SEQ ID NO: 127 - protein, 378 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESHHHHHH

SEQ ID NO: 128 - protein, 258 aa, synthetic construct; identical to SEQ ID NO: 1

SEQ ID NO: 129 - protein, 5 aa, synthetic construct
GQAGQ

SEQ ID NO: 130 - protein, 107 aa, synthetic construct; identical to SEQ ID NO: 117

SEQ ID NO: 131 - protein, 6 aa, synthetic construct
HHHHHH

SEQ ID NO: 132 - protein, 378 aa, synthetic construct
AIQLTQSPSSLSASVGDRVTITCSGGDSYAGSYYYGWYQQKPGKAPKTLIYYNDKRPSGVPSRFSGSTSGTDSTLTISSLQPEDFATYYCGSIDTSSGTGIFGQGTKLEIKGQSSRSSGGGGSSGGGGSEVQLLESGGGLVQPGGSLRLSCAASGFTFSSYQMFWVRQAPGKGLEYVSEISGSGLSTWYAPAVKGRFTISRDNSKNTLYLQMNSLRAEDTAVYYCAKAAGSCGVGTCAGGIVDAIDAWGQGTTVTVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESHHHHHH

SEQ ID NO: 133 - protein, 258 aa, synthetic construct; identical to SEQ ID NO: 19

SEQ ID NO: 134 - protein, 5 aa, synthetic construct
GQAGQ

SEQ ID NO: 135 - protein, 107 aa, synthetic construct; identical to SEQ ID NO: 117

SEQ ID NO: 136 - protein, 6 aa, synthetic construct
HHHHHH

SEQ ID NO: 137 - protein, 276 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQGGGGSHHHHHH

SEQ ID NO: 138 - protein, 258 aa, synthetic construct; identical to SEQ ID NO: 1

SEQ ID NO: 139 - protein, 5 aa, synthetic construct
GQAGQ

SEQ ID NO: 140 - protein, 5 aa, synthetic construct
GGGGS

SEQ ID NO: 141 - protein, 6 aa, synthetic construct
HHHHHH

SEQ ID NO: 142 - protein, 382 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESEQKLISEEDL

SEQ ID NO: 143 - protein, 258 aa, synthetic construct; identical to SEQ ID NO: 1

SEQ ID NO: 144 - protein, 5 aa, synthetic construct
GQAGQ

SEQ ID NO: 145 - protein, 107 aa, synthetic construct; identical to SEQ ID NO: 117

SEQ ID NO: 146 - protein, 10 aa, synthetic construct
EQKLISEEDL

SEQ ID NO: 147 - protein, 387 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSEQKLISEEDL

SEQ ID NO: 148 - protein, 258 aa, synthetic construct; identical to SEQ ID NO: 1

SEQ ID NO: 149 - protein, 5 aa, synthetic construct
GQAGQ

SEQ ID NO: 150 - protein, 107 aa, synthetic construct; identical to SEQ ID NO: 117

SEQ ID NO: 151 - protein, 5 aa, synthetic construct
GGGGS

SEQ ID NO: 152 - protein, 10 aa, synthetic construct
EQKLISEEDL

SEQ ID NO: 153 - protein, 391 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGSEQKLISEEDL

SEQ ID NO: 154 - protein, 258 aa, synthetic construct; identical to SEQ ID NO: 1

SEQ ID NO: 155 - protein, 5 aa, synthetic construct
GQAGQ

SEQ ID NO: 156 - protein, 107 aa, synthetic construct; identical to SEQ ID NO: 117

SEQ ID NO: 157 - protein, 10 aa, synthetic construct
GGGGSGGGGS

SEQ ID NO: 158 - protein, 10 aa, synthetic construct
EQKLISEEDL

SEQ ID NO: 159 - protein, 387 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLGQSSRSSGGGGSSGGGGSAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSGQAGQGGGGSEQKLISEEDLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGES

SEQ ID NO: 160 - protein, 258 aa, synthetic construct; identical to SEQ ID NO: 1

SEQ ID NO: 161 - protein, 5 aa, synthetic construct
GQAGQ

SEQ ID NO: 162 - protein, 5 aa, synthetic construct
GGGGS

SEQ ID NO: 163 - protein, 10 aa, synthetic construct
EQKLISEEDL

SEQ ID NO: 164 - protein, 107 aa, synthetic construct; identical to SEQ ID NO: 112

SEQ ID NO: 165 - protein, 378 aa, synthetic construct
AVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSSTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEQKLISEEDLAVTLDESGGGLQTPGGALSLVCKGSGFTFSSYQMFWVRQAPGKGLEYVAEISGSGLSTWYAPAVKGRATISRDNGQSTVRLQLNNLRAEDTGTYYCAKAAGSCGVGTCAGGIVDGIDAWGHGTEVIVSS

SEQ ID NO: 166 - protein, 129 aa, synthetic construct; identical to SEQ ID NO: 2

SEQ ID NO: 167 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 168 - protein, 5 aa, synthetic construct
GGGGS

SEQ ID NO: 169 - protein, 10 aa, synthetic construct
EQKLISEEDL

SEQ ID NO: 170 - protein, 233 aa, synthetic construct
QAALTQPSSVSANPGETVKITCSGGDSYAGSYYYGWFQQKAPGSAPVTVIYYNDKRPSDIPSRFSGSTSGSTSTLTITGVQAEDEAVYYCGSIDTSSGTGIFGAGTTLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEQKLISEEDL

SEQ ID NO: 171 - protein, 111 aa, synthetic construct; identical to SEQ ID NO: 3

SEQ ID NO: 172 - protein, 107 aa, synthetic construct [sequence omitted]

SEQ ID NO: 173 - protein, 5 aa, synthetic construct
GGGGS

SEQ ID NO: 174 - protein, 10 aa, synthetic construct
EQKLISEEDL

SEQ ID NO: 175 - protein, 387 aa, synthetic construct
QAALTQPSSVSANLGETVKITCSGSSGNNYGWYQQKSPGSAPVTVIYYNDNRPSDIPSRFSGSKSGSTHTLTITGVQADDEAVYFCGSGDSSSNPGIFGAGTTLTVLGQSSRSSGGGGSSGGGGSAVTLDESGGGLQTPGGALSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSHHHHHH

SEQ ID NO: 176 - protein, 257 aa, synthetic construct; identical to SEQ ID NO: 82

SEQ ID NO: 177 - protein, 5 aa, synthetic construct
GQAGQ

SEQ ID NO: 178 - protein, 107 aa, synthetic construct; identical to SEQ ID NO: 117

SEQ ID NO: 179 - protein, 10 aa, synthetic construct
GGGGSGGGGS

SEQ ID NO: 180 - protein, 385 aa, synthetic construct
QAALTQPSSVSANPGETVKITCSGGGSYYGWYQQKSPGSAPVTVIYGNDKRPSNIPSRFSGSKSGSTATLTITGVQVEDEAVYFCGGWDSTDAGTFGAGTTLTVLGQSSRSSGGGGSSGGGGSAVTLDESGGGLQTPGGGLSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSHHHHHH

SEQ ID NO: 181 - protein, 255 aa, synthetic construct; identical to SEQ ID NO: 91

SEQ ID NO: 182 - protein, 5 aa, synthetic construct
GQAGQ

SEQ ID NO: 183 - protein, 107 aa, synthetic construct; identical to SEQ ID NO: 117

SEQ ID NO: 184 - protein, 10 aa, synthetic construct
GGGGSGGGGS

SEQ ID NO: 185 - protein, 6 aa, synthetic construct
HHHHHH

SEQ ID NO: 186 - protein, 385 aa, synthetic construct
QAALTQPSSVSANPGETVKITCSGGGNYGWYQQKSPGSALVTVIYSNDNRPSNIPSRFSGSKSGSTGTLTITGVQAEDEAVYFCGSYEDNSNPGIFGAGTTLTVLGQSSRSSGGGGSSGGGGSAVTLDESGGGLQTPGGALSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSSTSGQAGQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSHHHHHH

SEQ ID NO: 187 - protein, 255 aa, synthetic construct
QAALTQPSSVSANPGETVKITCSGGGNYGWYQQKSPGSALVTVIYSNDNRPSNIPSRFSGSKSGSTGTLTITGVQAEDEAVYFCGSYEDNSNPGIFGAGTTLTVLGQSSRSSGGGGSSGGGGSAVTLDESGGGLQTPGGALSLVCKASGFTFSSHGMAWVRQAPGKGLEWVAGIDDSGSSTGYGSAVKGRATISRDNGQSTVRLQLNNLRAEDTGIYYCAKAYDTGCGWAGYNVYDCSADQIDAWGHGTEVIVSS

SEQ ID NO: 188 - protein, 5 aa, synthetic construct
GQAGQ

SEQ ID NO: 189 - protein, 107 aa, synthetic construct; identical to SEQ ID NO: 117

SEQ ID NO: 190 - protein, 10 aa, synthetic construct
GGGGSGGGGS

SEQ ID NO: 191 - protein, 6 aa, synthetic construct
HHHHHH

SEQ ID NO: 192 - protein, 21 aa, synthetic construct
MDFQVQIFSFLLISASVIMSR

SEQ ID NO: 193 - protein, 464 aa, synthetic construct
LTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQTPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSSTSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 194 - protein, 247 aa, synthetic construct
LTQPSSVSANPGETVKITCSGGSSNYGWYQQKSPGSAPATVIYDNTNRPSDIPSRFSGALSGSTATLTITGVQVEDEAVYYCGGYDSGTYAGIFGAGTTLTVLGQSSRSSGGGGSSGGGGSAVTLDESGGGLQTPGGTLSLACKGSGFTFSSVNMQWVRQAPGKGLEWVAGISSDGSYTAYGAAVKGRATISRDNGQSTVRLQLNNLRAEDTAIYYCAKIAGGYYWDAAYSIDMWGHGTEVIVSSTS

SEQ ID NO: 195 - protein, 104 aa, synthetic construct
IEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRP

SEQ ID NO: 196 - protein, 21 aa, synthetic construct
MDFQVQIFSFLLISASVIMSR

SEQ ID NO: 197 - protein, 462 aa, synthetic construct
ELDLTQTPASVSAAVGGTVTINCQSSQSPYSNEWLSWYQQKPGQAPKVLISRISTLASGVSSRFKGSGSGTQFTLTISDLECGDAATYFCAGGYNFGLFPFGGGTELEILSSGGGGSGGGGGGSSRSSQSVKESEGRLVTPGGSLTLTCTVSGIDLSRDWMNWVRQAPGEGLEWIGAIGRSGDTYYATWAKGRFTISKTSSRTVTLTVTDLQRSDTATYFCARIPYFGWNNGDIWGPGTLVTISSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 198 - protein, 245 aa, synthetic construct
ELDLTQTPASVSAAVGGTVTINCQSSQSPYSNEWLSWYQQKPGQAPKVLISRISTLASGVSSRFKGSGSGTQFTLTISDLECGDAATYFCAGGYNFGLFPFGGGTELEILSSGGGGSGGGGGGSSRSSQSVKESEGRLVTPGGSLTLTCTVSGIDLSRDWMNWVRQAPGEGLEWIGAIGRSGDTYYATWAKGRFTISKTSSRTVTLTVTDLQRSDTATYFCARIPYFGWNNGDIWGPGTLVTISS

SEQ ID NO: 199 - protein, 104 aa, synthetic construct; identical to SEQ ID NO: 195

SEQ ID NO: 200 - protein, 113 aa, synthetic construct
RAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 201 - protein, 21 aa, synthetic construct
MDFQVQIFSFLLISASVIMSR

SEQ ID NO: 202 - protein, 464 aa, synthetic construct
DILMTQTPSSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSGGGGSQVQLQQSGPEDVKPGASVKISCKASGYTFTDYYMNWVKQSPGKGLEWIGDINPNNGGTSYNQKFKGRATLTVDKSSSTAYMELRSLTSEDSSVYYCESQSGAYWGQGTTVTVSAIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 203 - protein, 247 aa, synthetic construct; identical to SEQ ID NO: 28

SEQ ID NO: 204 - protein, 104 aa, synthetic construct; identical to SEQ ID NO: 195

SEQ ID NO: 205 - protein, 113 aa, synthetic construct; identical to SEQ ID NO: 200

SEQ ID NO: 206 - protein, 27 aa, synthetic construct
MASPLTRFLSLNLLLLGESIILGSGEA

SEQ ID NO: 207 - protein, 469 aa, synthetic construct
DILMTQTPSSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSGGGGSQVQLQQSGPEDVKPGASVKISCKASGYTFTDYYMNWVKQSPGKGLEWIGDINPNNGGTSYNQKFKGRATLTVDKSSSTAYMELRSLTSEDSSVYYCESQSGAYWGQGTTVTVSATTTKPVLRTPSPVHPTGTSQPQRPEDCRPRGSVKGTGLDFACDIYIWAPLAGICVALLLSLIITLICKRGRKKLLYIFKQPFMRPVQTTQEEDGCSCRFPEEEEGGCELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 208 - protein, 247 aa, synthetic construct; identical to SEQ ID NO: 28

SEQ ID NO: 209 - protein, 67 aa, synthetic construct
TTTKPVLRTPSPVHPTGTSQPQRPEDCRPRGSVKGTGLDFACDIYIWAPLAGICVALLLSLIITLIC

SEQ ID NO: 210 - protein, 42 aa, synthetic construct
KRGRKKLLYIFKQPFMRPVQTTQEEDGCSCRFPEEEEGGCEL

SEQ ID NO: 211 - protein, 113 aa, synthetic construct
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 212 - protein, 22 aa, synthetic construct
MLLLVTSLLLCELPHPAFLLIP

SEQ ID NO: 213 - protein, 467 aa, synthetic construct
ELDLTQTPASVSAAVGGTVTINCQSSQSPYSNEWLSWYQQKPGQAPKVLISRISTLASGVSSRFKGSGSGTQFTLTISDLECGDAATYFCAGGYNFGLFPFGGGTELEILSSGGGGSGGGGGGSSRSSQSVKESEGRLVTPGGSLTLTCTVSGIDLSRDWMNWVRQAPGEGLEWIGAIGRSGDTYYATWAKGRFTISKTSSRTVTLTVTDLQRSDTATYFCARIPYFGWNNGDIWGPGTLVTISSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 214 - protein, 245 aa, synthetic construct; identical to SEQ ID NO: 198

SEQ ID NO: 215 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 216 - protein, 41 aa, synthetic construct
RSKRSRLLHSDYMNMTPRRPGPTRKHYQPYAPPRDFAAYRS

SEQ ID NO: 217 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 218 - protein, 21 aa, synthetic construct
MALPVTALLLPLALLLHAARP

SEQ ID NO: 219 - protein, 470 aa, synthetic construct
DILMTQTPSSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSGGGGSQVQLQQSGPEDVKPGASVKISCKASGYTFTDYYMNWVKQSPGKGLEWIGDINPNNGGTSYNQKFKGRATLTVDKSSSTAYMELRSLTSEDSSVYYCESQSGAYWGQGTTVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 220 - protein, 247 aa, synthetic construct; identical to SEQ ID NO: 28

SEQ ID NO: 221 - protein, 42 aa, synthetic construct; identical to SEQ ID NO: 210

SEQ ID NO: 222 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 223 - protein, 21 aa, synthetic construct
MALPVTALLLPLALLLHAARP

SEQ ID NO: 224 - protein, 470 aa, synthetic construct
DIVMTQSPLSLPVTPGEPASISCRSSQSIVHSNRNTYLEWYLQKPGQSPQLLIYKVSNRFSGVPDRFSGSGSGTDFTLKISRVEAEDVGVYYCFQGSHVPFTFGQGTKLEIKRGGGGSGGGGSGGGGSGGGGSQVQLVQSGAEVKKPGASVKVSCKASGYTFTDYYMNWVRQAPGQGLEWMGDINPNHGGTYYNHKFKGRVTMTVDTSTSTAYMELRSLRSDDTAVYYCESQSGAYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 225 - protein, 247 aa, synthetic construct; identical to SEQ ID NO: 37

SEQ ID NO: 226 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 227 - protein, 42 aa, synthetic construct; identical to SEQ ID NO: 210

SEQ ID NO: 228 - protein, 112 aa, synthetic construct; identical to SEQ ID NO: 222

SEQ ID NO: 229 - protein, 21 aa, synthetic construct
MALPVTALLLPLALLLHAARP

SEQ ID NO: 230 - protein, 469 aa, synthetic construct
LTQPSSVSANLGGTVKITCSGGGGSDDGSYYYSWHQQKSPGSAPVTVIYENNNRPSDIPSRFSGSKSGSTATLTITGVQAEDEAVYYCGSRDSSYVDYVGIFGAGTTLTVLGQSSRSSGGGGSSGGGGSAVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 231 - protein, 69 aa, synthetic construct; identical to SEQ ID NO: 226

SEQ ID NO: 232 - protein, 42 aa, synthetic construct; identical to SEQ ID NO: 210

SEQ ID NO: 233 - protein, 112 aa, synthetic construct; identical to SEQ ID NO: 222

SEQ ID NO: 234 - protein, 21 aa, synthetic construct
MALPVTALLLPLALLLHAARP

SEQ ID NO: 235 - protein, 462 aa, synthetic construct
LTQPSSVSVNPGETVKITCSGGGSSSYYGWYQQKSPGSAPVTLIYQNTNRPSNIPSRFSGSTSGSTGTLTITGVQAEDEAVYFCGSRDSSGDGIFGAGTTLTVLGQSSRSSGGGGSSGGGGSAVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 236 - protein, 239 aa, synthetic construct
LTQPSSVSVNPGETVKITCSGGGSSSYYGWYQQKSPGSAPVTLIYQNTNRPSNIPSRFSGSTSGSTGTLTITGVQAEDEAVYFCGSRDSSGDGIFGAGTTLTVLGQSSRSSGGGGSSGGGGSAVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 237 - protein, 69 aa, synthetic construct; identical to SEQ ID NO: 226

SEQ ID NO: 238 - protein, 42 aa, synthetic construct; identical to SEQ ID NO: 210

SEQ ID NO: 239 - protein, 112 aa, synthetic construct; identical to SEQ ID NO: 222

SEQ ID NO: 240 - protein, 21 aa, synthetic construct
MALPVTALLLPLALLLHAARP

SEQ ID NO: 241 - protein, 462 aa, synthetic construct
LTQPSSVSANPGETVEITCSGGSSGYYGWHQQKSPGSAPVTVIYENTNRPSDIPSRFSGSKSGSTATLTITGVQADDEAVYFCGSYDSSSASGIFGAGTTLTVLGQSSRSSGGGGSSGGGGSAVTLDESGGGLQTPGGGLSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 242 - protein, 239 aa, synthetic construct
LTQPSSVSANPGETVEITCSGGSSGYYGWHQQKSPGSAPVTVIYENTNRPSDIPSRFSGSKSGSTATLTITGVQADDEAVYFCGSYDSSSASGIFGAGTTLTVLGQSSRSSGGGGSSGGGGSAVTLDESGGGLQTPGGGLSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS

SEQ ID NO: 243 - protein, 69 aa, synthetic construct; identical to SEQ ID NO: 226

SEQ ID NO: 244 - protein, 42 aa, synthetic construct; identical to SEQ ID NO: 210

SEQ ID NO: 245 - protein, 112 aa, synthetic construct; identical to SEQ ID NO: 222

SEQ ID NO: 246 - protein, 246 aa, synthetic construct
LTQPSSVSANLGGTVKITCSGGGGSDDGSYYYSWHQQKSPGSAPVTVIYENNNRPSDIPSRFSGSKSGSTATLTITGVQAEDEAVYYCGSRDSSYVDYVGIFGAGTTLTVLGQSSRSSGGGGSSGGGGSAVTLDESGGGLQTPGRALSLVCKASGFTFSSYDMAWVRQEPGKGLEYVASISRSGRYTRYGPAVKGRATISRDNGQSTVRLQLNNLRAEDTATYFCTRGGAVSIDTWGHGTEVIVSS